SEQUENCE LISTING (22 sequences)

SEQ ID NO: 1 - DNA, 20 bp, homo sapiens
aaaatctcatccaagaagcc

SEQ ID NO: 2 - DNA, 20 bp, homo sapiens
aatgttccaattcctactgc

SEQ ID NO: 3 - DNA, 20 bp, Homo sapiens
catctatccccattcctgag

SEQ ID NO: 4 - DNA, 20 bp, Homo sapiens
tttttccagtggtatcagtc

SEQ ID NO: 5 - DNA, 22 bp, homo sapiens
ctttgaagatctccccaataac

SEQ ID NO: 6 - DNA, 21 bp, homo sapiens
gatccattgatggaaactgtg

SEQ ID NO: 7 - DNA, 20 bp, homo sapiens
tacctctacacagatgatgc

SEQ ID NO: 8 - DNA, 21 bp, homo sapiens
cccaagatttgaataactccc

SEQ ID NO: 9 - DNA, 21 bp, homo sapiens
cctaaaaagcctaaggaaacc

SEQ ID NO: 10 - DNA, 21 bp, homo sapiens
gatctccacagcaaatgatag

SEQ ID NO: 11 - DNA, 20 bp, homo sapiens
aaagaagccaacactaaacc

SEQ ID NO: 12 - DNA, 19 bp, homo sapiens
tggtcatttcgttaaaggc

SEQ ID NO: 13 - DNA, 20 bp, homo sapiens
acctcaaatttcattgtggg

SEQ ID NO: 14 - DNA, 21 bp, homo sapiens
gaagatgaagaacagaaccag

SEQ ID NO: 15 - DNA, 20 bp, homo sapiens
ccattgttatcggcattctg

SEQ ID NO: 16 - DNA, 22 bp, homo sapiens
attctggatgatgtagaggtag

SEQ ID NO: 17 - DNA, 19 bp, homo sapiens
gaaactgggaagcttgatg

SEQ ID NO: 18 - DNA, 20 bp, homo sapiens
cttgtcagaattgggatgtg

SEQ ID NO: 19 - DNA, 20 bp, homo sapiens
attcaggtttcatttccagg

SEQ ID NO: 20 - DNA, 20 bp, homo sapiens
gattgttcagtactcagctc

SEQ ID NO: 21 - DNA, 20 bp, homo sapiens
acccactcctccacctttga

SEQ ID NO: 22 - DNA, 22 bp, homo sapiens
ctgttgctgtagccaaattcgt

(19) United States
(12) Patent Application Publication
(10) Pub. No.: US 2023/0348506 A1
(43) Pub. Date: Nov. 2, 2023

(54) MACROCYCLIC PANTETHEINE DERIVATIVES AND USES THEREOF

(71) Applicant: VectivBio Comet AG, Basel (CH)

(72) Inventors: Arthur George TAVERAS, Boston, MA (US); Enej KUSCER, Ljubljana-Smartno (SI); Angelina Roberta SEKIRNIK, Logatec (SI)

(21) Appl. No.: 17/780,325

(22) PCT Filed: Nov. 25, 2020
(86) PCT No.: PCT/US2020/062262

§ 371 Date: May 26, 2022

Related U.S. Application Data
(60) Provisional application No. 62/940,426, filed on Nov. 26, 2019.

(51) Int. Cl. C07F 9/6584; C07D 291/02; C07F 9/6536; A61P 25/00; A61P 35/00

(52) U.S. Cl. CPC C07F 9/6584; C07D 291/02; C07F 9/6536; A61P 25/00; A61P 35/00

(57) ABSTRACT

The present disclosure relates to compounds of Formulae (I) or (II):

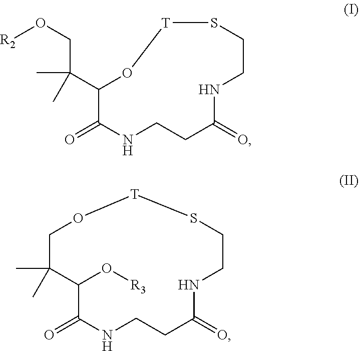

and pharmaceutically acceptable salts or solvates thereof. The present disclosure also relates to pharmaceutical compositions comprising the compounds and therapeutic and diagnostic uses of the compounds and pharmaceutical compositions.

### RELATED APPLICATIONS

This application claims priority to, and the benefit of, U.S. Provisional Application No. 62/940,426, filed Nov. 26, 2019, the entire contents of which is incorporated herein by reference.

### INCORPORATION-BY-REFERENCE OF SEQUENCE LISTING

The contents of the file named “TM3T-006_001WO_ST25.txt”, which was created on Nov. 25, 2020, and is 4 KB in size are hereby incorporated by reference in their entirety.

### BACKGROUND

Acetyl-Coenzyme A (acetyl-CoA) is a metabolite derived from glucose, fatty acid and amino acid catabolism. One primary function of acetyl-CoA is to deliver an acetyl group to the citric acid cycle (also known as the Krebs cycle) for energy production. Acetyl-CoA is also an important intermediate in other biological pathways, including, but not limited to fatty acid and amino acid metabolism, steroid synthesis, acetylcholine synthesis, melatonin synthesis and acetylation pathways (e.g. lysine acetylation, posttranslational acetylation). Acetyl-CoA concentrations also influence the activity or specificity of various enzymes, including, but not limited to pyruvate dehydrogenase kinase and pyruvate carboxylase, either in an allosteric manner or by altering substrate availability. Acetyl-CoA also controls key cellular processes, including energy metabolism, mitosis, and autophagy, both directly and via the epigenetic regulation of gene expression by influencing the acetylation profile of several proteins, including, but not limited to histones.

Acetyl-CoA is synthesized in vivo in several ways, including extramitochondrially and intramitochondrially. Intramitochondrially, when glucose levels are high, acetyl-CoA is produced as an end-product of glycolysis through a pyruvate dehydrogenase reaction, in which pyruvate undergoes oxidative decarboxylation to form acetyl-CoA. Other conversions between pyruvate and acetyl-CoA occur, including the disproportionation of pyruvate into acetyl-CoA and formic acid by pyruvate formate lyase. At lower glucose levels, acetyl-CoA is produced by β-oxidation of fatty acids. Fatty acids are first converted to an acyl-CoA, which is further degraded in a four-step cycle of dehydrogenation, hydration, oxidation and thiolysis to form acetyl-CoA. These four steps are performed by acyl-CoA dehydrogenase, enoyl-CoA hydratase, 3-hydroxyacyl-CoA dehydrogenase and thiolase respectively. Additionally, degradation of amino acids such as leucine, isoleucine, lysine, tryptophan, phenylalanine and tyrosine can also produce acetyl-CoA. For example, branched chain amino acids are converted to α-ketoacids by transamination in the cytosol, then transferred to mitochondria via a carnitine shuttle transport, and finally processed inside the mitochondrial matrix by an α-ketoacid dehydrogenase complex where an α-ketoacil-CoA undergoes a multi-step dehydrogenation, carboxylation and hydration to produce acetyl-CoA. Acetyl-CoA can also be synthesized intramitochondrially by acetyl-CoA synthetase, which is an enzyme that uses acetate and ATP to acetylate CoA. In addition, there are organ-specific pathways for mitochondrial acetyl-CoA generation. For instance, neurons can employ the ketone bodies D-b-hydroxybutyrate and acetoacetate to generate acetyl-CoA (Cahill, 2006) and hepatocytes can produce acetyl-CoA from ethanol as a carbon source through conversion via acetaldehyde and acetate.

Extramitochondrially, Acetyl-CoA can be produced by ATP citrate lyase, which converts citrate made by the tricarboxylic acid cycle into acetyl-CoA and oxaloacetate. Secondly, acetyl-CoA can also be produced in the cytosol from acetate in an ATP-dependent reaction catalyzed by acyl-CoA synthetase.

Decreased levels of acetyl-CoA can be caused by the inhibition, loss of, or decrease in activity of the various metabolic enzymes and pathways of acetyl-CoA biosynthesis. Diseases such as organic acidemias of deficient branched chain amino acid catabolism or fatty acid oxidation disorders, such as short chain acyl-CoA dehydrogenase deficiency (SCADD), medium chain acyl-CoA dehydrogenase deficiency (MCADD), long chain acyl-CoA dehydrogenase deficiency (LCADD) and very long chain acyl-CoA dehydrogenase deficiency (VLCADD) can lead to a decrease in acetyl-CoA levels and the accumulation of other CoA species including acyl-CoA species. These diseases can lead to symptoms such as hypoglycemia, liver dysfunction, lethargy, seizures, coma and even death. Thus, there is a need in the art for compositions and methods for the treatment of CoA deficiency, acetyl-CoA deficiency, and other acyl-CoA deficiencies.

### SUMMARY

In some aspects, the present disclosure provides, inter alia, a compound of Formula (I) or (II):

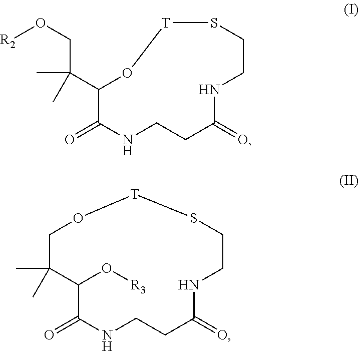

or a pharmaceutically acceptable salt or solvate thereof, wherein:
T is:
   —C(═O)—**,
   —C(═O)—(CH═CH)ₙ—C(═O)—**,
   —C(═O)—(CHR_{1b})ₙ—C(═O)—**,
   —C(═O)CH₂—[C(═O)CH₂]ₚ—(CH₂)_{q}—C(═O)—**,
   —C(═O)CH₂—[CH(OR_{1c})—CH₂]ₚ—(CH₂)_{q}—C(═O)—**,
   —C(═O)CH₂—[C(═O)CH₂]ₚ—[CH(OR_{1c})—CH₂]ᵣ—(CH₂)_{q}—C(═O)—**,
   —C(═O)CH₂—[CH(OR_{1c})—CH₂]ᵣ—[C(═O)CH₂]ₚ—(CH₂]_{q}—C(═O)—**,
   —C(═O)—(CHR_{1b})ₙ—[C(═O)CH₂]ₚ—(CH₂)_{q}—C(═O)—**,
   —C(═O)CH₂—[C(═O)—(CHR_{1b})ₙ]ₚ—(CH₂)_{q}—C(═O)—**,
   —C(═O)CH₂—[C(═O)CH₂]ₚ—(CHR_{1b})_{q}—C(═O)—**,
   —C(═O)—(CHR_{1b})ₙ—[C(═O)CH₂]ₚ—(CHR_{1b})_{q}—C(═O)—**,
   *—C(═O)CH₂—[C(═O)CH₂]ₚ—(CH₂)_{q}—C(═O)—*,
   *—C(═O)CH₂—[CH(OR_{1c})—CH₂]ₚ—(CH₂)_{q}—C(═O)—*,
   *—C(═O)CH₂—[C(═O)CH₂]ₚ—[CH(OR_{1c})—CH₂]ᵣ—(CH₂)_{q}—C(═O)—*,
   *—C(═O)CH₂—[CH(OR_{1c})—CH₂]ᵣ—[C(═O)CH₂]ₚ—(CH₂)_{q}—C(═O)—*,
   *—C(═O)—(CHR_{1b})ₙ—[C(═O)CH₂]ₚ—(CH₂)_{q}—C(═O)—*,
   *—C(═O)CH₂—[C(═O)—(CHR_{1b})ₙ]ₚ—(CH₂)_{q}—C(═O)—*,
   *—C(═O)CH₂—[C(═O)CH₂]ₚ—(CHR_{1b})_{q}—C(═O)—*,
   *—C(═O)—(CHR_{1b})ₙ—[C(═O)CH₂]ₚ—(CHR_{1b})_{q}—C(═O)—*,

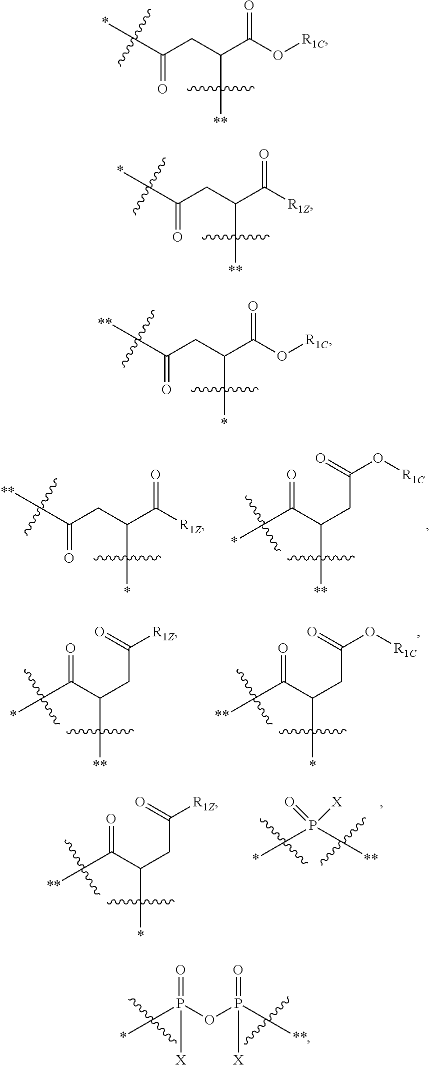

wherein * denotes an attachment of T to the sulfur atom, and ** denotes an attachment of T to the oxygen atom;
n is an integer ranging from 0 to 20;
p is an integer ranging from 0 to 20;
q is an integer ranging from 0 to 20;
r is an integer ranging from 0 to 20;
R₂ is H, —C(═O)R_{1b}, —C(═O)OR_{1c}, —C(═O)N(R_{1c})₂, —C(═O)R_{1z}, —C(═O)—(C═O)R_{1b}, —C(═O)—C(═O)OR_{1c}, —C(═O)—C(═O)N(R_{1c})₂, —C(═O)—C(═O)R_{1z}, —C(═O)—CH═CH—C(═O)OR_{1c}, —C(═O)—CH₂—CH₂—C(═O)OR_{1c},

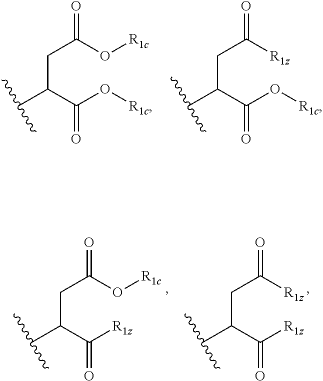

—C(═O)—CH═CH—C(═O)—R_{1z}, —C(═O)—CH₂—CH₂—C(═O)—R_{1z}, Si(R_{1g})₃,

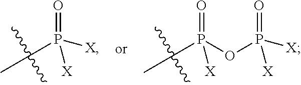

R₃ is H, —C(═O)R_{1b}, —C(═O)OR_{1c}, —C(═O)N(R_{1c})₂, —C(═O)R_{1z}, —C(═O)—(C═O)R_{1b}, —C(═O)—C(═O)OR_{1c}, —C(═O)—C(═O)N(R_{1c})₂, —C(═O)—C(═O)R_{1z}, —C(═O)—CH═CH—C(═O)OR_{1c}. —C(═O)—CH₂—CH₂—C(═O)OR_{1c},

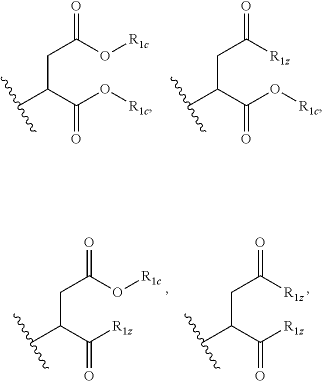

—C(═O)—CH═CH—C(═O)—R_{1z}, —C(═O)—CH₂—CH₂—C(═O)—R_{1z}, Si(R_{1g})₃,

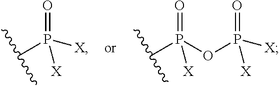

each R_{1b} is independently H, C₁-C₂₀ alkyl, C₂-C₂₀ alkenyl, C₂-C₂₀ alkynyl, —(CH₂)_{q}—C(═O)OR_{1c}, —CH₂—C(═O)—(CH₂)_{q}—C(═O)OR_{1c}, —CH₂—[C(═O)CH₂]ₚ—[CH₂]_{q}—C(═O)OR_{1c}, —CH═CH—C(═O)OR_{1c}. —C(═O)OR_{1c}, —C(═O)N(R_{1c})₂, or R_{1z}, wherein the C₁-C₂₀ alkyl, C₂-C₂₀ alkenyl, or C₂-C₂₀ alkynyl is optionally substituted with one or more R_{1c};
each R_{1c} is independently H, C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl), wherein the C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₂ heteroaryl) is optionally substituted with one or more R₁ₑ;
each R_{1d} is independently H, C₁-C₂₀ alkyl, C₂-C₂₀ alkenyl, C₂-C₂₀ alkynyl, C₃-C₁₀ cycloalkyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl), wherein the C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₂ heteroaryl) is optionally substituted with one or more R₁ₑ;
each R_{1c} is independently H, halogen, C₁-C₂₀ alkyl, C₂-C₂₀ alkenyl, C₂-C₂₀ alkynyl, —OR_{1g}, —C(═O)OR_{1g}, —C(═O)N(R_{1g})₂, —N(R_{1g})₂, —N(R_{1g})C(═O)R_{1f}, —N(R_{1g})C(═O)R_{1z}, —N(R_{1g})C(═O)OR_{1g}, —OC(═O)R_{1f}, —OC(═O)R_{1z}, —OC(═O)OR_{1g}, —SR_{1g}, —N⁺(R_{1g})₃, —SC(═O)R_{1f}, —SC(═O)R_{1z}, —SC(═O)OR_{1g}, —SC(═O)N(R_{1g})₂, —C(═O)R_{1f}, —C(═O)R_{1z},

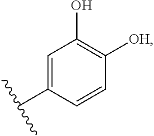 [SMILES: *c1ccc(O)c(O)c1]

or R_{1z}, wherein the C₁-C₂₀ alkyl, C₂-C₂₀ alkenyl, or C₂-C₂₀ alkynyl is optionally substituted with one or more R_{1z};
each R_{1f} is independently H, C₁-C₂₀ alkyl, C₂-C₂₀ alkenyl, C₂-C₂₀ alkynyl, —CH₂C(═O)OR_{1g}, —CH═CH—C(═O)OR_{1g}, —C(═O)OR_{1g}, —C(═O)N(R_{1g})₂,

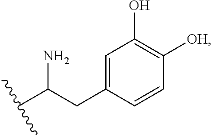 [SMILES: *C(N)Cc1ccc(O)c(O)c1]

or R_{1z}, wherein the C₁-C₂₀ alkyl, C₂-C₂₀ alkenyl, or C₂-C₂₀ alkynyl is optionally substituted with one or more R_{1z};
each R_{1g} is independently H, C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl), wherein the C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) is optionally substituted with one or more R_{1z};
each R_{1z} is independently

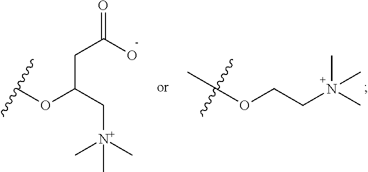

and
each X is independently —OR_{1c}, —SR_{1c}, —N(R_{1c})₂,

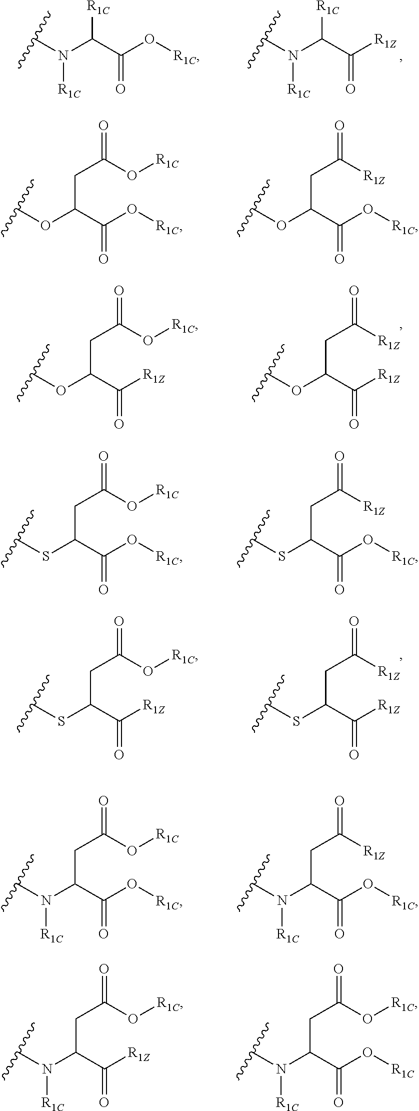

### or R_{1z}.

In some aspects, the present disclosure provides, inter alia, a compound of Formula (I) or (II):

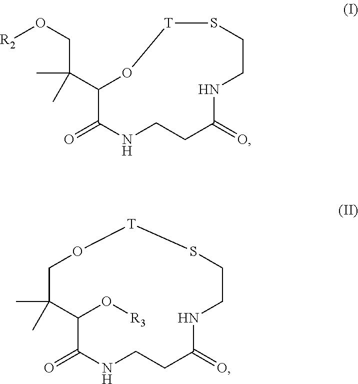

or a pharmaceutically acceptable salt or solvate thereof, wherein:
T is *—C(═O)—(CHR_{1b})ₙ—C(═O)—**;
wherein * denotes an attachment of T to the sulfur atom, and ** denotes an attachment of T to the oxygen atom;
n is an integer ranging from 0 to 20;
R₂ is H, —C(═O)R_{1b}, or Si(R_{1g})₃;
R₃ is H, —C(═O)R_{1b}, or Si(R_{1g})₃;
each R_{1b} is independently H or C₁-C₂₀ alkyl, wherein the C₁-C₂₀ alkyl is optionally substituted with one or more R_{1c};
each R₁ₑ is independently H, C₁-C₂₀ alkyl, or —N(R_{1g})₂;
each R_{1g} is independently H or C₁-C₂₀ alkyl.

In some aspects, the present disclosure provides a method of treating or preventing a disease in a subject, comprising administering to the subject a therapeutically effective amount of at least one compound of the present disclosure.

In some aspects, the present disclosure provides at least one compound of the present disclosure for use in treating or preventing a disease in a subject, wherein the at least one compound of the present disclosure is for administration to the subject in at least one therapeutically effective amount.

In some aspects, the present disclosure provides use of at least one compound of the present disclosure for the manufacture of a medicament for treating or preventing a disease in a subject, wherein the at least one compound of the present disclosure is for administration to the subject in at least one therapeutically effective amount.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF THE FIGURES

FIG. 1 is a schematic overview of fatty acid oxidation and the synthesis of acetyl-CoA.

FIG. 2 is a schematic overview of a compound of the present disclosure being converted into more than two equivalents of acetyl-CoA.

FIG. 3 is an image showing slight elongation and networking of mitochondria in MMA Patient fibroblasts (GM01673, Coriell Institute for Medical Research) promoted by Compound 61 (right panel) compared to fragmented mitochondria in vehicle control (left panel). Images were obtained according to the methods in Example 24.

FIG. 4 is an image showing good elongation and networking of mitochondria in MMA Patient fibroblasts (GM01673, Coriell Institute for Medical Research) promoted by Compound 206 (right panel) compared to fragmented mitochondria in vehicle control (left panel). Images were obtained according to the methods in Example 24.

FIG. 5 is an image showing mild elongation and networking of mitochondria in PA Patient fibroblasts (Tsi 3618 Trans-Hit Bio) promoted by Compound 206 (right panel) compared to fragmented mitochondria in vehicle control (left panel). Images were obtained according to the methods in Example 24.

### DETAILED DESCRIPTION

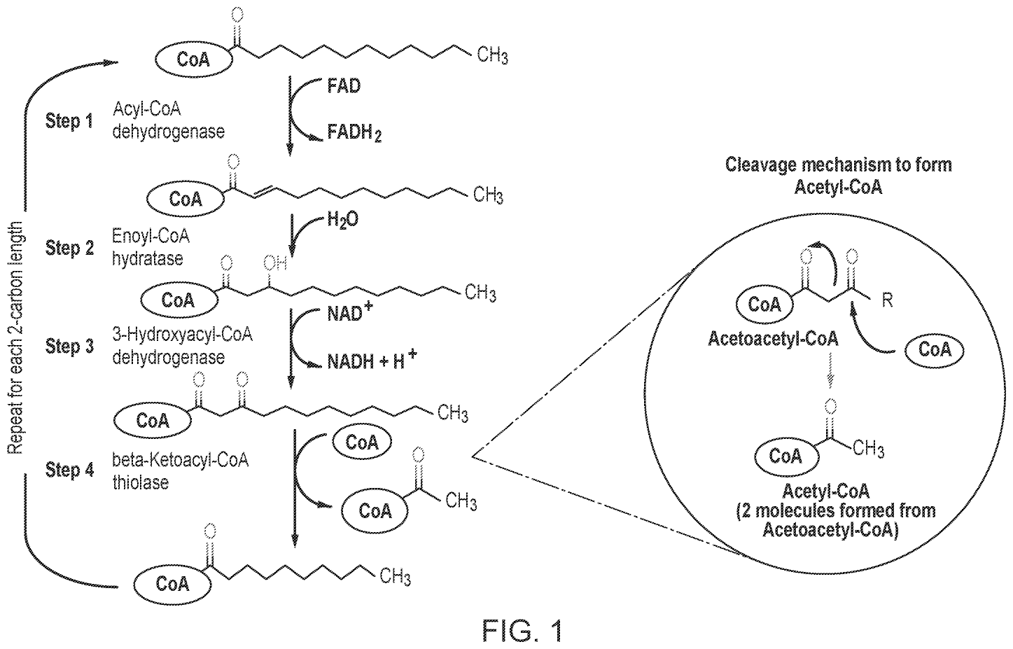

The present disclosure is based on, inter alia, the discovery that the administration of a compound disclosed herein to a subject can increase the concentration of CoA, acetyl-CoA, and/or another Acyl-CoA (such as succinyl-CoA) in the subject by bypassing the normal mechanism of Acyl-CoA biosynthesis and augmenting the production of acetyl-CoA by various biosynthetic pathways. These pathways include, but are not limited to, fatty acid oxidation. As shown in FIG. 1, fatty acid oxidation (FAO) results in the production of acetyl-CoA through a cascade of enzymes that convert an acyl-CoA molecule into at least one acetyl-CoA molecule and a shorter acyl-CoA molecule.

Step 1 of FIG. 1 shows that acyl-CoA, which is formed when a CoA molecule is attached to the end of a fatty acid by acyl-CoA synthetase, is converted into enoyl-CoA molecule by an acyl-CoA dehydrogenase (ACAD) enzyme. There are various ACAD enzymes, with preferences for acyl-CoA attached to different length fatty acid chains. For example, the are very long chain ACAD enzymes, long chain ACAD enzymes, medium chain ACAD enzymes and short chain ACAD enzymes.

Following formation of the enoyl-CoA molecule, step 2 of FIG. 1 shows that during FAO, an enoyl-CoA molecule is then converted to a hydroxyacyl-CoA molecule by a enoyl-CoA hydratase enzyme. There are various enoyl-CoA hydratase enzymes, with preferences for a enoyl-CoA with different length fatty acid chains. For example, the are very long chain enoyl-CoA hydratase enzymes, long chain enoyl-CoA hydratase enzymes, medium chain enoyl-CoA hydratase enzymes and short chain enoyl-CoA hydratase enzymes.

Following formation of the hydroxyacyl-CoA molecule, step 3 of FIG. 1 shows that during FAO the hydroxyacyl-CoA molecule is converted into a ketoacyl-CoA molecule by a 3-hydroxyacyl-CoA dehydrogenase (HAD) enzyme. There are various HAD enzymes, with preferences for a hydroxyacyl-CoA molecules with different length fatty acid chains. For example, there are very long chain HAD enzymes, long chain HAD enzymes, medium chain HAD enzymes and short chain HAD enzymes.

Following formation of the ketoacyl-CoA molecule, step 4 of FIG. 1 shows that during FAO, the ketoacyl-CoA molecule is converted into one acetyl-CoA molecule and one acyl-CoA molecule by a beta-ketoacyl-CoA thiolase enzyme. The entire cycle shown in FIG. 1 can then be repeated using the newly generated acyl-CoA molecule, that now has a shorter fatty acid chain, as a substrate in step 1.

It is discovered that the concentration of acetyl-CoA and/or Acyl-CoA (such as, but not limited to, succinyl-CoA) can be increased in a subject by administering to the subject a compound of the present disclosure. In some embodiments, the compound can be used as a substrate by any enzyme in a biosynthetic pathway that produces acetyl-CoA and/or Acyl-CoA (such as, but not limited to, succinyl-CoA). The biosynthetic pathway can include, but is not limited to, the FAO enzyme cascade depicted in FIG. 1. In some embodiments, the compound can be a substrate for a beta-ketoacyl-CoA thiolase enzyme, such that beta-Ketoacyl-CoA thiolase converts the administered compound into at least one molecule of acetyl-CoA. In some embodiments, the compound can be converted into more than one acetyl-CoA molecule. Further, in some embodiments, the compound can be converted into a succinyl-CoA molecule. In some additional embodiments, the compound can be converted into one or more acetyl-CoA molecules and a succinyl-CoA molecule.

The present disclosure is also based on, inter alia, the discovery that certain molecules, including, but not limited to, pantetheine, pantetheine derivatives, phosphopantetheine and phosphopantetheine derivatives, can act as a carrier molecule to aid in the delivery of a cargo molecule to a particular tissue, cell and/or organelle within a subject. These carrier molecules are able to traverse a biological membrane, helping to deliver cargo molecules to specific tissues, cells and/or organelles.

Various compositions, kits and methods of the present disclosure are described in full detail herein.

The compounds of the present disclosure, when administered to a subject, can be transformed either directly or indirectly into at least one acetyl-CoA molecule. In a non-limiting example the compounds of the present disclosure a substrate for an enzyme within an enzyme cascade that produces acetyl-CoA. These enzymes, include, but are not limited to, the enzymes in the FAO enzymatic cascade depicted in FIG. 1. The compounds of the present disclosure can be a substrate for an acyl-CoA dehydrogenase enzyme, or an enoyl-CoA hydratase enzyme, or a 3-hydroxyacyl-CoA dehydrogenase enzyme, or a beta-ketoacyl-CoA thiolase enzyme. The compounds of the present disclosure can be a substrate for a very long chain ACAD enzyme, or a long chain ACAD enzyme, or a medium chain ACAD enzyme, or a short chain ACAD enzymes, or a very long chain enoyl-CoA hydratase enzyme, or a long chain enoyl-CoA hydratase enzyme, or a medium chain enoyl-CoA hydratase enzyme, or a short chain enoyl-CoA hydratase enzyme, or a very long chain HAD enzyme, or a long chain HAD enzyme, or a medium chain HAD enzyme, or a short chain HAD enzyme.

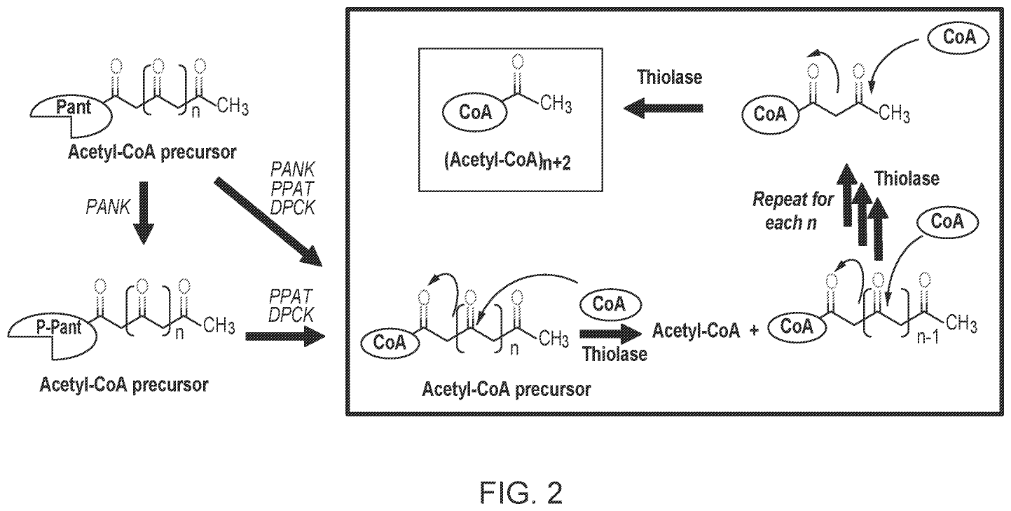

In some aspects, one equivalent of the compounds of the present disclosure can be directly or indirectly converted into at least two, or at least three, or at least four, or at least five, or at least six, or at least seven, or at least eight, or at least nine, or at least ten, or at least 11, or at least 12, or at least 13, or at least 14, or at least 15, or at least 16, or at least 17, or at least 18, or at least 19, or at least 20, or at least 21, or at least 22, or at least 23, or at least 24, or at least 25, or at least 26, or at least 27, or at least 28, or at least 29, or at least 30, or at least 40, or at least 50, or at least 60, or at least 70, or at least 80, or at least 90 or at least 100 equivalents of acetyl-CoA. The non-limiting example depicted in FIG. 2 shows an exemplary molecule of the present disclosure that is directly converted into more than two equivalents of acetyl-CoA by beta-ketoacyl-CoA thiolase.

In some aspects, the compounds of the present disclosure can cross a cellular membrane. A cellular membrane can include, but is not limited to, the plasma membrane, the nuclear envelope, the outer membrane of the nuclear envelope, the inner membrane of the nuclear envelope, the mitochondrial membrane, the outer mitochondrial membrane, the inner mitochondrial membrane, the lysosomal membrane, the peroxisome membrane, the golgi apparatus membrane, or the endoplasmic reticulum membrane. In some aspects, the compounds of the present disclosure of the present disclosure can cross a cellular membrane without the aid of a protein such as a transporter. This is in contrast to CoA and Acyl-CoA species, which cannot cross a cellular membrane.

In some aspects, the compounds of the present disclosure can deliver an acyl moiety into the matrix of a mitochondrion. The compounds of the present disclosure can deliver an acyl moiety into the matrix of the mitochondrion without the aid of a carnitine transporter protein.

In some aspects, the compounds of the present disclosure can activate or enhance acetyl-CoA synthesis in a subject.

In some aspects, the compounds of the present disclosure can increase acetyl-CoA concentrations in a subject.

In some aspects, the compounds of the present disclosure can increase Acetyl-CoA biosynthesis in a subject.

In some aspects the compounds of the present disclosure can decrease degradation of CoA in a subject.

In some aspects, the compounds of the present disclosure can increase the half-life of CoA in a subject.

In some aspects, the compounds of the present disclosure can prolong the availability of CoA in a subject.

In some aspects, the compounds of the present disclosure can prolong the utilization of CoA in a subject.

In some aspects, the compounds of the present disclosure can deliver an acyl moiety into the mitochondrial matrix of a mitochondrion of a subject.

In some aspects the compounds of the present disclosure can decrease the concentration of reactive oxygen species (ROS) in a subject.

In some aspects, the compounds of the present disclosure can decrease the concentration of an at least one acyl-CoA species in a subject.

In some aspects, the compounds of the present disclosure can increase the fatty acid metabolism in a subject.

In some aspects, the compounds of the present disclosure can increase the amino acid metabolism in a subject.

In some aspects, the compounds of the present disclosure can increase mitochondrial respiration in a subject.

In some aspects, the compounds of the present disclosure can increase ATP concentration in a subject.

In some aspects, the compounds of the present disclosure can increase the post-translational modification of proteins in a subject.

In some aspects, the compounds of the present disclosure can increase acetylation of proteins in a subject. In some aspects, the compounds of the present disclosure can increase acetylation of histones in a subject. In some aspects, the compounds of the present disclosure can increase acetylation of tubulin in a subject.

In some aspects, the compounds of the present disclosure can induce tumor cell apoptosis in a subject.

In some aspects, the compounds of the present disclosure can induce cell cycle arrest in a tumor cell in a subject.

In some aspects, the compounds of the present disclosure can induce differentiation of a cell in a subject.

In some aspects, the compounds of the present disclosure can induce senescence in a cell in a subject.

In some aspects, the compounds of the present disclosure can enhance an immune response against cancer in a subject.

In some aspects, the compounds of the present disclosure can inhibit angiogenesis in a subject.

In some aspects, the compounds of the present disclosure can enhance the apoptotic effect of an anti-cancer agent.

In some aspects, the compounds of the present disclosure can reverse acetylation patterns induced by major depressive disorder in a subject.

In some aspects, the compounds of the present disclosure can augment the therapeutic effect of an anti-depressant compound in a subject.

In some aspects, the compounds of the present disclosure can prevent an inappropriate shift to fatty acid biosynthesis in a subject

In some aspects, the compounds of the present disclosure can reduce inflammation in a subject.

In some aspects, the compounds of the present disclosure can stimulate the activity of regulatory T cells in a subject.

In some aspects, the compounds of the present disclosure can reduce fibrosis in a subject.

In some aspects, the compounds of the present disclosure can reactivate latent HIV in a subject. In some aspects, the compounds of the present disclosure can reactivate latent HIV without inducing global T cell activation in a subject.

In some aspects, the compounds of the present disclosure can prevent ischemic stroke in a subject. In some aspects, the compounds of the present disclosure can prevent reinfarction in a subject.

In some aspects, the compounds of the present disclosure can increase the survival of cardiac cells in a subject.

In some aspects, the compounds of the present disclosure can prevent ischemic stroke in a subject. In some aspects, an acetyl-CoA precursor of the present disclosure can prevent reinfarction in a subject.

In some aspects, the compounds of the present disclosure can reduce damage to damage to cardiac cells in a subject. In some aspects, an acetyl-CoA precursor of the present disclosure can reduce damage imparted by ischemia, inflammation, fibrotic remodeling or any combination thereof in a subject.

In some aspects, the compound of the present disclosure may serve as a cargo-carrier complex or a portion thereof. A cargo-carrier complex can comprise a cargo molecule that is covalently linked to a carrier molecule. The carrier molecule can be used to target the cargo-carrier complex to a specific tissue, cell, and/or organelle in a subject. In some aspects, the carrier molecule can allow the cargo-carrier complex to traverse a biological membrane. In some aspects, once the cargo-carrier complex has reached its target destination in a subject, the cargo molecule will be released from the carrier molecule, i.e. the covalent linkage between the cargo molecule and the carrier molecule will be broken. The covalent linkage between the cargo molecule and the carrier molecule can be broken by an enzyme. In some aspects, following release of the cargo molecule from the carrier molecule, the cargo molecule, the carrier molecule or the cargo and the carrier molecule can be further modified by biosynthetic pathways.

In some aspects, a carrier molecule can comprise pantetheine or a pantetheine derivative. In some aspects, a carrier molecule can comprise a phosphopantetheine or a phosphopantetheine derivative. In some aspects, a carrier molecule can comprise any compound of the present disclosure, as described in full detail herein.

In some aspects, a cargo molecule can comprise an acyl group, or an succinyl group, or an acetyl group, or any combination thereof. In some aspects, a cargo molecule can comprise an acetyl-CoA precursor. In some aspects, a cargo molecule can comprise an acyl-CoA precursor. In some aspects a cargo molecule can comprise a succinyl-CoA precursor. In some aspects, a cargo molecule can comprise fumarate or a fumarate-derivative. In some aspects, a cargo molecule can comprise Tecfidera (dimethyl fumarate. In some aspects, a cargo molecule can comprise any drug. In some aspects, a cargo molecule can comprise any small molecule therapeutic. In some aspects, a cargo molecule can comprise any compound of the present disclosure, as described in full detail herein.

In some aspects, a cargo-carrier complex can comprise any compound of the present disclosure, as described in full detail herein.

In some aspects, any compound of the present disclosure can deliver an acyl moiety into the matrix of a mitochondrion. In some aspects, any compound of the present disclosure can deliver an acyl moiety into the matrix of the mitochondrion without the aid of a carnitine transporter protein.

### Compounds of the Present Disclosure

In some aspects, the present disclosure provides a compound of Formula (I) or (II).

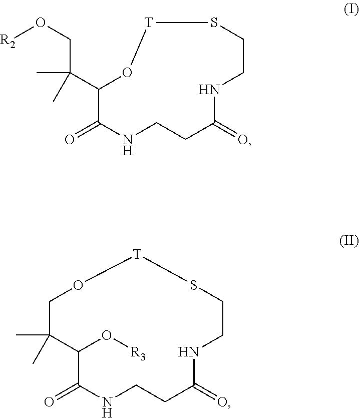

or a pharmaceutically acceptable salt or solvate thereof, wherein:
T is:
   *—C(═O)—(CH═CH)ₙ—C(═O)—**,
   *—C(═O)—(CHR_{1b})ₙ—C(═O)—**,
   *—C(═O)CH₂—[C(═O)CH₂]ₚ—(CH₂)_{q}—C(═O)—**,
   *—C(═O)CH₂—[CH(OR_{1c})—CH₂]ₚ—(CH₂)_{q}—C(═O)—**,
   *—C(═O)CH₂—[C(═O)CH₂]ₚ—[CH(OR_{1c})—CH₂]ᵣ—(CH₂)_{q}—C(═O)—**,
   *—C(═O)CH₂—[CH(OR_{1c})—CH₂]ᵣ—[C(═O)CH₂]ₚ—(CH₂]_{q}—C(═O)—**,
   *—C(═O)—(CHR_{1b})ₙ—[C(═O)CH₂]ₚ—(CH₂)_{q}—C(═O)—**,
   *—C(═O)CH₂—[C(═O)—(CHR_{1b})ₙ]ₚ—(CH₂)_{q}—C(═O)—**,
   *—C(═O)CH₂—[C(═O)CH₂]ₚ—(CHR_{1b})_{q}—C(═O)—**,
   *—C(═O)—(CHR_{1b})ₙ—[C(═O)CH₂]ₚ—(CHR_{1b})_{q}—C(═O)—**,
   **—C(═O)CH₂—[C(═O)CH₂]ₚ—(CH₂)_{q}—C(═O)—*,
   **—C(═O)CH₂—[CH(OR_{1c})—CH₂]ₚ—(CH₂)_{q}—C(═O)—*,
   **—C(═O)CH₂—[C(═O)CH₂]ₚ—[CH(OR_{1c})—CH₂]ᵣ—(CH₂)_{q}—C(═O)—*,
   **—C(═O)CH₂—[CH(OR_{1c})—CH₂]ᵣ—[C(═O)CH₂]ₚ—(CH₂)_{q}—C(═O)—*,
   **—C(═O)—(CHR_{1b})ₙ—[C(═O)CH₂]ₚ—(CH₂)_{q}—C(═O)—*,
   **—C(═O)CH₂—[C(═O)—(CHR_{1b})ₙ]ₚ—(CH₂)_{q}—C(═O)—*,
   **—C(═O)CH₂—[C(═O)CH₂]ₚ—(CHR_{1b})_{q}—C(═O)—*,
   **—C(═O)—(CHR_{1b})ₙ—[C(═O)CH₂]ₚ—(CHR_{1b})_{q}—C(═O)—*,

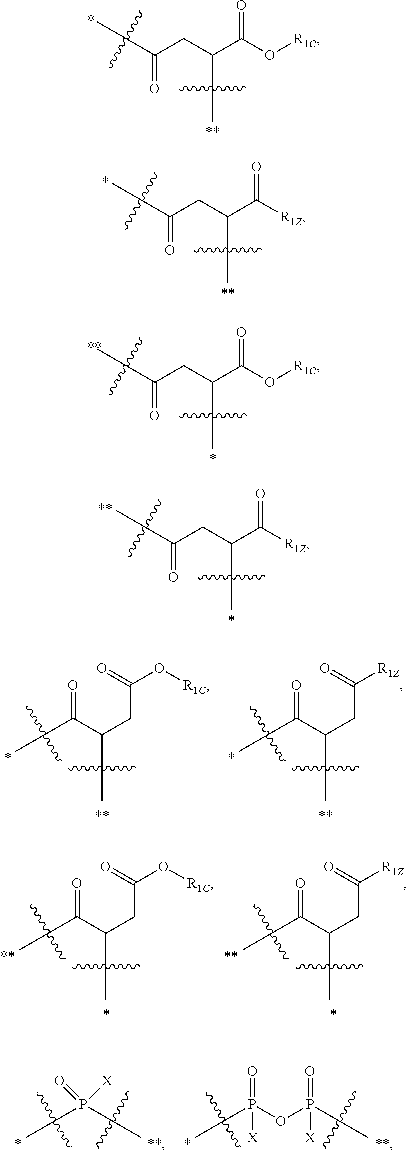

wherein * denotes an attachment of T to the sulfur atom, and ** denotes an attachment of T to the oxygen atom;
n is an integer ranging from 0 to 20;
p is an integer ranging from 0 to 20;
q is an integer ranging from 0 to 20;
r is an integer ranging from 0 to 20;
R₂ is H, —C(═O)R_{1b}, —C(═O)OR_{1c}, —C(═O)N(R_{1c})₂, —C(═O)R_{1z}, —C(═O)—(C═O)R_{1b}, —C(═O)—C(═O)OR_{1c}, —C(═O)—C(═O)N(R_{1c})₂, —C(═O)—C(═O)R_{1z}, —C(═O)—CH═CH—C(═O)OR_{1c}. —C(═O)—CH₂—CH₂—C(═O)OR_{1c},

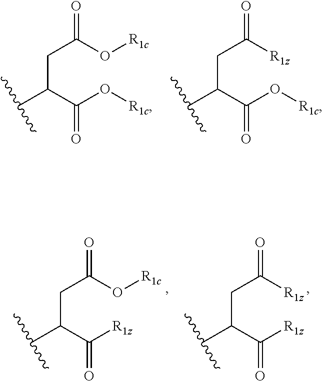

—C(═O)—CH═CH—C(═O)—R_{1z}, —C(═O)—CH₂—CH₂—C(═O)—R_{1z}, Si(R_{1g})₃,

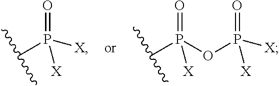

R₃ is H, —C(═O)R_{1b}, —C(═O)OR_{1c}, —C(═O)N(R_{1c})₂, —C(═O)R_{1z}, —C(═O)—(C═O)R_{1b}, —C(═O)—C(═O)OR_{1c}, —C(═O)—C(═O)N(R_{1c})₂, —C(═O)—C(═O)R_{1z}, —C(═O)—CH═CH—C(═O)OR_{1c}, —C(═O)—CH₂—CH₂—C(═O)OR_{1c},

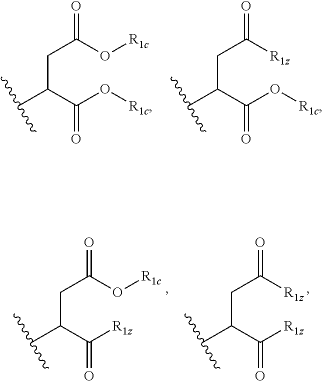

—C(═O)—CH═CH—C(═O)—R_{1z}, —C(═O)—CH₂—CH₂—C(═O)—R_{1z}, Si(R_{1g})₃,

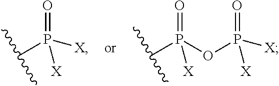

each R_{1b} is independently H, C₁-C₂₀ alkyl, C₂-C₂₀ alkenyl, C₂-C₂₀ alkynyl, —(CH₂)_{q}—C(═O)OR_{1c}, —CH₂—C(═O)—(CH₂)_{q}—C(═O)OR_{1c}, —CH₂—[C(═O)CH₂]ₚ—[CH₂]_{q}—C(═O)OR_{1c}, —CH═CH—C(═O)OR_{1c}, —C(═O)OR_{1c}, —C(═O)N(R_{1c})₂, or R_{1z}, wherein the C₁-C₂₀ alkyl, C₂-C₂₀ alkenyl, or C₂-C₂₀ alkynyl is optionally substituted with one or more R_{1c};
each R_{1c} is independently H, C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl), wherein the C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) is optionally substituted with one or more R_{1c};
each R_{1d} is independently H, C₁-C₂₀ alkyl, C₂-C₂₀ alkenyl, C₂-C₂₀ alkynyl, C₃-C₁₀ cycloalkyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl), wherein the C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₁₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) is optionally substituted with one or more R₁ₑ;
each R₁ₑ is independently H, halogen, C₁-C₂₀ alkyl, C₂-C₂₀ alkenyl, C₂-C₂₀ alkynyl, —OR_{1g}, —C(═O)OR_{1g}, —C(═O)N(R_{1g})₂, —N(R_{1g})₂, —N(R_{1g})C(═O)R_{1f}, —N(R_{1g})C(═O)R_{1z}, —N(R_{1g})C(═O)OR_{1g}, —OC(═O)R_{1f}, —OC(═O)R_{1z}, —OC(═O)OR_{1g}, —SR_{1g}, —N⁺(R_{1g})₃, —SC(═O)R_{1f}, —SC(═O)R_{1z}, —SC(═O)OR_{1g}, —SC(═O)N(R_{1g})₂, —C(═O)R_{1f}, —C(═O)R_{1z},

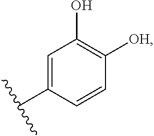 [SMILES: *c1ccc(O)c(O)c1]

or R_{1z}, wherein the C₁-C₂₀ alkyl, C₂-C₂₀ alkenyl, or C₂-C₂₀ alkynyl is optionally substituted with one or more R_{1z};
each R_{1f} is independently H, C₁-C₂₀ alkyl, C₂-C₂₀ alkenyl, C₂-C₂₀ alkynyl, —CH₂C(═O)OR_{1g}, —CH═CH—C(═O)OR_{1g}, —C(═O)OR_{1g}, —C(═O)N(R_{1g})₂,

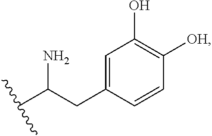 [SMILES: *C(N)Cc1ccc(O)c(O)c1]

or R_{1z}, wherein the C₁-C₂₀ alkyl, C₂-C₂₀ alkenyl, or C₂-C₂₀ alkynyl is optionally substituted with one or more R_{1z};
each R_{1g} is independently H, C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₂ heteroaryl), wherein the C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) is optionally substituted with one or more R_{1z};
each R_{1z} is independently

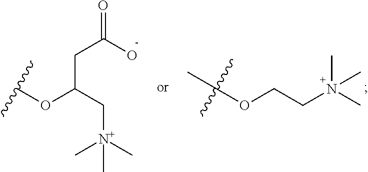

and
each X is independently —OR_{1c}, —SR_{1c}, —N(R_{1c})₂,

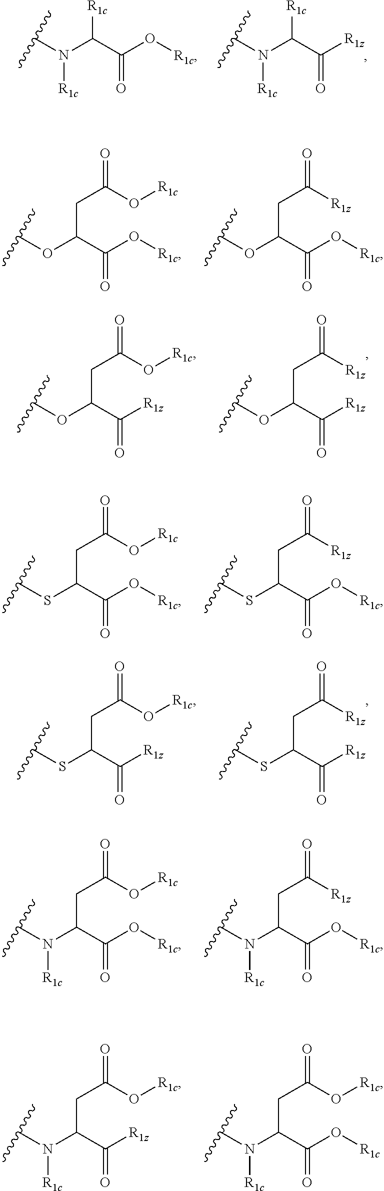

In some aspects, the present disclosure provides a compound of Formula (I) or (II):

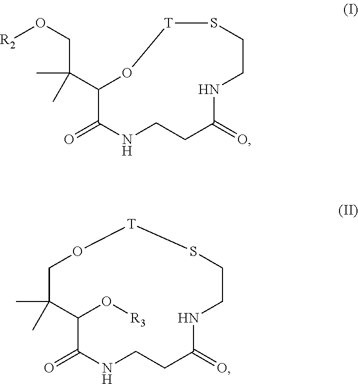

or a pharmaceutically acceptable salt or solvate thereof, wherein:
T is *—C(═O)—(CHR_{1b})ₙ—C(═O)—**;
wherein * denotes an attachment of T to the sulfur atom, and ** denotes an attachment of T to the oxygen atom;
n is an integer ranging from 0 to 20;
R₂ is H, —C(═O)R_{1b}, or Si(R_{1g})₃;
R₃ is H, —C(═O)R_{1b}, or Si(R_{1g})₃;
each R_{1b} is independently H or C₁-C₂₀ alkyl, wherein the C₁-C₂₀ alkyl is optionally substituted with one or more R_{1c};
each R₁ₑ is independently H, C₁-C₂₀ alkyl, or —N(R_{1g})₂;
each R_{1g} is independently H or C₁-C₂₀ alkyl.

It is understood that, for a compound of Formula (I) or (II), T, R_{1b}, R_{1c}, R_{1d}, R₁ₑ, R_{1f}, R_{1g}, R_{1z}, R₂, R₃, X, n, p, q, and r can each be, where applicable, selected from the groups described herein, and any group described herein for any of T, R_{1b}, R_{1c}, R_{1d}, R₁ₑ, R_{1f}, R_{1g}, R_{1z}, R₂, R₃, X, n, p, q, and r can be combined, where applicable, with any group described herein for one or more of the remainder of T, R_{1b}, R_{1c}, R_{1d}, R₁ₑ, R_{1f}, R_{1g}, R_{1z}, R₂, R₃, X, n, p, q, and r.

### Variable T

In some embodiments, T is *—C(═O)—**, *—C(═O)—(CH═CH)ₙ—C(═O)—**, *—C(═O)—(CHR_{1b})ₙ—C(═O)—**, *—C(═O)CH₂—[C(═O)CH₂]ₚ—(CH₂)_{q}—C(═O)—**, *—C(═O)CH₂—[CH(OR_{1c})—CH₂]ₚ—(CH₂)_{q}—C(═O)—**, *—C(═O)CH₂—[C(═O)CH₂]ₚ—[CH(OR_{1c})—CH₂]ᵣ—(CH₂)_{q}—C(═O)—**, *—C(═O)CH₂—[CH(OR_{1c})—CH₂]ᵣ—[C(═O)CH₂]ₚ—(CH₂)_{q}—C(═O)—**, *—C(═O)—(CHR_{1b})ₙ—[C(═O)CH₂]ₚ—(CH₂)_{q}—C(═O)—**, *—C(═O)CH₂—[C(═O)—(CHR_{1b})ₙ]ₚ—(CH₂)_{q}—C(═O)—**, *—C(═O)CH₂—[C(═O)CH₂]ₚ—(CHR_{1b})_{q}—C(═O)—**, *—C(═O)—(CHR_{1b})ₙ—[C(═O)CH₂]ₚ—(CHR_{1b})_{q}—C(═O)—**, **—C(═O)CH₂—[C(═O)CH₂]ₚ—(CH₂)_{q}—C(═O)—*, **—C(═O)CH₂—[CH(OR_{1c})—CH₂]ₚ—(CH₂)_{q}—C(═O)—*, **—C(═O)CH₂—[C(═O)CH₂]ₚ—[CH(OR_{1c})—CH₂]ᵣ—(CH₂)_{q}—C(═O)—*, **—C(═O)CH₂—[CH(OR_{1c})—CH₂]ᵣ—[C(═O)CH₂]ₚ—(CH₂)_{q}—C(═O)—*, **—C(═O)—(CHR_{1b})ₙ—[C(═O)CH₂]ₚ—(CH₂)_{q}—C(═O)—*, **—C(═O)CH₂—[C(═O)—(CHR_{1b})ₙ]ₚ—(CH₂)_{q}—C(═O)—*, **—C(═O)CH₂—[C(═O)CH₂]ₚ—(CHR_{1b})_{q}—C(═O)—*, **—C(═O)—(CHR_{1b})ₙ—[C(═O)CH₂]ₚ—(CHR_{1b})_{q}—C(═O)—*,

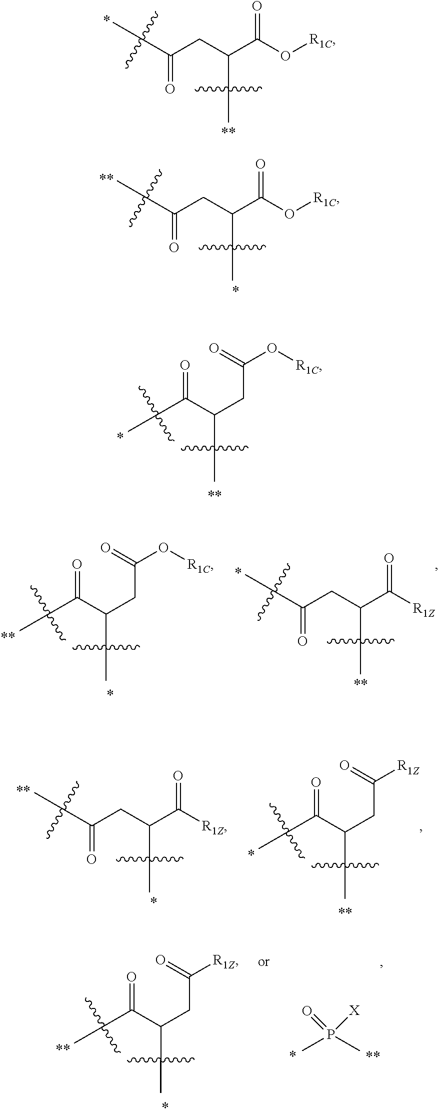

or wherein * denotes an attachment of T to the sulfur atom, and ** denotes an attachment of T to the oxygen atom.

In some embodiments, T is *—C(═O)—**.

In some embodiments, T is *—C(═O)—(CH═CH)ₙ—C(═O)—**. In some embodiments, T is *—C(═O)—C(═O)—**.

In some embodiments, T is *—C(═O)—(CH═CH)—C(═O)—**.

In some embodiments, T is *—C(═O)—(CHR_{1b})ₙ—C(═O)—**.

In some embodiments, T is *—C(═O)—CHR_{1b}—C(═O)—**.

In some embodiments, T is *—C(═O)—CH₂—C(═O)—**.

In some embodiments, T is *—C(═O)—(CHR_{1b})(CHR_{1b})—C(═O)—**. In some embodiments, T is *—C(═O)—CH₂CH₂C(═O)—**.

In some embodiments, T is *—C(═O)CH₂—[C(═O)CH₂]ₚ—(CH₂)_{q}—C(═O)—**. In some embodiments, T is *—C(═O)CH₂—C(═O)CH₂—CH₂—C(═O)—**. In some embodiments, T is *—C(═O)CH₂—(CH₂)_{q}—C(═O)—**. In some embodiments, T is *—C(═O)CH₂—[C(═O)CH₂]ₚ—C(═O)—**.

In some embodiments, T is *—C(═O)CH₂—[CH(OR_{1c})—CH₂]ₚ—(CH₂)_{q}—C(═O)—**. In some embodiments, T is *—C(═O)CH₂—(CH₂)_{q}—C(═O)—**. In some embodiments, T is *—C(═O)CH₂—[CH(OR_{1c})—CH₂]ₚ—C(═O)—**.

In some embodiments, T is *—C(═O)CH₂—[C(═O)CH₂]ₚ—[CH(OR_{1c})—CH₂]ᵣ—(CH₂)_{q}—C(═O)—**. In some embodiments, T is *—C(═O)CH₂—[CH(OR_{1c})—CH₂]ᵣ—(CH₂)_{q}—C(═O)—**. In some embodiments, T is *—C(═O)CH₂—[C(═O)CH₂]ₚ—[CH(OR_{1c})—CH₂]ᵣ—C(═O)—**. In some embodiments, T is *—C(═O)CH₂—[CH(OR_{1c})—CH₂]r-C(═O)—**.

In some embodiments, T is *—C(═O)CH₂—[CH(OR_{1c})—CH₂]ᵣ—[C(═O)CH₂]ₚ—(CH₂)_{q}—C(═O)—**. In some embodiments, T is *—C(═O)CH₂—[CH(OR_{1c})—CH₂]ᵣ—[C(═O)CH₂]ₚ—C(═O)—**. In some embodiments, T is *—C(═O)CH₂—[CH(OR_{1c})—CH₂]ᵣ—C(═O)—**.

In some embodiments, T is *—C(═O)—(CHR_{1b})ₙ—[C(═O)CH₂]ₚ—(CH₂)_{q}—C(═O)—**. In some embodiments, T is *—C(═O)—(CHR_{1b})ₙ—(CH₂)_{q}—C(═O)—**. In some embodiments, T is *—C(═O)—(CHR_{1b})ₙ—[C(═O)CH₂]ₚ—C(═O)—**. In some embodiments, T is *—C(═O)—[C(═O)CH₂]ₚ—(CH₂)_{q}—C(═O)—**. In some embodiments, T is *—C(═O)—(CHR_{1b})ₙ—C(═O)—**. In some embodiments, T is *—C(═O)—[C(═O)CH₂]ₚ—C(═O)—**. In some embodiments, T is *—C(═O)—(CH₂)_{q}—C(═O)—**.

In some embodiments, T is *—C(═O)CH₂—[C(═O)—(CHR_{1b})ₙ]ₚ—(CH₂)_{q}—C(═O)—**. In some embodiments, T is *—C(═O)CH₂—[C(═O)]ₚ—(CH₂)_{q}—C(═O)—**. In some embodiments, T is *—C(═O)CH₂—[C(═O)—(CHR_{1b})ₙ]ₚ—C(═O)—**. In some embodiments, T is *—C(═O)CH₂—[C(═O)]ₚ—C(═O)—**.

In some embodiments, T is *—C(═O)CH₂—[C(═O)CH₂]ₚ—(CHR_{1b})_{q}—C(═O)—**. In some embodiments, T is *—C(═O)CH₂—(CHR_{1b})_{q}—C(═O)—**. In some embodiments, T is *—C(═O)CH₂—[C(═O)CH₂]ₚ—C(═O)—**.

In some embodiments, T is *—C(═O)—(CHR_{1b})ₙ—[C(═O)CH₂]ₚ—(CHR_{1b})_{q}—C(═O)—**. In some embodiments, T is *—C(═O)—[C(═O)CH₂]ₚ—(CHR_{1b})_{q}—C(═O)—**. In some embodiments, T is *—C(═O)—(CHR_{1b})ₙ—(CHR_{1b})_{q}—C(═O)—**. In some embodiments, T is *—C(═O)—[C(═O)CH₂]ₚ—C(═O)—**. In some embodiments, T is *—C(═O)—(CHR_{1b})_{q}—C(═O)—**.

In some embodiments, T is **—C(═O)CH₂—[C(═O)CH₂]ₚ—(CH₂)_{q}—C(═O)—*. In some embodiments, T is **—C(═O)CH₂—C(═O)CH₂—CH₂—C(═O)—*. In some embodiments, T is **—C(═O)CH₂—(CH₂)_{q}—C(═O)—*. In some embodiments, T is **—C(═O)CH₂—[C(═O)CH₂]ₚ—C(═O)—*.

In some embodiments, T is **—C(═O)CH₂—[CH(OR_{1c})—CH₂]ₚ—(CH₂)_{q}—C(═O)—*. In some embodiments, T is **—C(═O)CH₂—[CH(OR_{1c})—CH₂]ₚ—C(═O)—*.

In some embodiments, T is **—C(═O)CH₂—[C(═O)CH₂]ₚ—[CH(OR_{1c})—CH₂](CH₂)_{q}—C(═O)—*. In some embodiments, T is **—C(═O)CH₂—[CH(OR_{1c})—CH₂]ᵣ—(CH₂)_{q}—C(═O)—*. In some embodiments, T is **—C(═O)CH₂—[C(═O)CH₂]ₚ—[CH(OR_{1c})—CH₂]ᵣ—C(═O)—*. In some embodiments, T is **—C(═O)CH₂—[CH(OR_{1c})—CH₂]ᵣ—C(═O)—*.

In some embodiments, T is **—C(═O)CH₂—[CH(OR_{1c})—CH₂]ᵣ—[C(═O)CH₂]ₚ—(CH₂)_{q}—C(═O)—*. In some embodiments, T is **—C(═O)CH₂—[CH(OR_{1c})—CH₂]ᵣ—[C(═O)CH₂]ₚ—C(═O)—*.

In some embodiments, T is **—C(═O)CH₂—[CH(OR_{1c})—CH₂]r-C(═O)—*. In some embodiments, T is **—C(═O)CH₂ᵣ—[C(═O)CH₂]ₚ—C(═O)—*. In some embodiments, T is **—C(═O)CH₂—(CH₂)_{q}—C(═O)—*.

In some embodiments, T is **—C(═O)—(CHR_{1b})ₙ—[C(═O)CH₂]ₚ—(CH₂)_{q}—C(═O)—*. In some embodiments, T is **—C(═O)—(CHR_{1b})ₙ—(CH₂)_{q}—C(═O)—*. In some embodiments, T is **—C(═O)—(CHR_{1b})ₙ—[C(═O)CH₂]ₚ—C(═O)—*. In some embodiments, T is **—C(═O)—[C(═O)CH₂]ₚ—(CH₂)_{q}—C(═O)—*. In some embodiments, T is **—C(═O)—[C(═O)CH₂]ₚ—C(═O)—*.

In some embodiments, T is **—C(═O)CH₂—[C(═O)—(CHR_{1b})ₙ]ₚ—(CH₂)_{q}—C(═O)—*. In some embodiments, T is **—C(═O)CH₂—[C(═O)]ₚ—(CH₂)_{q}—C(═O)—*. In some embodiments, T is **—C(═O)CH₂—(CH₂)_{q}—C(═O)—*. In some embodiments, T is **—C(═O)CH₂—[C(═O)—(CHR_{1b})ₙ]ₚ—C(═O)—*. In some embodiments, T is **—C(═O)CH₂—[C(═O)]ₚ—C(═O)—*.

In some embodiments, T is **—C(═O)CH₂—[C(═O)CH₂]ₚ—(CHR_{1b})_{q}—C(═O)—*. In some embodiments, T is **—C(═O)CH₂—(CHR_{1b})_{q}—C(═O)—*. In some embodiments, T is **—C(═O)CH₂—[C(═O)CH₂]ₚ—C(═O)—*.

In some embodiments, T is **—C(═O)—(CHR_{1b})ₙ—[C(═O)CH₂]ₚ—(CHR_{1b})_{q}—C(═O)—*. In some embodiments, T is **—C(═O)—(CHR_{1b})ₙ—(CHR_{1b})_{q}—C(═O)—*. In some embodiments, T is **—C(═O)—(CHR_{1b})ₙ—[C(═O)CH₂]ₚ—C(═O)—*. In some embodiments, T is **—C(═O)—[C(═O)CH₂]ₚ—(CHR_{1b})_{q}—C(═O)—*. In some embodiments, T is **—C(═O)—(CHR_{1b})ₙ—C(═O)—*.

In some embodiments, T is

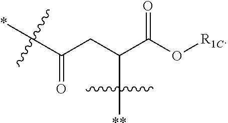

In some embodiments, T is

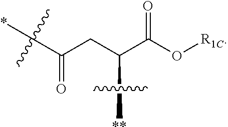

In some embodiments, T is

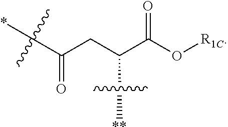

In some embodiments, T is

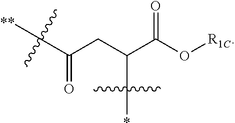

In some embodiments, T is

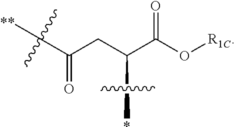

In some embodiments, T is

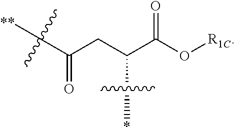

In some embodiments, T is

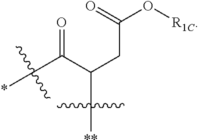

In some embodiments, T is

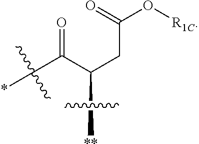

In some embodiments, T is

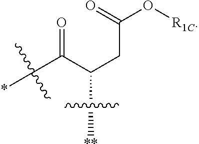

In some embodiments, T is

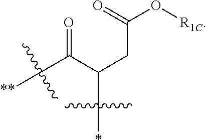

In some embodiments, T is

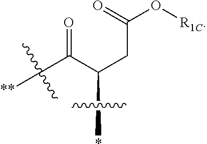

In some embodiments, T is

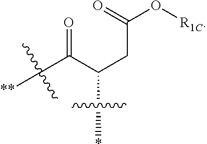

In some embodiments, T is

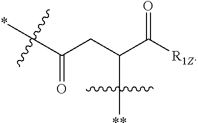

In some embodiments, T is

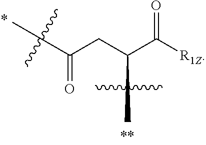

In some embodiments, T is

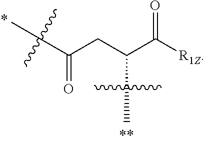

In some embodiments, T is

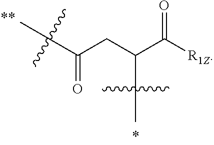

In some embodiments, T is

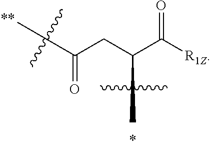

In some embodiments, T is

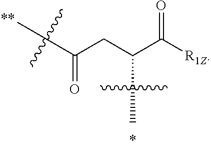

In some embodiments, T is

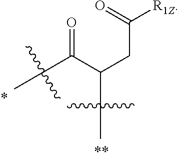

In some embodiments, T is

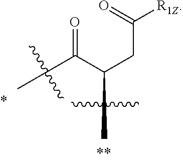

In some embodiments, T is

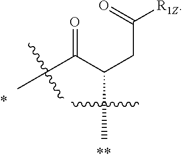

In some embodiments, T is

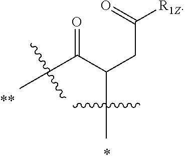

In some embodiments, T is

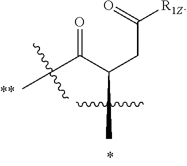

In some embodiments, T is

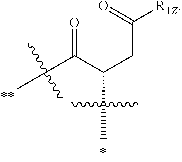

In some embodiments, T is

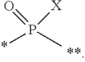

In some embodiments, T is

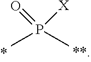

wherein X is —OR_{1c}. In some embodiments, T is

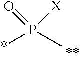

wherein X is —SR_{1c}. In some embodiments, T is

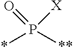

wherein X is —N(R_{1c})₂. In some embodiments, T is

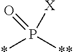

wherein X is

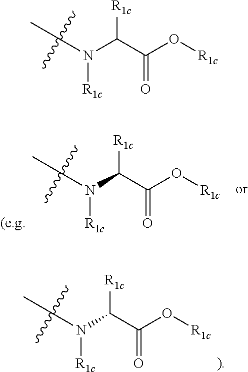

In some embodiments, T is

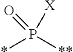

wherein X is

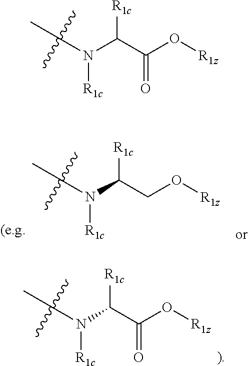

In some embodiments, T is

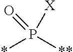

wherein X is

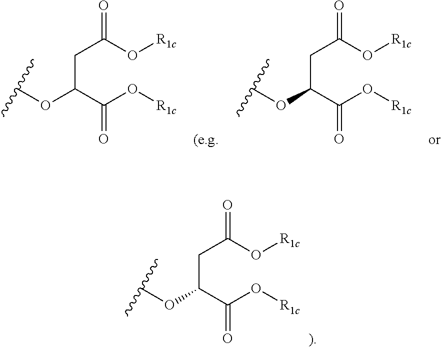

In some embodiments, T is

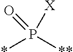

wherein X is

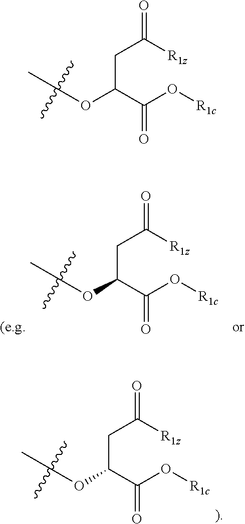

In some embodiments, T is

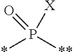

wherein X is

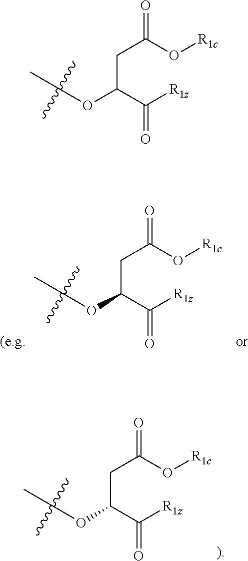

In some embodiments, T is

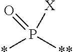

wherein X is

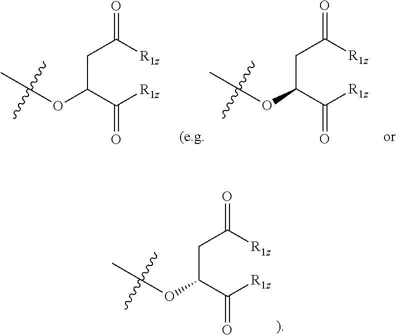

In some embodiments, T is

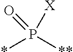

wherein X is

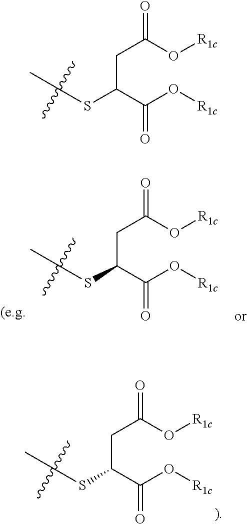

In some embodiments, T is

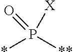

wherein X is

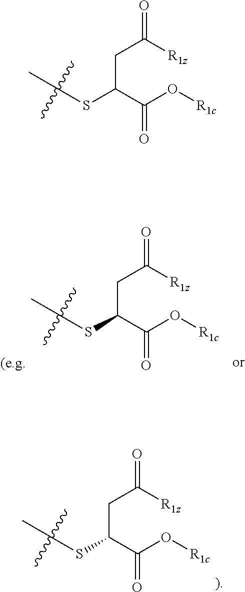

In some embodiments, T is

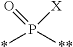

wherein X is

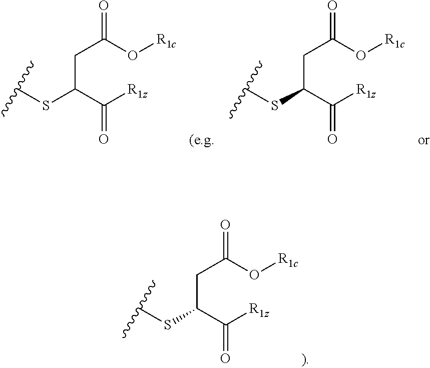

In some embodiments, T is

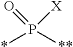

wherein X is

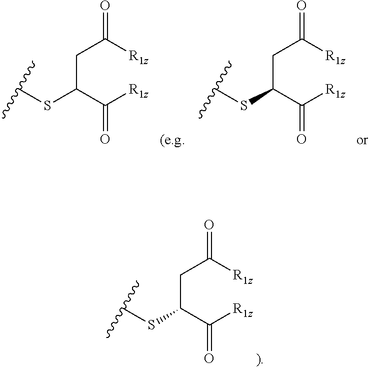

In some embodiments, T is

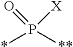

wherein X is

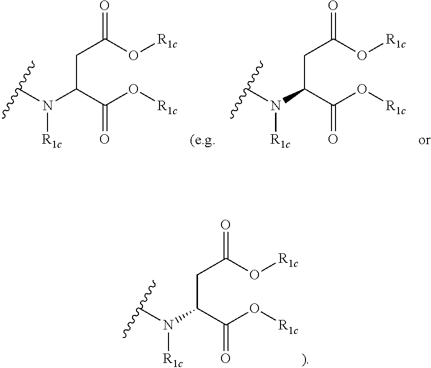

In some embodiments, T is

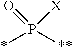

wherein X is

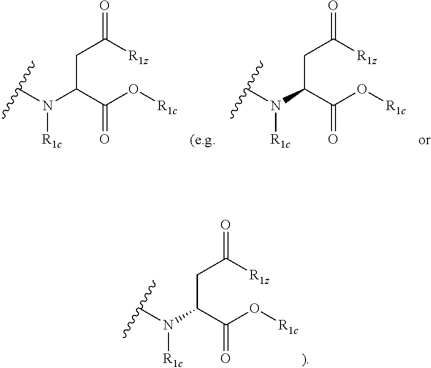

In some embodiments, T is

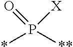

wherein X is

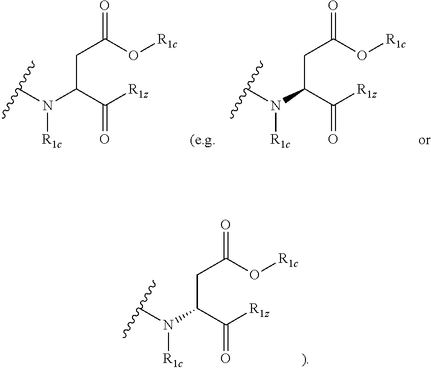

In some embodiments, T is

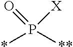

wherein X

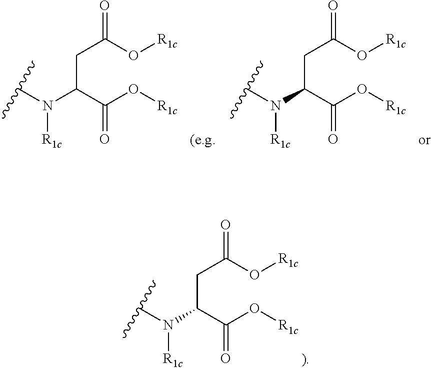

In some embodiments, T is

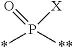

wherein X is R_{1z}.

In some embodiments, T is

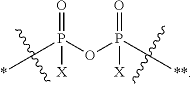

In some embodiments, T is

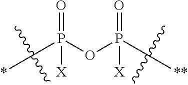

wherein at least one X (e.g. one or both) is —OR_{1c}. In some embodiments, T is

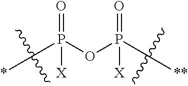

wherein at least one X (e.g. one or both) is —SR_{1c}. In some embodiments, T is

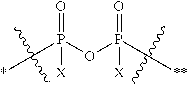

wherein at least one X (e.g. one or both) is —N(R_{1c})₂. In some embodiments, T is

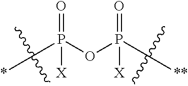

wherein at least one X (e.g. one or both) is

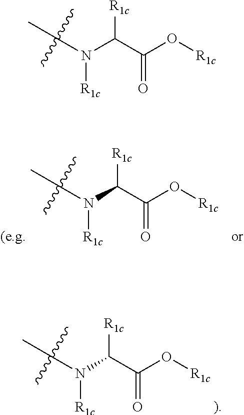

In some embodiments, T is

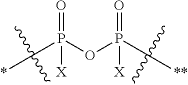

wherein at least one X (e.g. one or both) is

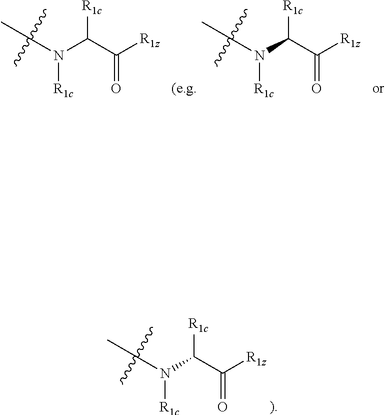

In some embodiments, T is

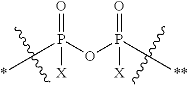

wherein at least one X (e.g. one or both) is

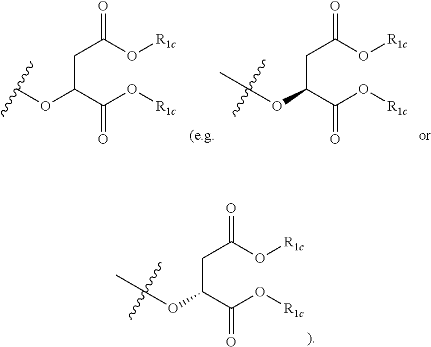

In some embodiments, T is

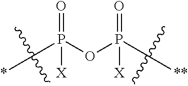

wherein at least one X (e.g. one or both) is

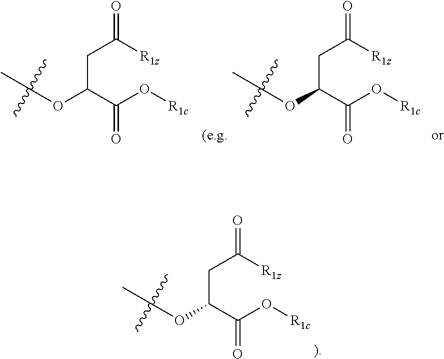

In some embodiments, T is

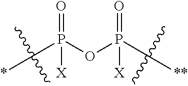

wherein at least one X (e.g. one or both) is

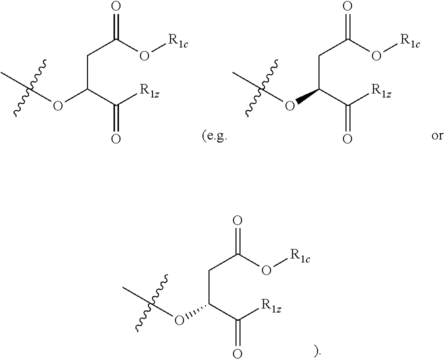

In some embodiments, T is

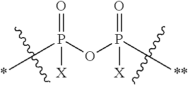

wherein at least one X (e.g. one or both) is

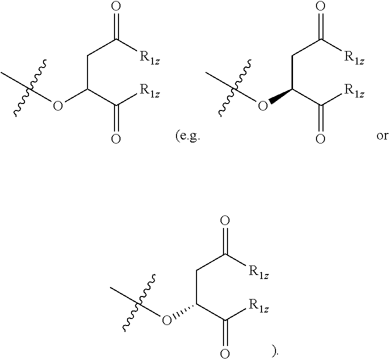

In some embodiments, T is

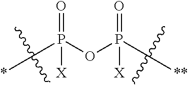

wherein at least one X (e.g. one or both) is

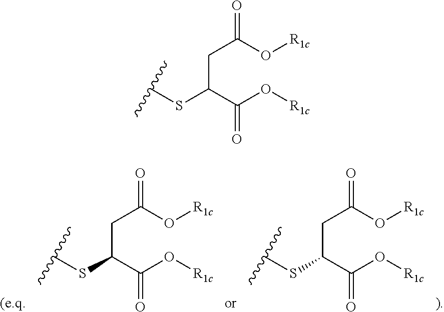

In some embodiments, T is

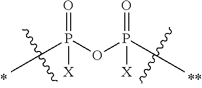

wherein at least one X (e.g. one or both) is

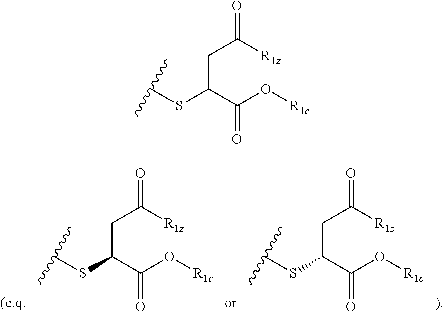

In some embodiments, T is

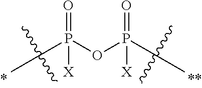

wherein at least one X (e.g. one or both) is

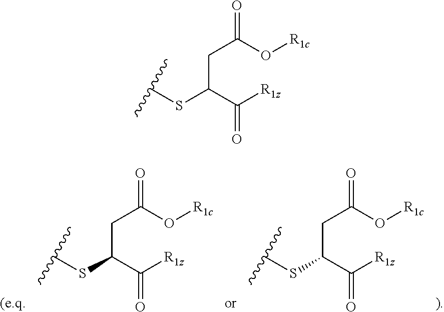

In some embodiments, T is

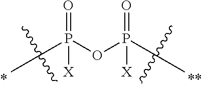

wherein at least one X (e.g. one or both) is

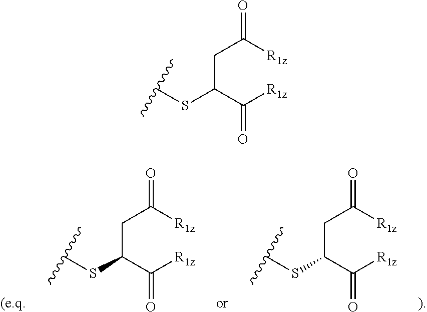

In some embodiments, T is

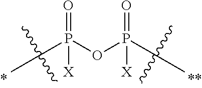

wherein at least one X (e.g. one or both) is

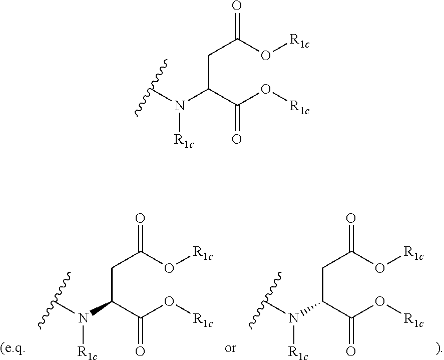

In some embodiments, T is

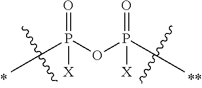

wherein at least one X (e.g. one or both) is

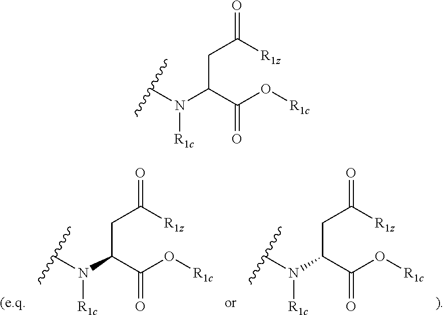

In some embodiments, T is

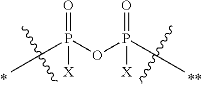

wherein at least one X (e.g. one or both) is

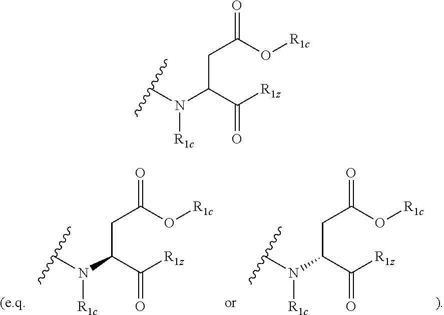

In some embodiments, T is

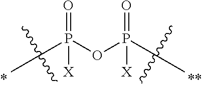

wherein at least one X (e.g. one or both) is

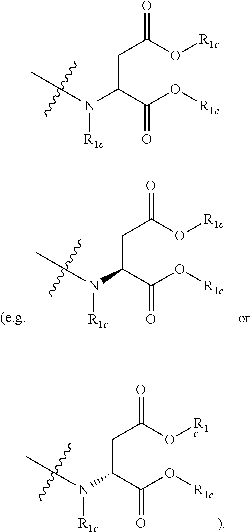

In some embodiments, T is

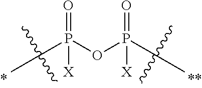

wherein at least one X (e.g. one or both) is R_{1z}.

### Variable R_{1b}

In some embodiments, at least one R_{1b} is H.

In some embodiments, at least one R_{1b} is C₁-C₂₀ alkyl, C₂-C₂₀ alkenyl, or C₂-C₂₀ alkynyl optionally substituted with one or more R₁ₑ.

In some embodiments, at least one R_{1b} is C₁-C₂₀ alkyl (e.g., methyl, ethyl, propyl, butyl, pentyl, hexyl, or heptyl).

In some embodiments, at least one R_{1b} is C₁-C₂₀ alkyl (e.g., methyl, ethyl, propyl, butyl, pentyl, hexyl, or heptyl) substituted with one or more R₁ₑ.

In some embodiments, at least one R_{1b} is C₁-C₂₀ alkyl (e.g., methyl, ethyl, propyl, butyl, pentyl, hexyl, or heptyl) substituted with one or more R_{1z}.

In some embodiments, at least one R_{1b} is C₂-C₂₀ alkenyl (e.g., ethenyl, propenyl, butenyl, pentenyl, or hexenyl).

In some embodiments, at least one R_{1b} is C₂-C₂₀ alkenyl (e.g., ethenyl, propenyl, butenyl, pentenyl, or hexenyl) substituted with one or more R₁ₑ.

In some embodiments, at least one R_{1b} is C₂-C₂₀ alkenyl (e.g., ethenyl, propenyl, butenyl, pentenyl, or hexenyl) substituted with one or more R_{1z}.

In some embodiments, at least one R_{1b} is C₂-C₂₀ alkynyl (e.g., ethynyl, propynyl, butynyl, pentynyl, or hexynyl).

In some embodiments, at least one R_{1b} is C₂-C₂₀ alkynyl (e.g., ethynyl, propynyl, butynyl, pentynyl, or hexynyl) substituted with one or more R₁ₑ.

In some embodiments, at least one R_{1b} is C₂-C₂₀ alkynyl (e.g., ethynyl, propynyl, butynyl, pentynyl, or hexynyl) substituted with one or more R_{1z}.

In some embodiments, at least one R_{1b} is —(CH₂)_{q}—C(═O)OR_{1c}, —CH₂—C(═O)—(CH₂)_{q}—C(═O)OR_{1c}, —CH₂—[C(═O)CH₂]ₚ—[CH₂]_{q}—C(═O)OR_{1c}, —CH═CH—C(═O)OR_{1c}. —C(═O)OR_{1c}, —C(═O)N(R_{1c})₂, or R_{1z}.

In some embodiments, at least one R_{1b} is —(CH₂)_{q}—C(═O)OR_{1c}.

In some embodiments, at least one R_{1b} is —(CH₂)_{q}—C(═O)OH.

In some embodiments, at least one R_{1b} is —(CH₂)_{q}—C(═O)OR_{1c}, wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₁₀ alkyl)-(C₃-C₁₂ heteroaryl) optionally substituted with one or more R₁ₑ.

In some embodiments, at least one R_{1b} is —(CH₂)_{q}—C(═O)OR_{1c}, wherein R_{1c} is C₁-C₂₀ alkyl, C₂-C₂₀ alkenyl, or C₂-C₂₀ alkynyl optionally substituted with one or more R₁ₑ.

In some embodiments, at least one R_{1b} is —(CH₂)_{q}—C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, at least one R_{1b} is —(CH₂)_{q}—C(═O)OR_{1c}, wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —CH₂CH₂—C(═O)OR_{1c}.

In some embodiments, at least one R_{1b} is —CH₂CH₂—C(═O)OH.

In some embodiments, at least one R_{1b} is —CH₂CH₂—C(═O)OR_{1c}, wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —CH₂CH₂—C(═O)OR_{1c}, wherein R_{1c} is C₁-C₂₀ alkyl, C₂-C₂₀ alkenyl, or C₂-C₂₀ alkynyl optionally substituted with one or more R₁ₑ.

In some embodiments, at least one R_{1b} is —CH₂CH₂—C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, at least one R_{1b} is —CH₂CH₂—C(═O)OR_{1c}, wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —CH₂—C(═O)—(CH₂)_{q}—C(═O)OR_{1c}.

In some embodiments, at least one R_{1b} is —CH₂—C(═O)—(CH₂)_{q}—C(═O)OH.

In some embodiments, at least one R_{1b} is —CH₂—C(═O)—(CH₂)_{q}—C(═O)OR_{1c}, wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —CH₂—C(═O)—(CH₂)_{q}—C(═O)OR_{1c}, wherein R_{1c} is C₁-C₂₀ alkyl, C₂-C₂₀ alkenyl, or C₂-C₂₀ alkynyl optionally substituted with one or more R₁ₑ.

In some embodiments, at least one R_{1b} is —CH₂—C(═O)—(CH₂)_{q}—C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, at least one R_{1b} is —CH₂—C(═O)—(CH₂)_{q}—C(═O)OR_{1c}, wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —CH₂—[C(═O)CH₂]ₚ—[CH₂]_{q}—C(═O)OR_{1c}.

In some embodiments, at least one R_{1b} is —CH₂—[C(═O)CH₂]ₚ—[CH₂]_{q}—C(═O)OH.

In some embodiments, at least one R_{1b} is —CH₂—[C(═O)CH₂]ₚ—[CH₂]_{q}—C(═O)OR_{1c}, wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —CH₂—[C(═O)CH₂]ₚ—[CH₂]_{q}—C(═O)OR_{1c}, wherein R_{1c} is C₁-C₂₀ alkyl, C₂-C₂₀ alkenyl, or C₂-C₂₀ alkynyl optionally substituted with one or more R₁ₑ.

In some embodiments, at least one R_{1b} is —CH₂—[C(═O)CH₂]ₚ—[CH₂]_{q}—C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, at least one R_{1b} is —CH₂—[C(═O)CH₂]ₚ—[CH₂]_{q}—C(═O)OR_{1c}, wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —CH₂—C(═O)—CH₂CH₂—C(═O)OR_{1c}.

In some embodiments, at least one R_{1b} is —CH₂—C(═O)—CH₂CH₂—C(═O)OH.

In some embodiments, at least one R_{1b} is —CH₂—C(═O)—CH₂CH₂—C(═O)OR_{1c}, wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —CH₂—C(═O)—CH₂CH₂—C(═O)OR_{1c}, wherein R_{1c} is C₁-C₂₀ alkyl, C₂-C₂₀ alkenyl, or C₂-C₂₀ alkynyl optionally substituted with one or more R₁ₑ.

In some embodiments, at least one R_{1b} is —CH₂—C(═O)—CH₂CH₂—C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, at least one R_{1b} is —CH₂—C(═O)—CH₂CH₂—C(═O)OR_{1c}, wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —CH═CH—C(═O)OR_{1c}.

In some embodiments, at least one R_{1b} is —CH═CH—C(═O)OH.

In some embodiments, at least one R_{1b} is —CH═CH—C(═O)OR_{1c}, wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —CH═CH—C(═O)OR_{1c}, wherein R_{1c} is C₁-C₂₀ alkyl, C₂-C₂₀ alkenyl, or C₂-C₂₀ alkynyl optionally substituted with one or more R₁ₑ.

In some embodiments, at least one R_{1b} is —CH═CH—C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, at least one R_{1b} is —CH═CH—C(═O)OR_{1c}, wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —C(═O)OR_{1c}.

In some embodiments, at least one R_{1b} is —C(═O)OH.

In some embodiments, at least one R_{1b} is —C(═O)OR_{1c}, wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —C(═O)OR_{1c}, wherein R_{1c} is C₁-C₂₀ alkyl, C₂-C₂₀ alkenyl, or C₂-C₂₀ alkynyl optionally substituted with one or more R₁ₑ.

In some embodiments, at least one R_{1b} is —C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, at least one R_{1b} is —C(═O)OR_{1c}, wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —C(═O)N(R_{1c})₂.

In some embodiments, at least one R_{1b} is —C(═O)NHR₁ₑ.

In some embodiments, at least one R_{1b} is —C(═O)NH₂.

In some embodiments, at least one R_{1b} is —C(═O)N(R_{1c})₂, wherein at least one R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is —C(═O)N(R_{1c})₂, wherein at least one R_{1c} is C₁-C₂₀ alkyl, C₂-C₂₀ alkenyl, or C₂-C₂₀ alkynyl optionally substituted with one or more R₁ₑ.

In some embodiments, at least one R_{1b} is —C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, at least one R_{1b} is —C(═O)N(R_{1c})₂, wherein at least one R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1b} is R_{1z}.

In some embodiments, at least one R_{1b} is

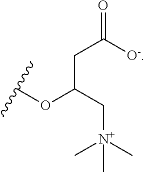

In some embodiments, at least one R_{1b} is

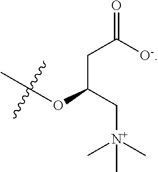

In some embodiments, at least one R_{1b} is

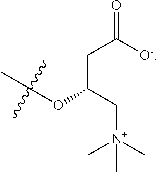

In some embodiments, at least one R_{1b} is

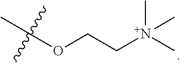 [SMILES: *OCC[N+](C)(C)C]

### Variable R_{1c}

In some embodiments, at least one R_{1c} is H.

In some embodiments, at least one R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) is optionally substituted with one or more R₁ₑ.

In some embodiments, at least one R_{1c} is C₁-C₂₀ alkyl, C₂-C₂₀ alkenyl, or C₂-C₂₀ alkynyl optionally substituted with one or more R₁ₑ.

In some embodiments, at least one R_{1c} is C₁-C₂₀ alkyl (e.g., methyl, ethyl, propyl, butyl, pentyl, hexyl, or heptyl).

In some embodiments, at least one R_{1c} is C₁-C₂₀ alkyl (e.g., methyl, ethyl, propyl, butyl, pentyl, hexyl, or heptyl) substituted with one or more R₁ₑ.

In some embodiments, at least one R_{1c} is C₁-C₂₀ alkyl (e.g., methyl, ethyl, propyl, butyl, pentyl, hexyl, or heptyl) substituted with one or more R_{1z}.

In some embodiments, at least one R_{1c} is C₂-C₂₀ alkenyl (e.g., ethenyl, propenyl, butenyl, pentenyl, or hexenyl).

In some embodiments, at least one R_{1c} is C₂-C₂₀ alkenyl (e.g., ethenyl, propenyl, butenyl, pentenyl, or hexenyl) substituted with one or more R₁ₑ.

In some embodiments, at least one R_{1c} is C₂-C₂₀ alkenyl (e.g., ethenyl, propenyl, butenyl, pentenyl, or hexenyl) substituted with one or more R_{1z}.

In some embodiments, at least one R_{1c} is C₂-C₂₀ alkynyl (e.g., ethynyl, propynyl, butynyl, pentynyl, or hexynyl).

In some embodiments, at least one R_{1c} is C₂-C₂₀ alkynyl (e.g., ethynyl, propynyl, butynyl, pentynyl, or hexynyl) substituted with one or more R₁ₑ.

In some embodiments, at least one R_{1c} is C₂-C₂₀ alkynyl (e.g., ethynyl, propynyl, butynyl, pentynyl, or hexynyl) substituted with one or more R_{1z}.

In some embodiments, at least one R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, at least one R_{1c} is C₃-C₁₂ cycloalkyl optionally substituted with one or more R₁ₑ. In some embodiments, at least one R_{1c} is C₃-C₁₂ cycloalkyl. In some embodiments, at least one R_{1c} is C₃-C₁₂ cycloalkyl substituted with one or more R₁ₑ. In some embodiments, at least one R_{1c} is C₃-C₁₂ cycloalkyl substituted with one or more R_{1z}.

In some embodiments, at least one R_{1c} is C₃-C₁₂ heterocycloalkyl optionally substituted with one or more R₁ₑ. In some embodiments, at least one R_{1c} is C₃-C₁₂ heterocycloalkyl. In some embodiments, at least one R_{1c} is C₃-C₁₂ heterocycloalkyl substituted with one or more R₁ₑ. In some embodiments, at least one R_{1c} is C₃-C₁₂ heterocycloalkyl substituted with one or more R_{1z}.

In some embodiments, at least one R_{1c} is C₃-C₁₂ aryl optionally substituted with one or more R₁ₑ. In some embodiments, at least one R_{1c} is C₃-C₁₂ aryl. In some embodiments, at least one R_{1c} is C₃-C₁₂ aryl substituted with one or more R₁ₑ. In some embodiments, at least one R_{1c} is C₃-C₁₂ aryl substituted with one or more R_{1z}.

In some embodiments, at least one R_{1c} is C₃-C₁₂ heteroaryl optionally substituted with one or more R₁ₑ. In some embodiments, at least one R_{1c} is C₃-C₁₂ heteroaryl. In some embodiments, at least one R_{1c} is C₃-C₁₂ heteroaryl substituted with one or more R₁ₑ. In some embodiments, at least one R_{1c} is C₃-C₁₂ heteroaryl substituted with one or more R_{1z}.

In some embodiments, at least one R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₁₀ alkyl)-(C₃-C₁₂ heteroaryl) optionally substituted with one or more R₁ₑ.

In some embodiments, at least one R_{1c} is —(C₁-C₂₀ alkyl)-(C₃-C₁₂ cycloalkyl) optionally substituted with one or more R₁ₑ. In some embodiments, at least one R_{1c} is —(C₁-C₂₀ alkyl)-(C₃-C₁₂ cycloalkyl). In some embodiments, at least one R_{1c} is —(C₁-C₂₀ alkyl)-(C₃-C₁₂ cycloalkyl) substituted with one or more R₁ₑ. In some embodiments, at least one R_{1c} is —(C₁-C₂₀ alkyl)-(C₃-C₁₂ cycloalkyl) substituted with one or more R_{1z}.

In some embodiments, at least one R_{1c} is —(C₁-C₂₀ alkyl)-(C₃-C₁₂ heterocycloalkyl) optionally substituted with one or more R₁ₑ. In some embodiments, at least one R_{1c} is —(C₁-C₂₀ alkyl)-(C₃-C₁₂ heterocycloalkyl). In some embodiments, at least one R_{1c} is —(C₁-C₂₀ alkyl)-(C₃-C₁₂ heterocycloalkyl) substituted with one or more R₁ₑ. In some embodiments, at least one R_{1c} is —(C₁-C₂₀ alkyl)-(C₃-C₁₂ heterocycloalkyl) substituted with one or more R_{1z}.

In some embodiments, at least one R_{1c} is —(C₁-C₂₀ alkyl)-(C₃-C₁₂ aryl) optionally substituted with one or more R₁ₑ. In some embodiments, at least one R_{1c} is —(C₁-C₂₀ alkyl)-(C₃-C₁₂ aryl). In some embodiments, at least one R_{1c} is —(C₁-C₂₀ alkyl)-(C₃-C₁₂ aryl) substituted with one or more R₁ₑ. In some embodiments, at least one R_{1c} is —(C₁-C₂₀ alkyl)-(C₃-C₁₂ aryl) substituted with one or more R_{1z}.

In some embodiments, at least one R_{1c} is —(C₁-C₂₀ alkyl)-(C₃-C₁₂ heteroaryl) optionally substituted with one or more R₁ₑ. In some embodiments, at least one R_{1c} is —(C₁-C₂₀ alkyl)-(C₃-C₁₂ heteroaryl). In some embodiments, at least one R_{1c} is —(C₁-C₂₀ alkyl)-(C₃-C₁₂ heteroaryl) substituted with one or more R₁ₑ. In some embodiments, at least one R_{1c} is —(C₁-C₂₀ alkyl)-(C₃-C₁₂ heteroaryl) substituted with one or more R_{1z}.

### Variable R_{1d}

In some embodiments, at least one R_{1d} is H.

In some embodiments, at least one R_{1d} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) is optionally substituted with one or more R₁ₑ.

In some embodiments, at least one R_{1d} is C₁-C₂₀ alkyl, C₂-C₂₀ alkenyl, or C₂-C₂₀ alkynyl optionally substituted with one or more R₁ₑ.

In some embodiments, at least one R_{1d} is C₁-C₂₀ alkyl (e.g., methyl, ethyl, propyl, butyl, pentyl, hexyl, or heptyl).

In some embodiments, at least one R_{1d} is C₁-C₂₀ alkyl (e.g., methyl, ethyl, propyl, butyl, pentyl, hexyl, or heptyl) substituted with one or more R₁ₑ.

In some embodiments, at least one R_{1d} is C₁-C₂₀ alkyl (e.g., methyl, ethyl, propyl, butyl, pentyl, hexyl, or heptyl) substituted with one or more R_{1z}.

In some embodiments, at least one R_{1d} is C₂-C₂₀ alkenyl (e.g., ethenyl, propenyl, butenyl, pentenyl, or hexenyl).

In some embodiments, at least one R_{1d} is C₂-C₂₀ alkenyl (e.g., ethenyl, propenyl, butenyl, pentenyl, or hexenyl) substituted with one or more R₁ₑ.

In some embodiments, at least one R_{1d} is C₂-C₂₀ alkenyl (e.g., ethenyl, propenyl, butenyl, pentenyl, or hexenyl) substituted with one or more R_{1z}.

In some embodiments, at least one R_{1d} is C₂-C₂₀ alkynyl (e.g., ethynyl, propynyl, butynyl, pentynyl, or hexynyl).

In some embodiments, at least one R_{1d} is C₂-C₂₀ alkynyl (e.g., ethynyl, propynyl, butynyl, pentynyl, or hexynyl) substituted with one or more R₁ₑ.

In some embodiments, at least one R_{1d} is C₂-C₂₀ alkynyl (e.g., ethynyl, propynyl, butynyl, pentynyl, or hexynyl) substituted with one or more R_{1z}.

In some embodiments, at least one R_{1d} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, at least one R_{1d} is C₃-C₂ cycloalkyl optionally substituted with one or more R₁ₑ. In some embodiments, at least one R_{1d} is C₃-C₁₂ cycloalkyl. In some embodiments, at least one R_{1d} is C₃-C₁₂ cycloalkyl substituted with one or more R₁ₑ. In some embodiments, at least one R_{1d} is C₃-C₁₂ cycloalkyl substituted with one or more R_{1z}.

In some embodiments, at least one R_{1d} is C₃-C₁₂ heterocycloalkyl optionally substituted with one or more R₁ₑ. In some embodiments, at least one R_{1d} is C₃-C₁₂ heterocycloalkyl. In some embodiments, at least one R_{1d} is C₃-C₁₂ heterocycloalkyl substituted with one or more R₁ₑ.

In some embodiments, at least one R_{1d} is C₃-C₁₂ heterocycloalkyl substituted with one or more R_{1z}.

In some embodiments, at least one R_{1d} is C₃-C₁₂ aryl optionally substituted with one or more R₁ₑ. In some embodiments, at least one R_{1d} is C₃-C₁₂ aryl. In some embodiments, at least one R_{1d} is C₃-C₁₂ aryl substituted with one or more R₁ₑ. In some embodiments, at least one R_{1d} is C₃-C₁₂ aryl substituted with one or more R_{1z}.

In some embodiments, at least one R_{1d} is C₃-C₁₂ heteroaryl optionally substituted with one or more R₁ₑ. In some embodiments, at least one R_{1d} is C₃-C₁₂ heteroaryl. In some embodiments, at least one R_{1d} is C₃-C₁₂ heteroaryl substituted with one or more R₁ₑ. In some embodiments, at least one R_{1d} is C₃-C₁₂ heteroaryl substituted with one or more R_{1z}.

In some embodiments, at least one R_{1d} is —(C₁-C₂₀ alkyl)-(C₃-C₁₂ cycloalkyl), —(C₁-C₂₀ alkyl)-(C₃-C₁₂ heterocycloalkyl), —(C₁-C₂₀(alkyl)-(C₃-C₁₂ aryl), or —(C₁-C₂₀ alkyl)-(C₃-C₁₂ heteroaryl) optionally substituted with one or more R₁ₑ.

In some embodiments, at least one R_{1d} is —(C₁-C₂₀ alkyl)-(C₃-C₁₂ cycloalkyl) optionally substituted with one or more R₁ₑ. In some embodiments, at least one R_{1d} is —(C₁-C₂₀ alkyl)-(C₃-C₁₂ cycloalkyl). In some embodiments, at least one R_{1d} is —(C₁-C₂₀ alkyl)-(C₃-C₁₂ cycloalkyl) substituted with one or more R₁ₑ. In some embodiments, at least one R_{1d} is —(C₁-C₂₀ alkyl)-(C₃-C₁₂ cycloalkyl) substituted with one or more R_{1z}.

In some embodiments, at least one R_{1d} is —(C₁-C₂₀ alkyl)-(C₃-C₁₂ heterocycloalkyl) optionally substituted with one or more R₁ₑ. In some embodiments, at least one R_{1d} is —(C₁-C₂₀ alkyl)-(C₃-C₁₂ heterocycloalkyl). In some embodiments, at least one R_{1d} is —(C₁-C₂₀ alkyl)-(C₃-C₁₂ heterocycloalkyl) substituted with one or more R₁ₑ. In some embodiments, at least one R_{1d} is —(C₁-C₂₀ alkyl)-(C₃-C₁₂ heterocycloalkyl) substituted with one or more R_{1z}.

In some embodiments, at least one R_{1d} is —(C₁-C₂₀ alkyl)-(C₃-C₁₂ aryl) optionally substituted with one or more R₁ₑ. In some embodiments, at least one R_{1d} is —(C₁-C₂₀ alkyl)-(C₃-C₁₂ aryl). In some embodiments, at least one R_{1d} is —(C₁-C₂₀ alkyl)-(C₃-C₁₂ aryl) substituted with one or more R₁ₑ. In some embodiments, at least one R_{1d} is —(C₁-C₂₀ alkyl)-(C₃-C₁₂ aryl) substituted with one or more R_{1z}.

In some embodiments, at least one R_{1d} is —(C₁-C₂₀ alkyl)-(C₃-C₁₂ heteroaryl) optionally substituted with one or more R₁ₑ. In some embodiments, at least one R_{1d} is —(C₁-C₂₀ alkyl)-(C₃-C₁₂ heteroaryl). In some embodiments, at least one R_{1d} is —(C₁-C₂₀ alkyl)-(C₃-C₁₂ heteroaryl) substituted with one or more R₁ₑ. In some embodiments, at least one R_{1d} is —(C₁-C₂₀ alkyl)-(C₃-C₁₂ heteroaryl) substituted with one or more R_{1z}.

### Variable R₁ₑ

In some embodiments, at least one R_{1c} is H.

In some embodiments, at least one R₁ₑ is halogen, C₁-C₂₀ alkyl, C₂-C₂₀ alkenyl, C₂-C₂₀ alkynyl, —OR_{1g}, —C(═O)OR_{1g}, —C(═O)N(R_{1g})₂, —N(R_{1g})₂, —N(R_{1g})C(═O)R_{1f}, —N(R_{1g})C(═O)R_{1z}, —N(R_{1g})C(═O)OR_{1g}, —OC(═O)R_{1f}, —OC(═O)R_{1z}, —OC(═O)OR_{1g}, —SR_{1g}, —N+(R_{1g})₃, —SC(═O)R_{1f}, —SC(═O)R_{1z}, —SC(═O)OR_{1g}, —SC(═O)N(R_{1g})₂, —C(═O)R_{1f}, —C(═O)R_{1z},

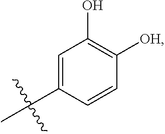 [SMILES: *c1ccc(O)c(O)c1]

or R_{1z}, wherein the C₁-C₂₀ alkyl, C₂-C₂₀ alkenyl, or C₂-C₂₀ alkynyl is optionally substituted with one or more R_{1z}.

In some embodiments, at least one R_{1c} is halogen (e.g., F, Cl, Br, I).

In some embodiments, at least one R₁ₑ is F or C₁. In some embodiments, at least one R₁ₑ is F. In some embodiments, at least one R₁ₑ is C₁.

In some embodiments, at least one R₁ₑ is C₁-C₂₀ alkyl, C₂-C₂₀ alkenyl, or C₂-C₂₀ alkynyl optionally substituted with one or more R_{1z}.

In some embodiments, at least one R₁ₑ is C₁-C₂₀ alkyl (e.g., methyl, ethyl, propyl, butyl, pentyl, hexyl, or heptyl).

In some embodiments, at least one R₁ₑ is C₁-C₂₀ alkyl (e.g., methyl, ethyl, propyl, butyl, pentyl, hexyl, or heptyl) substituted with one or more R_{1z}.

In some embodiments, at least one R_{1c} is C₂-C₂₀ alkenyl (e.g., ethenyl, propenyl, butenyl, pentenyl, or hexenyl).

In some embodiments, at least one R₁ₑ is C₂-C₂₀ alkenyl (e.g., ethenyl, propenyl, butenyl, pentenyl, or hexenyl) substituted with one or more R_{1z}.

In some embodiments, at least one R₁ₑ is C₂-C₂₀ alkynyl (e.g., ethynyl, propynyl, butynyl, pentynyl, or hexynyl).

In some embodiments, at least one R₁ₑ is C₂-C₂₀ alkynyl (e.g., ethynyl, propynyl, butynyl, pentynyl, or hexynyl) substituted with one or more R_{1z}.

In some embodiments, at least one R₁ₑ is —OR_{1g}, —C(═O)OR_{1g}, —C(═O)N(R_{1g})₂, —N(R_{1g})₂, —N(R_{1g})C(═O)R_{1f}, —N(R_{1g})C(═O)R_{1z}, —N(R_{1g})C(═O)OR_{1g}, —OC(═O)R_{1f}, —OC(═O)R_{1z}, —OC(═O)OR_{1g}, —SR_{1g}, —N⁺(R_{1g})₃, —SC(═O)R_{1f}, —SC(═O)R_{1z}, —SC(═O)OR_{1g}, —SC(═O)N(R_{1g})₂, —C(═O)R_{1f}, —C(═O)R_{1z},

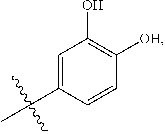 [SMILES: *c1ccc(O)c(O)c1]

### or R_{1z}.

In some embodiments, at least one R_{1c} is —OR_{1g}, —C(═O)OR_{1g}, —C(═O)N(R_{1g})₂, —N(R_{1g})₂, —N(R_{1g})C(═O)R_{1f}, —N(R_{1g})C(═O)R_{1z}, —N(R_{1g})C(═O)OR_{1g}, —OC(═O)R_{1f}, —OC(═O)R_{1z}, —OC(═O)OR_{1g}, —SR_{1g}, —N⁺(R_{1g})₃, —SC(═O)R_{1f}, —SC(═O)R_{1z}, —SC(═O)OR_{1g}, —SC(═O)N(R_{1g})₂, —C(═O)R_{1f}, or —C(═O)R_{1z}.

In some embodiments, at least one R_{1c} is —OR_{1g}.

In some embodiments, at least one R_{1c} is —OH.

In some embodiments, at least one R_{1c} is —OR_{1g}, wherein R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —OR_{1g}, wherein R_{1g} is C₁-C₂₀ alkyl, C₂-C₂₀ alkenyl, or C₂-C₂₀ alkynyl optionally substituted with one or more R_{1z}.

In some embodiments, at least one R₁ₑ is —OR_{1g}, wherein R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R_{1c} is —OR_{1g}, wherein R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —C(═O)OR_{1g}.

In some embodiments, at least one R_{1c} is —C(═O)OH.

In some embodiments, at least one R₁ₑ is —C(═O)OR_{1g}, wherein R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —C(═O)OR_{1g}, wherein R_{1g} is C₁-C₂₀ alkyl, C₂-C₂₀ alkenyl, or C₂-C₂₀ alkynyl optionally substituted with one or more R_{1z}.

In some embodiments, at least one R_{1c} is —C(═O)OR_{1g}, wherein R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R_{1c} is —C(═O)OR_{1g}, wherein R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R_{1c} is —C(═O)N(R_{1g})₂.

In some embodiments, at least one R_{1c} is —C(═O)NHR_{1g}.

In some embodiments, at least one R_{1c} is —C(═O)NH₂.

In some embodiments, at least one R_{1c} is —C(═O)N(R_{1g})₂, wherein at least one R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —C(═O)N(R_{1g})₂, wherein at least one R_{1g} is C₁-C₂₀ alkyl, C₂-C₂₀ alkenyl, or C₂-C₂₀ alkynyl optionally substituted with one or more R_{1z}.

In some embodiments, at least one R₁ₑ is —C(═O)N(R_{1g})₂, wherein at least one R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R_{1c} is —C(═O)N(R_{1g})₂, wherein at least one R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —N(R_{1g})₂.

In some embodiments, at least one R_{1c} is —NHR_{1g}.

In some embodiments, at least one R₁ₑ is —NH₂.

In some embodiments, at least one R₁ₑ is —N(R_{1g})₂, wherein at least one R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —N(R_{1g})₂, wherein at least one R_{1g} is C₁-C₂₀ alkyl, C₂-C₂₀ alkenyl, or C₂-C₂₀ alkynyl optionally substituted with one or more R_{1z}.

In some embodiments, at least one R₁ₑ is —N(R_{1g})₂, wherein at least one R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R₁ₑ is —N(R_{1g})₂, wherein at least one R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —N(R_{1g})C(═O)R_{1f}.

In some embodiments, at least one R_{1c} is —NHC(═O)R_{1f}.

In some embodiments, at least one R₁ₑ is —N(R_{1g})C(═O)H.

In some embodiments, at least one R_{1c} is —NHC(═O)H.

In some embodiments, at least one R₁ₑ is —N(R_{1g})C(═O)R_{1f}, wherein R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —N(R_{1g})C(═O)R_{1f}, wherein R_{1g} is C₁-C₂₀ alkyl, C₂-C₂₀ alkenyl, or C₂-C₂₀ alkynyl optionally substituted with one or more R_{1z}.

In some embodiments, at least one R₁ₑ is —N(R_{1g})C(═O)R_{1f}, wherein R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R_{1c} is —N(R_{1g})C(═O)R_{1f}, wherein R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —N(R_{1g})C(═O)R_{1f}, wherein R_{1f} is C₁-C₂₀ alkyl, C₂-C₂₀ alkenyl, C₂-C₂₀ alkynyl, —CH₂C(═O)OR_{1g}, —CH═CH—C(═O)OR_{1g}, —C(═O)OR_{1g}, —C(═O)N(R_{1g})₂, or R_{1z}, wherein the C₁-C₂₀ alkyl, C₂-C₂₀ alkenyl, or C₂-C₂₀ alkynyl is optionally substituted with one or more R_{1z}.

In some embodiments, at least one R₁ₑ is —N(R_{1g})C(═O)R_{1f}, wherein R_{1f} is C₁-C₂₀ alkyl, C₂-C₂₀ alkenyl, or C₂-C₂₀ alkynyl optionally substituted with one or more R_{1z}.

In some embodiments, at least one R₁ₑ is —N(R_{1g})C(═O)R_{1f}, wherein R_{1f} —CH₂C(═O)OR_{1g}, —CH═CH—C(═O)OR_{1g}, —C(═O)OR_{1g}, —C(═O)N(R_{1g})₂, or R_{1z}.

In some embodiments, at least one R₁ₑ is —N(R_{1g})C(═O)R_{1f}, wherein R_{1f} —CH₂C(═O)OR_{1g}, —CH═CH—C(═O)OR_{1g}, —C(═O)OR_{1g}, or —C(═O)N(R_{1g})₂.

In some embodiments, at least one R₁ₑ is —N(R_{1g})C(═O)R_{1z}.

In some embodiments, at least one R₁ₑ is

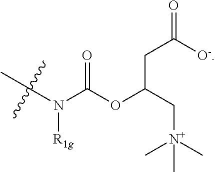

In some embodiments, at least one R₁ₑ is

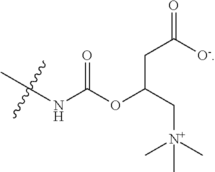

In some embodiments, at least one R₁ₑ is

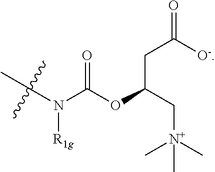

In some embodiments, at least one R₁ₑ is

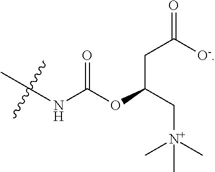

In some embodiments, at least one R₁ₑ is

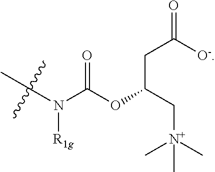

In some embodiments, at least one R₁ₑ is

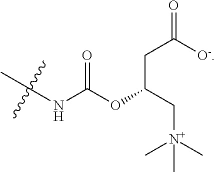

In some embodiments, at least one R₁ₑ is

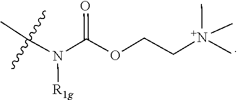

In some embodiments, at least one R₁ₑ is

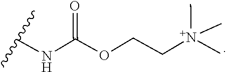 [SMILES: *NC(=O)OCC[N+](C)(C)C]

In some embodiments, at least one R₁ₑ is —N(R_{1g})C(O)OR_{1g}.

In some embodiments, at least one R₁ₑ is —N(R_{1g})C(═O)OH.

In some embodiments, at least one R₁ₑ is —NHC(═O)OR_{1g}.

In some embodiments, at least one R₁ₑ is —NHC(═O)OH.

In some embodiments, at least one R₁ₑ is —N(R_{1g})C(═O)OR_{1g}, wherein at least one R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —N(R_{1g})C(═O)OR_{1g}, wherein at least one R_{1g} is C₁-C₂₀ alkyl, C₂-C₂₀ alkenyl, or C₂-C₂₀ alkynyl optionally substituted with one or more R_{1z}.

In some embodiments, at least one R₁ₑ is —N(R_{1g})C(═O)OR_{1g}, wherein at least one R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R_{1c} is —N(R_{1g})C(═O)OR_{1g}, wherein at least one R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —OC(═O)R_{1f}.

In some embodiments, at least one R₁ₑ is —OC(═O)H.

In some embodiments, at least one R₁ₑ is —OC(═O)R_{1f}, wherein R_{1f} is C₁-C₂₀ alkyl, C₂-C₂₀ alkenyl, C₂-C₂₀ alkynyl, —CH₂C(═O)OR_{1g}, —CH═CH—C(═O)OR_{1g}, —C(═O)OR_{1g}, —C(═O)N(R_{1g})₂, or R_{1z}, wherein the C₁-C₂₀ alkyl, C₂-C₂₀ alkenyl, or C₂-C₂₀ alkynyl is optionally substituted with one or more R_{1z}.

In some embodiments, at least one R₁ₑ is —OC(═O)R_{1f}, wherein R_{1f} is C₁-C₂₀ alkyl, C₂-C₂₀ alkenyl, or C₂-C₂₀ alkynyl optionally substituted with one or more R_{1z}.

In some embodiments, at least one R₁ₑ is —OC(═O)R_{1f}, wherein R_{1f} —CH₂C(═O)OR_{1g}, —CH═CH—C(═O)OR_{1g}, —C(═O)OR_{1g}, —C(═O)N(R_{1g})₂, or R_{1z}.

In some embodiments, at least one R₁ₑ is —OC(═O)R_{1f}, wherein R_{1f} —CH₂C(═O)OR_{1g}, —CH═CH—C(═O)OR_{1g}, —C(═O)OR_{1g}, or —C(═O)N(R_{1g})₂.

In some embodiments, at least one R₁ₑ is —OC(═O)R)_{1z}.

In some embodiments, at least one R₁ₑ is

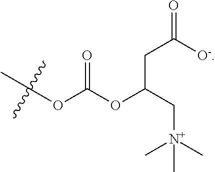

In some embodiments, at least one R₁ₑ is

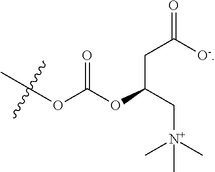

In some embodiments, at least one R₁ₑ is

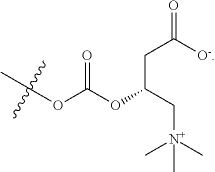

In some embodiments, at least one R₁ₑ is

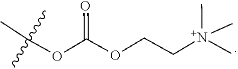 [SMILES: *OC(=O)OCC[N+](C)(C)C]

In some embodiments, at least one R₁ₑ is —OC(═O)OR_{1g}.

In some embodiments, at least one R₁ₑ is —OC(═O)OH.

In some embodiments, at least one R₁ₑ is —OC(═O)OR_{1g}, wherein R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —OC(═O)OR_{1g}, wherein R_{1g} is C₁-C₂₀ alkyl, C₂-C₂₀ alkenyl, or C₂-C₂₀ alkynyl optionally substituted with one or more R_{1z}.

In some embodiments, at least one R_{1c} is —OC(═O)OR_{1g}, wherein R_{1g} is C₃-C₁₂ cycloalkyl, C₃-C₁₂ heterocycloalkyl, C₃-C₂ aryl, or C₃-C₁₂ heteroaryl optionally substituted with one or more R_{1z}.

In some embodiments, at least one R₁ₑ is —OC(═O)OR_{1g}, wherein R_{1g} is —(C₁-C₂₀ alkyl)-(C₃-C₁₂ cycloalkyl), —(C₁-C₂₀ alkyl)-(C₃-C₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —SR_{1g}.

In some embodiments, at least one R₁ₑ is —SH.

In some embodiments, at least one R₁ₑ is —SR_{1g}, wherein R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —SR_{1g}, wherein R_{1g} is C₁-C₂₀ alkyl, C₂-C₂₀ alkenyl, or C₂-C₂₀ alkynyl optionally substituted with one or more R_{1z}.

In some embodiments, at least one R₁ₑ is —SR_{1g}, wherein R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R₁ₑ is —SR_{1g}, wherein R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₂ aryl), or —(C₁-C₂₀ alkyl)-(C₃-C₁₂ heteroaryl) optionally substituted with one or more R_{1z}.

In some embodiments, at least one R₁ₑ is —N⁺(R_{1g})₃.

In some embodiments, at least one R₁ₑ is —N⁺H(R_{1g})₂.

In some embodiments, at least one R₁ₑ is —N⁺H₂R_{1g}.

In some embodiments, at least one R₁ₑ is —N⁺H₃.

In some embodiments, at least one R_{1c} is —N⁺(R_{1g})₃, wherein at least one R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₁₀ alkyl)-(C₃-C₁₂ heteroaryl) optionally substituted with one or more R_{1z}.

In some embodiments, at least one R₁ₑ is —N⁺(R_{1g})₃, wherein at least one R_{1g} is C₁-C₂₀ alkyl, C₂-C₂₀ alkenyl, or C₂-C₂₀ alkynyl optionally substituted with one or more R_{1z}.

In some embodiments, at least one R₁ₑ is —N⁺(R_{1g})₃, wherein at least one R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R_{1c} is —N⁺(R_{1g})₃, wherein at least one R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —SC(═O)R_{1f}.

In some embodiments, at least one R₁ₑ is —SC(═O)H.

In some embodiments, at least one R₁ₑ is —SC(═O)R_{1f}, wherein R_{1f} is C₁-C₂₀ alkyl, C₂-C₂₀ alkenyl, C₂-C₂₀ alkynyl, —CH₂C(═O)OR_{1g}, —CH═CH—C(═O)OR_{1g}, —C(═O)OR_{1g}, —C(═O)N(R_{1g})₂, or R_{1z}, wherein the C₁-C₂₀ alkyl, C₂-C₂₀ alkenyl, or C₂-C₂₀ alkynyl is optionally substituted with one or more R_{1z}.

In some embodiments, at least one R₁ₑ is —SC(═O)R_{1f}, wherein R_{1f} is C₁-C₂₀ alkyl, C₂-C₂₀ alkenyl, or C₂-C₂₀ alkynyl optionally substituted with one or more R_{1z}.

In some embodiments, at least one R₁ₑ is —SC(═O)R_{1f}, wherein R_{1f} —CH₂C(═O)OR_{1g}, —CH═CH—C(═O)OR_{1g}, —C(═O)OR_{1g}, —C(═O)N(R_{1g})₂, or R_{1z}.

In some embodiments, at least one R₁ₑ is —SC(═O)R_{1f}, wherein R_{1f} —CH₂C(═O)OR_{1g}, —CH═CH—C(═O)OR_{1g}, —C(═O)OR_{1g}, or —C(═O)N(R_{1g})₂.

In some embodiments, at least one R₁ₑ is —SC(═O)R_{1z}.

In some embodiments, at least one R₁ₑ is

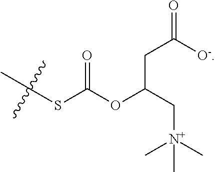

In some embodiments, at least one R₁ₑ is

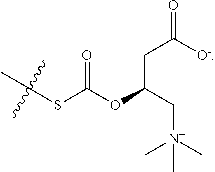

In some embodiments, at least one R₁ₑ is

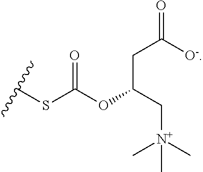

In some embodiments, at least one R₁ₑ is

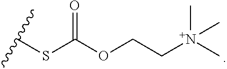 [SMILES: *SC(=O)OCC[N+](C)(C)C]

In some embodiments, at least one R_{1c} is —SC(═O)OR_{1g}.

In some embodiments, at least one R_{1c} is —SC(═O)OH.

In some embodiments, at least one R_{1c} is —SC(═O)OR_{1g}, wherein R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R_{1c} is —SC(═O)OR_{1g}, wherein R_{1g} is C₁-C₂₀ alkyl, C₂-C₂₀ alkenyl, or C₂-C₂₀ alkynyl optionally substituted with one or more R_{1z}.

In some embodiments, at least one R_{1c} is —SC(═O)OR_{1g}, wherein R_{1g} is C₃-C₁₂ cycloalkyl, C₃-C₁₂ heterocycloalkyl, C₂-C₁₂ aryl, or C₃-C₁₂ heteroaryl optionally substituted with one or more R_{1z}.

In some embodiments, at least one R_{1c} is —SC(═O)OR_{1g}, wherein R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —SC(═O)N(R_{1g})₂.

In some embodiments, at least one R_{1c} is —SC(═O)NHR_{1g}.

In some embodiments, at least one R_{1c} is —SC(═O)NH₂.

In some embodiments, at least one R_{1c} is —SC(═O)N(R_{1g})₂, wherein at least one R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —SC(═O)N(R_{1g})₂, wherein at least one R_{1g} is C₁-C₂₀ alkyl, C₂-C₂₀ alkenyl, or C₂-C₂₀ alkynyl optionally substituted with one or more R_{1z}.

In some embodiments, at least one R_{1c} is —SC(═O)N(R_{1g})₂, wherein at least one R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R_{1c} is —SC(═O)N(R_{1g})₂, wherein at least one R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₁₀ alkyl)-(C₃-C₁₂ aryl), or —(C₁-C₁₀ alkyl)-(C₃-C₁₂ heteroaryl) optionally substituted with one or more R_{1z}.

In some embodiments, at least one R₁ₑ is —C(═O)R_{1f}.

In some embodiments, at least one R₁ₑ is —C(═O)H.

In some embodiments, at least one R₁ₑ is —C(═O)R_{1f}, wherein R_{1f} is C₁-C₂₀ alkyl, C₂-C₂₀ alkenyl, C₂-C₂₀ alkynyl, —CH₂C(═O)OR_{1g}, —CH═CH—C(═O)OR_{1g}, —C(═O)OR_{1g}, —C(═O)N(R_{1g})₂, or R_{1z}, wherein the C₁-C₂₀ alkyl, C₂-C₂₀ alkenyl, or C₂-C₂₀ alkynyl is optionally substituted with one or more R_{1z}.

In some embodiments, at least one R₁ₑ is —C(═O)R_{1f}, wherein R_{1f} is C₁-C₂₀ alkyl, C₂-C₂₀ alkenyl, or C₂-C₂₀ alkynyl optionally substituted with one or more R_{1z}.

In some embodiments, at least one R₁ₑ is —C(═O)R_{1f}, wherein R_{1f} —CH₂C(═O)OR_{1g}, —CH═CH—C(═O)OR_{1g}, —C(═O)OR_{1g}, —C(═O)N(R_{1g})₂, or R_{1z}.

In some embodiments, at least one R₁ₑ is —SC(═O)R_{1f}, wherein R_{1f} —CH₂C(═O)OR_{1g}, —CH═CH—C(═O)OR_{1g}, —C(═O)OR_{1g}, or —C(═O)N(R_{1g})₂.

In some embodiments, at least one R₁ₑ is —C(═O)R_{1z}.

In some embodiments, at least one R₁ₑ is is

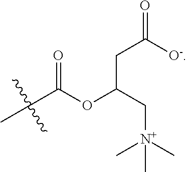

In some embodiments, at least one R₁ₑ is

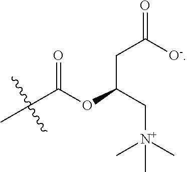

In some embodiments, at least one R₁ₑ is

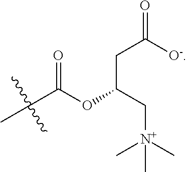

In some embodiments, at least one R₁ₑ is

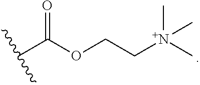 [SMILES: *C(=O)OCC[N+](C)(C)C]

In some embodiments, at least one R₁ₑ is

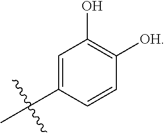 [SMILES: *c1ccc(O)c(O)c1]

In some embodiments, at least one R₁ₑ is R_{1z}.

In some embodiments, at least one R_{1c} is

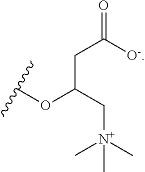

In some embodiments, at least one R₁ₑ is

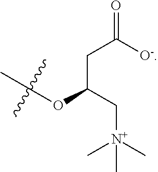

In some embodiments, at least one R₁ₑ is

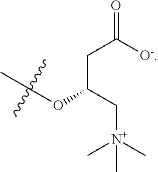

In some embodiments, at least one R₁ₑ is

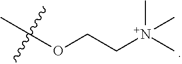 [SMILES: *OCC[N+](C)(C)C]

### Variable R_{1f}

In some embodiments, at least one R_{1f} is H.

In some embodiments, at least one R_{1f} is C₁-C₂₀ alkyl, C₂-C₂₀ alkenyl, C₂-C₂₀ alkynyl, —CH₂C(═O)OR_{1g}, —CH═CH—C(═O)OR_{1g}, —C(═O)OR_{1g}, —C(═O)N(R_{1g})₂,

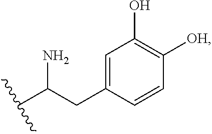 [SMILES: *C(N)Cc1ccc(O)c(O)c1]

or R_{1z}, wherein the C₁-C₂₀ alkyl, C₂-C₂₀ alkenyl, or C₂-C₂₀ alkynyl is optionally substituted with one or more R_{1z}.

In some embodiments, at least one R_{1f} is C₁-C₂₀ alkyl, C₂-C₂₀ alkenyl, or C₂-C₂₀ alkynyl optionally substituted with one or more R_{1z}.

In some embodiments, at least one R_{1f} is C₁-C₂₀ alkyl, C₂-C₂₀ alkenyl, or C₂-C₂₀ alkynyl optionally substituted with one or more R_{1z}.

In some embodiments, at least one R_{1f} is C₁-C₂₀ alkyl (e.g., methyl, ethyl, propyl, butyl, pentyl, hexyl, or heptyl).

In some embodiments, at least one R_{1f} is C₁-C₂₀ alkyl (e.g., methyl, ethyl, propyl, butyl, pentyl, hexyl, or heptyl) substituted with one or more R_{1z}.

In some embodiments, at least one R_{1f} is C₂-C₂₀ alkenyl (e.g., ethenyl, propenyl, butenyl, pentenyl, or hexenyl).

In some embodiments, at least one R_{1f} is C₂-C₂₀ alkenyl (e.g., ethenyl, propenyl, butenyl, pentenyl, or hexenyl) substituted with one or more R_{1z}.

In some embodiments, at least one R_{1f} is C₂-C₂₀ alkynyl (e.g., ethynyl, propynyl, butynyl, pentynyl, or hexynyl).

In some embodiments, at least one R_{1f} is C₂-C₂₀ alkynyl (e.g., ethynyl, propynyl, butynyl, pentynyl, or hexynyl) substituted with one or more R_{1z}.

In some embodiments, at least one R_{1f} is —CH₂C(═O)OR_{1g}, —CH═CH—C(═O)OR_{1g}, —C(═O)OR_{1g}, —C(═O)N(R_{1g})₂,

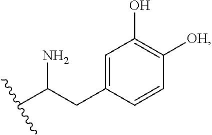 [SMILES: *C(N)Cc1ccc(O)c(O)c1]

### or R_{1z}.

In some embodiments, at least one R_{1f} is —CH₂C(═O)OR_{1g}, —CH═CH—C(═O)OR_{1g}, —C(═O)OR_{1g}, or —C(═O)N(R_{1g})₂.

In some embodiments, at least one R_{1f} is —CH₂C(═O)OR_{1g}.

In some embodiments, at least one R₁ₑ is —CH₂C(═O)OH.

In some embodiments, at least one R₁ₑ is —CH₂C(═O)OR_{1g}, wherein R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —CH₂C(═O)OR_{1g}, wherein R_{1g} is C₁-C₂₀ alkyl, C₂-C₂₀ alkenyl, or C₂-C₂₀ alkynyl optionally substituted with one or more R_{1z}.

In some embodiments, at least one R₁ₑ is —CH₂C(═O)OR_{1g}, wherein R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R₁ₑ is —CH₂C(═O)OR_{1g}, wherein R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R_{1f} is —CH═CH—C(═O)OR_{1g}.

In some embodiments, at least one R₁ₑ is —CH═CH—C(═O)OH.

In some embodiments, at least one R_{1c} is —CH═CH—C(═O)OR_{1g}, wherein R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R₁ₑ is —CH═CH—C(═O)OR_{1g}, wherein R_{1g} is C₁-C₂₀ alkyl, C₂-C₂₀ alkenyl, or C₂-C₂₀ alkynyl optionally substituted with one or more R_{1z}.

In some embodiments, at least one R₁ₑ is —CH═CH—C(═O)OR_{1g}, wherein R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R_{1c} is —CH═CH—C(═O)OR_{1g}, wherein R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R_{1f} is —C(═O)OR_{1g}.

In some embodiments, at least one R_{1c} is —C(═O)OH.

In some embodiments, at least one R_{1c} is —C(═O)OR_{1g}, wherein R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R_{1c} is —C(═O)OR_{1g}, wherein R_{1g} is C₁-C₂₀ alkyl, C₂-C₂₀ alkenyl, or C₂-C₂₀ alkynyl optionally substituted with one or more R_{1z}.

In some embodiments, at least one R₁ₑ is —C(═O)OR_{1g}, wherein R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R₁ₑ is —C(═O)OR_{1g}, wherein R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R_{1f} is —C(═O)N(R_{1g})₂.

In some embodiments, at least one R_{1f} is —C(═O)NHR_{1g}.

In some embodiments, at least one R_{1f} is —C(═O)NH₂.

In some embodiments, at least one R_{1f} is —C(═O)N(R_{1g})₂, wherein at least one R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R_{1f} is —C(═O)N(R_{1g})₂, wherein at least one R_{1g} is C₁-C₂₀ alkyl, C₂-C₂₀ alkenyl, or C₂-C₂₀ alkynyl optionally substituted with one or more R_{1z}.

In some embodiments, at least one R_{1f} is —C(═O)N(R_{1g})₂, wherein at least one R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R_{1f} is —C(═O)N(R_{1g})₂, wherein at least one R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R_{1f} is

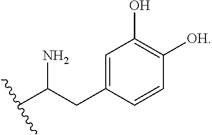 [SMILES: *C(N)Cc1ccc(O)c(O)c1]

In some embodiments, at least one R_{1f} is

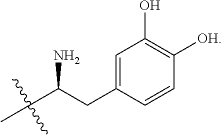 [SMILES: *[C@@H](N)Cc1ccc(O)c(O)c1]

In some embodiments, at least one R_{1f} is

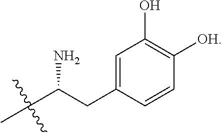 [SMILES: *[C@H](N)Cc1ccc(O)c(O)c1]

In some embodiments, at least one R_{1f} is R_{1z}.

In some embodiments, at least one R_{1f} is

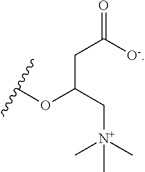

In some embodiments, at least one R_{1f} is

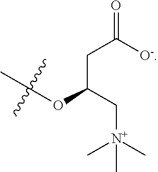

In some embodiments, at least one R_{1f} is

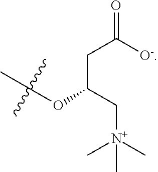

In some embodiments, at least one R_{1f} is

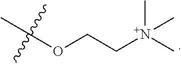 [SMILES: *OCC[N+](C)(C)C]

### Variable R_{1g}

In some embodiments, at least one R_{1g} is H.

In some embodiments, at least one R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) is optionally substituted with one or more R_{1z}.

In some embodiments, at least one R_{1g} is C₁-C₂₀ alkyl, C₂-C₂₀ alkenyl, or C₂-C₂₀ alkynyl optionally substituted with one or more R_{1z}.

In some embodiments, at least one R_{1g} is C₁-C₂₀ alkyl (e.g., methyl, ethyl, propyl, butyl, tert-butyl, pentyl, hexyl, or heptyl).

In some embodiments, at least one R_{1g} is methyl.

In some embodiments, at least two R_{1g} are methyl.

In some embodiments, at least one R_{1g} is tert-butyl.

In some embodiments, at least one R_{1g} is C₁-C₂₀ alkyl (e.g., methyl, ethyl, propyl, butyl, tert-butyl, pentyl, hexyl, or heptyl) substituted with one or more R_{1z}.

In some embodiments, at least one R_{1g} is C₂-C₂₀ alkenyl (e.g., ethenyl, propenyl, butenyl, pentenyl, or hexenyl).

In some embodiments, at least one R_{1g} is C₂-C₂₀ alkenyl (e.g., ethenyl, propenyl, butenyl, pentenyl, or hexenyl) substituted with one or more R_{1z}.

In some embodiments, at least one R_{1g} is C₂-C₂₀ alkynyl (e.g., ethynyl, propynyl, butynyl, pentynyl, or hexynyl).

In some embodiments, at least one R_{1g} is C₂-C₂₀ alkynyl (e.g., ethynyl, propynyl, butynyl, pentynyl, or hexynyl) substituted with one or more R_{1z}.

In some embodiments, at least one R_{1g} is C₃-C₁₂ cycloalkyl, C₃-C₁₂ heterocycloalkyl, C₃-C₁₂ aryl, or C₃-C₁₂ heteroaryl optionally substituted with one or more R_{1z}.

In some embodiments, at least one R_{1g} is C₃-C₁₂ cycloalkyl optionally substituted with one or more R_{1z}. In some embodiments, at least one R_{1g} is C₃-C₁₂ cycloalkyl. In some embodiments, at least one R_{1g} is C₃-C₁₂ cycloalkyl substituted with one or more R_{1z}.

In some embodiments, at least one R_{1g} is C₃-C₁₂ heterocycloalkyl optionally substituted with one or more R_{1z}. In some embodiments, at least one R_{1g} is C₃-C₁₂ heterocycloalkyl. In some embodiments, at least one R_{1g} is C₃-C₁₂ heterocycloalkyl substituted with one or more R_{1z}.

In some embodiments, at least one R_{1g} is C₃-C₁₂ aryl optionally substituted with one or more R_{1z}. In some embodiments, at least one R_{1g} is C₃-C₁₂ aryl. In some embodiments, at least one R_{1g} is C₃-C₁₂ aryl substituted with one or more R_{1z}.

In some embodiments, at least one R_{1g} is C₃-C₁₂ heteroaryl optionally substituted with one or more R_{1z}. In some embodiments, at least one R_{1g} is C₃-C₁₂ heteroaryl. In some embodiments, at least one R_{1g} is C₃-C₁₂ heteroaryl substituted with one or more R_{1z}.

In some embodiments, at least one R_{1g} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R_{1z}.

In some embodiments, at least one R_{1g} is —(C₁-C₂₀ alkyl)-(C₃-C₁₂ cycloalkyl) optionally substituted with one or more R_{1z}. In some embodiments, at least one R_{1g} is —(C₁-C₂₀ alkyl)-(C₃-C₁₂ cycloalkyl). In some embodiments, at least one R_{1g} is —(C₁-C₂₀ alkyl)-(C₃-C₁₂ cycloalkyl) substituted with one or more R_{1z}.

In some embodiments, at least one R_{1g} is —(C₁-C₂₀ alkyl)-(C₃-C₁₂ heterocycloalkyl) optionally substituted with one or more R_{1z}. In some embodiments, at least one R_{1g} is —(C₁-C₂₀ alkyl)-(C₃-C₁₂ heterocycloalkyl). In some embodiments, at least one R_{1g} is —(C₁-C₂₀ alkyl)-(C₃-C₁₂ heterocycloalkyl) substituted with one or more R_{1z}.

In some embodiments, at least one R_{1g} is —(C₁-C₂₀ alkyl)-(C₃-C₁₂ aryl) optionally substituted with one or more R_{1z}. In some embodiments, at least one R_{1g} is —(C₁-C₂₀ alkyl)-(C₃-C₁₂ aryl). In some embodiments, at least one R_{1g} is —(C₁-C₂₀ alkyl)-(C₃-C₁₂ aryl) substituted with one or more R_{1z}.

In some embodiments, at least one R_{1g} is —(C₁-C₂₀ alkyl)-(C₃-C₁₂ heteroaryl) optionally substituted with one or more R_{1z}. In some embodiments, at least one R_{1g} is —(C₁-C₂₀ alkyl)-(C₃-C₁₂ heteroaryl). In some embodiments, at least one R_{1g} is —(C₁-C₂₀ alkyl)-(C₃-C₁₂ heteroaryl) substituted with one or more R_{1z}.

### Variable R_{1z}

In some embodiments, at least one R_{1z} is

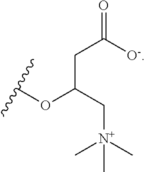

In some embodiments, at least one R_{1z} is

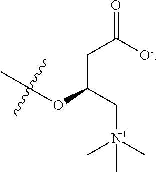

In some embodiments, at least one R_{1z} is

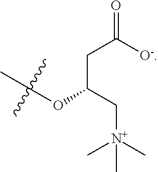

In some embodiments, at least one R_{1z} is

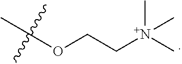 [SMILES: *OCC[N+](C)(C)C]

In some embodiments, all of the one or more R_{1z} is

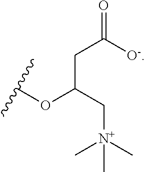

In some embodiments, all of the one or more R_{1z} is

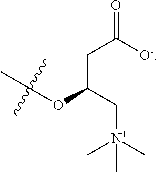

In some embodiments, all of the one or more R_{1z} is

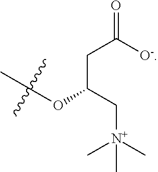

In some embodiments, all of the one or more R_{1z} is

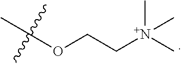 [SMILES: *OCC[N+](C)(C)C]

In some embodiments, at least one of the two or more R_{1z} is

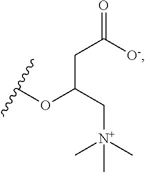

and at least one of the two or more R_{1z} is

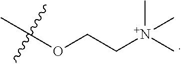 [SMILES: *OCC[N+](C)(C)C]

In some embodiments, at least one of the two or more R_{1z} is

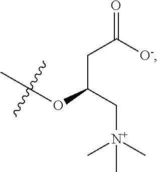

and at least one of the two or more R_{1z} is

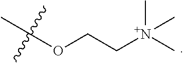 [SMILES: *OCC[N+](C)(C)C]

In some embodiments, at least one of the two or more R_{1z} is

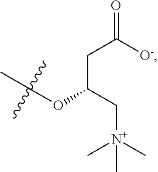

and at least one of the two or more R_{1z} is

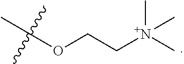 [SMILES: *OCC[N+](C)(C)C]

### Variables n, p, q, and r

In some embodiments, n is from 0 to 20, from 0 to 15, from 0 to 10, from 0 to 6, from 0 to 4, or from 0 to 2.

In some embodiments, n is from 1 to 20, from 2 to 20, from 3 to 20, from 4 to 20, from 5 to 20, from 6 to 20, from 7 to 20, from 8 to 20, from 9 to 20, from 10 to 20, from 11 to 20, from 12 to 20, from 13 to 20, from 14 to 20, from 15 to 20, from 16 to 20, from 17 to 20, from 18 to 20, or from 19 to 20.

In some embodiments, n is 0.

In some embodiments, n is from 1 to 10 (e.g., 1, 2, 3, 4, 5, 6, 7, 8, 9, or 10). In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, n is from 11 to 20 (e.g., 11, 12, 13, 14, 15, 16, 17, 18, 19, or 20). In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18. In some embodiments, n is 19. In some embodiments, n is 20.

In some embodiments, p is from 0 to 20, from 0 to 15, from 0 to 10, from 0 to 6, from 0 to 4, or from 0 to 2.

In some embodiments, p is from 1 to 20, from 2 to 20, from 3 to 20, from 4 to 20, from 5 to 20, from 6 to 20, from 7 to 20, from 8 to 20, from 9 to 20, from 10 to 20, from 11 to 20, from 12 to 20, from 13 to 20, from 14 to 20, from 15 to 20, from 16 to 20, from 17 to 20, from 18 to 20, or from 19 to 20.

In some embodiments, p is 0.

In some embodiments, p is from 1 to 10 (e.g., 1, 2, 3, 4, 5, 6, 7, 8, 9, or 10). In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10.

In some embodiments, p is from 11 to 20 (e.g., 11, 12, 13, 14, 15, 16, 17, 18, 19, or 20). In some embodiments, p is 11. In some embodiments, p is 12. In some embodiments, p is 13. In some embodiments, p is 14. In some embodiments, p is 15. In some embodiments, p is 16. In some embodiments, p is 17. In some embodiments, p is 18. In some embodiments, p is 19. In some embodiments, p is 20.

In some embodiments, q is from 0 to 20, from 0 to 15, from 0 to 10, from 0 to 6, from 0 to 4, or from 0 to 2.

In some embodiments, q is from 1 to 20, from 2 to 20, from 3 to 20, from 4 to 20, from 5 to 20, from 6 to 20, from 7 to 20, from 8 to 20, from 9 to 20, from 10 to 20, from 11 to 20, from 12 to 20, from 13 to 20, from 14 to 20, from 15 to 20, from 16 to 20, from 17 to 20, from 18 to 20, or from 19 to 20.

In some embodiments, q is 0.

In some embodiments, q is from 1 to 10 (e.g., 1, 2, 3, 4, 5, 6, 7, 8, 9, or 10). In some embodiments, q is 1. In some embodiments, q is 2. In some embodiments, q is 3. In some embodiments, q is 4. In some embodiments, q is 5. In some embodiments, q is 6. In some embodiments, q is 7. In some embodiments, q is 8. In some embodiments, q is 9. In some embodiments, q is 10.

In some embodiments, r is from 11 to 20 (e.g., 11, 12, 13, 14, 15, 16, 17, 18, 19, or 20). In some embodiments, r is 11. In some embodiments, r is 12. In some embodiments, r is 13. In some embodiments, r is 14. In some embodiments, r is 15. In some embodiments, r is 16. In some embodiments, r is 17. In some embodiments, r is 18. In some embodiments, r is 19. In some embodiments, r is 20.

In some embodiments, r is from 0 to 20, from 0 to 15, from 0 to 10, from 0 to 6, from 0 to 4, or from 0 to 2.

In some embodiments, r is from 1 to 20, from 2 to 20, from 3 to 20, from 4 to 20, from 5 to 20, from 6 to 20, from 7 to 20, from 8 to 20, from 9 to 20, from 10 to 20, from 11 to 20, from 12 to 20, from 13 to 20, from 14 to 20, from 15 to 20, from 16 to 20, from 17 to 20, from 18 to 20, or from 19 to 20.

In some embodiments, r is 0.

In some embodiments, r is from 1 to 10 (e.g., 1, 2, 3, 4, 5, 6, 7, 8, 9, or 10). In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, r is from 11 to 20 (e.g., 11, 12, 13, 14, 15, 16, 17, 18, 19, or 20). In some embodiments, r is 11. In some embodiments, r is 12. In some embodiments, r is 13. In some embodiments, r is 14. In some embodiments, r is 15. In some embodiments, r is 16. In some embodiments, r is 17. In some embodiments, r is 18. In some embodiments, r is 19. In some embodiments, r is 20.

### Variable R₂

In some embodiments, R₂ is H.

In some embodiments, R₂ is —C(═O)R_{1b}, —C(═O)OR_{1c}, —C(═O)N(R_{1c})₂, —C(═O)R_{1z}, —C(═O)—(C═O)R_{1b}, —C(═O)—C(═O)OR_{1c}, —C(═O)—C(═O)N(R_{1c})₂, —C(═O)—C(═O)R_{1z}, —C(═O)—CH═CH—C(═O)OR_{1c}. —C(═O)—CH₂—CH₂—C(═O)OR_{1c},

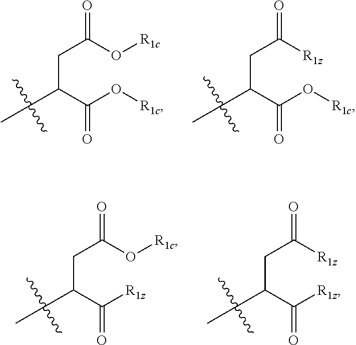

—C(═O)—CH═CH—C(═O)—R_{1z}, —C(═O)—CH₂—CH₂—C(═O)—R_{1z}, Si(R_{1g})₃,

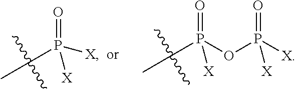

In some embodiments, R₂ is —C(═O)R_{1b}.

In some embodiments, R₂ is —C(═O)H.

In some embodiments, R₂ is —C(═O)R_{1b}, wherein R_{1b} is C₁-C₂₀ alkyl, C₂-C₂₀ alkenyl, C₂-C₂₀ alkynyl, —(CH₂)_{q}—C(═O)OR_{1c}, —CH₂—C(═O)—(CH₂)_{q}—C(═O)OR_{1c}, —CH₂—[C(═O)CH₂]ₚ—[CH₂]_{q}—C(═O)OR_{1c}, —CH═CH—C(═O)OR_{1c}. —C(═O)OR_{1c}, —C(═O)N(R_{1c})₂, or R_{1z}, wherein the C₁-C₂₀ alkyl, or C₂-C₂₀ alkenyl or C₂-C₂₀ alkynyl is optionally substituted with one or more R₁ₑ.

In some embodiments, R₂ is —C(═O)R_{1b}, wherein R_{1b} is C₁-C₂₀ alkyl, C₂-C₂₀ alkenyl, or C₂-C₂₀ alkynyl optionally substituted with one or more R₁ₑ.

In some embodiments, R₂ is —C(═O)R_{1b}, wherein R_{1b} is C₁-C₂₀ alkyl.

In some embodiments, R₂ is —C(═O)R_{1b}, wherein R_{1b} is C₁ alkyl.

In some embodiments, R₂ is

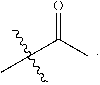 [SMILES: *C(C)=O]

In some embodiments, R₂ is —C(═O)R_{1b}, wherein R_{1b} is C₁-C₂₀ alkyl substituted with one or more R₁ₑ.

In some embodiments, R₂ is —C(═O)R_{1b}, wherein R_{1b} is C₁-C₂₀ alkyl substituted with one or more R₁ₑ, and wherein R₁ₑ is —N(R_{1g})₂.

In some embodiments, R₂, is —C(═O)R_{1b}, wherein R_{1b} is C₁-C₂₀ alkyl substituted with one or more R₁ₑ, wherein R₁ₑ is —N(R_{1g})₂, and wherein each R_{1g} is H.

In some embodiments, R₂ is

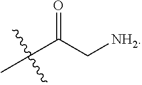 [SMILES: *C(=O)CN]

In some embodiments, R₂ is —C(═O)R_{1b}, wherein R_{1b} is C₁-C₂₀ alkyl substituted with one or more R₁ₑ, and wherein R_{1c} is C₁-C₂₀ alkyl.

In some embodiments, R₂ is —C(═O)R_{1b}, wherein R_{1b} is C₁-C₂₀ alkyl substituted with one or more R₁ₑ, and wherein R_{1c} is —N(R_{1g})₂ and C₁-C₂₀ alkyl.

In some embodiments, R₂ is —C(═O)R_{1b}, wherein R_{1b} is C₁-C₂₀ alkyl substituted with one or more R₁ₑ, wherein R₁ₑ is —N(R_{1g})₂ and C₁-C₂₀ alkyl, and wherein each R_{1g} is H.

In some embodiments, R₂ is

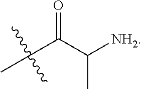 [SMILES: *C(=O)C(C)N]

In some embodiments, R₂ is

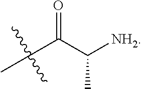 [SMILES: *C(=O)[C@@H](C)N]

In some embodiments, R₂ is

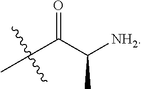 [SMILES: *C(=O)[C@H](C)N]

In some embodiments, R₂ is —C(═O)R_{1b}, wherein R_{1b} is —(CH₂)_{q}—C(═O)OR_{1c}, —CH₂—C(═O)—(CH₂)_{q}—C(═O)OR_{1c}, —CH₂—[C(═O)CH₂]ₚ—[CH₂]_{q}—C(═O)OR_{1c}, —CH═CH—C(═O)OR_{1c}, —C(═O)OR_{1c}, or —C(═O)N(R_{1c})₂.

In some embodiments, R₂ is —(CH₂)_{q}—C(═O)OR_{1c}.

In some embodiments, R₂ is —CH₂CH₂—C(═O)OR_{1c}.

In some embodiments, R₂ is —CH₂—C(═O)—(CH₂)_{q}—C(═O)OR_{1c}.

In some embodiments, R₂ is —CH₂—C(═O)—CH₂CH₂—C(═O)OR_{1c}.

In some embodiments, R₂ is —C(═O)—CH═CH—C(═O)OR_{1c}.

In some embodiments, R₂ is —C(═O)R_{1z}.

In some embodiments, R₂ is —C(═O)—(C═O)R_{1b}.

In some embodiments, R₂ is —C(═O)—C(═O)OR_{1c}.

In some embodiments, R₂ is —C(═O)—C(═O)N(R_{1c})₂.

In some embodiments, R₂ is —C(═O)—C(═O)R_{1z},

In some embodiments, R₂ is

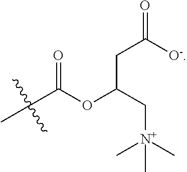

In some embodiments, R₂ is

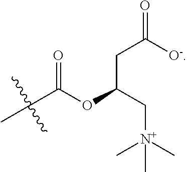

In some embodiments, R₂ is

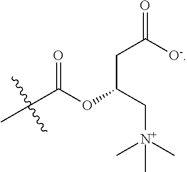

In some embodiments, R₂, is

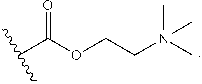 [SMILES: *C(=O)OCC[N+](C)(C)C]

In some embodiments, R₂ is —C(═O)OR_{1c}.

In some embodiments, R₂ is —C(═O)OH.

In some embodiments, R₂ is —C(═O)OR_{1c}, wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₂ is —C(═O)OR_{1c}, wherein R_{1c} is C₁-C₂₀ alkyl, C₂-C₂₀ alkenyl, or C₂-C₂₀ alkynyl optionally substituted with one or more R₁ₑ.

In some embodiments, R₂ is —C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₂ is —C(═O)OR_{1c}, wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₂ is —C(═O)N(R_{1c})₂.

In some embodiments, R₂ is —C(═O)N(R_{1c})₂, wherein at least one R_{1c} is H.

In some embodiments, R₂ is —C(═O)N(R_{1c})₂, wherein at least one R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₂ is —C(═O)N(R_{1c})₂, wherein at least one R_{1c} is C₁-C₂₀ alkyl, C₂-C₂₀ alkenyl, or C₂-C₂₀ alkynyl optionally substituted with one or more R₁ₑ.

In some embodiments, R₂ is —C(═O)N(R_{1c})₂, wherein at least one R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₂ is —C(═O)N(R_{1c})₂, wherein at least one R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₂ is —C(═O)—CH═CH—C(═O)OR_{1c}.

In some embodiments, R₂ is —C(═O)—CH═CH—C(═O)OH.

In some embodiments, R₂ is —C(═O)—CH═CH—C(═O)OR_{1c}, wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₂ is —C(═O)—CH═CH—C(═O)OR_{1c}, wherein R_{1c} is C₁-C₂₀ alkyl, C₂-C₂₀ alkenyl, or C₂-C₂₀ alkynyl optionally substituted with one or more R₁ₑ.

In some embodiments, R₂ is —C(═O)—CH═CH—C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₂ is —C(═O)—CH═CH—C(═O)OR_{1c}, wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₂ is —C(═O)—CH₂—CH₂—C(═O)OR_{1c}.

In some embodiments, R₂ is —C(═O)—CH₂—CH₂—C(═O)OH.

In some embodiments, R₂ is —C(═O)—CH₂—CH₂—C(═O)OR_{1c}, wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₂ is —C(═O)—CH₂—CH₂—C(═O)OR_{1c}, wherein R_{1c} is C₁-C₂₀ alkyl, C₂-C₂₀ alkenyl, or C₂-C₂₀ alkynyl optionally substituted with one or more R₁ₑ.

In some embodiments, R₂ is —C(═O)—CH₂—CH₂—C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₂ is —C(═O)—CH₂—CH₂—C(═O)OR_{1c}, wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₂ is

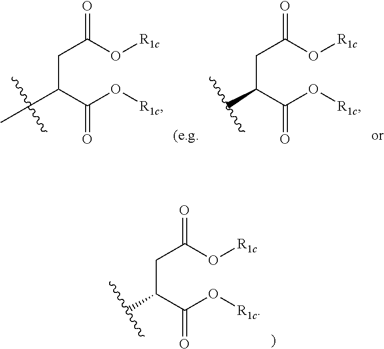

In some embodiments, R₂ is

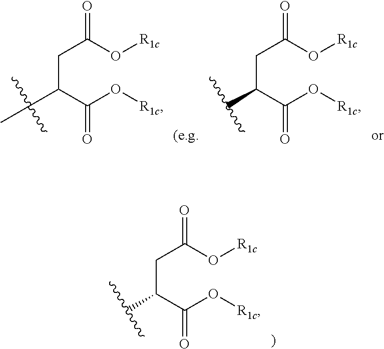

wherein at least one R_{1c} is H.

In some embodiments, R₂ is

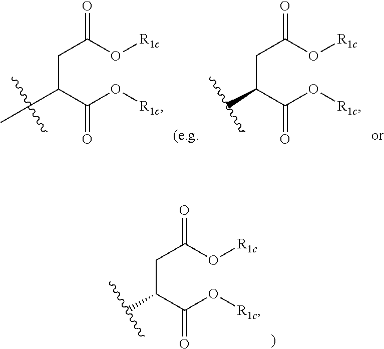

wherein at least one R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₂ is

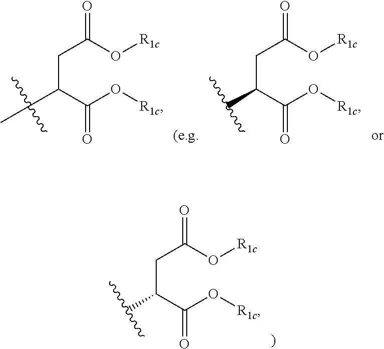

wherein at least one R_{1c} is C₁-C₂₀ alkyl, C₂-C₂₀ alkenyl, or C₂-C₂₀ alkynyl optionally substituted with one or more R₁ₑ.

In some embodiments, R₂ is

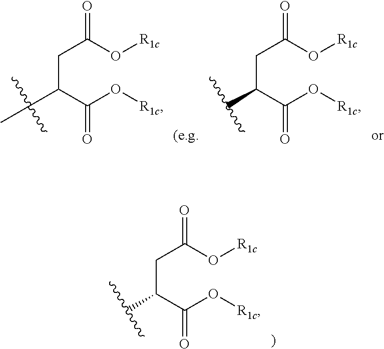

wherein at least one R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₂ is

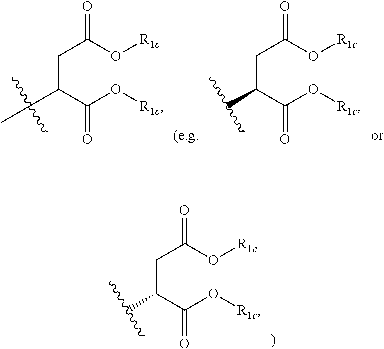

wherein at least one R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₂ is

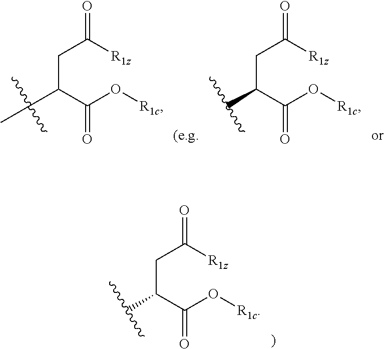

In some embodiments, R₂ is

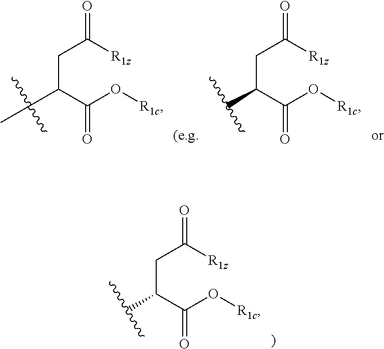

wherein R_{1c} is H.

In some embodiments, R₂ is

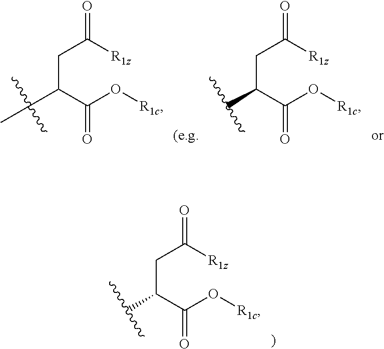

wherein R_{1c} is C₁-C₂₀ alkyl, C₂-C₂₀ alkenyl, C₂-C₂₀ alkynyl, C₃-C₁₂ cycloalkyl, C₃-C₁₂ tse heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₂ is

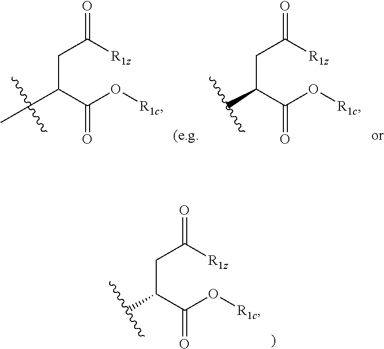

wherein R_{1c} is C₁-C₂₀ alkyl, C₂-C₂₀ alkenyl, or C₂-C₂₀ alkynyl optionally substituted with one or more R₁ₑ.

In some embodiments, R₂ is

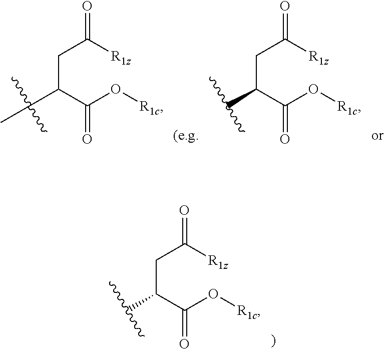

wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₂ is

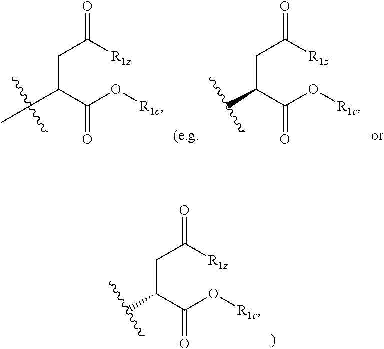

wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₂ heteroaryl) optionally substituted with one or more R₁ₑ.

In some embodiments, R₂ is

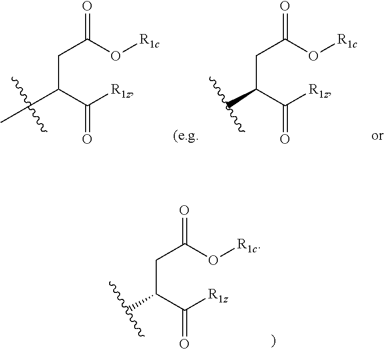

In some embodiments, R₂ is

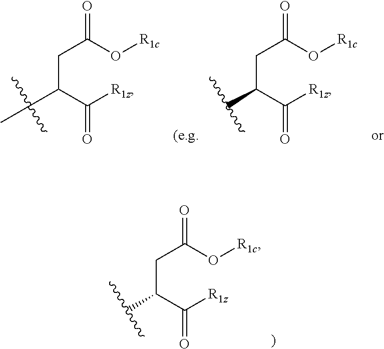

wherein R_{1c} is H.

In some embodiments, R₂ is

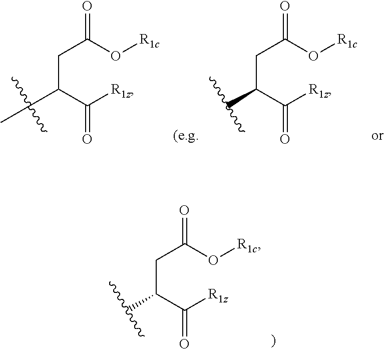

wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₂ is

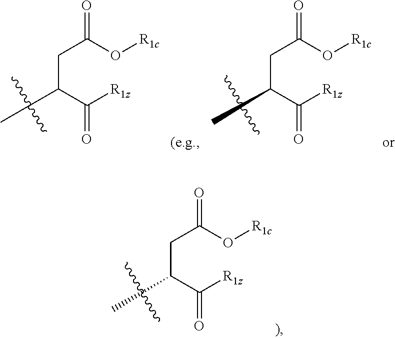

wherein R_{1c} is C₁-C₂₀ alkyl, C₂-C₂₀ alkenyl, or C₂-C₂₀ alkynyl optionally substituted with one or more R₁ₑ.

In some embodiments, R₂ is

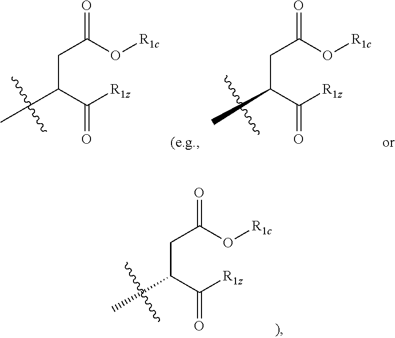

wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₂ is

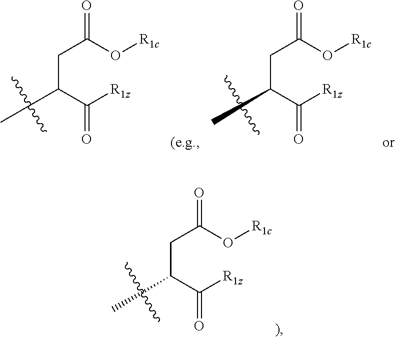

wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₂ is

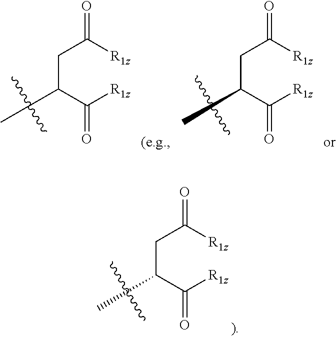

In some embodiments, R₂ is —C(═O)—CH═CH—C(═O)—R_{1z}.

In some embodiments, R₂ is —C(═O)—CH═CH—C(═O)—R_{1z}, wherein R_{1z} is

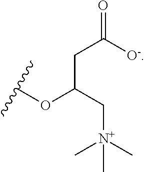

In some embodiments, R₂ is —C(═O)—CH═CH—C(═O)—R_{1z}, wherein R_{1z} is

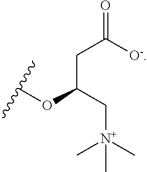

In some embodiments, R₂ is —C(═O)—CH═CH—C(═O)—R_{1z}, wherein R_{1z} is

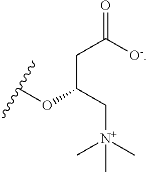

In some embodiments, R₂ is —C(═O)—CH═CH—C(═O)—R_{1z}, wherein R_{1z} is

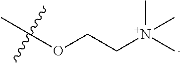 [SMILES: *OCC[N+](C)(C)C]

In some embodiments, R₂ is —C(═O)—CH₂—CH₂—C(═O)—R_{1z}.

In some embodiments, R₂ is —C(═O)—CH₂—CH₂—C(═O)—R_{1z}, wherein R_{1z} is

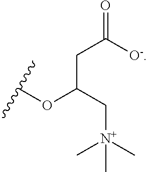

In some embodiments, R₂ is —C(═O)—CH₂—CH₂—C(═O)—R_{1z}, wherein R_{1z} is

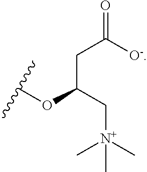

In some embodiments, R₂ is —C(═O)—CH₂—CH₂—C(═O)—R_{1z}, wherein R_{1z} is

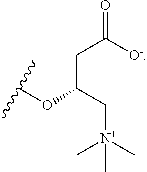

In some embodiments, R₂ is —C(═O)—CH₂—CH₂—C(═O)—R_{1z}, wherein R_{1z} is

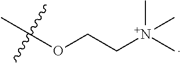 [SMILES: *OCC[N+](C)(C)C]

In some embodiments, R₂ is Si(R_{1g})₃.

In some embodiments, R₂ is Si(R_{1g})₃, wherein at least one R_{1g} is C₁-C₂₀ alkyl.

In some embodiments, R₂ is Si(R_{1g})₃, wherein at least two R_{1g} are C₁-C₂₀ alkyl.

In some embodiments, R₂ is Si(R_{1g})₃, wherein all R_{1g} are C₁-C₂₀ alkyl.

In some embodiments, R₂ is

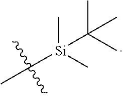 [SMILES: *[Si](C)(C)C(C)(C)C]

In some embodiments, R₂ is

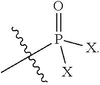

In some embodiments, R₂ is

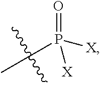

wherein at least one X is —OR_{1c}, —SR_{1c}, or —N(R_{1c})₂.

In some embodiments, R₂ is

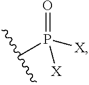

wherein one of the two X is —OR_{1c}, —SR_{1c}, or —N(R_{1c})₂.

In some embodiments, R₂ is

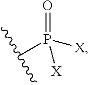

wherein each X is independently —OR_{1c}, —SR_{1c}, or —N(R_{1c})₂.

In some embodiments, R₂ is

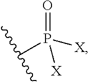

wherein at least one X is —OR_{1c}.

In some embodiments, R₂ is

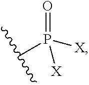

wherein one of the two X is —OR_{1c}.

In some embodiments, R₂ is

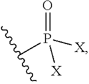

wherein each X is independently —OR_{1c}.

In some embodiments, R₂ is

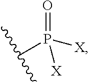

wherein at least one X is —SR_{1c}.

In some embodiments, R₂ is

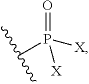

wherein one of the two X is —SR_{1c}.

In some embodiments, R₂ is

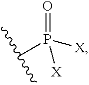

wherein each X is independently —SR_{1c}.

In some embodiments, R₂ is

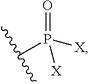

wherein at least one X is —N(R_{1c})₂.

In some embodiments, R₂ is

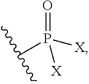

wherein one of the two X is —N(R_{1c})₂.

In some embodiments, R₂ is

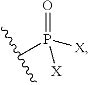

wherein each X is independently —N(R_{1c})₂.

In some embodiments, R₂ is

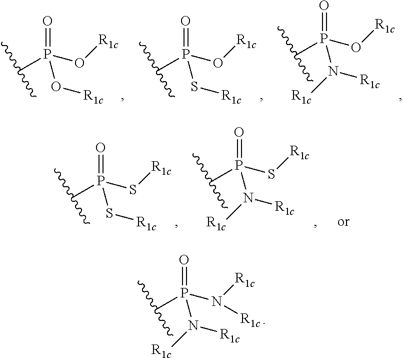

In some embodiments, R₂ is

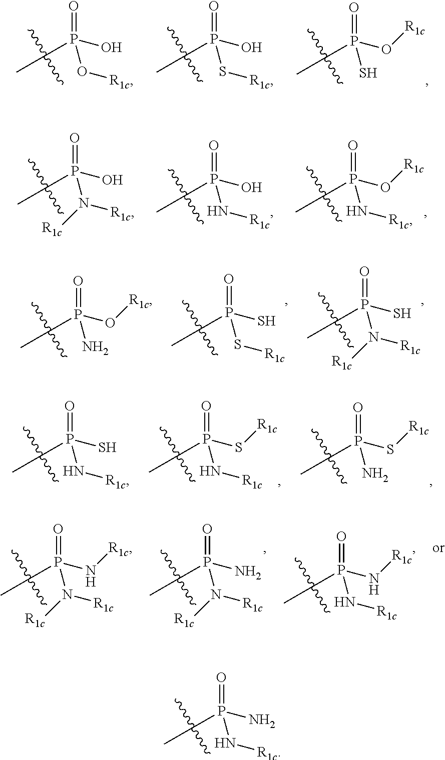

In some embodiments, R₂ is

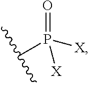

wherein at least one X is

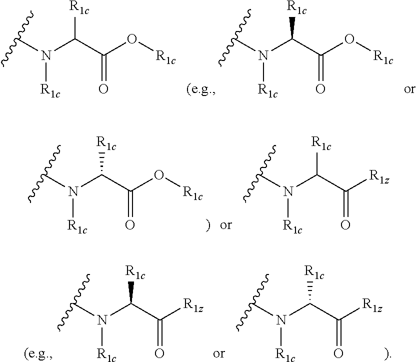

In some embodiments, R₂ is

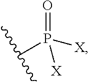

wherein one of the two X is

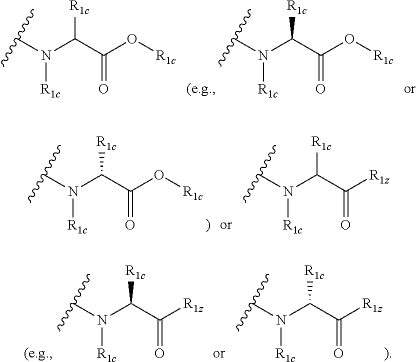

In some embodiments, R₂ is

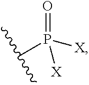

wherein each X independently is

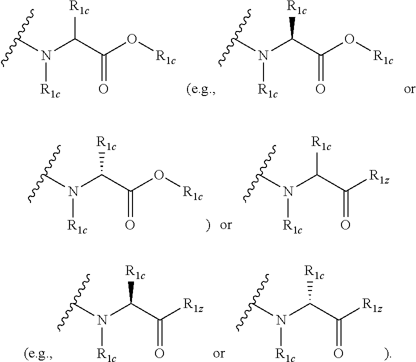

In some embodiments, R₂ is

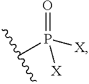

wherein at least one X is

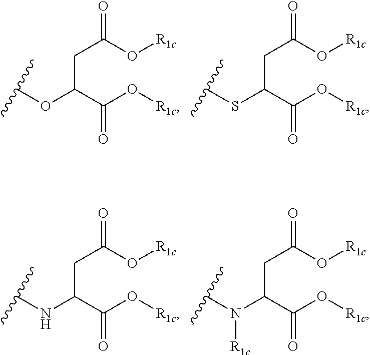

### or R_{1z}.

In some embodiments, R₂ is

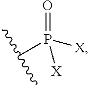

wherein one of the two X is

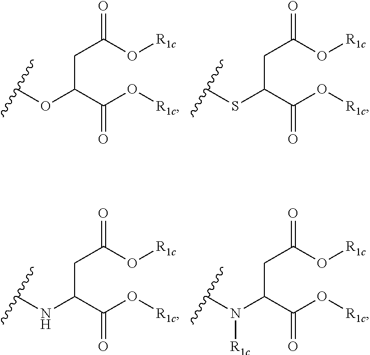

### or R_{1z}.

In some embodiments, R₂ is

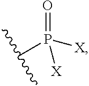

wherein each X is

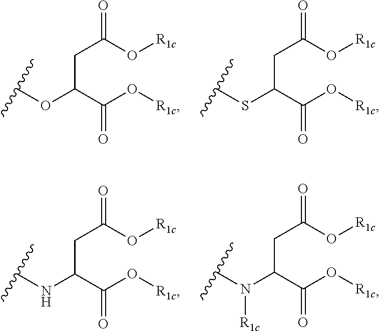

### or R_{1z}.

In some embodiments, R₂ is

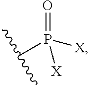

wherein at least one X is

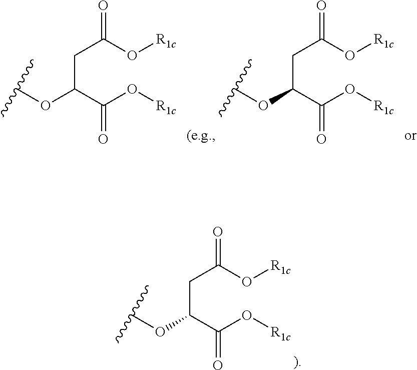

In some embodiments, R₂ is

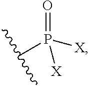

wherein one of the two X is

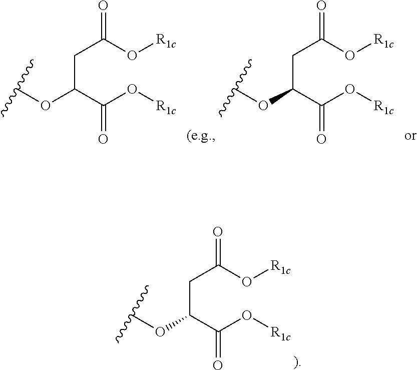

In some embodiments, R₂ is

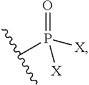

wherein each X is

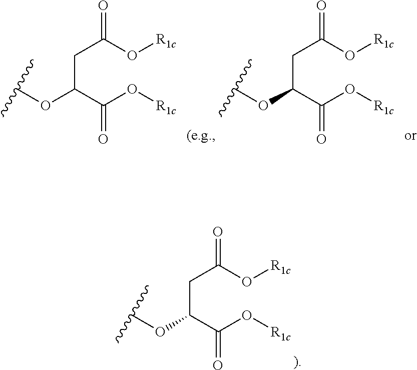

In some embodiments, R₂ is

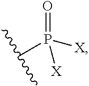

wherein at least one X is

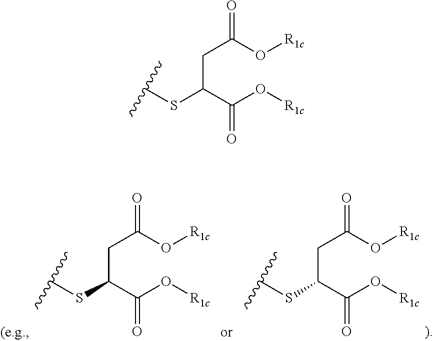

In some embodiments, R₂ is

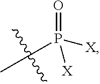

wherein one of the two X is

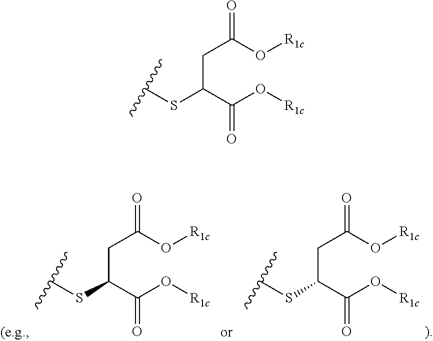

In some embodiments, R₂ is

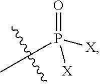

wherein each X is

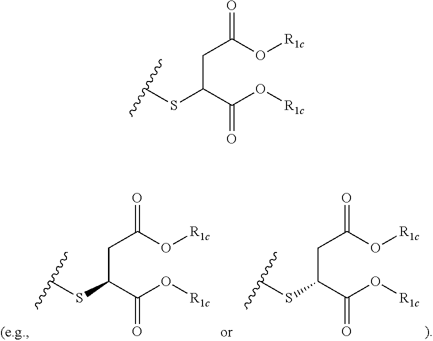

In some embodiments, R₂ is

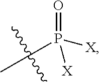

wherein at least one X is

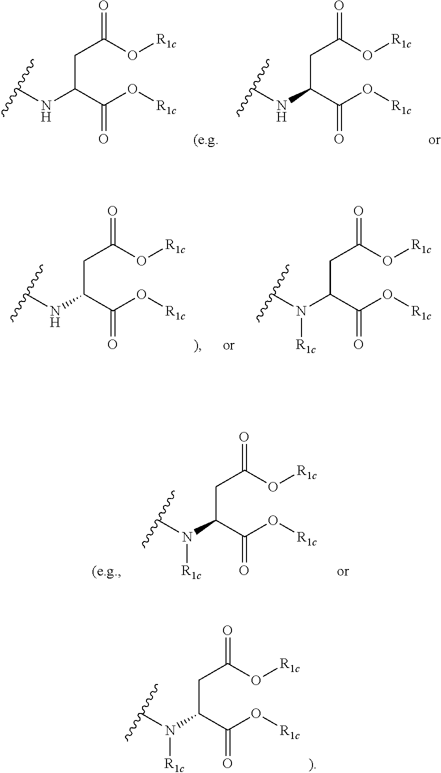

In some embodiments, R₂ is

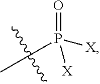

wherein one of the two X is

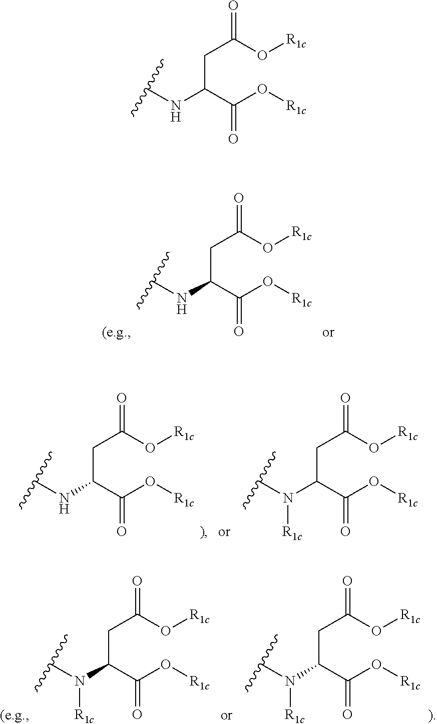

In some embodiments, R₂ is

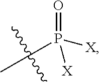

wherein each X is

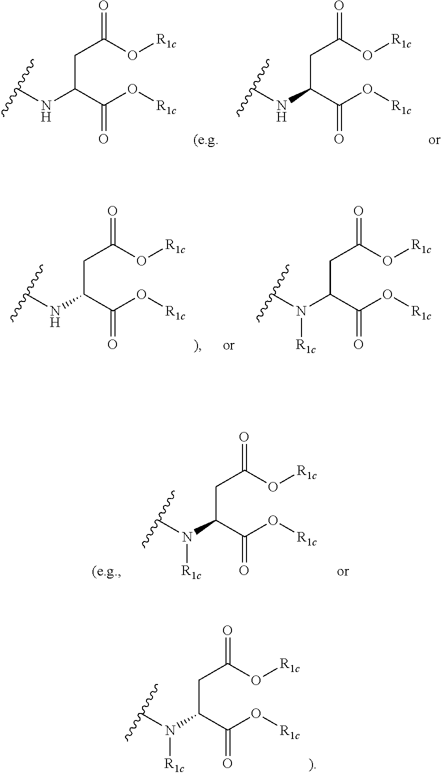

In some embodiments, R₂ is

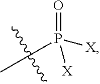

wherein at least one X is R_{1z}

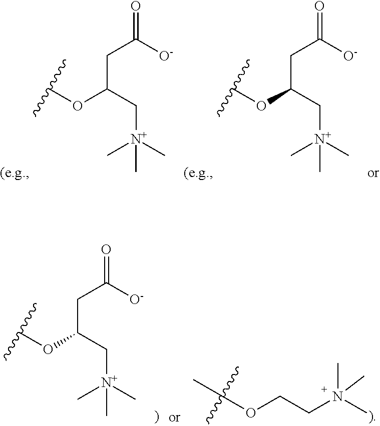

In some embodiments, R₂ is

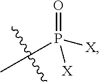

wherein one of the two X is R_{1z}

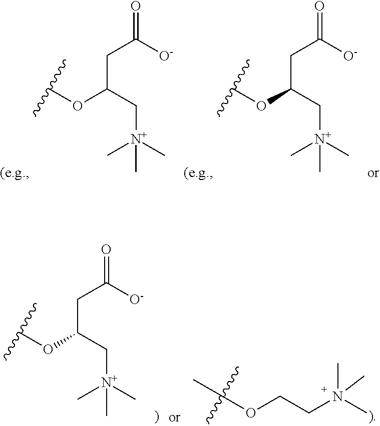

In some embodiments, R₂ is

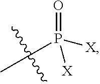

wherein each X is R_{1z}

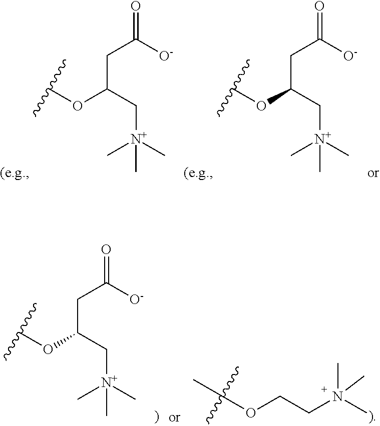

In some embodiments, R₂ is

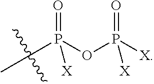

In some embodiments, R₂ is

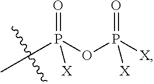

wherein at least one X is —OR_{1c}, —SR_{1c}, or —N(R_{1c})₂.

In some embodiments, R₂ is

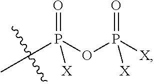

wherein two of the three X is —OR_{1c}, —SR_{1c}, or —N(R_{1c})₂.

In some embodiments, R₂ is

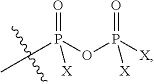

wherein each X is independently —OR_{1c}, —SR_{1c}, or —N(R_{1c})₂.

In some embodiments, R₂ is

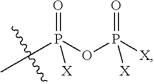

wherein at least one X is —OR_{1c}.

In some embodiments, R₂ is

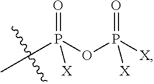

wherein two of the three X is —OR_{1c}.

In some embodiments, R₂ is

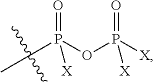

wherein each X is independently —OR_{1c}.

In some embodiments, R₂ is

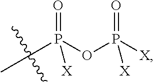

wherein at least one X is —SR_{1c}.

In some embodiments, R₂ is

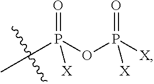

wherein two of the three X is —SR_{1c}.

In some embodiments, R₂ is

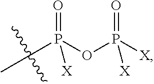

wherein each X is independently —SR_{1c}.

In some embodiments, R₂ is

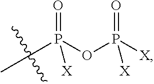

wherein at least one X is —N(R_{1c})₂.

In some embodiments, R₂ is

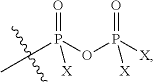

wherein two of the three X is —N(R_{1c})₂.

In some embodiments, R₂ is

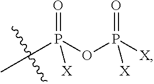

wherein each X is independently —N(R_{1c})₂.

In some embodiments, R₂ is

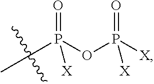

wherein at least one X is

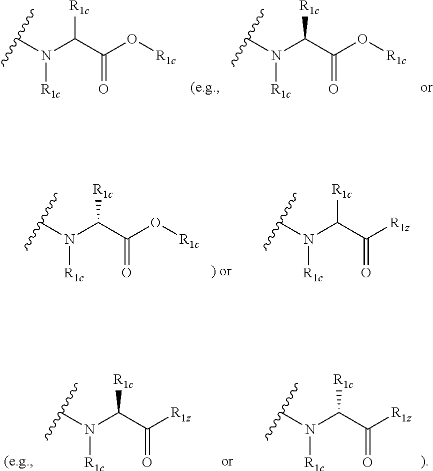

In some embodiments, R₂ is

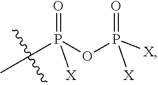

wherein one of the two X is

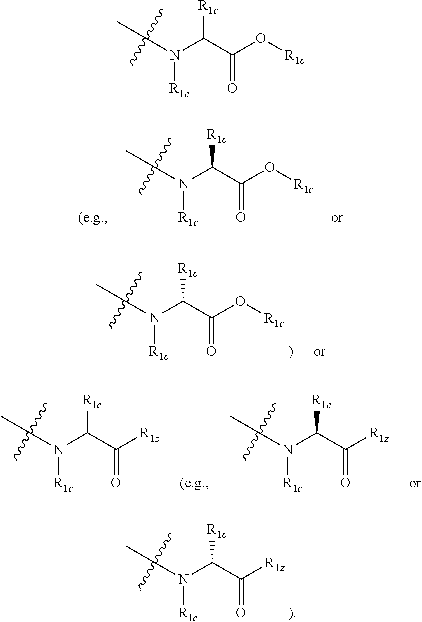

In some embodiments, R₂ is

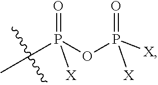

wherein each X independently is

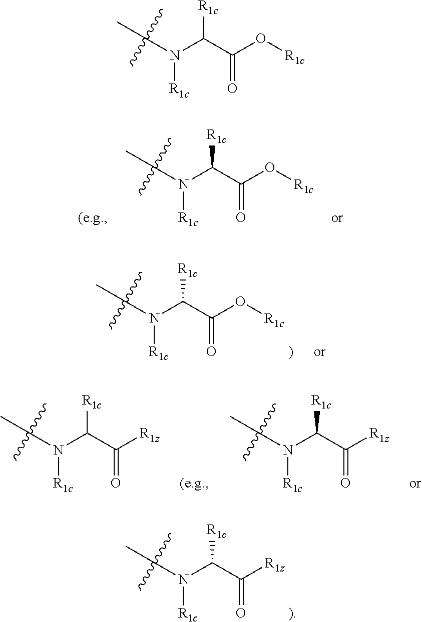

In some embodiments, R₂ is

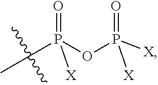

wherein at least one X is

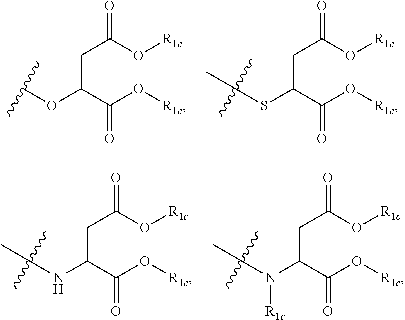

### or R_{1z}.

In some embodiments, R₂ is

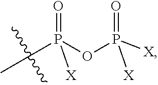

wherein two of the three X is

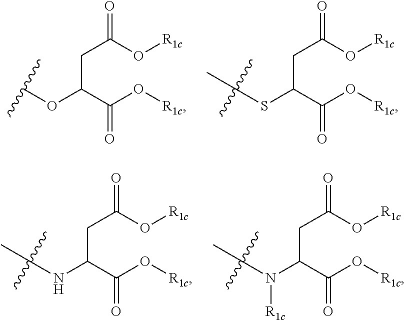

In some embodiments, R₂ is

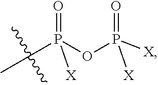

wherein each X is

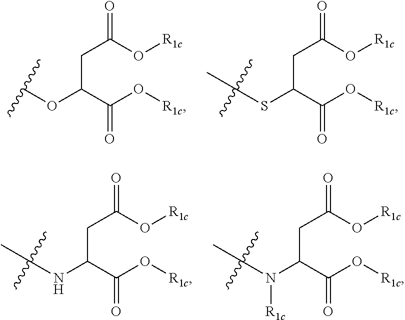

### or R_{1z}.

In some embodiments, R₂ is

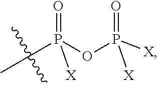

wherein at least one X is

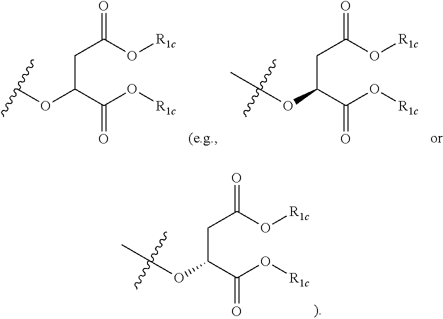

In some embodiments, R₂ is

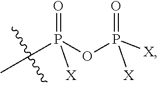

wherein two of the three X is

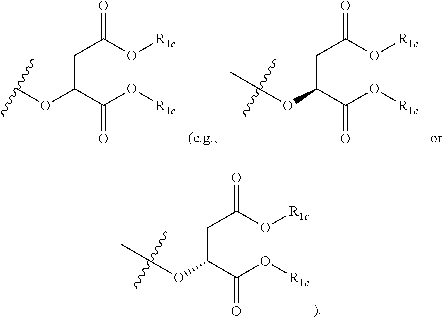

In some embodiments, R₂ is

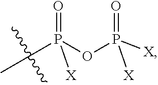

wherein each X is

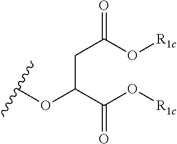

In some embodiments, R₂ is

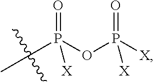

wherein at least one X is

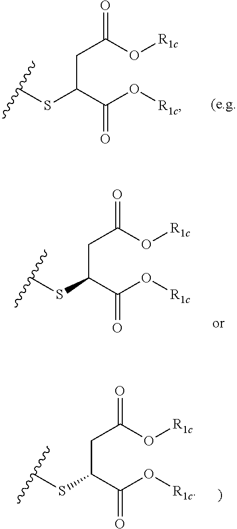

In some embodiments, R₂ is

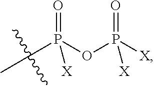

wherein two of the three X is

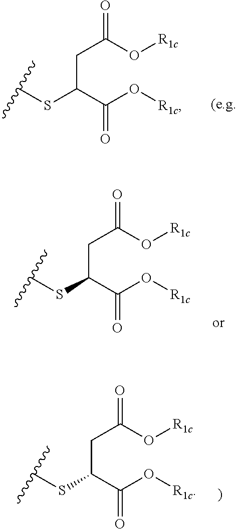

In some embodiments, R₂ is

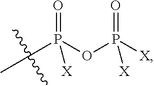

wherein each X is

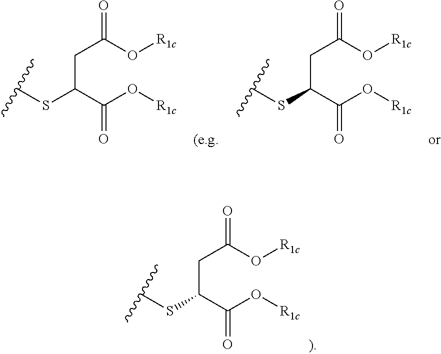

In some embodiments, R₂ is

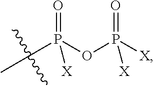

wherein at least one X is

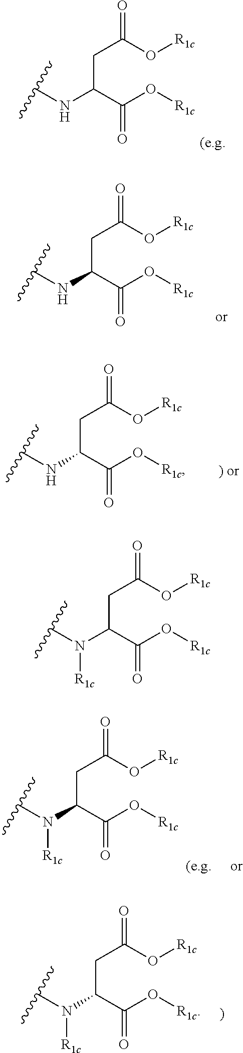

In some embodiments, R₂ is

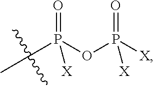

wherein two of the three X is

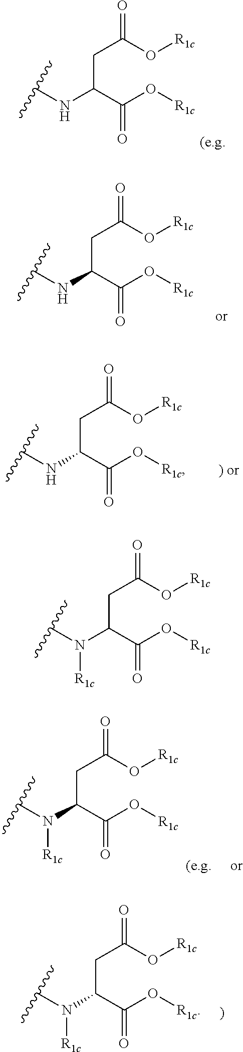

In some embodiments, R₂ is

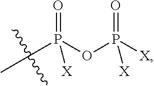

wherein each X is

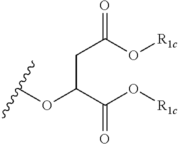

In some embodiments, R₂ is

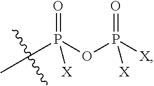

wherein at least one X is R_{1z}

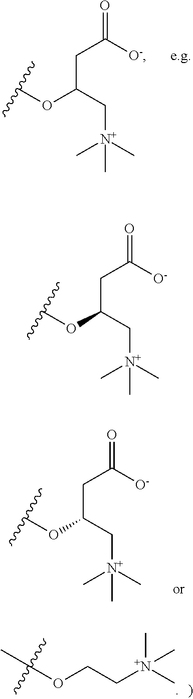

In some embodiments, R₂ is

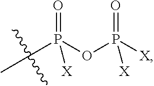

wherein two of the three X is R_{1z}

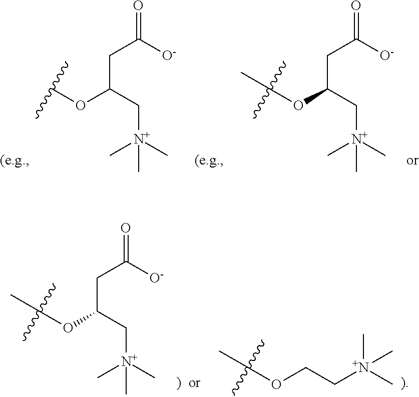

In some embodiments, R₂ is

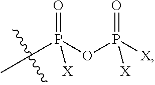

wherein each X is R_{1z}

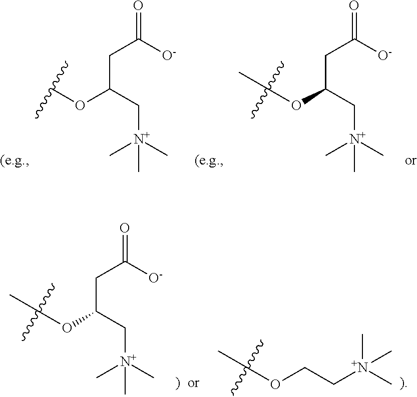

### Variable R₃

In some embodiments, R₃ is H.

In some embodiments, R₃ is —C(═O)R_{1b}, —C(═O)OR_{1c}, —C(═O)N(R_{1c})₂, —C(═O)R_{1z}, —C(═O)—(C═O)R_{1b}, —C(═O)—C(═O)OR_{1c}, —C(═O)—C(═O)N(R_{1c})₂, —C(═O)—C(═O)R_{1z}, —C(═O)—CH═CH—C(═O)OR_{1c}, —C(═O)—CH₂—CH₂—C(═O)OR_{1c},

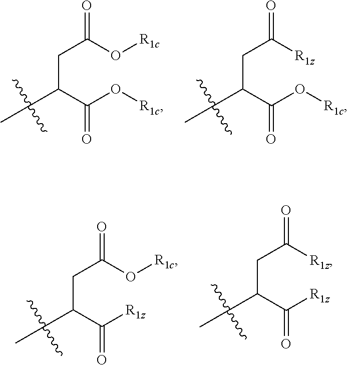

—C(═O)—CH═CH—C(═O)—R_{1z}, —C(═O)—CH₂—CH₂—C(═O)—R_{1z}, Si(R_{1g})₃,

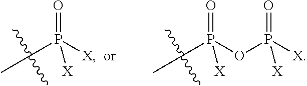

In some embodiments, R₃ is —C(═O)R_{1b}.

In some embodiments, R₃ is —C(═O)H.

In some embodiments, R₃ is —C(═O)R_{1b}, wherein R_{1b} is C₁-C₂₀ alkyl, C₂-C₂₀ alkenyl, C₂-C₂₀ alkynyl, —(CH₂)_{q}—C(═O)OR_{1c}, —CH₂—C(═O)—(CH₂)_{q}—C(═O)OR_{1c}, —CH₂—[C(═O)CH₂]ₚ—[CH₂]_{q}—C(═O)OR_{1c}, —CH═CH—C(═O)OR_{1c}, —C(═O)OR_{1c}, —C(═O)N(R_{1c})₂, or R_{1z}, wherein the C₁-C₂₀ alkyl, or C₂-C₂₀ alkenyl or C₂-C₂₀ alkynyl is optionally substituted with one or more R₁ₑ.

In some embodiments, R₃ is —C(═O)R_{1b}, wherein R_{1b} is C₁-C₂₀ alkyl, C₂-C₂₀ alkenyl, or C₂-C₂₀ alkynyl optionally substituted with one or more R₁ₑ.

In some embodiments, R₃ is —C(═O)R_{1b}, wherein R_{1b} is —(CH₂)_{q}—C(═O)OR_{1c}, —CH₂—C(═O)—(CH₂)_{q}—C(═O)OR_{1c}, —CH₂—[C(═O)CH₂]ₚ—[CH₂]_{q}—C(═O)OR_{1c}, —CH═CH—C(═O)OR_{1c}, —C(═O)OR_{1c}, or —C(═O)N(R_{1c})₂.

In some embodiments, R₃ is —(CH₂)_{q}—C(═O)OR_{1c}.

In some embodiments, R₃ is —CH₂CH₂—C(═O)OR_{1c}.

In some embodiments, R₃ is —CH₂—C(═O)—(CH₂)_{q}—C(═O)OR_{1c}.

In some embodiments, R₃ is —CH₂—C(═O)—CH₂CH₂—C(═O)OR_{1c}.

In some embodiments, R₃ is —C(═O)—CH═CH—C(═O)OR_{1c}.

In some embodiments, R₃ is —C(═O)R_{1z}.

In some embodiments, R₃ is —C(═O)—(C═O)R_{1b}.

In some embodiments, R₃ is —C(═O)—C(═O)OR_{1c}.

In some embodiments, R₂ is —C(═O)—C(═O)N(R_{1c})₂.

In some embodiments, R₃ is —C(═O)—C(═O)R_{1z},

In some embodiments, R₃ is

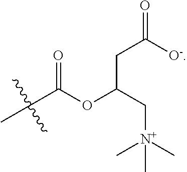

In some embodiments, R₃ is

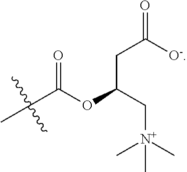

In some embodiments, R₃ is

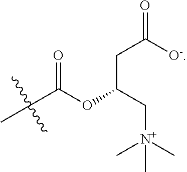

In some embodiments, R₃ is

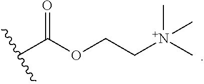 [SMILES: *C(=O)OCC[N+](C)(C)C]

In some embodiments, R₃ is —C(═O)OR_{1c}.

In some embodiments, R₃ is —C(═O)OH.

In some embodiments, R₃ is —C(═O)OR_{1c}, wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is —C(═O)OR_{1c}, wherein R_{1c} is C₁-C₂₀ alkyl, C₂-C₂₀ alkenyl, or C₂-C₂₀ alkynyl optionally substituted with one or more R₁ₑ.

In some embodiments, R₃ is —C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₃ is —C(═O)OR_{1c}, wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is —C(═O)N(R_{1c})₂.

In some embodiments, R₃ is —C(═O)N(R_{1c})₂, wherein at least one R_{1c} is H.

In some embodiments, R₃ is —C(═O)N(R_{1c})₂, wherein at least one R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is —C(═O)N(R_{1c})₂, wherein at least one R_{1c} is C₁-C₂₀ alkyl, C₂-C₂₀ alkenyl, or C₂-C₂₀ alkynyl optionally substituted with one or more R₁ₑ.

In some embodiments, R₃ is —C(═O)N(R_{1c})₂, wherein at least one R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₃ is —C(═O)N(R_{1c})₂, wherein at least one R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is —C(═O)—CH═CH—C(═O)OR_{1c}.

In some embodiments, R₃ is —C(═O)—CH═CH—C(═O)OH.

In some embodiments, R₃ is —C(═O)—CH═CH—C(═O)OR_{1c}, wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is —C(═O)—CH═CH—C(═O)OR_{1c}, wherein R_{1c} is C₁-C₂₀ alkyl, C₂-C₂₀ alkenyl, or C₂-C₂₀ alkynyl optionally substituted with one or more R₁ₑ.

In some embodiments, R₃ is —C(═O)—CH═CH—C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₃ is —C(═O)—CH═CH—C(═O)OR_{1c}, wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is —C(═O)—CH₂—CH₂—C(═O)OR_{1c}.

In some embodiments, R₃ is —C(═O)—CH₂—CH₂—C(═O)OH.

In some embodiments, R₃ is —C(═O)—CH₂—CH₂—C(═O)OR_{1c}, wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is —C(═O)—CH₂—CH₂—C(═O)OR_{1c}, wherein R_{1c} is C₁-C₂₀ alkyl, C₂-C₂₀ alkenyl, or C₂-C₂₀ alkynyl optionally substituted with one or more R₁ₑ.

In some embodiments, R₃ is —C(═O)—CH₂—CH₂—C(═O)OR_{1c}, wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₃ is —C(═O)—CH₂—CH₂—C(═O)OR_{1c}, wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is

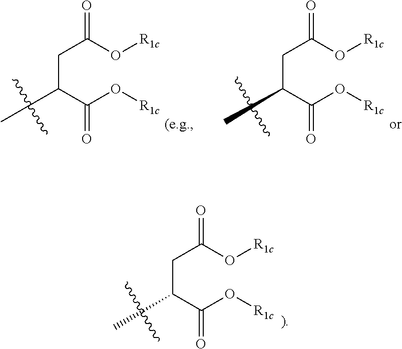

In some embodiments, R₃ is

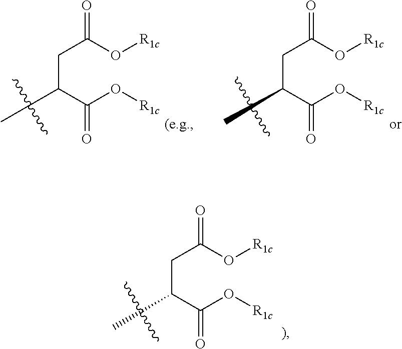

wherein at least one R_{1c} is H.

In some embodiments, R₃ is

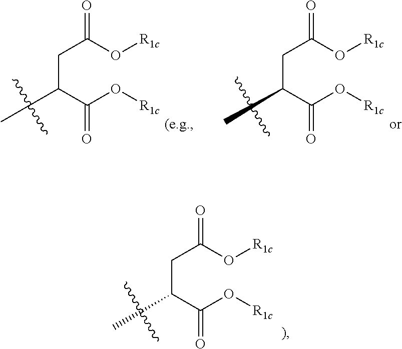

wherein at least one R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is

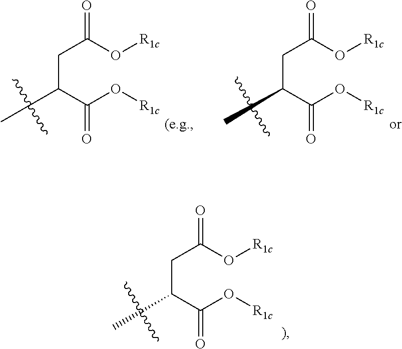

wherein at least one R_{1c} is C₁-C₂₀ alkyl, C₂-C₂₀ alkenyl, or C₂-C₂₀ alkynyl optionally substituted with one or more R₁ₑ.

In some embodiments, R₃ is

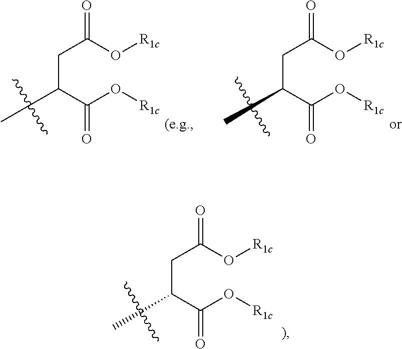

wherein at least one R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₃ is

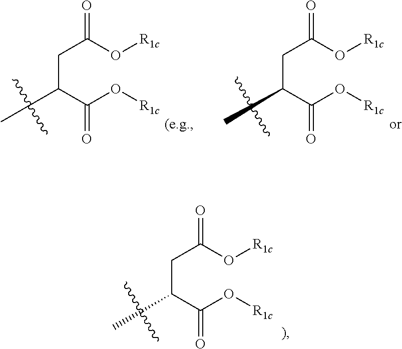

wherein at least one R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is

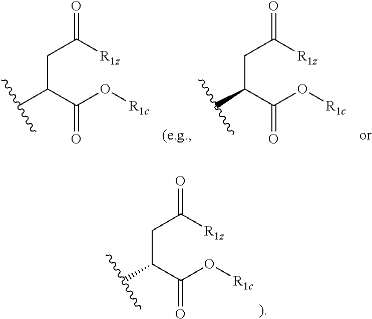

In some embodiments, R₃ is

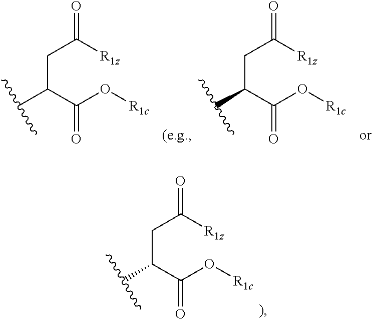

wherein R_{1c} is H.

In some embodiments, R₃ is

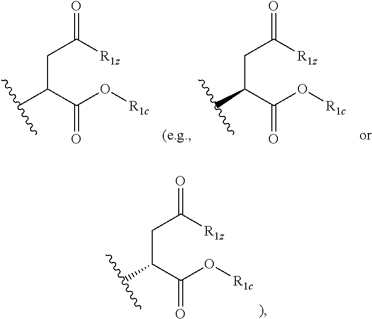

wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is

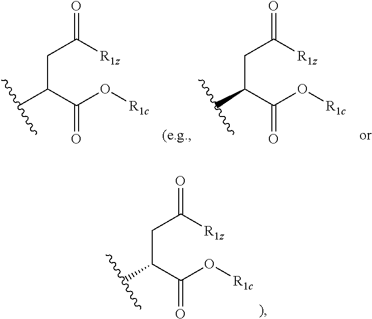

wherein R_{1c} is C₁-C₂₀ alkyl, C₂-C₂₀ alkenyl, or C₂-C₂₀ alkynyl optionally substituted with one or more R₁ₑ.

In some embodiments, R₃ is

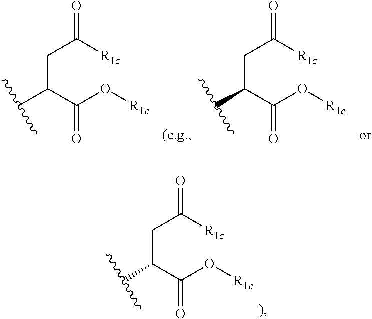

wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₃ is

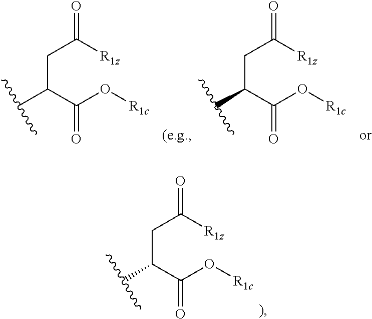

wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is

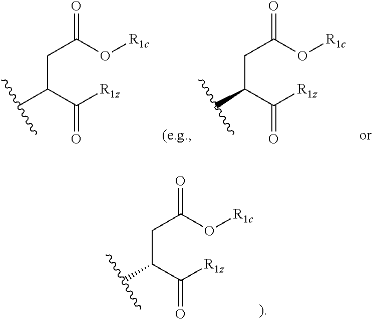

In some embodiments, R₃ is

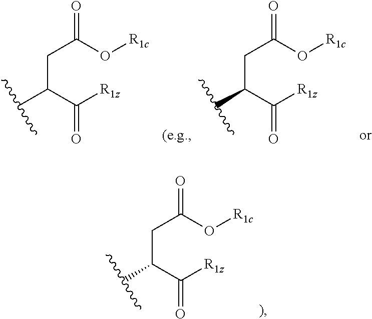

wherein R_{1c} is H.

In some embodiments, R₃ is

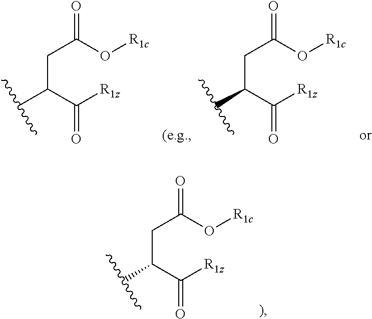

wherein R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is

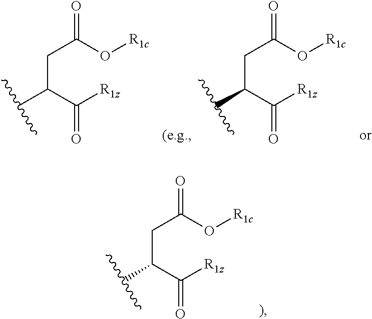

wherein R_{1c} is C₁-C₂₀ alkyl, C₂-C₂₀ alkenyl, or C₂-C₂₀ alkynyl optionally substituted with one or more R₁ₑ.

In some embodiments, R₃ is

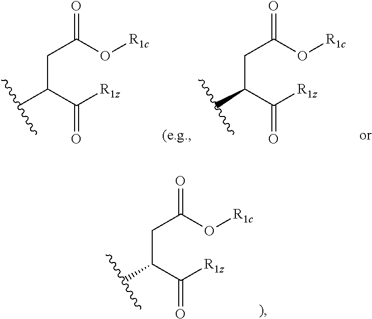

wherein R_{1c} is C₃-C₁₂ cycloalkyl, C₃-C₁₂ heterocycloalkyl, C₃-C₁₂ aryl, or C₃-C₁₂ heteroaryl optionally substituted with one or more R₁ₑ.

In some embodiments, R₃ is

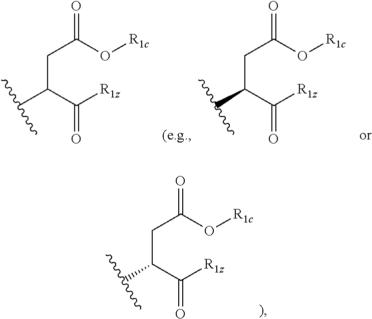

wherein R_{1c} is —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) optionally substituted with one or more R₁ₑ.

In some embodiments, R₃ is

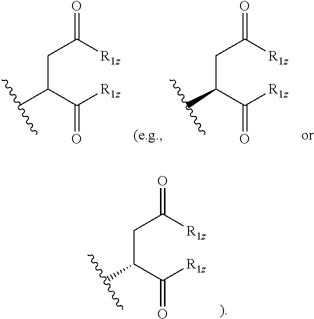

In some embodiments, R₃ is —C(═O)—CH═CH—C(═O)—R_{1z}.

In some embodiments, R₃ is —C(═O)—CH═CH—C(═O)—R_{1z}, wherein R_{1z} is

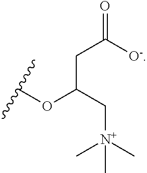

In some embodiments, R₃ is —C(═O)—CH═CH—C(═O)—R_{1z}, wherein R_{1z} is

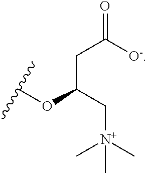

In some embodiments, R₃ is —C(═O)—CH═CH—C(═O)—R_{1z}, wherein R_{1z} is

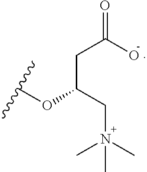

In some embodiments, R₃ is —C(═O)—CH═CH—C(═O)—R_{1z}, wherein R_{1z} is

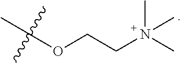

In some embodiments, R₃ is —C(═O)—CH₂—CH₂—C(═O)—R_{1z}.

In some embodiments, R₃ is —C(═O)—CH₂—CH₂—C(═O)—R_{1z}, wherein R_{1z} is

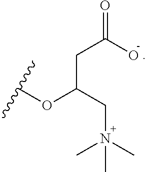

In some embodiments, R₃ is —C(═O)—CH₂—CH₂—C(═O)—R_{1z}, wherein R_{1z} is

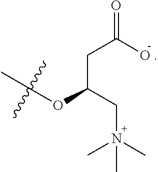

In some embodiments, R₃ is —C(═O)—CH₂—CH₂—C(═O)—R_{1z}, wherein R_{1z} is

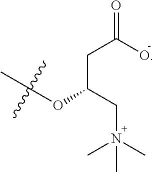

In some embodiments, R₃ is —C(═O)—CH₂—CH₂—C(═O)—R_{1z}, wherein R_{1z} is

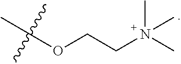

In some embodiments, R₃ is Si(R_{1g})₃.

In some embodiments, R₂ is Si(R_{1g})₃, wherein at least one R_{1g} is C₁-C₂₀ alkyl.

In some embodiments, R₃ is Si(R_{1g})₃, wherein at least two R_{1g} are C₁-C₂₀ alkyl.

In some embodiments, R₃ is Si(R_{1g})₃, wherein all R_{1g} are C₁-C₂₀ alkyl.

In some embodiments, R₃ is

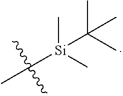 [SMILES: *[Si](C)(C)C(C)(C)C]

In some embodiments, R₃ is

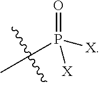

In some embodiments, R₃ is

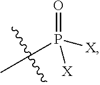

wherein at least one X is —OR_{1c}, —SR_{1c}, or —N(R_{1c})₂.

In some embodiments, R₃ is

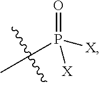

wherein one of the two X is —OR_{1c}, —SR_{1c}, or —N(R_{1c})₂.

In some embodiments, R₃ is

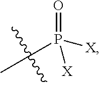

wherein each X is independently —OR_{1c}, —SR_{1c}, or —N(R_{1c})₂.

In some embodiments, R₃ is

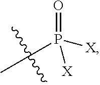

wherein at least one X is —OR_{1c}.

In some embodiments, R₃ is

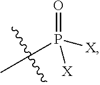

wherein one of the two X is —OR_{1c}.

In some embodiments, R₃ is

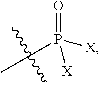

wherein each X is independently —OR_{1c}.

In some embodiments, R₃ is

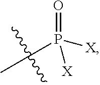

wherein at least one X is —SR_{1c}.

In some embodiments, R₃ is

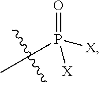

wherein one of the two X is —SR_{1c}.

In some embodiments, R₃ is

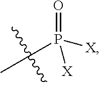

wherein each X is independently —SR_{1c}.

In some embodiments, R₃ is

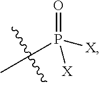

wherein at least one X is —N(R_{1c})₂.

In some embodiments, R₃ is

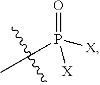

wherein one of the two X is —N(R_{1c})₂.

In some embodiments, R₃ is

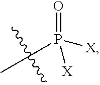

wherein each X is independently —N(R_{1c})₂.

In some embodiments, R₃ is

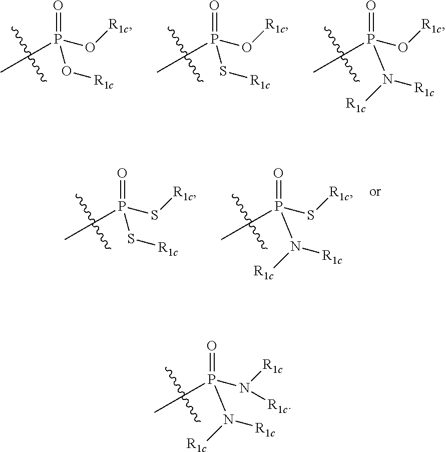

In some embodiments, R₃ is

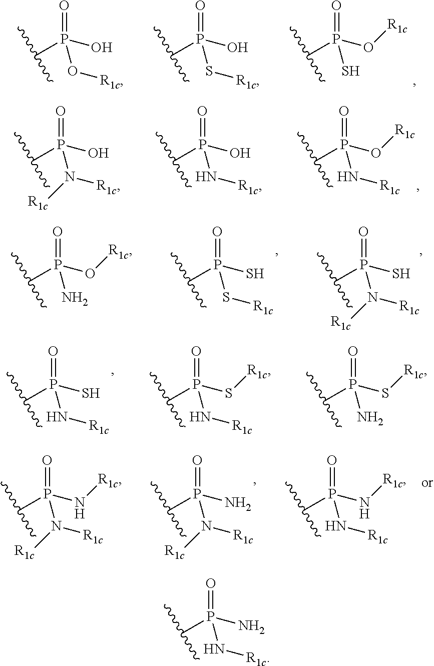

In some embodiments, R₃ is

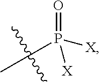

wherein at least one X is R₁ is

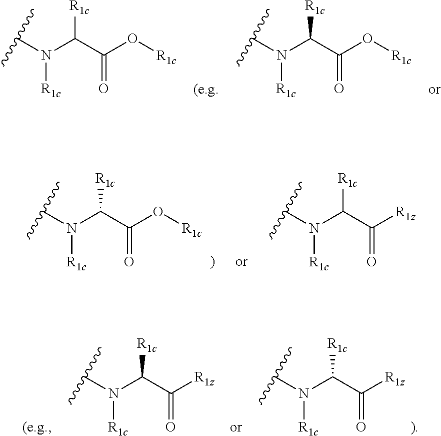

In some embodiments, R₃ is

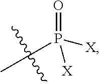

wherein one of the two X is

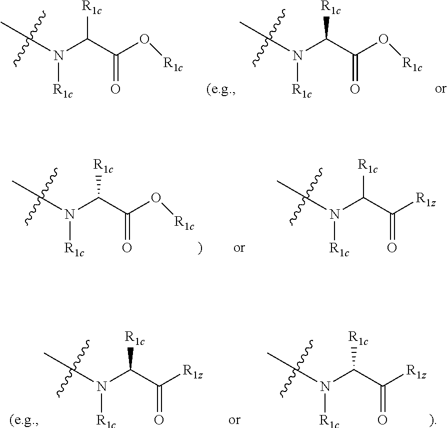

In some embodiments, R₃ is

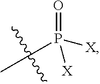

wherein each X independently is

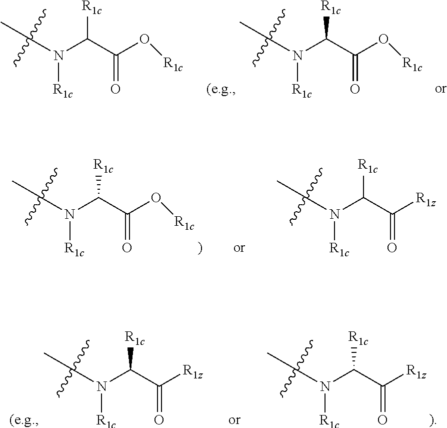

In some embodiments, R₃ is

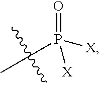

wherein at least one X is

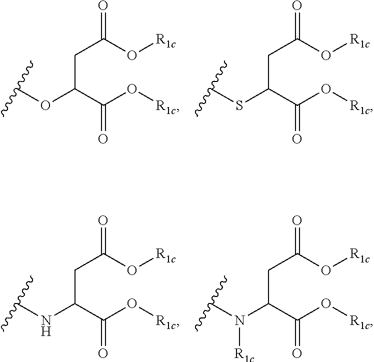

### or R_{1z}.

In some embodiments, R₃ is

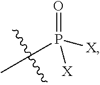

wherein one of the two X is

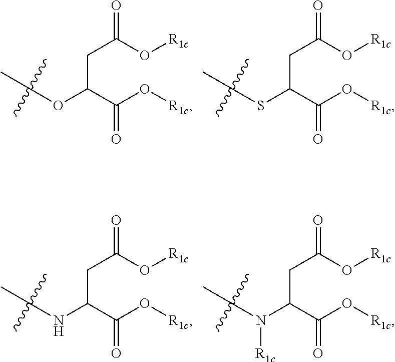

### or R_{1z}.

In some embodiments, R₃ is

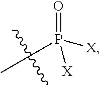

wherein each X is

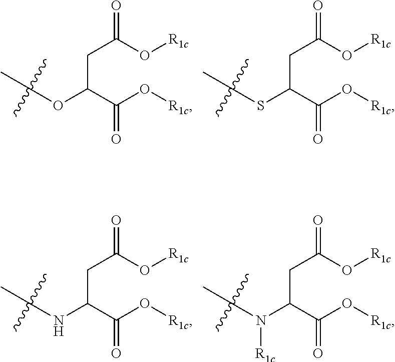

### or R_{1z}.

In some embodiments, R₃ is

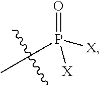

wherein at least one X is

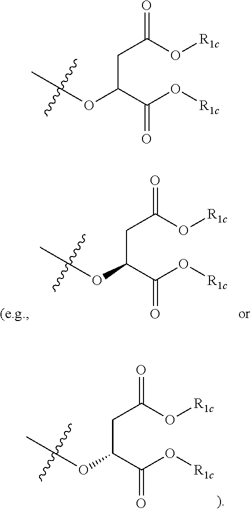

In some embodiments, R₃ is

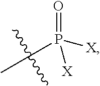

wherein one of the two X is

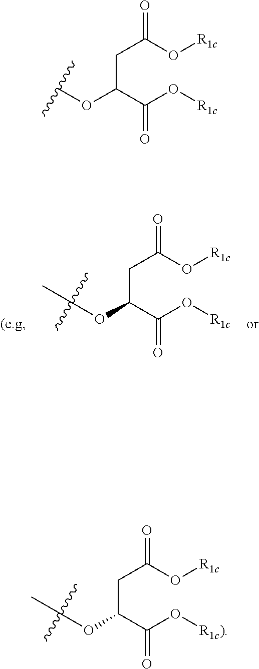

In some embodiments, R₃ is

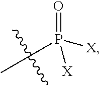

wherein each X is

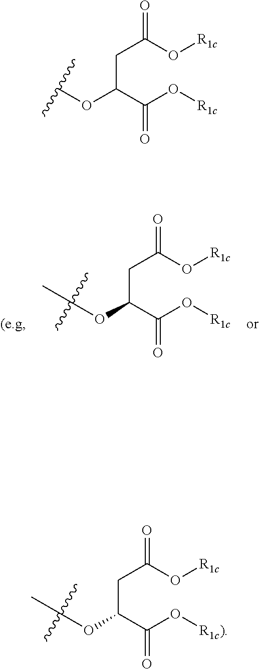

In some embodiments, R₃ is

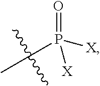

wherein at least one X is

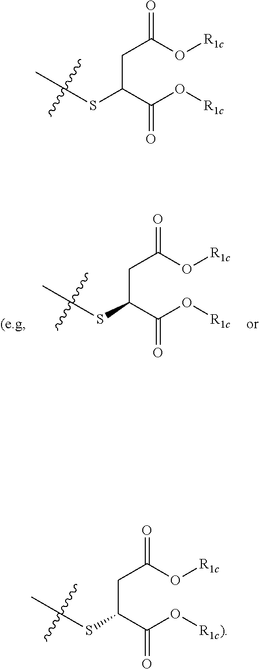

In some embodiments, R₃ is

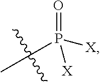

wherein one of the two X is

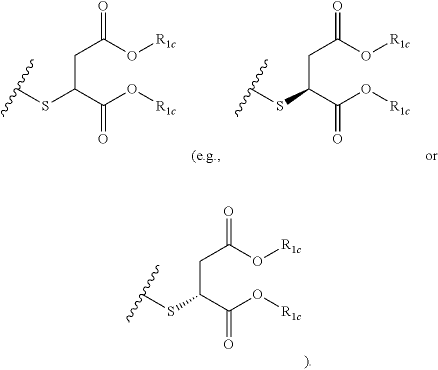

In some embodiments, R₃ is

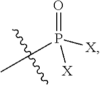

wherein each X is

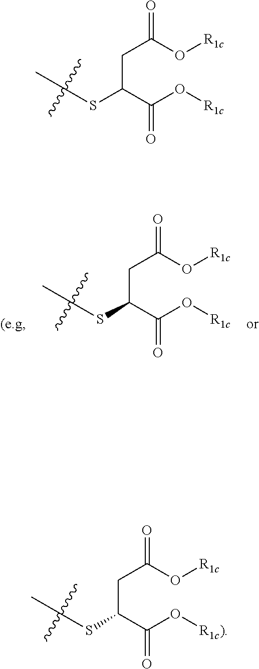

In some embodiments, R₃ is

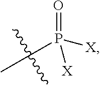

wherein at least one X is

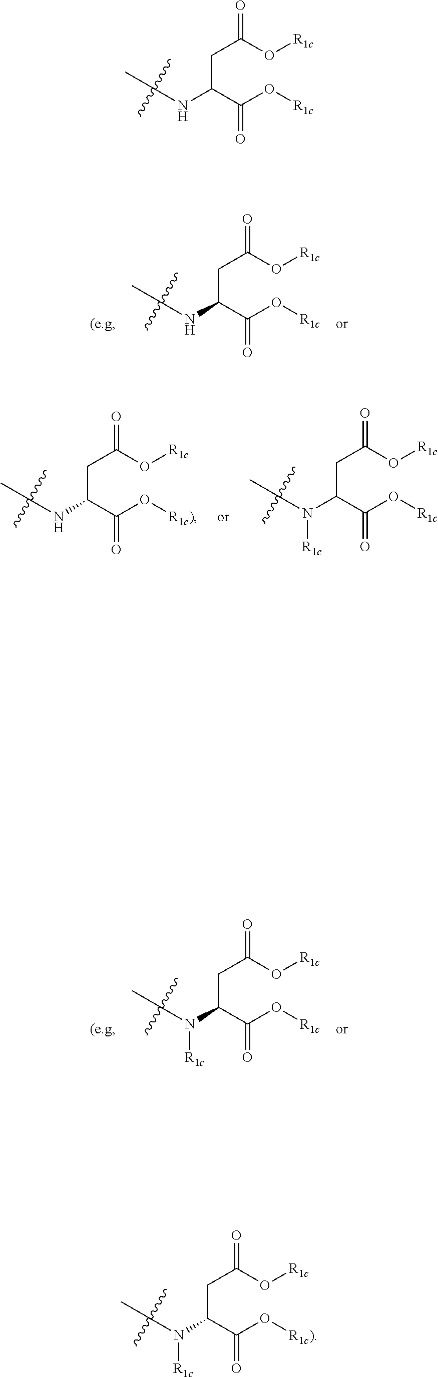

In some embodiments, R₃ is

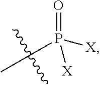

wherein one of the two X is

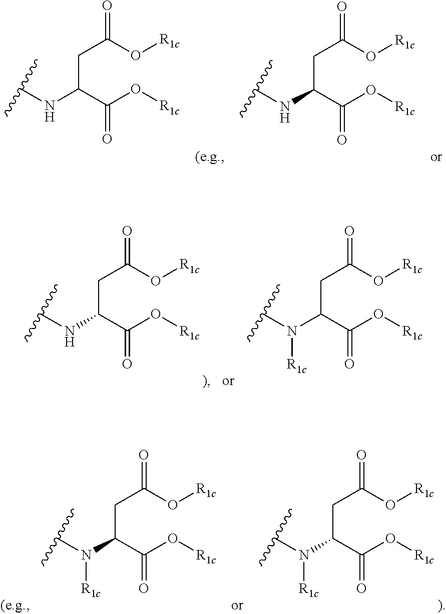

In some embodiments, R₃ is

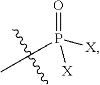

wherein each X is

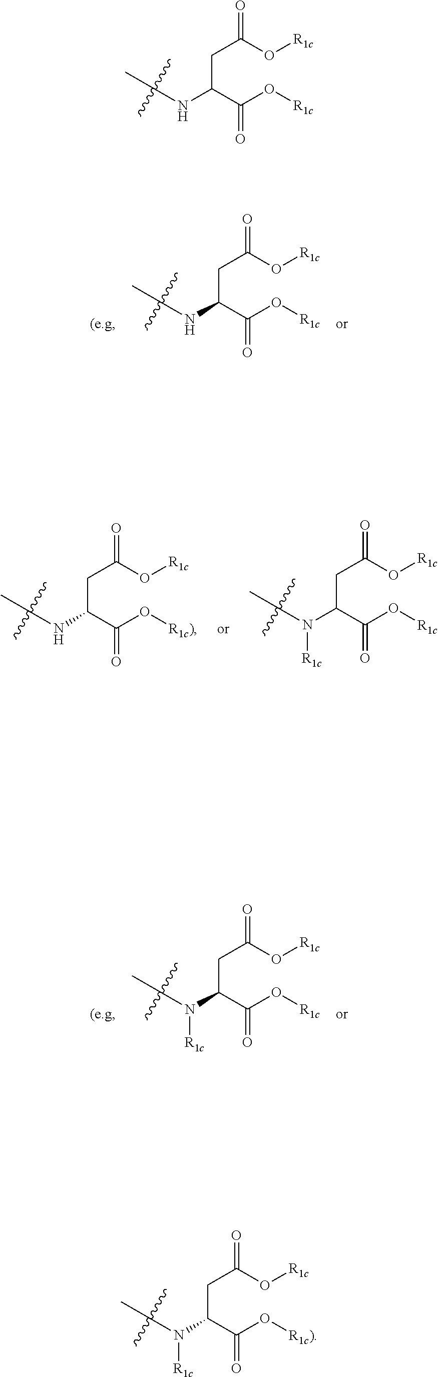

In some embodiments, R₃ is

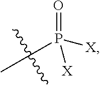

wherein at least one X is R_{1z}

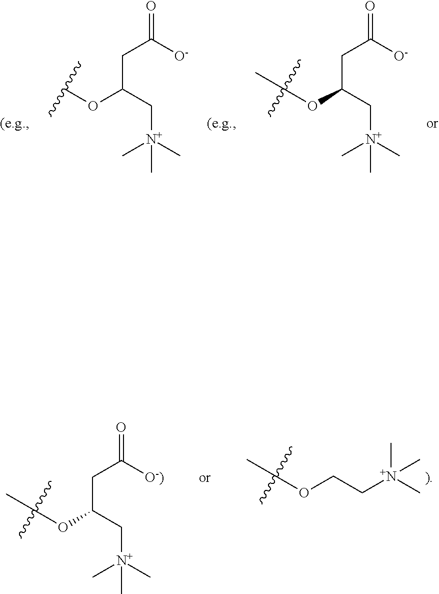

In some embodiments, R₃ is

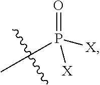

wherein one of the two X is R_{1z}

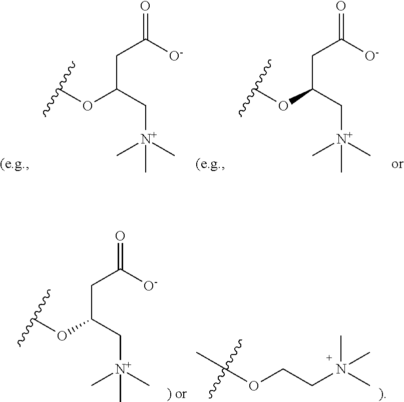

In some embodiments, R₃ is

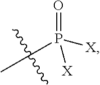

wherein each X is R_{1z}

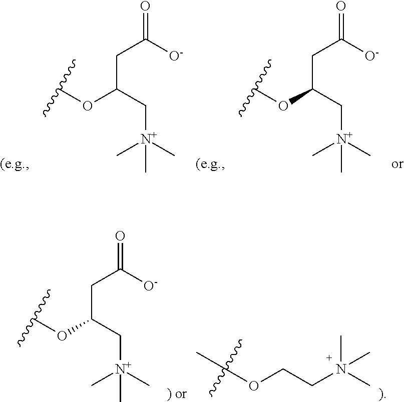

In some embodiments, R₃ is

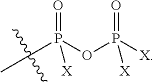

In some embodiments, R₃ is

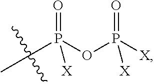

wherein at least one X is —OR_{1c}, —SR_{1c}, or —N(R_{1c})₂.

In some embodiments, R₃ is

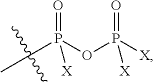

wherein two of the three X is —OR_{1c}, —SR_{1c}, or —N(R_{1c})₂.

In some embodiments, R₃ is

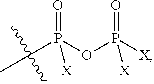

wherein each X is independently —OR_{1c}, —SR_{1c}, or —N(R_{1c})₂.

In some embodiments, R₃ is

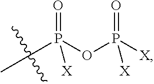

wherein at least one X is —OR_{1c}.

In some embodiments, R₃ is

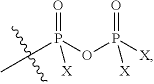

wherein two of the three X is —OR_{1c}.

In some embodiments, R₃ is

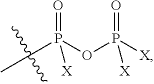

wherein each X is independently —OR_{1c}.

In some embodiments, R₃ is

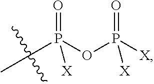

wherein at least one X is —SR_{1c}.

In some embodiments, R₃ is

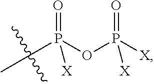

wherein two of the three X is —SR_{1c}.

In some embodiments, R₃ is

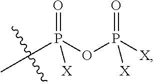

wherein each X is independently —SR_{1c}.

In some embodiments, R₃ is

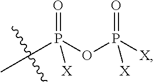

wherein at least one X is —N(R_{1c})₂.

In some embodiments, R₃ is

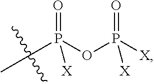

wherein two of the three X is —N(R_{1c})₂.

In some embodiments, R₃ is

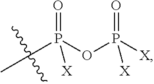

wherein each X is independently —N(R_{1c})₂.

In some embodiments, R₃ is

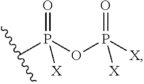

wherein at least one X is

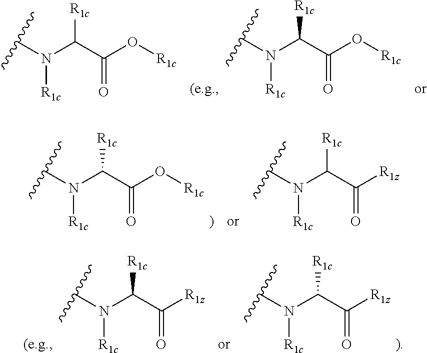

In some embodiments, R₃ is

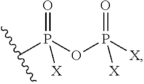

wherein one of the two X is

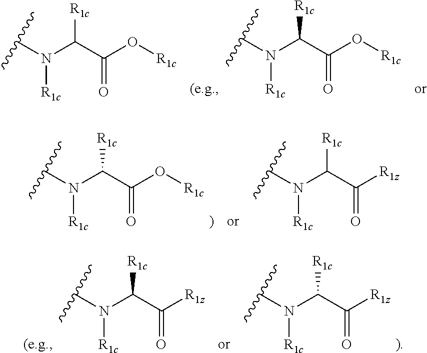

In some embodiments, R₃ is

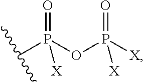

wherein each X independently is

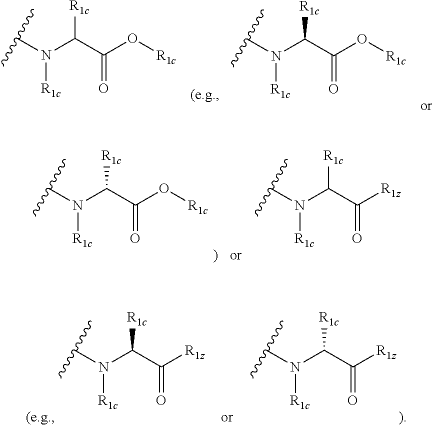

In some embodiments, R₃ is

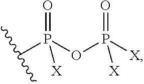

wherein at least one X is

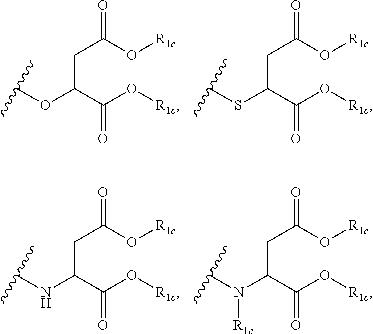

### or R_{1z}.

In some embodiments, R₃ is

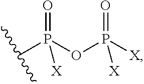

wherein two of the three X is

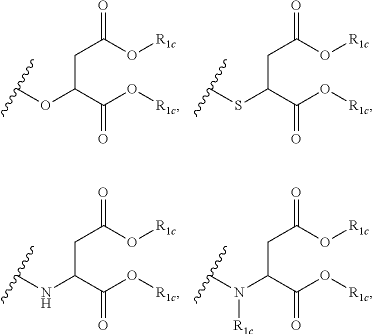

In some embodiments, R₃ is

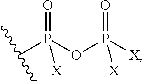

wherein each X is

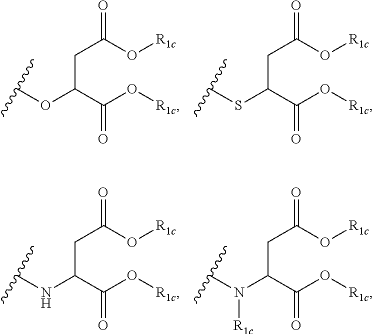

### or R_{1z}.

In some embodiments, R₃ is

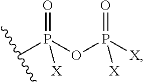

wherein at least one X is

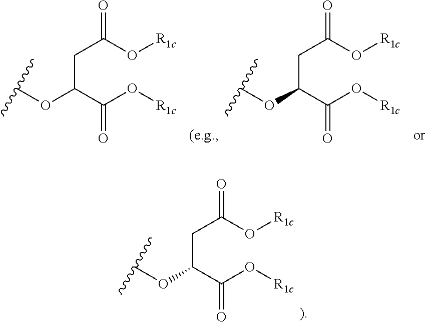

In some embodiments, R₃ is

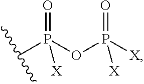

wherein two of the three X is

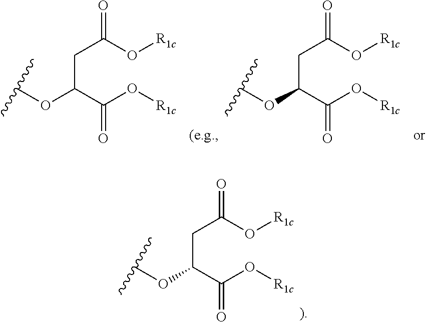

In some embodiments, R₃ is

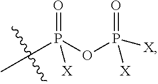

wherein each X is

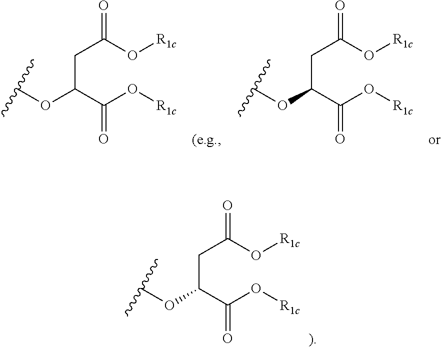

In some embodiments, R₃ is

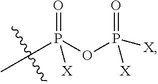

wherein at least one X is

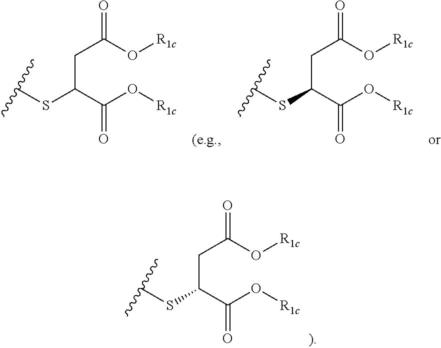

In some embodiments, R₃ is

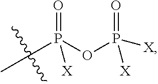

wherein two of the three X is

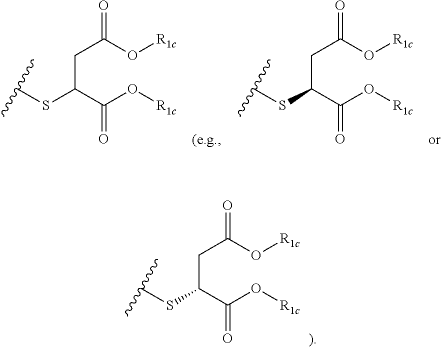

In some embodiments, R₃ is

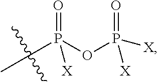

wherein each X is

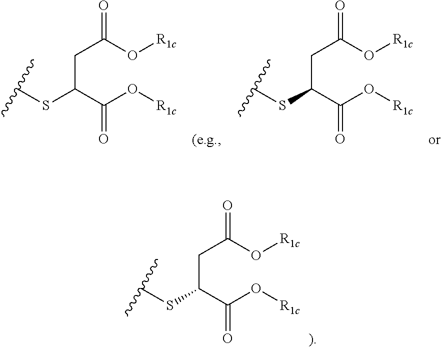

In some embodiments, R₃ is

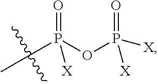

wherein at least one X is

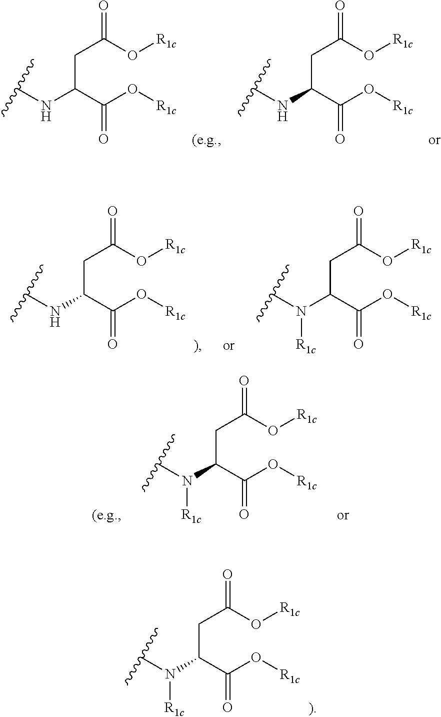

In some embodiments, R₃ is

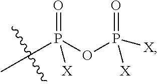

wherein two of the three X is

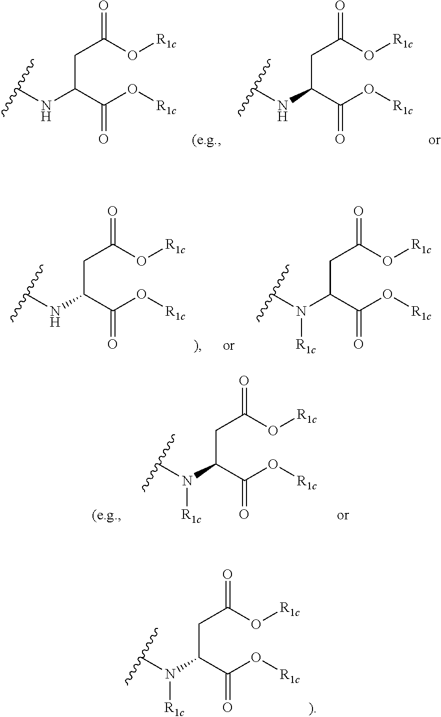

In some embodiments, R₃ is

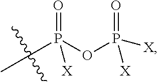

wherein each X is

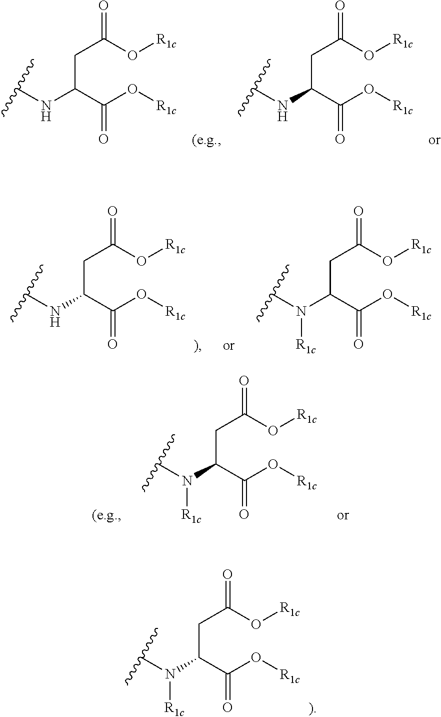

In some embodiments, R₃ is

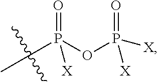

wherein at least one X is R_{1z} (e.g.,

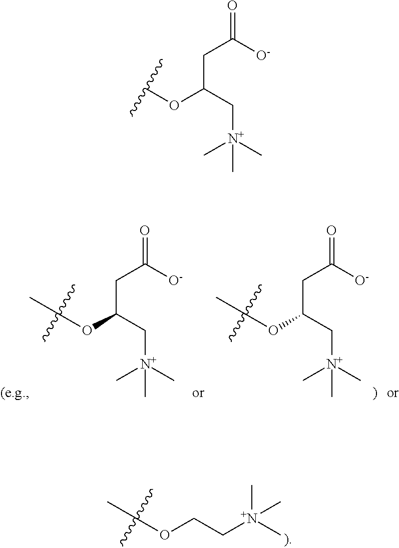

In some embodiments, R₃ is

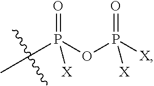

wherein two of the three X is R_{1z}

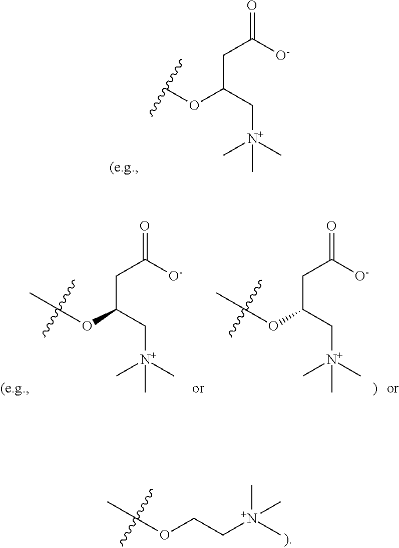

In some embodiments, R₃ is

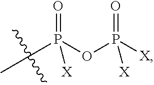

wherein each X is R_{1z}

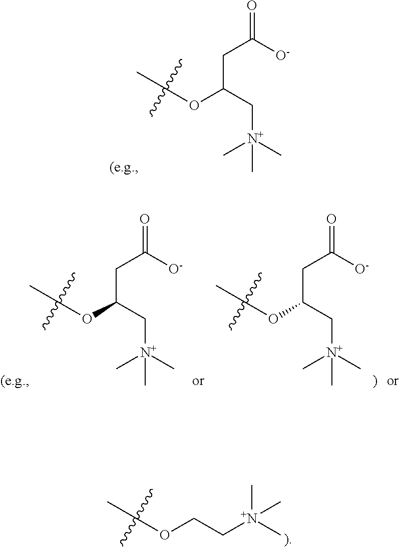

### Variable X

In some embodiments, at least one X is —OR_{1c}.

In some embodiments, at least one X is —SR_{1c}.

In some embodiments, at least one X is —N(R_{1c})₂.

In some embodiments, at least one X is

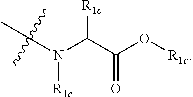

In some embodiments, at least one X is

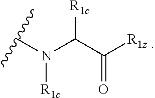

In some embodiments, at least one X is

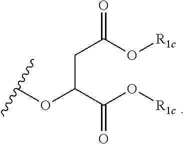

In some embodiments, at least one X is

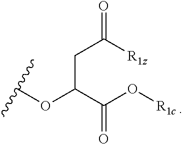

In some embodiments, at least one X is

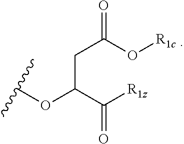

In some embodiments, at least one X is

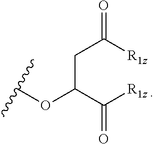

In some embodiments, at least one X is

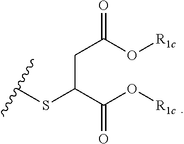

In some embodiments, at least one X is

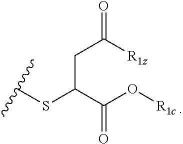

In some embodiments, at least one X is

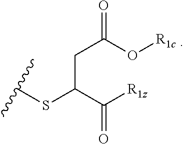

In some embodiments, at least one X is

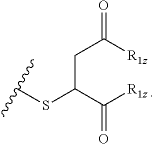

In some embodiments, at least one X is

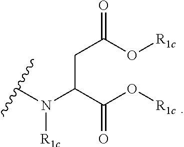

In some embodiments, at least one X is

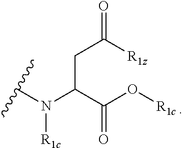

In some embodiments, at least one X is

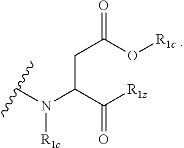

In some embodiments, at least one X is

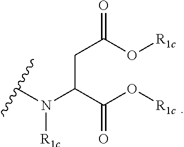

In some embodiments, at least one X is R_{1z}.

In some embodiments, at least one X is

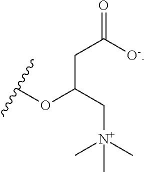

In some embodiments, at least one X is

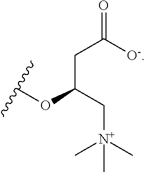

In some embodiments, at least one X is

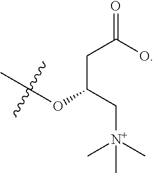

In some embodiments, at least one X is

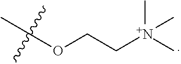 [SMILES: *OCC[N+](C)(C)C]

### Exemplary Formulae and Compounds

In some embodiments, the compound is of Formula (I-1) or (I-2):

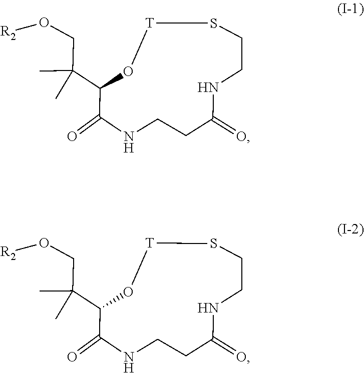

or a pharmaceutically acceptable salt or solvate thereof.

In some embodiments, the compound is of Formula (I-1) or a pharmaceutically acceptable salt or solvate thereof.

In some embodiments, the compound is of Formula (I-2) or a pharmaceutically acceptable salt or solvate thereof.

In some embodiments, the compound is of Formula (Ia):

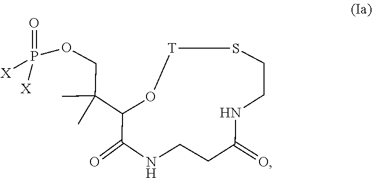

or a pharmaceutically acceptable salt or solvate thereof.

In some embodiments the compound is of Formula (Ia-1) or (Ia-2):

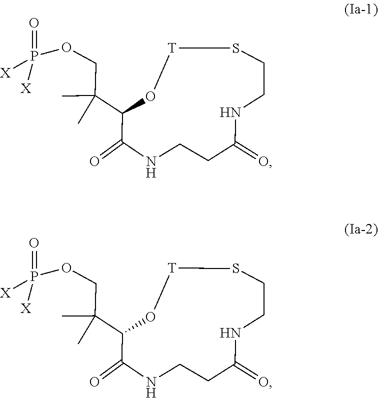

or a pharmaceutically acceptable salt or solvate thereof.

In some embodiments, the compound is of Formula (Ia-1) or a pharmaceutically acceptable salt or solvate thereof.

In some embodiments, the compound is of Formula (Ia-2) or a pharmaceutically acceptable salt or solvate thereof.

In some embodiments, the compound is of Formula (Ib), (Ic), (Id), or (Ie):

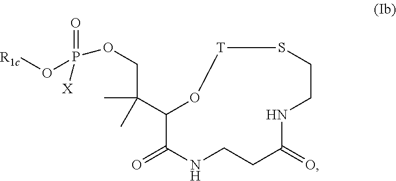

or a pharmaceutically acceptable salt or solvate thereof.

In some embodiments, the compound is of Formula (Ib) or a pharmaceutically acceptable salt or solvate thereof.

In some embodiments, the compound is of Formula (Ic) or a pharmaceutically acceptable salt or solvate thereof.

In some embodiments, the compound is of Formula (Id) or a pharmaceutically acceptable salt or solvate thereof.

In some embodiments, the compound is of Formula (Ie) or a pharmaceutically acceptable salt or solvate thereof.

In some embodiments, the compound is of Formula (Ib-1), (Ib-2), (Ic-1), (Ic-2), (Id-1), (Id-2), (Ie-1), or (Ie-2):

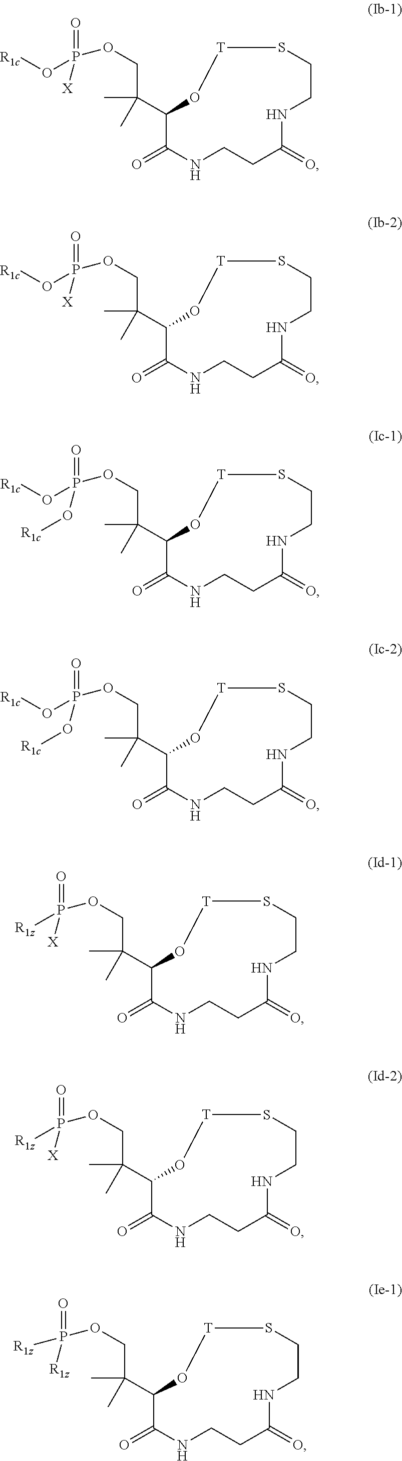

or a pharmaceutically acceptable salt or solvate thereof.

In some embodiments, the compound is of Formula (Ib-1) or a pharmaceutically acceptable salt or solvate thereof.

In some embodiments, the compound is of Formula (Ib-2) or a pharmaceutically acceptable salt or solvate thereof.

In some embodiments, the compound is of Formula (Ic-1) or a pharmaceutically acceptable salt or solvate thereof.

In some embodiments, the compound is of Formula (Ic-2) or a pharmaceutically acceptable salt or solvate thereof.

In some embodiments, the compound is of Formula (Id-1) or a pharmaceutically acceptable salt or solvate thereof.

In some embodiments, the compound is of Formula (Id-2) or a pharmaceutically acceptable salt or solvate thereof.

In some embodiments, the compound is of Formula (Ie-1) or a pharmaceutically acceptable salt or solvate thereof.

In some embodiments, the compound is of Formula (Ie-2) or a pharmaceutically acceptable salt or solvate thereof.

In some embodiments, the compound is of Formula (If), (Ig), (Ih), or (Ii):

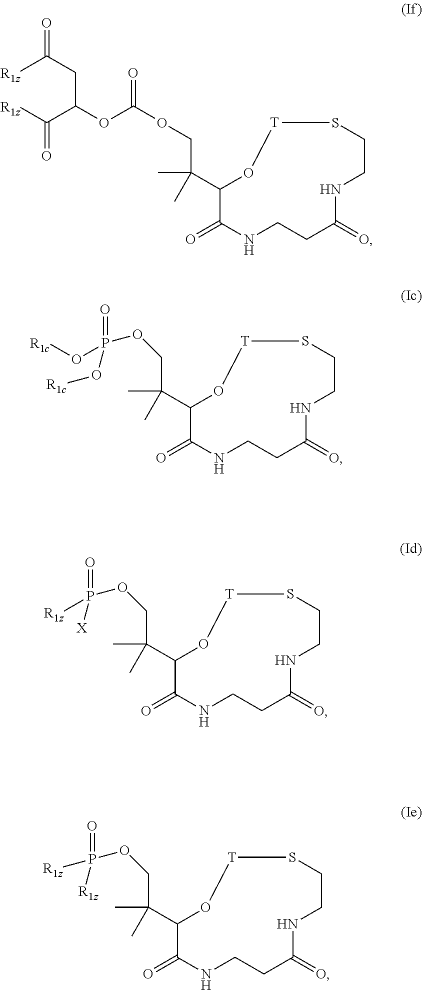

or pharmaceutically acceptable salt or solvate thereof.

In some embodiments, the compound is of Formula (If-1), (If-2), (Ig-1), (Ig-2), (Ih-1), (Ih-2), (Ii-1), (Ii-2):

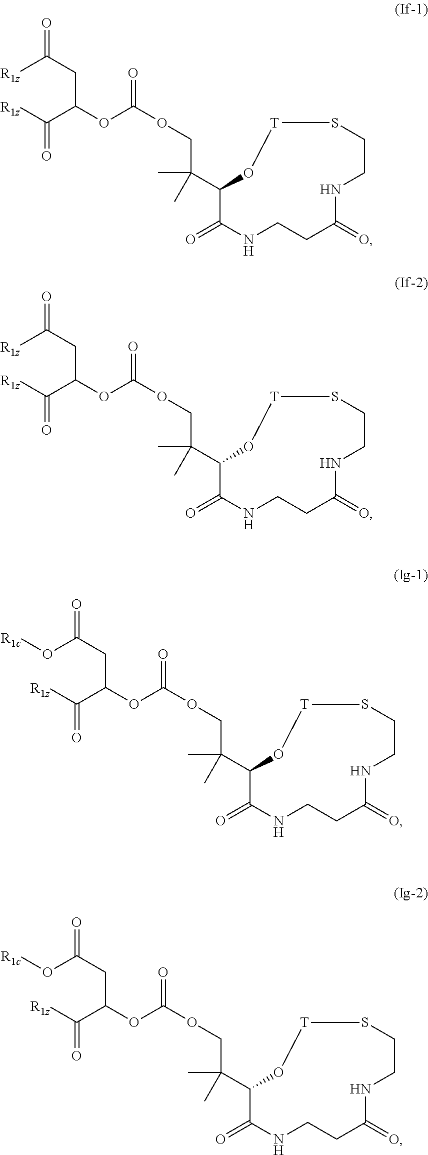

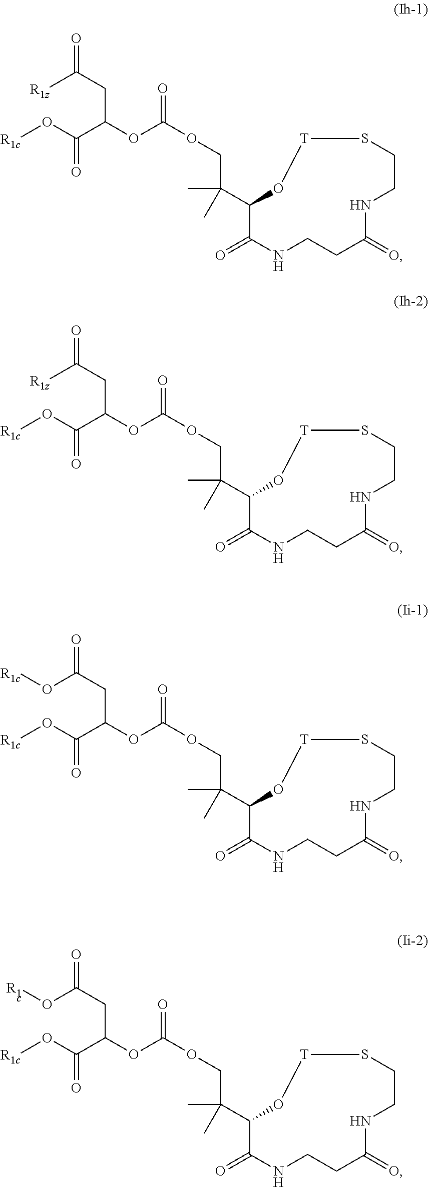

or a pharmaceutically acceptable salt or solvate thereof.

In some embodiments, the compound is of Formula (If-1) or a pharmaceutically acceptable salt or solvate thereof.

In some embodiments, the compound is of Formula (If-2) or a pharmaceutically acceptable salt or solvate thereof.

In some embodiments, the compound is of Formula (Ig-1) or a pharmaceutically acceptable salt or solvate thereof.

In some embodiments, the compound is of Formula (Ig-2) or a pharmaceutically acceptable salt or solvate thereof.

In some embodiments, the compound is of Formula (Ih-1) or a pharmaceutically acceptable salt or solvate thereof.

In some embodiments, the compound is of Formula (Ih-2) or a pharmaceutically acceptable salt or solvate thereof.

In some embodiments, the compound is of Formula (Ii-1) or a pharmaceutically acceptable salt or solvate thereof.

In some embodiments, the compound is of Formula (Ii-2) or a pharmaceutically acceptable salt or solvate thereof.

In some embodiments, the compound is of Formula (Ij).

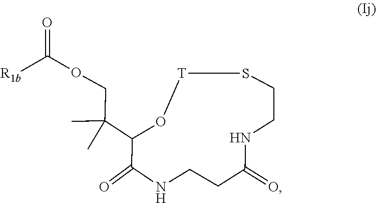

or a pharmaceutically acceptable salt or solvate thereof.

In some embodiments, the compound is of Formula (Ij-1) or (Ij-2):

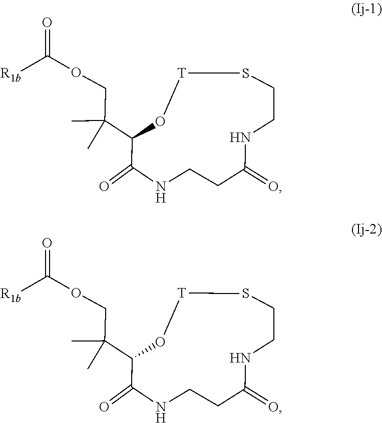

or a pharmaceutically acceptable salt or solvate thereof.

In some embodiments, the compound is of Formula (Ij-1) or a pharmaceutically acceptable salt or solvate thereof.

In some embodiments, the compound is of Formula (Ij-2) or a pharmaceutically acceptable salt or solvate thereof.

In some embodiments, the compound is of Formula (Ik):

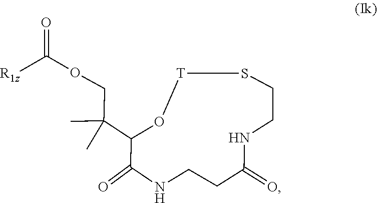

or a pharmaceutically acceptable salt or solvate thereof.

In some embodiments, the compound is of Formula (Ik-1) or (Ik-2):

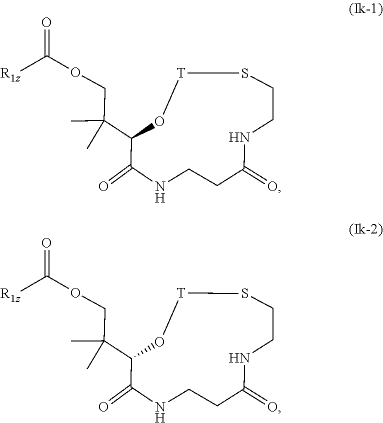

or a pharmaceutically acceptable salt or solvate thereof.

In some embodiments, the compound is of Formula (Ik-1) or a pharmaceutically acceptable salt or solvate thereof.

In some embodiments, the compound is of Formula (Ik-2) or a pharmaceutically acceptable salt or solvate thereof.

In some embodiments, the compound is of Formula (Il):

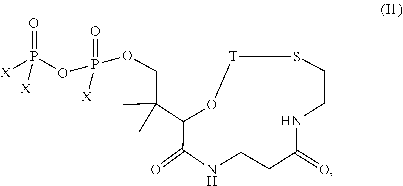

or a pharmaceutically acceptable salt or solvate thereof.

In some embodiments, the compound is of Formula (Il-1) or (Il-2):

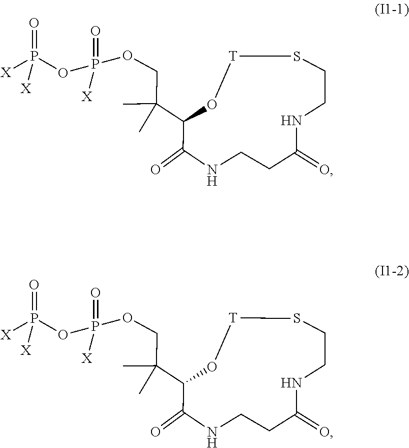

or a pharmaceutically acceptable salt or solvate thereof.

In some embodiments, the compound is of Formula (Il-1) or a pharmaceutically acceptable salt or solvate thereof.

In some embodiments, the compound is of Formula (Il-2) or a pharmaceutically acceptable salt or solvate thereof.

In some embodiments, the compound is of Formula (Im):

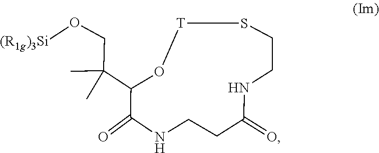

or a pharmaceutically acceptable salt or solvate thereof.

In some embodiments, the compound is of Formula (Im-1) or (Im-2):

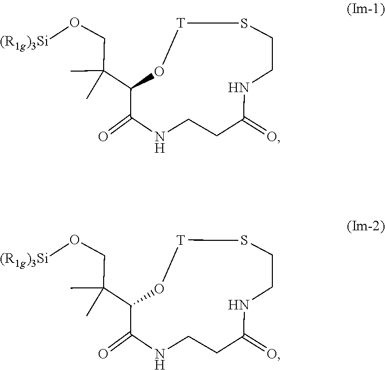

or a pharmaceutically acceptable salt or solvate thereof.

In some embodiments, the compound is of Formula (Im-1) or a pharmaceutically acceptable salt or solvate thereof.

In some embodiments, the compound is of Formula (Im-2) or a pharmaceutically acceptable salt or solvate thereof.

In some embodiments, the compound is of Formula (In):

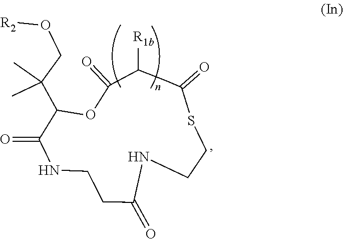

or a pharmaceutically acceptable salt or solvate thereof.

In some embodiments, the compound is of Formula (In-1) or (In-2):

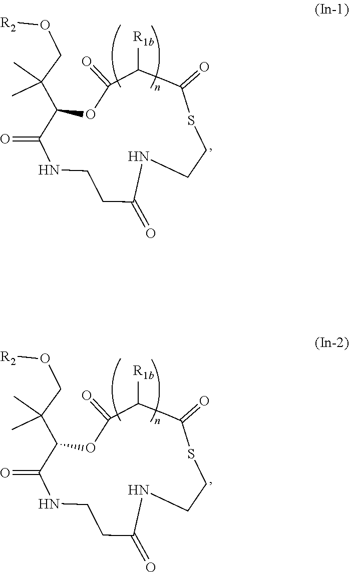

or a pharmaceutically acceptable salt or solvate thereof.

In some embodiments, the compound is of Formula (In-1) or a pharmaceutically acceptable salt or solvate thereof.

In some embodiments, the compound is of Formula (In-2) or a pharmaceutically acceptable salt or solvate thereof.

In some embodiments, the compound is of Formula (Io):

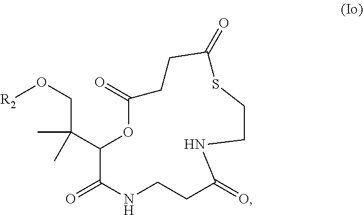

or a pharmaceutically acceptable salt or solvate thereof.

In some embodiments, the compound is of Formula (Io-1) or (Io-2):

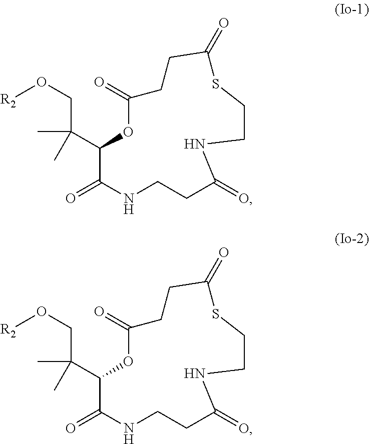

or a pharmaceutically acceptable salt or solvate thereof.

In some embodiments, the compound is of Formula (Io-1) or a pharmaceutically acceptable salt or solvate thereof.

In some embodiments, the compound is of Formula (Io-2) or a pharmaceutically acceptable salt or solvate thereof.

In some embodiments the compound is of Formula (II-1) or (II-2):

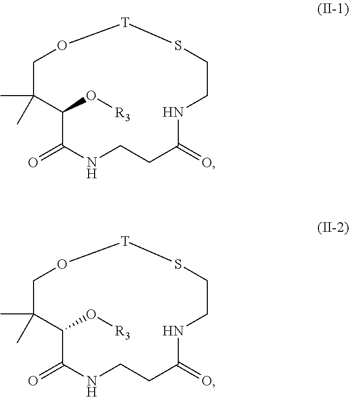

or a pharmaceutically acceptable salt or solvate thereof.

In some embodiments, the compound is of Formula (II-1) or a pharmaceutically acceptable salt or solvate thereof.

In some embodiments, the compound is of Formula (II-2) or a pharmaceutically acceptable salt or solvate thereof.

In some embodiments, the compound is of Formula (IIa):

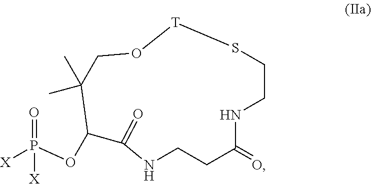

or a pharmaceutically acceptable salt or solvate thereof.

In some embodiments, the compound is of Formula (IIa-1) or (IIa-2):

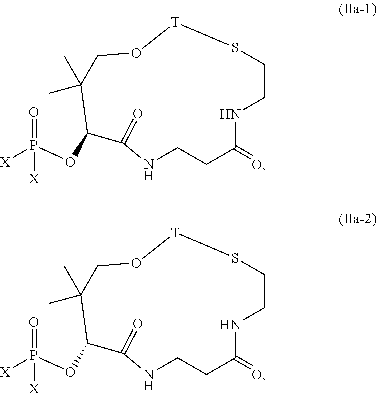

or a pharmaceutically acceptable salt or solvate thereof.

In some embodiments, the compound is of Formula (IIa-1) or a pharmaceutically acceptable salt or solvate thereof.

In some embodiments, the compound is of Formula (IIa-2) or a pharmaceutically acceptable salt or solvate thereof.

In some embodiments, the compound is of Formula (IIb), (IIc), (IId), or (IIe):

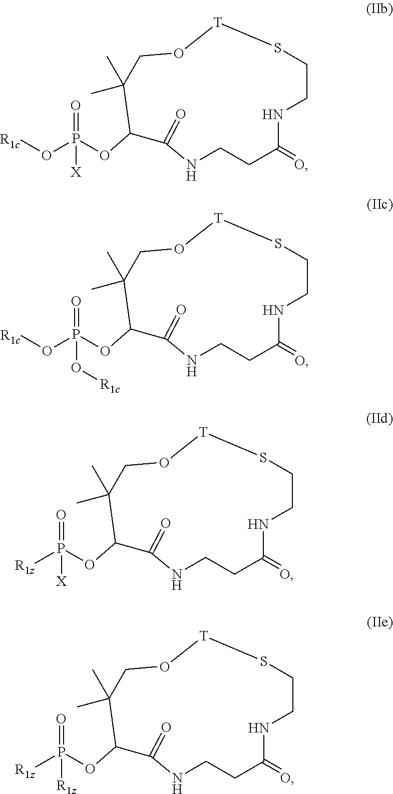

or a pharmaceutically acceptable salt or solvate thereof.

In some embodiments, the compound is of Formula (IIb) or a pharmaceutically acceptable salt or solvate thereof.

In some embodiments, the compound is of Formula (IIc) or a pharmaceutically acceptable salt or solvate thereof.

In some embodiments, the compound is of Formula (IId) or a pharmaceutically acceptable salt or solvate thereof.

In some embodiments, the compound is of Formula (IIe) or a pharmaceutically acceptable salt or solvate thereof.

In some embodiments, the compound is of Formula (IIb-1), (IIb-2), (IIc-1), (IIc-2), (IId-1), (IId-2), (IIe-1), or (IIe-2):

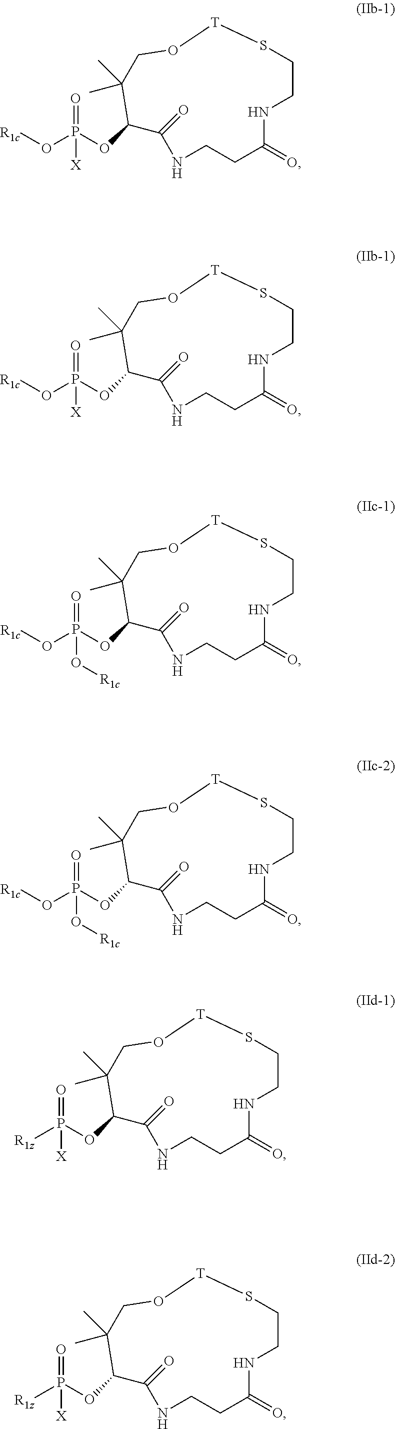

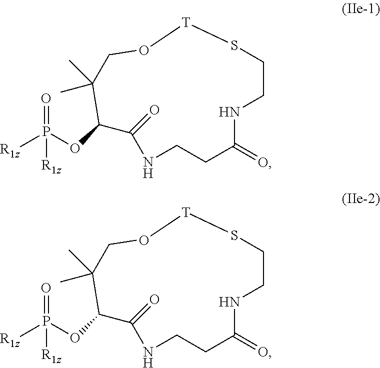

or a pharmaceutically acceptable salt or solvate thereof.

In some embodiments, the compound is of Formula (IIb-1) or a pharmaceutically acceptable salt or solvate thereof.

In some embodiments, the compound is of Formula (IIb-2) or a pharmaceutically acceptable salt or solvate thereof.

In some embodiments, the compound is of Formula (IIc-1) or a pharmaceutically acceptable salt or solvate thereof.

In some embodiments, the compound is of Formula (IIc-2) or a pharmaceutically acceptable salt or solvate thereof.

In some embodiments, the compound is of Formula (Id-1) or a pharmaceutically acceptable salt or solvate thereof.

In some embodiments, the compound is of Formula (IId-2) or a pharmaceutically acceptable salt or solvate thereof.

In some embodiments, the compound is of Formula (IIe-1) or a pharmaceutically acceptable salt or solvate thereof.

In some embodiments, the compound is of Formula (IIe-2) or a pharmaceutically acceptable salt or solvate thereof.

In some embodiments, the compound is of Formula (IIf), (IIg), (IIh), or (IIi):

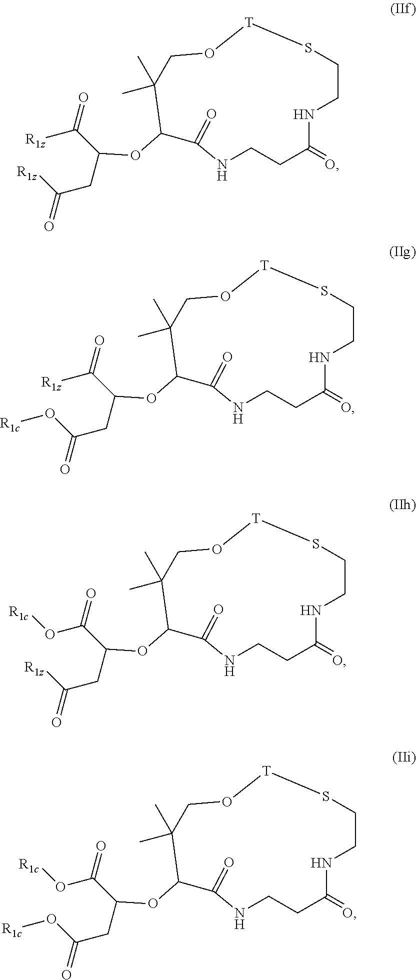

or a pharmaceutically acceptable salt or solvate thereof.

In some embodiments, the compound is of Formula (IIf-1), (IIf-2), (IIg-1), (IIg-2), (IIh-1), (IIh-2), (IIi-1), (IIi-2):

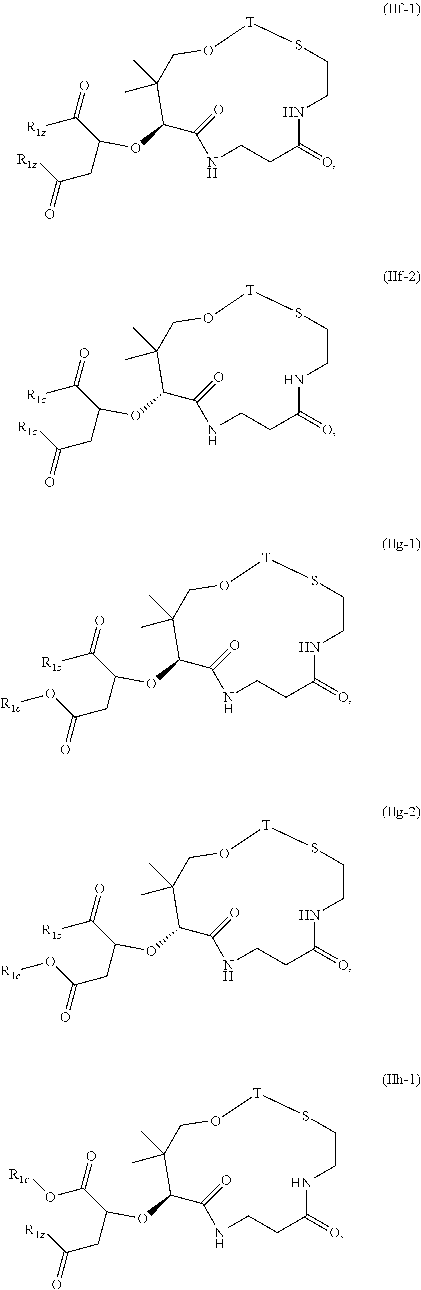

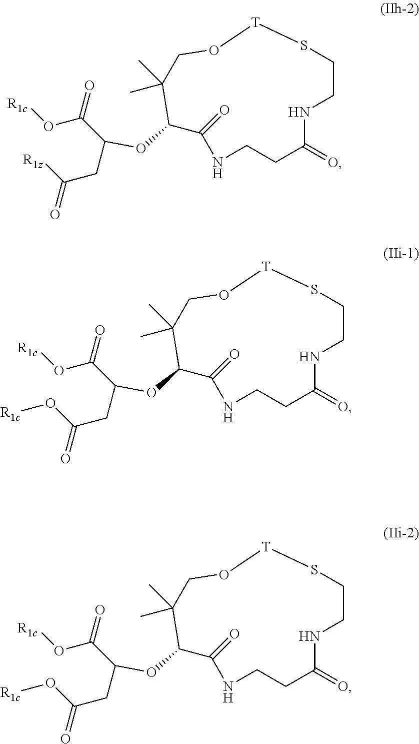

or a pharmaceutically acceptable salt or solvate thereof.

In some embodiments, the compound is of Formula (IIf-1) or a pharmaceutically acceptable salt or solvate thereof.

In some embodiments, the compound is of Formula (IIf-2) or a pharmaceutically acceptable salt or solvate thereof.

In some embodiments, the compound is of Formula (IIg-1) or a pharmaceutically acceptable salt or solvate thereof.

In some embodiments, the compound is of Formula (IIg-2) or a pharmaceutically acceptable salt or solvate thereof.

In some embodiments, the compound is of Formula (IIh-1) or a pharmaceutically acceptable salt or solvate thereof.

In some embodiments, the compound is of Formula (IIh-2) or a pharmaceutically acceptable salt or solvate thereof.

In some embodiments, the compound is of Formula (IIi-1) or a pharmaceutically acceptable salt or solvate thereof.

In some embodiments, the compound is of Formula (IIi-2) or a pharmaceutically acceptable salt or solvate thereof.

In some embodiments, the compound is of Formula (IIj):

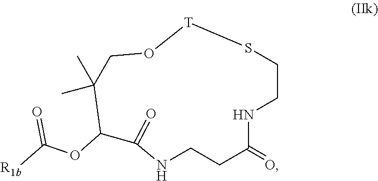

or a pharmaceutically acceptable salt or solvate thereof.

In some embodiments, the compound is of Formula (IIj-1) or (IIj-2):

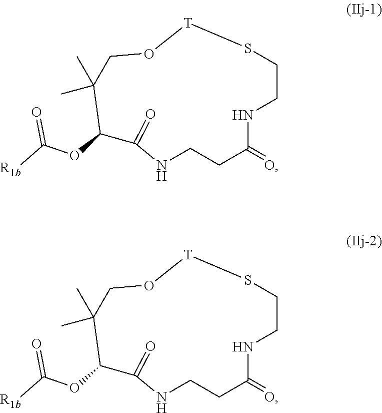

or a pharmaceutically acceptable salt or solvate thereof.

In some embodiments, the compound is of Formula (IIj-1) or a pharmaceutically acceptable salt or solvate thereof.

In some embodiments, the compound is of Formula (IIj-2) or a pharmaceutically acceptable salt or solvate thereof.

In some embodiments, the compound is of Formula (IIk):

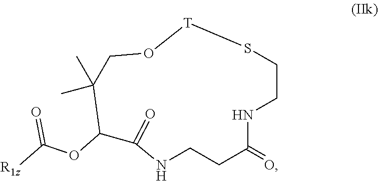

or a pharmaceutically acceptable salt or solvate thereof.

In some embodiments, the compound is of Formula (IIk-1) or (IIk-2):

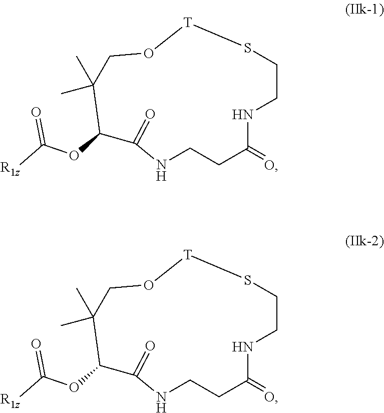

or a pharmaceutically acceptable salt or solvate thereof.

In some embodiments, the compound is of Formula (IIk-1) or a pharmaceutically acceptable salt or solvate thereof.

In some embodiments, the compound is of Formula (IIk-2) or a pharmaceutically acceptable salt or solvate thereof.

In some embodiments, the compound is of Formula (IIl).

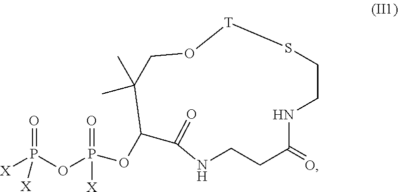

or a pharmaceutically acceptable salt or solvate thereof.

In some embodiments, the compound is of Formula (IIl-1) or (IIl-2):

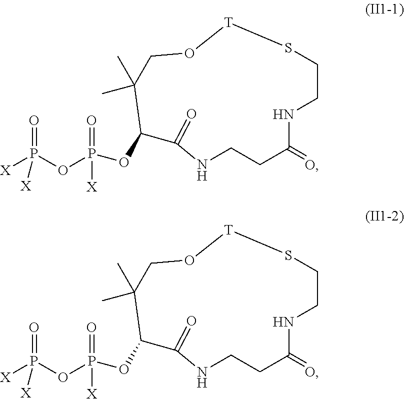

or a pharmaceutically acceptable salt or solvate thereof.

In some embodiments, the compound is of Formula (IIl-1) or a pharmaceutically acceptable salt or solvate thereof.

In some embodiments, the compound is of Formula (IIl-2) or a pharmaceutically acceptable salt or solvate thereof.

In some embodiments, the compound is of Formula (IIm):

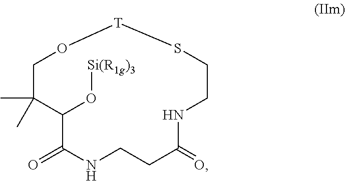

or a pharmaceutically acceptable salt or solvate thereof.

In some embodiments, the compound is of Formula (IIm-1) or (IIm-2):

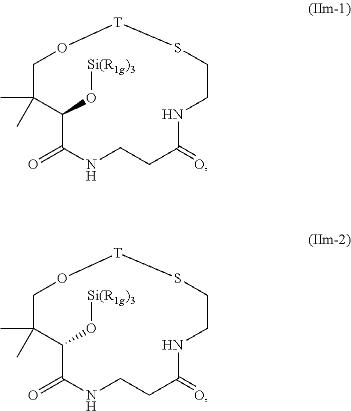

or a pharmaceutically acceptable salt or solvate thereof.

In some embodiments, the compound is of Formula (IIm-1) or a pharmaceutically acceptable salt or solvate thereof.

In some embodiments, the compound is of Formula (IIm-2) or a pharmaceutically acceptable salt or solvate thereof.

In some embodiments, the compound is of Formula (IIn):

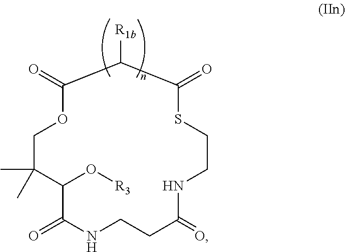

or a pharmaceutically acceptable salt or solvate thereof.

In some embodiments, the compound is of Formula (IIn-1) or (IIn-2):

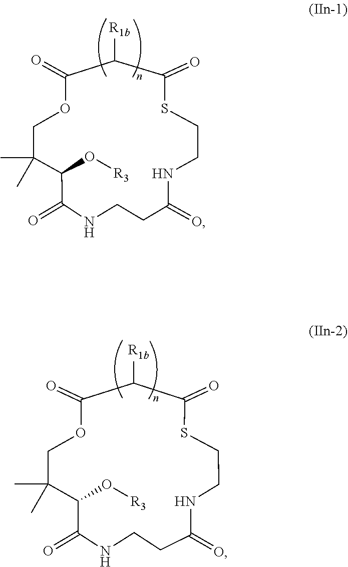

or a pharmaceutically acceptable salt or solvate thereof.

In some embodiments, the compound is of Formula (IIn-1) or a pharmaceutically acceptable salt or solvate thereof.

In some embodiments, the compound is of Formula (IIn-2) or a pharmaceutically acceptable salt or solvate thereof.

In some embodiments, the compound is of Formula (IIo):

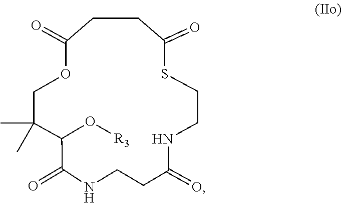

or a pharmaceutically acceptable salt or solvate thereof.

In some embodiments, the compound is of Formula (IIo-1) or (IIo-2):

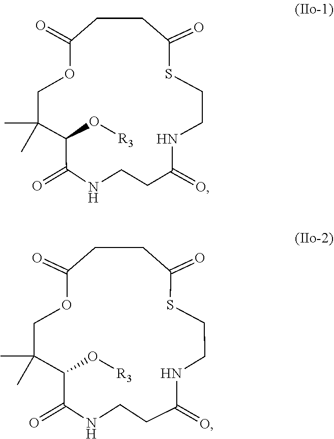

or a pharmaceutically acceptable salt or solvate thereof.

In some embodiments, the compound is of Formula (IIo-1) or a pharmaceutically acceptable salt or solvate thereof.

In some embodiments, the compound is of Formula (IIo-2) or a pharmaceutically acceptable salt or solvate thereof.

It is understood that, for a compound of any one of the Formulae disclosed herein, T, R_{1b}, R_{1c}, R_{1d}, R₁ₑ, R_{1f}, R_{1g}, R_{1z}, R₂, R₃, X, n, p, q, and r can each be, where applicable, selected from the groups described herein, and any group described herein for any of T, R_{1b}, R_{1c}, R_{1d}, R₁ₑ, R_{1f}, R_{1g}, R_{1z}, R₂, R₃, X, n, p, q, and r can be combined, where applicable, with any group described herein for one or more of the remainder of T, R_{1b}, R_{1c}, R_{1d}, R₁ₑ, R_{1f}, R_{1g}, R_{1z}, R₂, R₃, X, n, p, q, and r.

In some embodiments, the compound is of Formula (I), wherein T is *—C(═O)—(CHR_{1b})ₙ—C(═O)—**, n is 2, each R_{1b} is H, and R₂ is H.

In some embodiments, the compound is of Formula (I), wherein T is *—C(═O)—(CHR_{1b})ₙ—C(═O)—**, wherein n is 2 and each R_{1b} is H; and wherein R₂ is —C(═O)R_{1b}, wherein R_{1b} is C₁-C₂₀ alkyl.

In some embodiments, the compound is of Formula (I), wherein T is *—C(═O)—(CHR_{1b})ₙ—C(═O)—**, wherein n is 2 and each R_{1b} is H; wherein R₂ is —C(═O)R_{1b}, wherein R_{1b} is C₁-C₂₀ alkyl substituted with one R_{1c}; R_{1c} is —N(R_{1g})₂; and each R_{1g} is H.

In some embodiments, the compound is of Formula (I), wherein T is *—C(═O)—(CHR_{1b})ₙ—C(═O)—**, wherein n is 2 and each R_{1b} is H; wherein R₂ is —C(═O)R_{1b}, wherein R_{1b} is C₁-C₂₀ alkyl substituted with two R₁ₑ; R₁ₑ is —N(R_{1g})₂ and C₁-C₂₀ alkyl; and each R_{1g} is H.

In some embodiments, the compound is of Formula (II), wherein T is *—C(═O)—(CHR_{1b})ₙ—C(═O)—**, n is 2, each R_{1b} is H, and R₃ is H.

In some embodiments, the compound is of Formula (I), wherein T is *—C(═O)—(CHR_{1b})ₙ—C(═O)—**, n is 2, each R_{1b} is H, R₂ is Si(R_{1g})₃, and each R_{1g} is C₁-C₂₀ alkyl.

In some embodiments, the compound is of Formula (II), wherein T is *—C(═O)—(CHR_{1b})ₙ—C(═O)—**, n is 2, each R_{1b} is H, R₃ is Si(R_{1g})₃, and each R_{1g} is C₁-C₂₀ alkyl.

In some embodiments, the compound is selected from the compounds described in Tables 1-3, and pharmaceutically acceptable salts thereof.

In some embodiments, the compound is selected from the compounds described in Tables 1-3.

In some embodiments, the compound is selected from the compounds described in Table 1 and pharmaceutically acceptable salts thereof.

In some embodiments, the compound is selected from the compounds described in Table 1.

In some embodiments, the compound is selected from the compounds described in Table 2 and pharmaceutically acceptable salts thereof.

In some embodiments, the compound is selected from the compounds described in Table 2.

In some embodiments, the compound is selected from the compounds described in Table 3 and pharmaceutically acceptable salts thereof.

In some embodiments, the compound is selected from the compounds described in Table 3.

In some embodiments, the compound is selected from Compound Nos. 1-2, 16, 18, 41-42, 56, 58, 61-62, 76, 78, 101-102, 116, 118, 146-147, 161, 163, 186-187, 201, 203, 206, 209, 236, 240, 246-247, 261, 263, 339-340, and pharmaceutically acceptable salts thereof.

In some embodiments, the compound is selected from Compound Nos. 1-2, 16, 18, 41-42, 56, 58, 61-62, 76, 78, 101-102, 116, 118, 146-147, 161, 163, 186-187, 201, 203, 206, 209, 236, 240, 246-247, 261, 263, 339-340.

In some embodiments, the compound is selected from Compound Nos. 1-2, 58, 61-62, 76, 78, 101, 116, 118, 186, 206, 209, 339-340, and pharmaceutically acceptable salts thereof.

In some embodiments, the compound is selected from Compound Nos. 1-2, 58, 61-62, 76, 78, 101, 116, 118, 186, 206, 209, and 339-340.

In some embodiments, the compound is selected from Compound Nos. 1-2, 58, 101, 116, 118, 186, 209, and pharmaceutically acceptable salts thereof.

In some embodiments, the compound is selected from Compound Nos. 1-2, 58, 101, 116, 118, 186, and 209.

In some embodiments, the compound is selected from Compound Nos. 61-62, 76, 78, 206, 339-340, and pharmaceutically acceptable salts thereof.

In some embodiments, the compound is selected from Compound Nos. 61-62, 76, 78, 206, and 339-340.

In some embodiments, the compound is Compound 1 or a pharmaceutically acceptable salt thereof.

In some embodiments, the compound is Compound 1.

In some embodiments, the compound is Compound 2 or a pharmaceutically acceptable salt thereof.

In some embodiments, the compound is Compound 2.

In some embodiments, the compound is Compound 58 or a pharmaceutically acceptable salt thereof.

In some embodiments, the compound is Compound 58.

In some embodiments, the compound is Compound 61 or a pharmaceutically acceptable salt thereof.

In some embodiments, the compound is Compound 61.

In some embodiments, the compound is Compound 62 or a pharmaceutically acceptable salt thereof.

In some embodiments, the compound is Compound 62.

In some embodiments, the compound is Compound 76 or a pharmaceutically acceptable salt thereof.

In some embodiments, the compound is Compound 76.

In some embodiments, the compound is Compound 78 or a pharmaceutically acceptable salt thereof.

In some embodiments, the compound is Compound 78.

In some embodiments, the compound is Compound 101 or a pharmaceutically acceptable salt thereof.

In some embodiments, the compound is Compound 101.

In some embodiments, the compound is Compound 116 or a pharmaceutically acceptable salt thereof.

In some embodiments, the compound is Compound 116.

In some embodiments, the compound is Compound 118 or a pharmaceutically acceptable salt thereof.

In some embodiments, the compound is Compound 118.

In some embodiments, the compound is Compound 186 or a pharmaceutically acceptable salt thereof.

In some embodiments, the compound is Compound 186.

In some embodiments, the compound is Compound 206 or a pharmaceutically acceptable salt thereof.

In some embodiments, the compound is Compound 206.

In some embodiments, the compound is Compound 209 or a pharmaceutically acceptable salt thereof.

In some embodiments, the compound is Compound 209.

In some embodiments, the compound is Compound 339 or a pharmaceutically acceptable salt thereof.

In some embodiments, the compound is Compound 339.

In some embodiments, the compound is Compound 340 or a pharmaceutically acceptable salt thereof.

In some embodiments, the compound is Compound 340.

TABLE 1

| Compound No. | Structure |
|---|---|
| 1 | 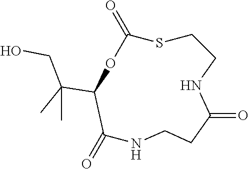 [SMILES: CC(C)(CO)[C@H]1OC(=O)SCCNC(=O)CCNC1=O] |
| 2 | 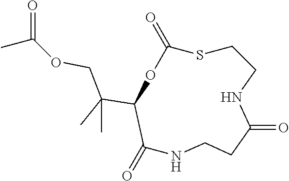 [SMILES: CC(=O)OCC(C)(C)[C@H]1OC(=O)SCCNC(=O)CCNC1=O] |
| 3 | 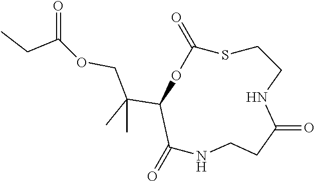 [SMILES: CCC(=O)OCC(C)(C)[C@H]1OC(=O)SCCNC(=O)CCNC1=O] |
| 4 | 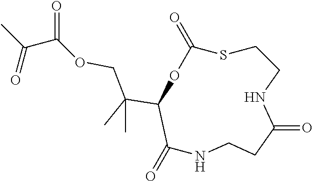 [SMILES: CC(=O)C(=O)OCC(C)(C)[C@H]1OC(=O)SCCNC(=O)CCNC1=O] |
| 5 | 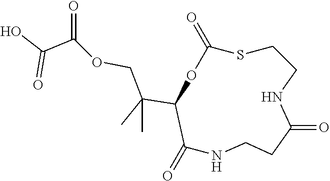 [SMILES: CC(C)(COC(=O)C(=O)O)[C@H]1OC(=O)SCCNC(=O)CCNC1=O] |
| 6 | 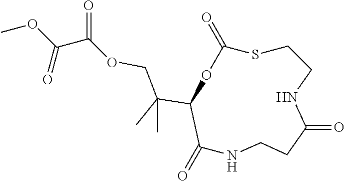 [SMILES: COC(=O)C(=O)OCC(C)(C)[C@H]1OC(=O)SCCNC(=O)CCNC1=O] |
| 7 | 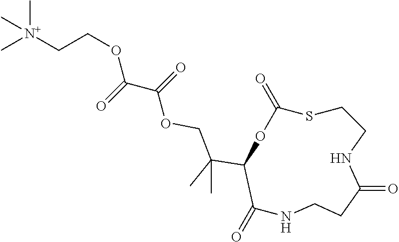 [SMILES: CC(C)(COC(=O)C(=O)OCC[N+](C)(C)C)[C@H]1OC(=O)SCCNC(=O)CCNC1=O] |
| 8 | 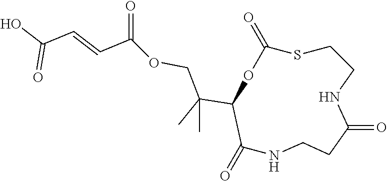 [SMILES: CC(C)(COC(=O)/C=C/C(=O)O)[C@H]1OC(=O)SCCNC(=O)CCNC1=O] |
| 9 | 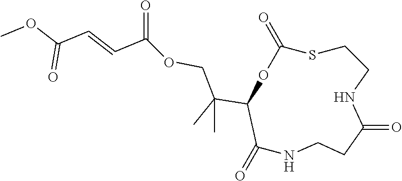 [SMILES: COC(=O)/C=C/C(=O)OCC(C)(C)[C@H]1OC(=O)SCCNC(=O)CCNC1=O] |
| 10 | 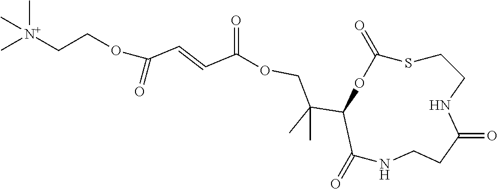 [SMILES: CC(C)(COC(=O)/C=C/C(=O)OCC[N+](C)(C)C)[C@H]1OC(=O)SCCNC(=O)CCNC1=O] |
| 11 | 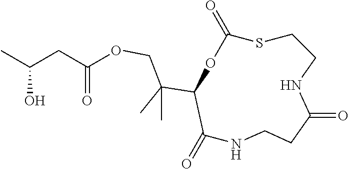 [SMILES: C[C@@H](O)CC(=O)OCC(C)(C)[C@H]1OC(=O)SCCNC(=O)CCNC1=O] |
| 12 | 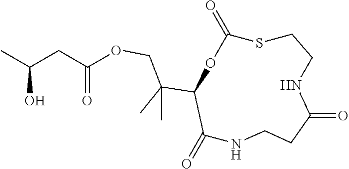 [SMILES: C[C@H](O)CC(=O)OCC(C)(C)[C@H]1OC(=O)SCCNC(=O)CCNC1=O] |
| 13 | 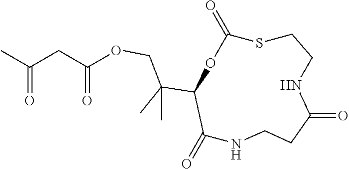 [SMILES: CC(=O)CC(=O)OCC(C)(C)[C@H]1OC(=O)SCCNC(=O)CCNC1=O] |
| 14 | 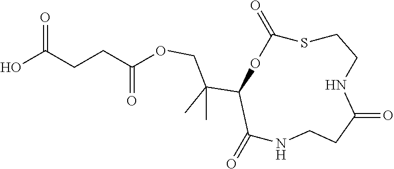 [SMILES: CC(C)(COC(=O)CCC(=O)O)[C@H]1OC(=O)SCCNC(=O)CCNC1=O] |
| 15 | 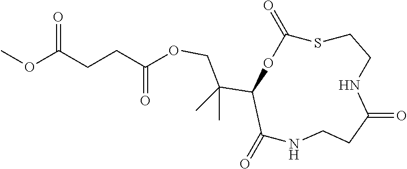 [SMILES: COC(=O)CCC(=O)OCC(C)(C)[C@H]1OC(=O)SCCNC(=O)CCNC1=O] |
| 16 | 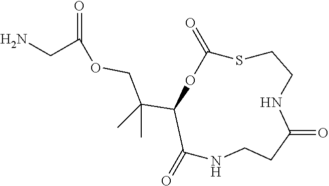 [SMILES: CC(C)(COC(=O)CN)[C@H]1OC(=O)SCCNC(=O)CCNC1=O] |
| 17 | 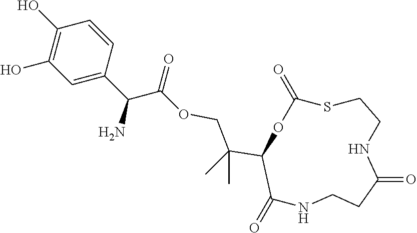 [SMILES: CC(C)(COC(=O)[C@@H](N)c1ccc(O)c(O)c1)[C@H]1OC(=O)SCCNC(=O)CCNC1=O] |
| 18 | 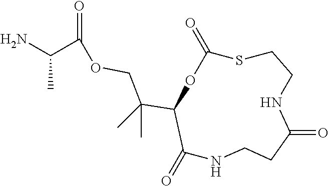 [SMILES: C[C@H](N)C(=O)OCC(C)(C)[C@H]1OC(=O)SCCNC(=O)CCNC1=O] |
| 19 | 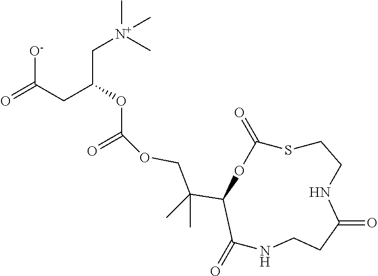 [SMILES: CC(C)(COC(=O)O[C@H](CC(=O)[O-])C[N+](C)(C)C)[C@H]1OC(=O)SCCNC(=O)CCNC1=O] |
| 20 | 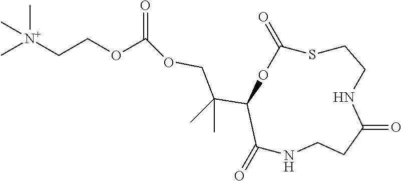 [SMILES: CC(C)(COC(=O)OCC[N+](C)(C)C)[C@H]1OC(=O)SCCNC(=O)CCNC1=O] |
| 21 | 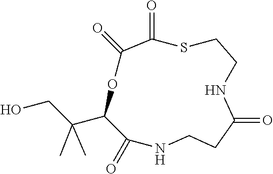 [SMILES: CC(C)(CO)[C@H]1OC(=O)C(=O)SCCNC(=O)CCNC1=O] |
| 22 | 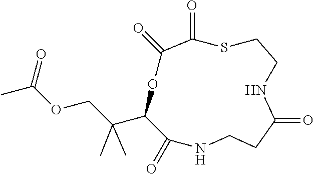 [SMILES: CC(=O)OCC(C)(C)[C@H]1OC(=O)C(=O)SCCNC(=O)CCNC1=O] |
| 23 | 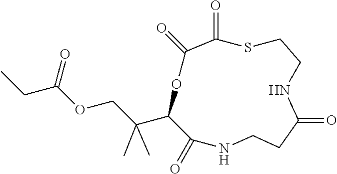 [SMILES: CCC(=O)OCC(C)(C)[C@H]1OC(=O)C(=O)SCCNC(=O)CCNC1=O] |
| 24 | 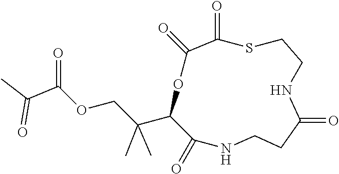 [SMILES: CC(=O)C(=O)OCC(C)(C)[C@H]1OC(=O)C(=O)SCCNC(=O)CCNC1=O] |
| 25 | 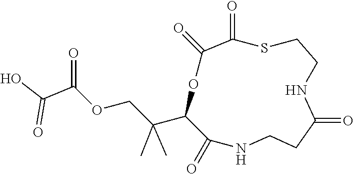 [SMILES: CC(C)(COC(=O)C(=O)O)[C@H]1OC(=O)C(=O)SCCNC(=O)CCNC1=O] |
| 26 | 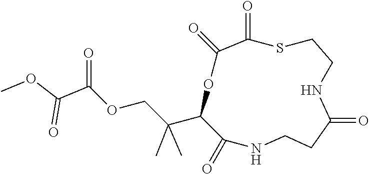 [SMILES: COC(=O)C(=O)OCC(C)(C)[C@H]1OC(=O)C(=O)SCCNC(=O)CCNC1=O] |
| 27 | 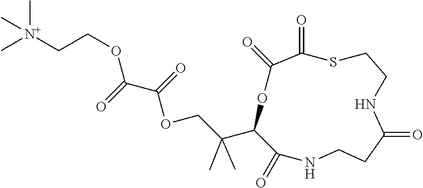 [SMILES: CC(C)(COC(=O)C(=O)OCC[N+](C)(C)C)[C@H]1OC(=O)C(=O)SCCNC(=O)CCNC1=O] |
| 28 | 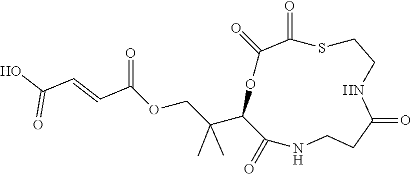 [SMILES: CC(C)(COC(=O)/C=C/C(=O)O)[C@H]1OC(=O)C(=O)SCCNC(=O)CCNC1=O] |
| 29 | 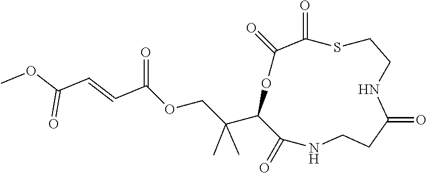 [SMILES: COC(=O)/C=C/C(=O)OCC(C)(C)[C@H]1OC(=O)C(=O)SCCNC(=O)CCNC1=O] |
| 30 | 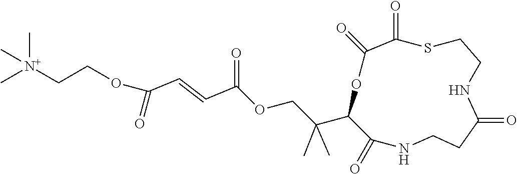 [SMILES: CC(C)(COC(=O)/C=C/C(=O)OCC[N+](C)(C)C)[C@H]1OC(=O)C(=O)SCCNC(=O)CCNC1=O] |
| 31 | 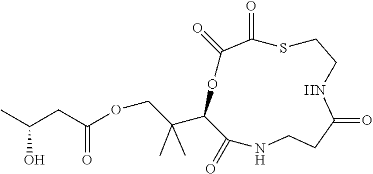 [SMILES: C[C@@H](O)CC(=O)OCC(C)(C)[C@H]1OC(=O)C(=O)SCCNC(=O)CCNC1=O] |
| 32 | 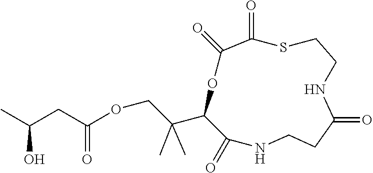 [SMILES: C[C@H](O)CC(=O)OCC(C)(C)[C@H]1OC(=O)C(=O)SCCNC(=O)CCNC1=O] |
| 33 | 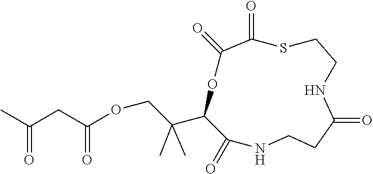 [SMILES: CC(=O)CC(=O)OCC(C)(C)[C@H]1OC(=O)C(=O)SCCNC(=O)CCNC1=O] |
| 34 | 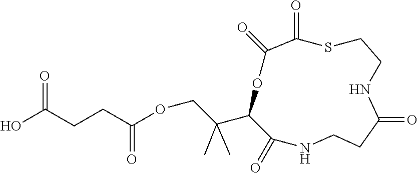 [SMILES: CC(C)(COC(=O)CCC(=O)O)[C@H]1OC(=O)C(=O)SCCNC(=O)CCNC1=O] |
| 35 | 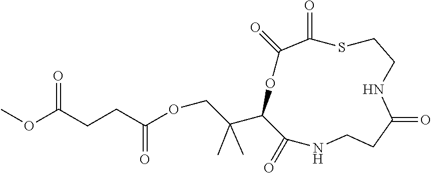 [SMILES: COC(=O)CCC(=O)OCC(C)(C)[C@H]1OC(=O)C(=O)SCCNC(=O)CCNC1=O] |
| 36 | 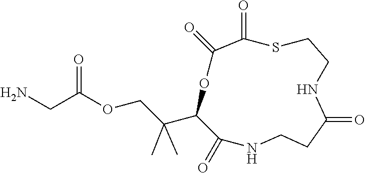 [SMILES: CC(C)(COC(=O)CN)[C@H]1OC(=O)C(=O)SCCNC(=O)CCNC1=O] |
| 37 | 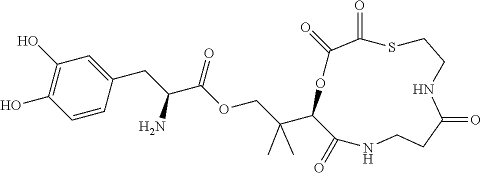 [SMILES: CC(C)(COC(=O)[C@@H](N)Cc1ccc(O)c(O)c1)[C@H]1OC(=O)C(=O)SCCNC(=O)CCNC1=O] |
| 38 | 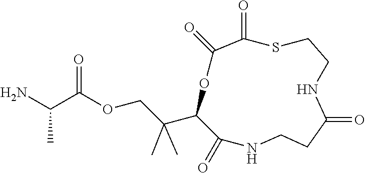 [SMILES: C[C@H](N)C(=O)OCC(C)(C)[C@H]1OC(=O)C(=O)SCCNC(=O)CCNC1=O] |
| 39 | 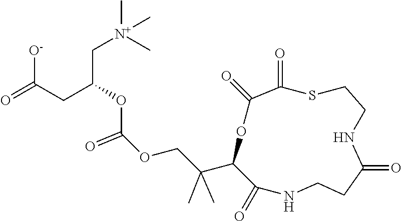 [SMILES: CC(C)(COC(=O)O[C@H](CC(=O)[O-])C[N+](C)(C)C)[C@H]1OC(=O)C(=O)SCCNC(=O)CCNC1=O] |
| 40 | 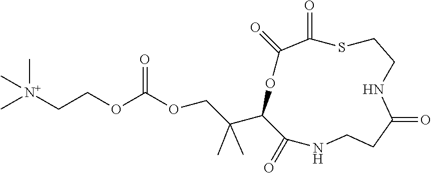 [SMILES: CC(C)(COC(=O)OCC[N+](C)(C)C)[C@H]1OC(=O)C(=O)SCCNC(=O)CCNC1=O] |
| 41 | 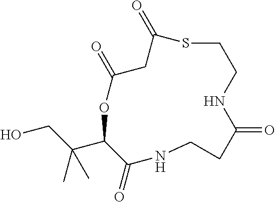 [SMILES: CC(C)(CO)[C@H]1OC(=O)CC(=O)SCCNC(=O)CCNC1=O] |
| 42 | 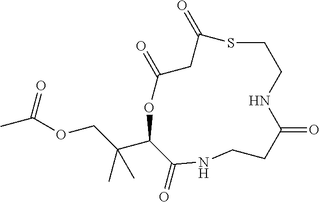 |
| 43 | 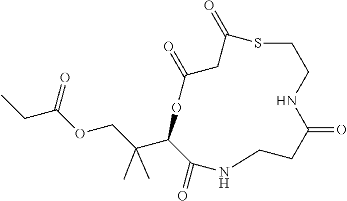 [SMILES: CCC(=O)OCC(C)(C)[C@H]1OC(=O)CC(=O)SCCNC(=O)CCNC1=O] |
| 44 | 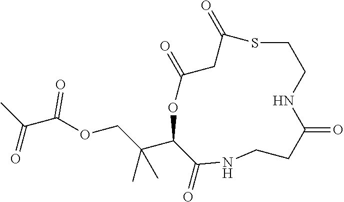 |
| 45 | 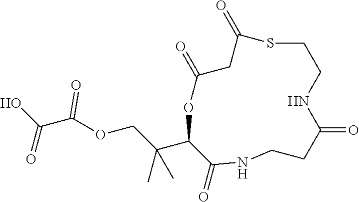 |
| 46 | 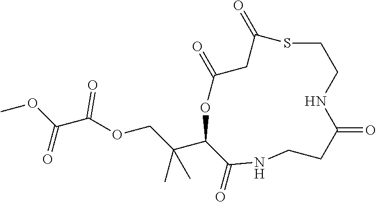 |
| 47 | 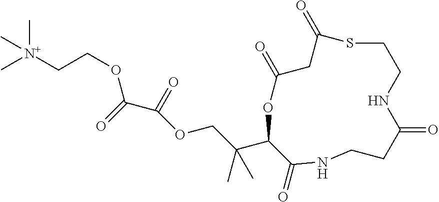 |
| 48 | 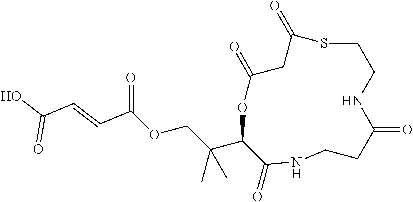 |
| 49 | 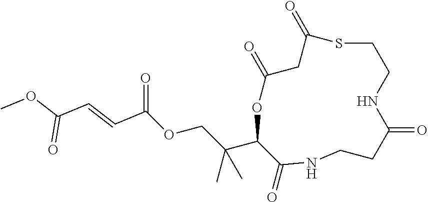 |
| 50 | 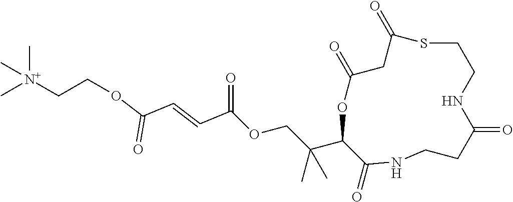 |
| 51 | 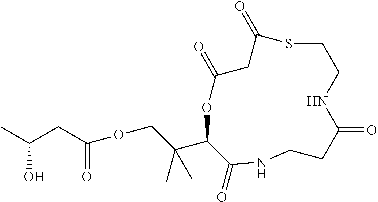 |
| 52 | 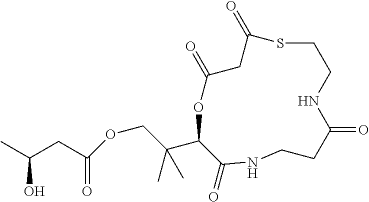 |
| 53 | 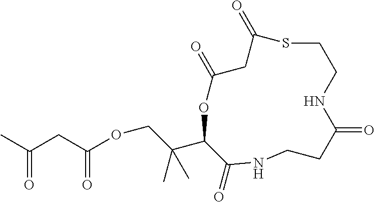 |
| 54 | 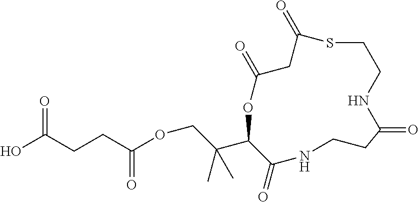 [SMILES: CC(C)(COC(=O)CCC(=O)O)[C@H]1OC(=O)CC(=O)SCCNC(=O)CCNC1=O] |
| 55 | 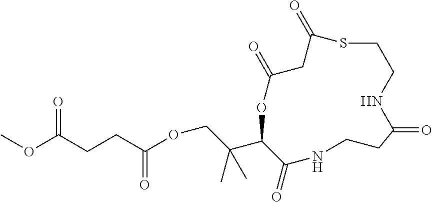 [SMILES: COC(=O)CCC(=O)OCC(C)(C)[C@H]1OC(=O)CC(=O)SCCNC(=O)CCNC1=O] |
| 56 | 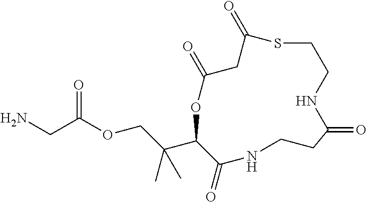 [SMILES: CC(C)(COC(=O)CN)[C@H]1OC(=O)CC(=O)SCCNC(=O)CCNC1=O] |
| 57 | 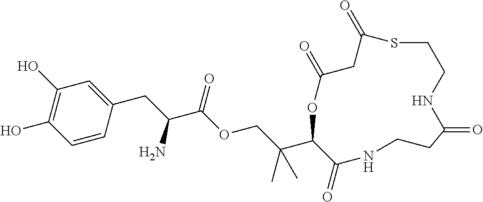 [SMILES: CC(C)(COC(=O)[C@@H](N)Cc1ccc(O)c(O)c1)[C@H]1OC(=O)CC(=O)SCCNC(=O)CCNC1=O] |
| 58 | 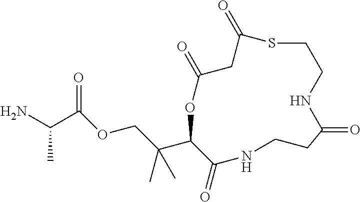 [SMILES: C[C@H](N)C(=O)OCC(C)(C)[C@H]1OC(=O)CC(=O)SCCNC(=O)CCNC1=O] |
| 59 | 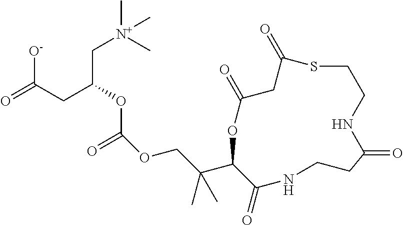 [SMILES: CC(C)(COC(=O)O[C@H](CC(=O)[O-])C[N+](C)(C)C)[C@H]1OC(=O)CC(=O)SCCNC(=O)CCNC1=O] |
| 60 | 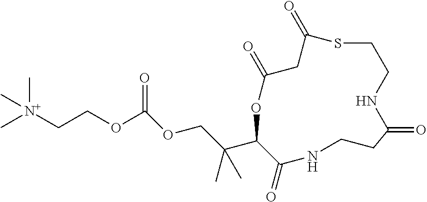 [SMILES: CC(C)(COC(=O)OCC[N+](C)(C)C)[C@H]1OC(=O)CC(=O)SCCNC(=O)CCNC1=O] |
| 61 | 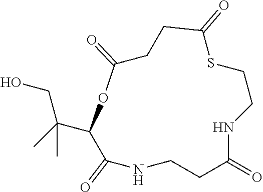 [SMILES: CC(C)(CO)[C@H]1OC(=O)CCC(=O)SCCNC(=O)CCNC1=O] |
| 62 | 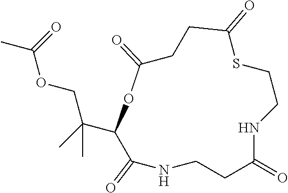 [SMILES: CC(=O)OCC(C)(C)[C@H]1OC(=O)CCC(=O)SCCNC(=O)CCNC1=O] |
| 63 | 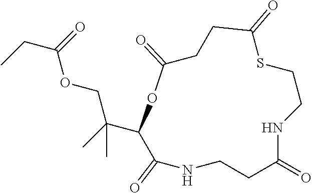 [SMILES: CCC(=O)OCC(C)(C)[C@H]1OC(=O)CCC(=O)SCCNC(=O)CCNC1=O] |
| 64 | 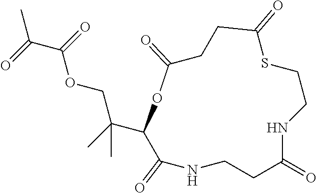 [SMILES: CC(=O)C(=O)OCC(C)(C)[C@H]1OC(=O)CCC(=O)SCCNC(=O)CCNC1=O] |
| 65 | 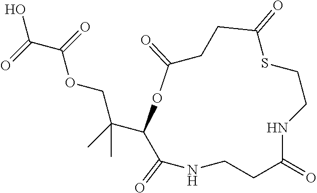 [SMILES: CC(C)(COC(=O)C(=O)O)[C@H]1OC(=O)CCC(=O)SCCNC(=O)CCNC1=O] |
| 66 | 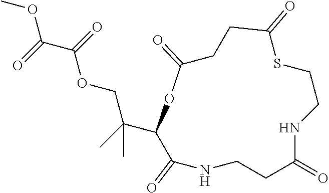 [SMILES: COC(=O)C(=O)OCC(C)(C)[C@H]1OC(=O)CCC(=O)SCCNC(=O)CCNC1=O] |
| 67 | 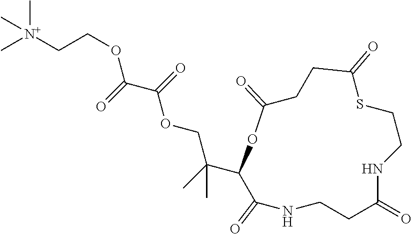 [SMILES: CC(C)(COC(=O)C(=O)OCC[N+](C)(C)C)[C@H]1OC(=O)CCC(=O)SCCNC(=O)CCNC1=O] |
| 68 | 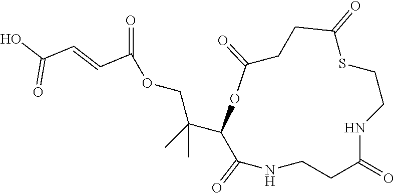 [SMILES: CC(C)(COC(=O)/C=C/C(=O)O)[C@H]1OC(=O)CCC(=O)SCCNC(=O)CCNC1=O] |
| 69 | 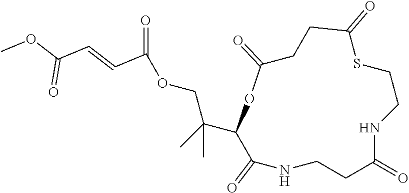 [SMILES: COC(=O)/C=C/C(=O)OCC(C)(C)[C@H]1OC(=O)CCC(=O)SCCNC(=O)CCNC1=O] |
| 70 | 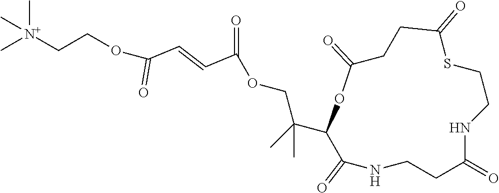 [SMILES: CC(C)(COC(=O)/C=C/C(=O)OCC[N+](C)(C)C)[C@H]1OC(=O)CCC(=O)SCCNC(=O)CCNC1=O] |
| 71 | 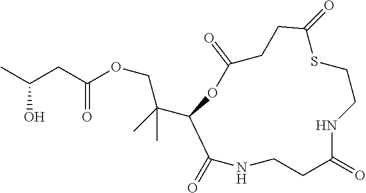 [SMILES: C[C@@H](O)CC(=O)OCC(C)(C)[C@H]1OC(=O)CCC(=O)SCCNC(=O)CCNC1=O] |
| 72 | 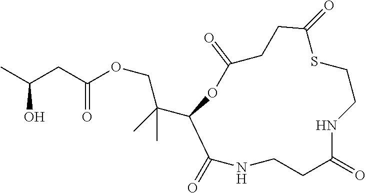 [SMILES: C[C@H](O)CC(=O)OCC(C)(C)[C@H]1OC(=O)CCC(=O)SCCNC(=O)CCNC1=O] |
| 73 | 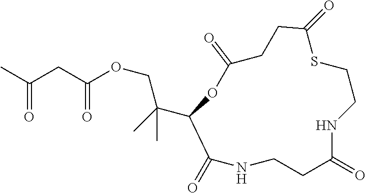 [SMILES: CC(=O)CC(=O)OCC(C)(C)[C@H]1OC(=O)CCC(=O)SCCNC(=O)CCNC1=O] |
| 74 | 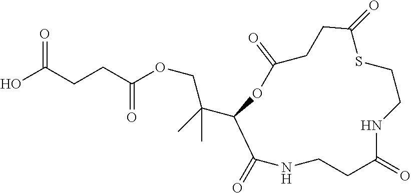 [SMILES: CC(C)(COC(=O)CCC(=O)O)[C@H]1OC(=O)CCC(=O)SCCNC(=O)CCNC1=O] |
| 75 | 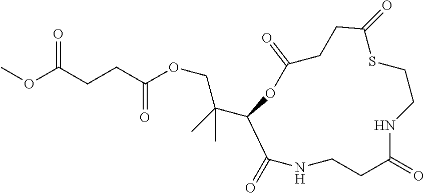 [SMILES: COC(=O)CCC(=O)OCC(C)(C)[C@H]1OC(=O)CCC(=O)SCCNC(=O)CCNC1=O] |
| 76 | 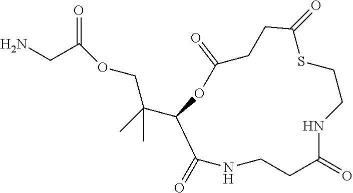 [SMILES: CC(C)(COC(=O)CN)[C@H]1OC(=O)CCC(=O)SCCNC(=O)CCNC1=O] |
| 77 | 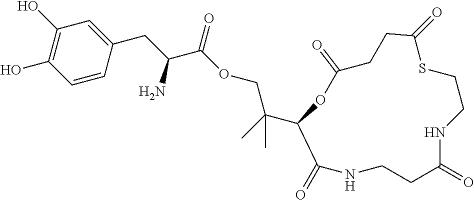 [SMILES: CC(C)(COC(=O)[C@@H](N)Cc1ccc(O)c(O)c1)[C@H]1OC(=O)CCC(=O)SCCNC(=O)CCNC1=O] |
| 78 | 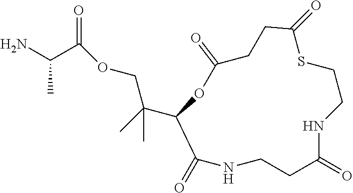 [SMILES: C[C@H](N)C(=O)OCC(C)(C)[C@H]1OC(=O)CCC(=O)SCCNC(=O)CCNC1=O] |
| 79 | 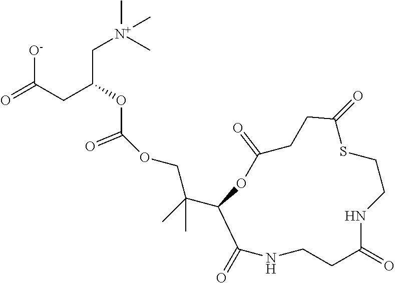 [SMILES: CC(C)(COC(=O)O[C@H](CC(=O)[O-])C[N+](C)(C)C)[C@H]1OC(=O)CCC(=O)SCCNC(=O)CCNC1=O] |
| 80 | 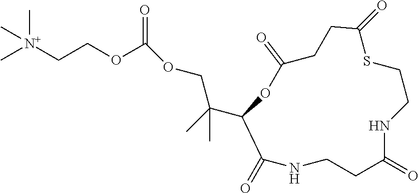 [SMILES: CC(C)(COC(=O)OCC[N+](C)(C)C)[C@H]1OC(=O)CCC(=O)SCCNC(=O)CCNC1=O] |
| 81 | 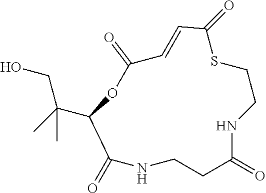 [SMILES: CC(C)(CO)[C@H]1OC(=O)/C=C/C(=O)SCCNC(=O)CCNC1=O] |
| 82 | 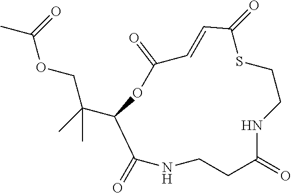 [SMILES: CC(=O)OCC(C)(C)[C@H]1OC(=O)/C=C/C(=O)SCCNC(=O)CCNC1=O] |
| 83 | 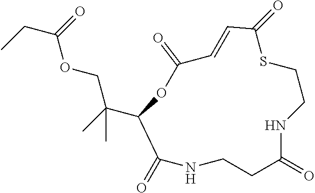 [SMILES: CCC(=O)OCC(C)(C)[C@H]1OC(=O)/C=C/C(=O)SCCNC(=O)CCNC1=O] |
| 84 | 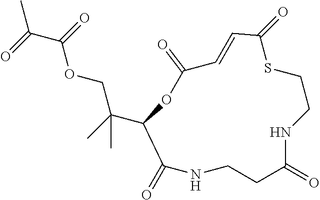 [SMILES: CC(=O)C(=O)OCC(C)(C)[C@H]1OC(=O)/C=C/C(=O)SCCNC(=O)CCNC1=O] |
| 85 | 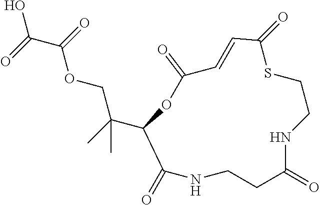 [SMILES: CC(C)(COC(=O)C(=O)O)[C@H]1OC(=O)/C=C/C(=O)SCCNC(=O)CCNC1=O] |
| 86 | 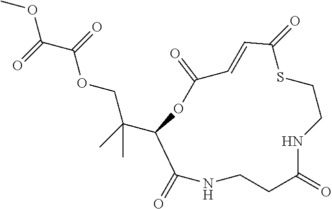 [SMILES: COC(=O)C(=O)OCC(C)(C)[C@H]1OC(=O)/C=C/C(=O)SCCNC(=O)CCNC1=O] |
| 87 | 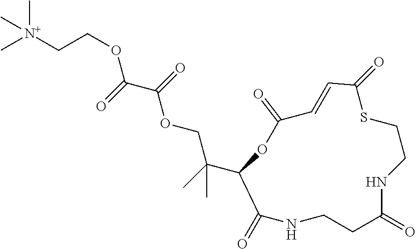 [SMILES: CC(C)(COC(=O)C(=O)OCC[N+](C)(C)C)[C@H]1OC(=O)/C=C/C(=O)SCCNC(=O)CCNC1=O] |
| 88 | 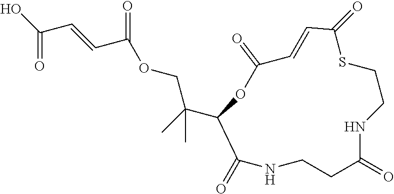 [SMILES: CC(C)(COC(=O)/C=C/C(=O)O)[C@H]1OC(=O)/C=C/C(=O)SCCNC(=O)CCNC1=O] |
| 89 | 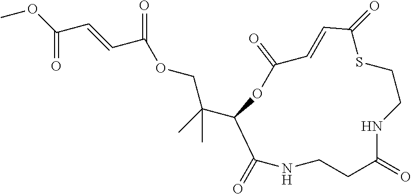 [SMILES: COC(=O)/C=C/C(=O)OCC(C)(C)[C@H]1OC(=O)/C=C/C(=O)SCCNC(=O)CCNC1=O] |
| 90 | 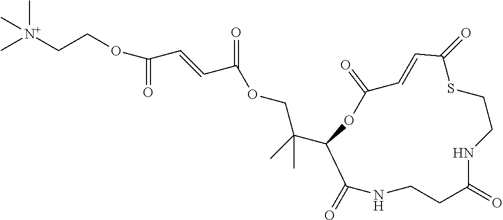 [SMILES: CC(C)(COC(=O)/C=C/C(=O)OCC[N+](C)(C)C)[C@H]1OC(=O)/C=C/C(=O)SCCNC(=O)CCNC1=O] |
| 91 | 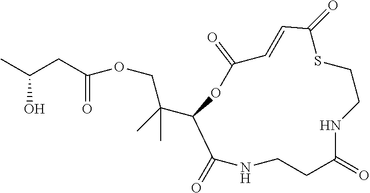 [SMILES: C[C@@H](O)CC(=O)OCC(C)(C)[C@H]1OC(=O)/C=C/C(=O)SCCNC(=O)CCNC1=O] |
| 92 | 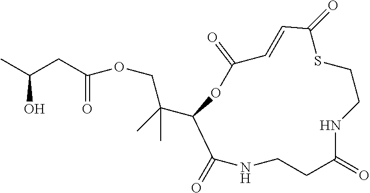 [SMILES: C[C@H](O)CC(=O)OCC(C)(C)[C@H]1OC(=O)/C=C/C(=O)SCCNC(=O)CCNC1=O] |
| 93 | 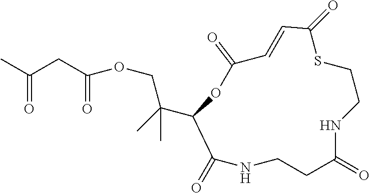 [SMILES: CC(=O)CC(=O)OCC(C)(C)[C@H]1OC(=O)/C=C/C(=O)SCCNC(=O)CCNC1=O] |
| 94 | 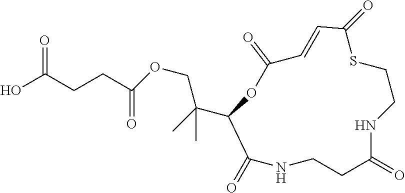 [SMILES: CC(C)(COC(=O)CCC(=O)O)[C@H]1OC(=O)/C=C/C(=O)SCCNC(=O)CCNC1=O] |
| 95 | 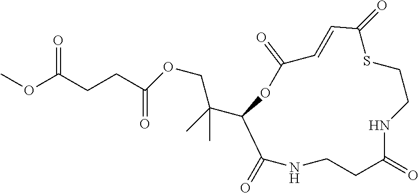 [SMILES: COC(=O)CCC(=O)OCC(C)(C)[C@H]1OC(=O)/C=C/C(=O)SCCNC(=O)CCNC1=O] |
| 96 | 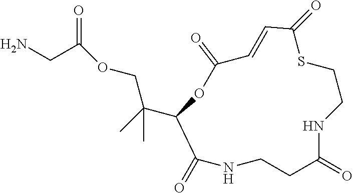 [SMILES: CC(C)(COC(=O)CN)[C@H]1OC(=O)/C=C/C(=O)SCCNC(=O)CCNC1=O] |
| 97 | 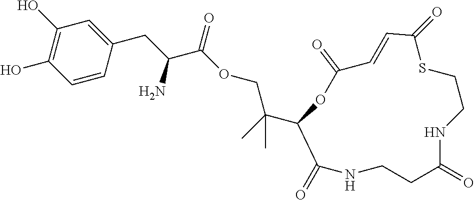 [SMILES: CC(C)(COC(=O)[C@@H](N)Cc1ccc(O)c(O)c1)[C@H]1OC(=O)/C=C/C(=O)SCCNC(=O)CCNC1=O] |
| 98 | 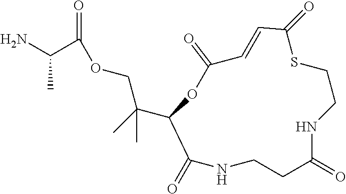 [SMILES: C[C@H](N)C(=O)OCC(C)(C)[C@H]1OC(=O)/C=C/C(=O)SCCNC(=O)CCNC1=O] |
| 99 | 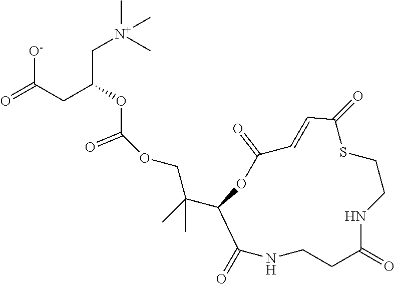 [SMILES: CC(C)(COC(=O)O[C@H](CC(=O)[O-])C[N+](C)(C)C)[C@H]1OC(=O)/C=C/C(=O)SCCNC(=O)CCNC1=O] |
| 100 | 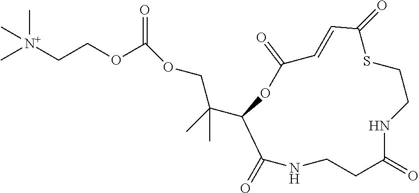 [SMILES: CC(C)(COC(=O)OCC[N+](C)(C)C)[C@H]1OC(=O)/C=C/C(=O)SCCNC(=O)CCNC1=O] |
| 101 | 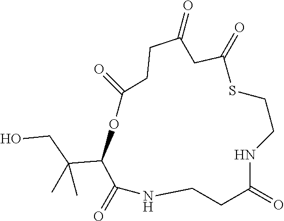 [SMILES: CC(C)(CO)[C@H]1OC(=O)CCC(=O)CC(=O)SCCNC(=O)CCNC1=O] |
| 102 | 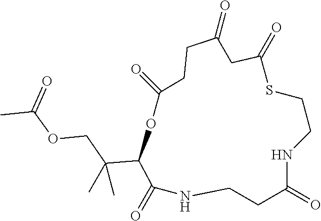 [SMILES: CC(=O)OCC(C)(C)[C@H]1OC(=O)CCC(=O)CC(=O)SCCNC(=O)CCNC1=O] |
| 103 | 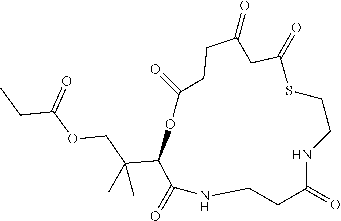 [SMILES: CCC(=O)OCC(C)(C)[C@H]1OC(=O)CCC(=O)CC(=O)SCCNC(=O)CCNC1=O] |
| 104 | 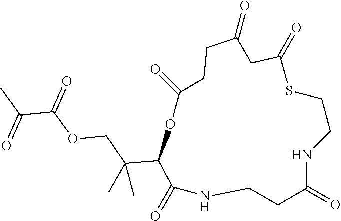 [SMILES: CC(=O)C(=O)OCC(C)(C)[C@H]1OC(=O)CCC(=O)CC(=O)SCCNC(=O)CCNC1=O] |
| 105 | 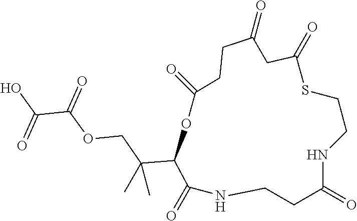 [SMILES: CC(C)(COC(=O)C(=O)O)[C@H]1OC(=O)CCC(=O)CC(=O)SCCNC(=O)CCNC1=O] |
| 106 | 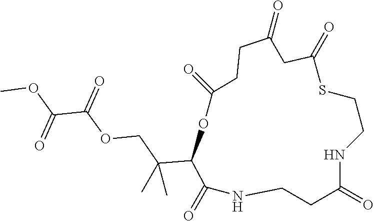 [SMILES: COC(=O)C(=O)OCC(C)(C)[C@H]1OC(=O)CCC(=O)CC(=O)SCCNC(=O)CCNC1=O] |
| 107 | 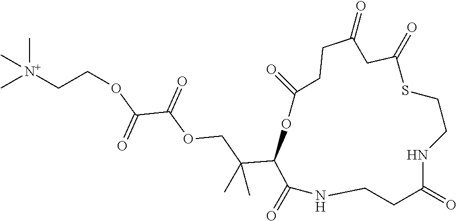 [SMILES: CC(C)(COC(=O)C(=O)OCC[N+](C)(C)C)[C@H]1OC(=O)CCC(=O)CC(=O)SCCNC(=O)CCNC1=O] |
| 108 | 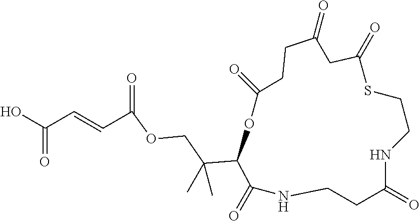 [SMILES: CC(C)(COC(=O)/C=C/C(=O)O)[C@H]1OC(=O)CCC(=O)CC(=O)SCCNC(=O)CCNC1=O] |
| 109 | 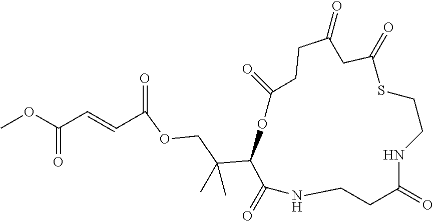 [SMILES: COC(=O)/C=C/C(=O)OCC(C)(C)[C@H]1OC(=O)CCC(=O)CC(=O)SCCNC(=O)CCNC1=O] |
| 110 | 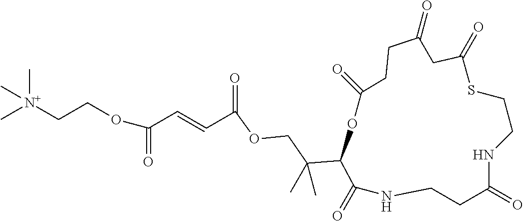 [SMILES: CC(C)(COC(=O)/C=C/C(=O)OCC[N+](C)(C)C)[C@H]1OC(=O)CCC(=O)CC(=O)SCCNC(=O)CCNC1=O] |
| 111 | 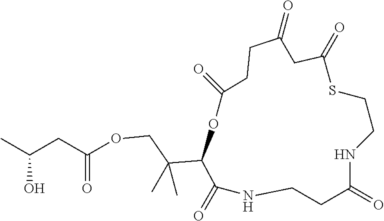 [SMILES: C[C@@H](O)CC(=O)OCC(C)(C)[C@H]1OC(=O)CCC(=O)CC(=O)SCCNC(=O)CCNC1=O] |
| 112 | 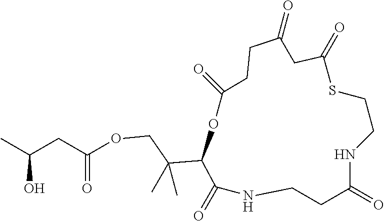 [SMILES: C[C@H](O)CC(=O)OCC(C)(C)[C@H]1OC(=O)CCC(=O)CC(=O)SCCNC(=O)CCNC1=O] |
| 113 | 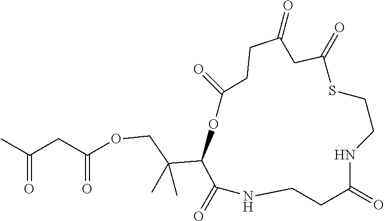 [SMILES: CC(=O)CC(=O)OCC(C)(C)[C@H]1OC(=O)CCC(=O)CC(=O)SCCNC(=O)CCNC1=O] |
| 114 | 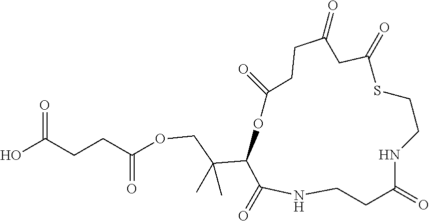 [SMILES: CC(C)(COC(=O)CCC(=O)O)[C@H]1OC(=O)CCC(=O)CC(=O)SCCNC(=O)CCNC1=O] |
| 115 | 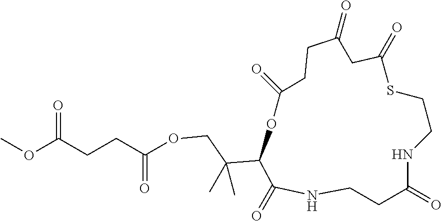 [SMILES: COC(=O)CCC(=O)OCC(C)(C)[C@H]1OC(=O)CCC(=O)CC(=O)SCCNC(=O)CCNC1=O] |
| 116 | 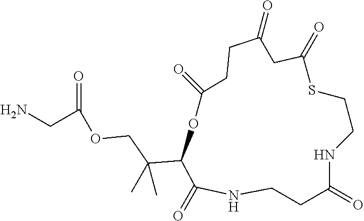 [SMILES: CC(C)(COC(=O)CN)[C@H]1OC(=O)CCC(=O)CC(=O)SCCNC(=O)CCNC1=O] |
| 117 | 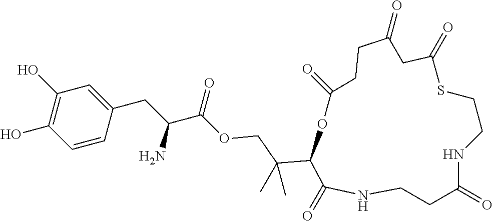 [SMILES: CC(C)(COC(=O)[C@@H](N)Cc1ccc(O)c(O)c1)[C@H]1OC(=O)CCC(=O)CC(=O)SCCNC(=O)CCNC1=O] |
| 118 | 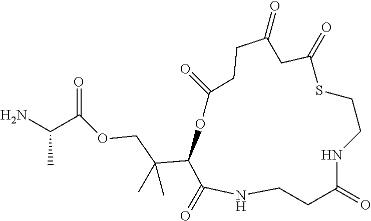 [SMILES: C[C@H](N)C(=O)OCC(C)(C)[C@H]1OC(=O)CCC(=O)CC(=O)SCCNC(=O)CCNC1=O] |
| 119 | 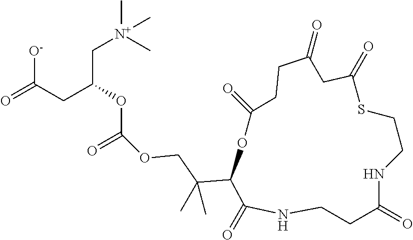 [SMILES: CC(C)(COC(=O)O[C@H](CC(=O)[O-])C[N+](C)(C)C)[C@H]1OC(=O)CCC(=O)CC(=O)SCCNC(=O)CCNC1=O] |
| 120 | 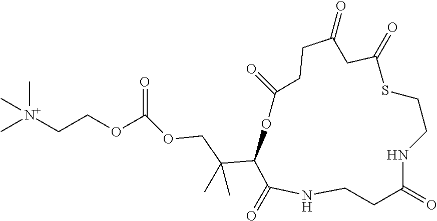 [SMILES: CC(C)(COC(=O)OCC[N+](C)(C)C)[C@H]1OC(=O)CCC(=O)CC(=O)SCCNC(=O)CCNC1=O] |
| 121 | 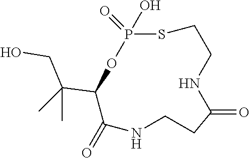 [SMILES: CC(C)(CO)[C@H]1OP(=O)(O)SCCNC(=O)CCNC1=O] |
| 122 | 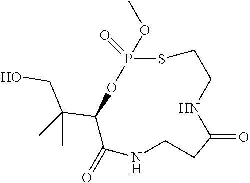 [SMILES: COP1(=O)O[C@H](C(C)(C)CO)C(=O)NCCC(=O)NCCS1] |
| 123 | 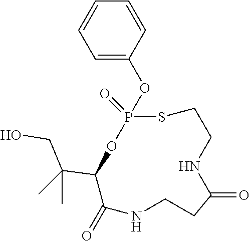 [SMILES: CC(C)(CO)[C@H]1OP(=O)(Oc2ccccc2)SCCNC(=O)CCNC1=O] |
| 124 | 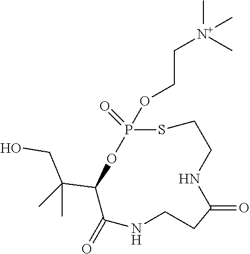 [SMILES: CC(C)(CO)[C@H]1OP(=O)(OCC[N+](C)(C)C)SCCNC(=O)CCNC1=O] |
| 125 | 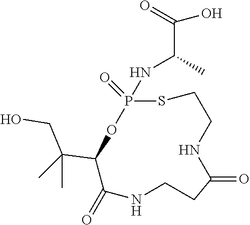 [SMILES: C[C@H](NP1(=O)O[C@H](C(C)(C)CO)C(=O)NCCC(=O)NCCS1)C(=O)O] |
| 126 | 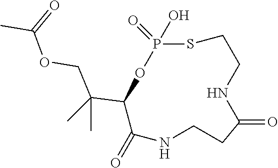 [SMILES: CC(=O)OCC(C)(C)[C@H]1OP(=O)(O)SCCNC(=O)CCNC1=O] |
| 127 | 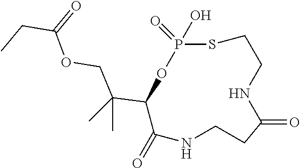 [SMILES: CCC(=O)OCC(C)(C)[C@H]1OP(=O)(O)SCCNC(=O)CCNC1=O] |
| 128 | 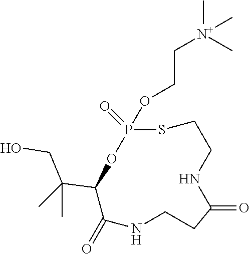 [SMILES: CC(C)(CO)[C@H]1OP(=O)(OCC[N+](C)(C)C)SCCNC(=O)CCNC1=O] |
| 129 | 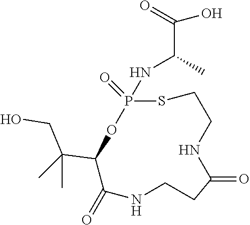 [SMILES: C[C@H](NP1(=O)O[C@H](C(C)(C)CO)C(=O)NCCC(=O)NCCS1)C(=O)O] |
| 130 | 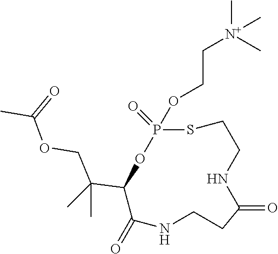 [SMILES: CC(=O)OCC(C)(C)[C@H]1OP(=O)(OCC[N+](C)(C)C)SCCNC(=O)CCNC1=O] |
| 131 | 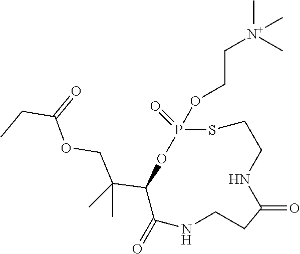 [SMILES: CCC(=O)OCC(C)(C)[C@H]1OP(=O)(OCC[N+](C)(C)C)SCCNC(=O)CCNC1=O] |
| 132 | 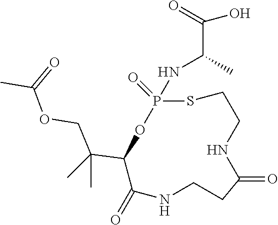 [SMILES: CC(=O)OCC(C)(C)[C@H]1OP(=O)(N[C@@H](C)C(=O)O)SCCNC(=O)CCNC1=O] |
| 133 | 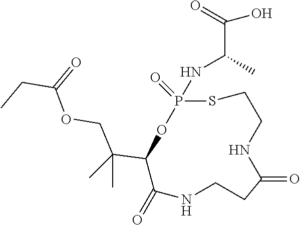 [SMILES: CCC(=O)OCC(C)(C)[C@H]1OP(=O)(N[C@@H](C)C(=O)O)SCCNC(=O)CCNC1=O] |
| 134 | 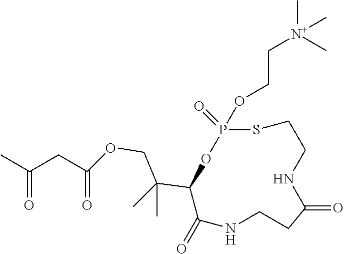 [SMILES: CC(=O)CC(=O)OCC(C)(C)[C@H]1OP(=O)(OCC[N+](C)(C)C)SCCNC(=O)CCNC1=O] |
| 135 | 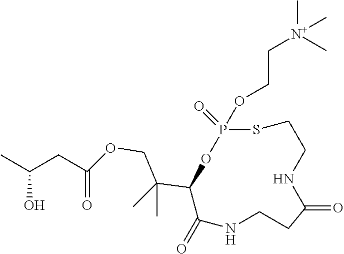 [SMILES: C[C@@H](O)CC(=O)OCC(C)(C)[C@H]1OP(=O)(OCC[N+](C)(C)C)SCCNC(=O)CCNC1=O] |
| 136 | 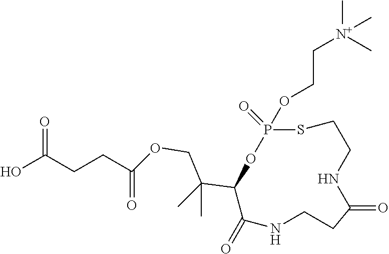 [SMILES: CC(C)(COC(=O)CCC(=O)O)[C@H]1OP(=O)(OCC[N+](C)(C)C)SCCNC(=O)CCNC1=O] |
| 137 | 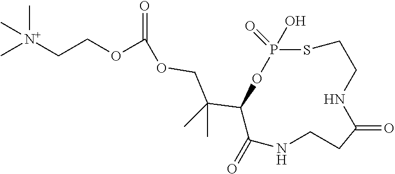 [SMILES: CC(C)(COC(=O)OCC[N+](C)(C)C)[C@H]1OP(=O)(O)SCCNC(=O)CCNC1=O] |
| 138 | 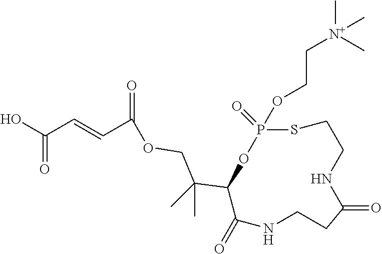 [SMILES: CC(C)(COC(=O)/C=C/C(=O)O)[C@H]1OP(=O)(OCC[N+](C)(C)C)SCCNC(=O)CCNC1=O] |
| 139 | 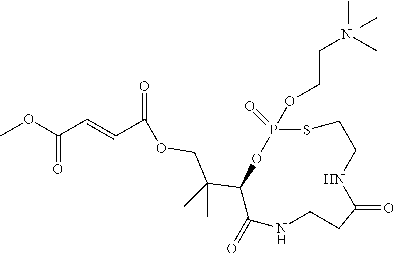 [SMILES: COC(=O)/C=C/C(=O)OCC(C)(C)[C@H]1OP(=O)(OCC[N+](C)(C)C)SCCNC(=O)CCNC1=O] |
| 140 | 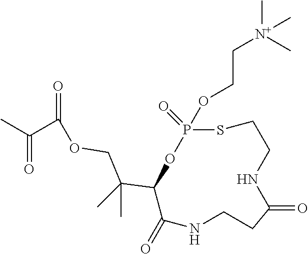 [SMILES: CC(=O)C(=O)OCC(C)(C)[C@H]1OP(=O)(OCC[N+](C)(C)C)SCCNC(=O)CCNC1=O] |
| 141 | 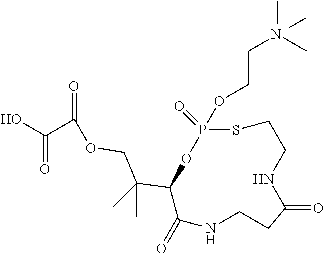 [SMILES: CC(C)(COC(=O)C(=O)O)[C@H]1OP(=O)(OCC[N+](C)(C)C)SCCNC(=O)CCNC1=O] |
| 142 | 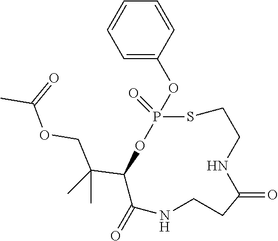 [SMILES: CC(=O)OCC(C)(C)[C@H]1OP(=O)(Oc2ccccc2)SCCNC(=O)CCNC1=O] |
| 143 | 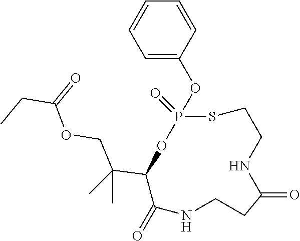 [SMILES: CCC(=O)OCC(C)(C)[C@H]1OP(=O)(Oc2ccccc2)SCCNC(=O)CCNC1=O] |
| 144 | 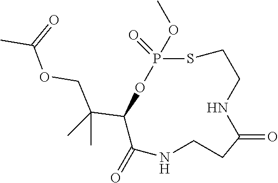 [SMILES: COP1(=O)O[C@H](C(C)(C)COC(C)=O)C(=O)NCCC(=O)NCCS1] |
| 145 | 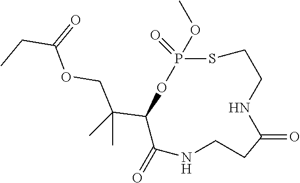 [SMILES: CCC(=O)OCC(C)(C)[C@H]1OP(=O)(OC)SCCNC(=O)CCNC1=O] |

TABLE 2

| Compound No. | Structure |
|---|---|
| 146 | 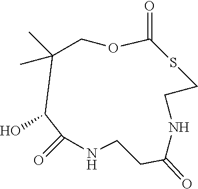 [SMILES: CC1(C)COC(=O)SCCNC(=O)CCNC(=O)[C@@H]1O] |
| 147 | 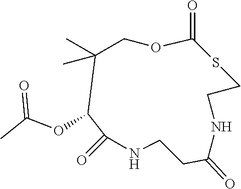 [SMILES: CC(=O)O[C@H]1C(=O)NCCC(=O)NCCSC(=O)OCC1(C)C] |
| 148 | 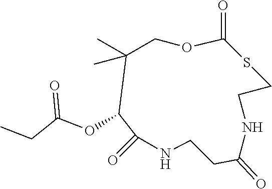 [SMILES: CCC(=O)O[C@H]1C(=O)NCCC(=O)NCCSC(=O)OCC1(C)C] |
| 149 | 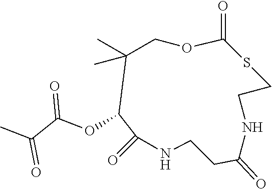 [SMILES: CC(=O)C(=O)O[C@H]1C(=O)NCCC(=O)NCCSC(=O)OCC1(C)C] |
| 150 | 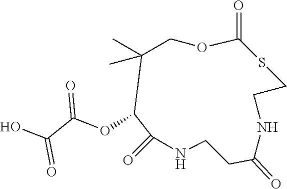 [SMILES: CC1(C)COC(=O)SCCNC(=O)CCNC(=O)[C@@H]1OC(=O)C(=O)O] |
| 151 | 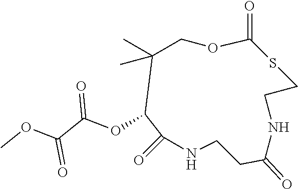 [SMILES: COC(=O)C(=O)O[C@H]1C(=O)NCCC(=O)NCCSC(=O)OCC1(C)C] |
| 152 | 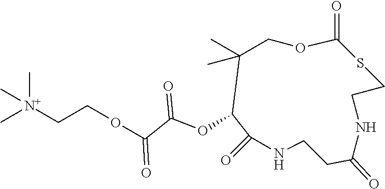 [SMILES: CC1(C)COC(=O)SCCNC(=O)CCNC(=O)[C@@H]1OC(=O)C(=O)OCC[N+](C)(C)C] |
| 153 | 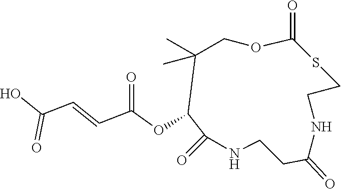 [SMILES: CC1(C)COC(=O)SCCNC(=O)CCNC(=O)[C@@H]1OC(=O)/C=C/C(=O)O] |
| 154 | 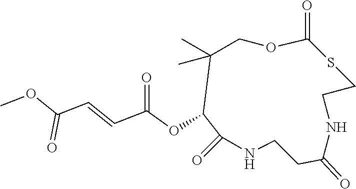 [SMILES: COC(=O)/C=C/C(=O)O[C@H]1C(=O)NCCC(=O)NCCSC(=O)OCC1(C)C] |
| 155 | 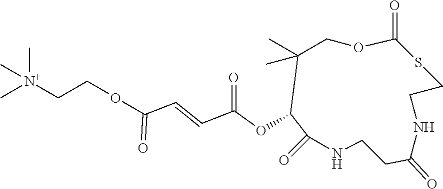 [SMILES: CC1(C)COC(=O)SCCNC(=O)CCNC(=O)[C@@H]1OC(=O)/C=C/C(=O)OCC[N+](C)(C)C] |
| 156 | 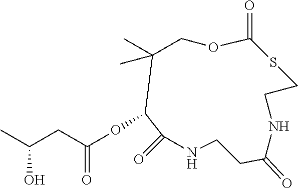 [SMILES: C[C@@H](O)CC(=O)O[C@H]1C(=O)NCCC(=O)NCCSC(=O)OCC1(C)C] |
| 157 | 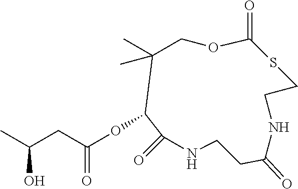 [SMILES: C[C@H](O)CC(=O)O[C@H]1C(=O)NCCC(=O)NCCSC(=O)OCC1(C)C] |
| 158 | 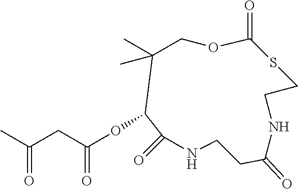 [SMILES: CC(=O)CC(=O)O[C@H]1C(=O)NCCC(=O)NCCSC(=O)OCC1(C)C] |
| 159 | 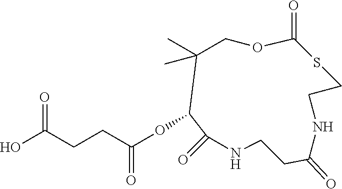 [SMILES: CC1(C)COC(=O)SCCNC(=O)CCNC(=O)[C@@H]1OC(=O)CCC(=O)O] |
| 160 | 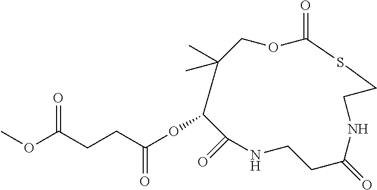 [SMILES: COC(=O)CCC(=O)O[C@H]1C(=O)NCCC(=O)NCCSC(=O)OCC1(C)C] |
| 161 | 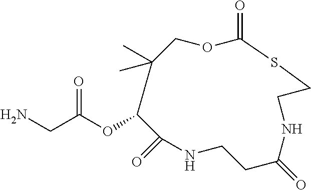 [SMILES: CC1(C)COC(=O)SCCNC(=O)CCNC(=O)[C@@H]1OC(=O)CN] |
| 162 | 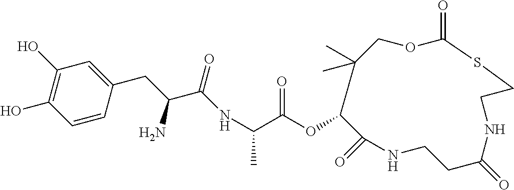 [SMILES: C[C@H](NC(=O)[C@@H](N)Cc1ccc(O)c(O)c1)C(=O)O[C@H]1C(=O)NCCC(=O)NCCSC(=O)OCC1(C)C] |
| 163 | 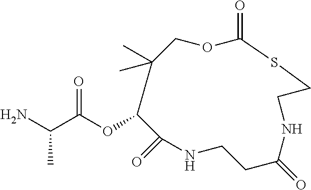 [SMILES: C[C@H](N)C(=O)O[C@H]1C(=O)NCCC(=O)NCCSC(=O)OCC1(C)C] |
| 164 | 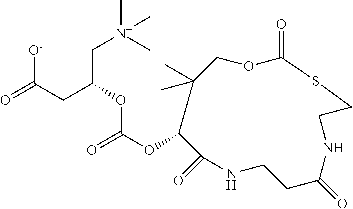 [SMILES: CC1(C)COC(=O)SCCNC(=O)CCNC(=O)[C@@H]1OC(=O)O[C@H](CC(=O)[O-])C[N+](C)(C)C] |
| 165 | 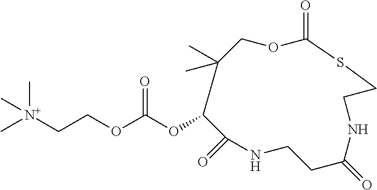 [SMILES: CC1(C)COC(=O)SCCNC(=O)CCNC(=O)[C@@H]1OC(=O)OCC[N+](C)(C)C] |
| 166 | 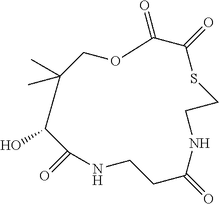 [SMILES: CC1(C)COC(=O)C(=O)SCCNC(=O)CCNC(=O)[C@@H]1O] |
| 167 | 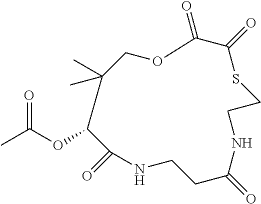 [SMILES: CC(=O)O[C@H]1C(=O)NCCC(=O)NCCSC(=O)C(=O)OCC1(C)C] |
| 168 | 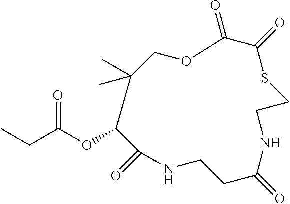 [SMILES: CCC(=O)O[C@H]1C(=O)NCCC(=O)NCCSC(=O)C(=O)OCC1(C)C] |
| 169 | 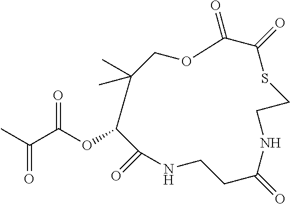 [SMILES: CC(=O)C(=O)O[C@H]1C(=O)NCCC(=O)NCCSC(=O)C(=O)OCC1(C)C] |
| 170 | 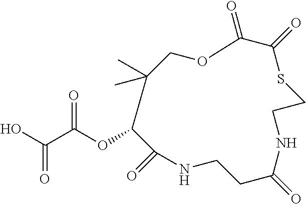 [SMILES: CC1(C)COC(=O)C(=O)SCCNC(=O)CCNC(=O)[C@@H]1OC(=O)C(=O)O] |
| 171 | 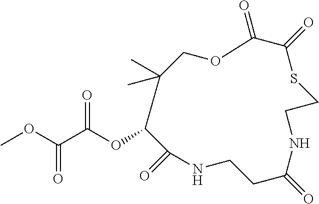 [SMILES: COC(=O)C(=O)O[C@H]1C(=O)NCCC(=O)NCCSC(=O)C(=O)OCC1(C)C] |
| 172 | 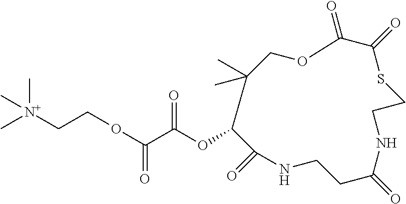 [SMILES: CC1(C)COC(=O)C(=O)SCCNC(=O)CCNC(=O)[C@@H]1OC(=O)C(=O)OCC[N+](C)(C)C] |
| 173 | 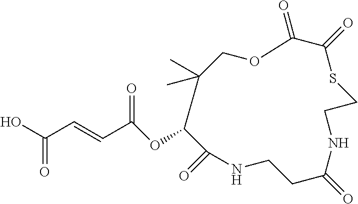 [SMILES: CC1(C)COC(=O)C(=O)SCCNC(=O)CCNC(=O)[C@@H]1OC(=O)/C=C/C(=O)O] |
| 174 | 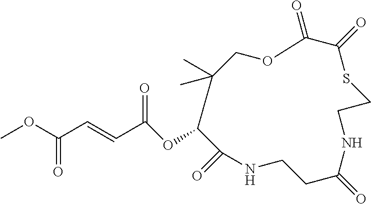 [SMILES: COC(=O)/C=C/C(=O)O[C@H]1C(=O)NCCC(=O)NCCSC(=O)C(=O)OCC1(C)C] |
| 175 | 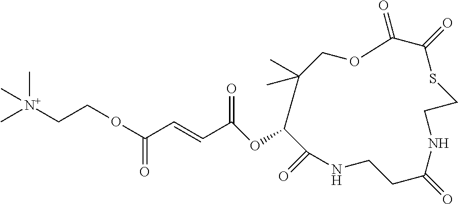 [SMILES: CC1(C)COC(=O)C(=O)SCCNC(=O)CCNC(=O)[C@@H]1OC(=O)/C=C/C(=O)OCC[N+](C)(C)C] |
| 176 | 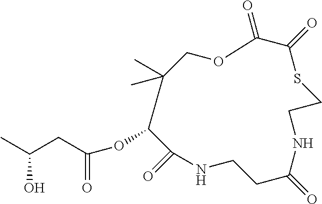 [SMILES: C[C@@H](O)CC(=O)O[C@H]1C(=O)NCCC(=O)NCCSC(=O)C(=O)OCC1(C)C] |
| 177 | 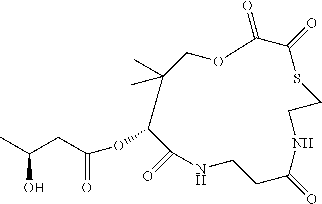 [SMILES: C[C@H](O)CC(=O)O[C@H]1C(=O)NCCC(=O)NCCSC(=O)C(=O)OCC1(C)C] |
| 178 | 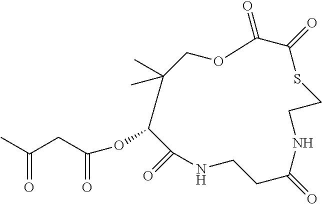 [SMILES: CC(=O)CC(=O)O[C@H]1C(=O)NCCC(=O)NCCSC(=O)C(=O)OCC1(C)C] |
| 179 | 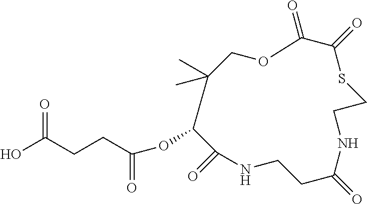 [SMILES: CC1(C)COC(=O)C(=O)SCCNC(=O)CCNC(=O)[C@@H]1OC(=O)CCC(=O)O] |
| 180 | 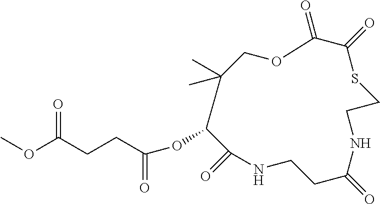 [SMILES: COC(=O)CCC(=O)O[C@H]1C(=O)NCCC(=O)NCCSC(=O)C(=O)OCC1(C)C] |
| 181 | 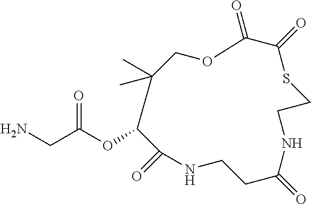 [SMILES: CC1(C)COC(=O)C(=O)SCCNC(=O)CCNC(=O)[C@@H]1OC(=O)CN] |
| 182 | 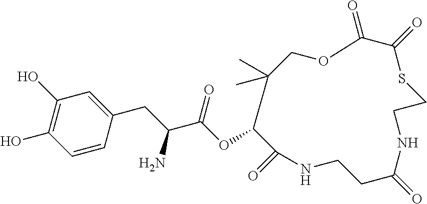 [SMILES: CC1(C)COC(=O)C(=O)SCCNC(=O)CCNC(=O)[C@@H]1OC(=O)[C@@H](N)Cc1ccc(O)c(O)c1] |
| 183 | 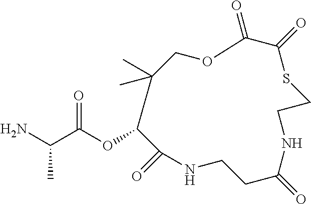 [SMILES: C[C@H](N)C(=O)O[C@H]1C(=O)NCCC(=O)NCCSC(=O)C(=O)OCC1(C)C] |
| 184 | 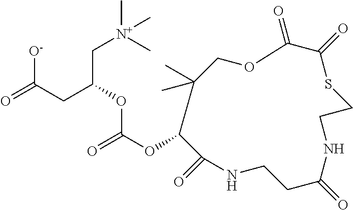 [SMILES: CC1(C)COC(=O)C(=O)SCCNC(=O)CCNC(=O)[C@@H]1OC(=O)O[C@H](CC(=O)[O-])C[N+](C)(C)C] |
| 185 | 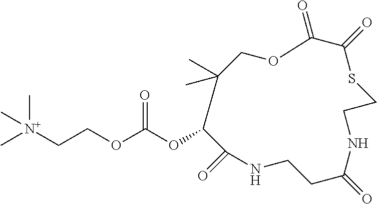 [SMILES: CC1(C)COC(=O)C(=O)SCCNC(=O)CCNC(=O)[C@@H]1OC(=O)OCC[N+](C)(C)C] |
| 186 | 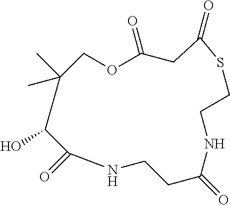 [SMILES: CC1(C)COC(=O)CC(=O)SCCNC(=O)CCNC(=O)[C@@H]1O] |
| 187 | 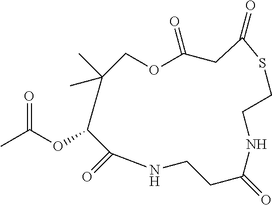 [SMILES: CC(=O)O[C@H]1C(=O)NCCC(=O)NCCSC(=O)CC(=O)OCC1(C)C] |
| 188 | 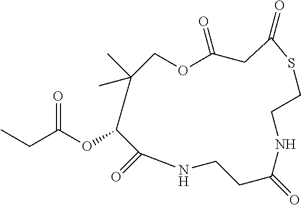 [SMILES: CCC(=O)O[C@H]1C(=O)NCCC(=O)NCCSC(=O)CC(=O)OCC1(C)C] |
| 189 | 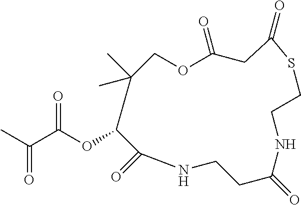 [SMILES: CC(=O)C(=O)O[C@H]1C(=O)NCCC(=O)NCCSC(=O)CC(=O)OCC1(C)C] |
| 190 | 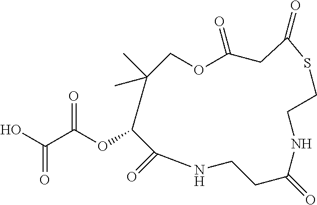 [SMILES: CC1(C)COC(=O)CC(=O)SCCNC(=O)CCNC(=O)[C@@H]1OC(=O)C(=O)O] |
| 191 | 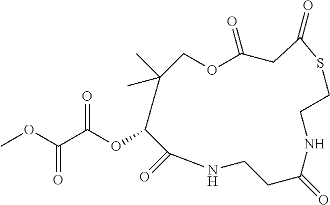 [SMILES: COC(=O)C(=O)O[C@H]1C(=O)NCCC(=O)NCCSC(=O)CC(=O)OCC1(C)C] |
| 192 | 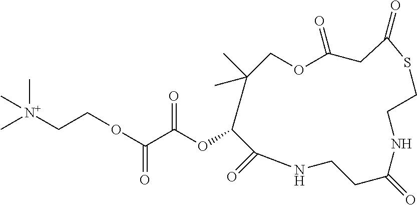 [SMILES: CC1(C)COC(=O)CC(=O)SCCNC(=O)CCNC(=O)[C@@H]1OC(=O)C(=O)OCC[N+](C)(C)C] |
| 193 | 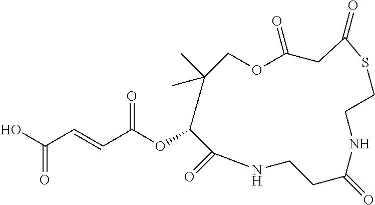 [SMILES: CC1(C)COC(=O)CC(=O)SCCNC(=O)CCNC(=O)[C@@H]1OC(=O)/C=C/C(=O)O] |
| 194 | 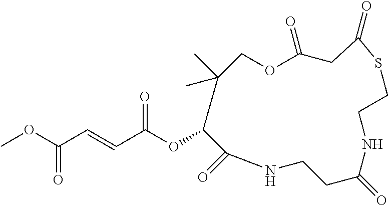 [SMILES: COC(=O)/C=C/C(=O)O[C@H]1C(=O)NCCC(=O)NCCSC(=O)CC(=O)OCC1(C)C] |
| 195 | 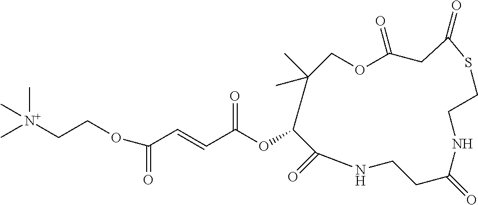 [SMILES: CC1(C)COC(=O)CC(=O)SCCNC(=O)CCNC(=O)[C@@H]1OC(=O)/C=C/C(=O)OCC[N+](C)(C)C] |
| 196 | 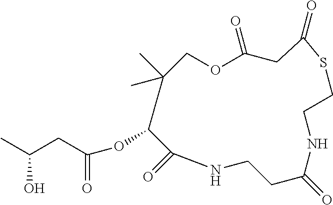 [SMILES: C[C@@H](O)CC(=O)O[C@H]1C(=O)NCCC(=O)NCCSC(=O)CC(=O)OCC1(C)C] |
| 197 | 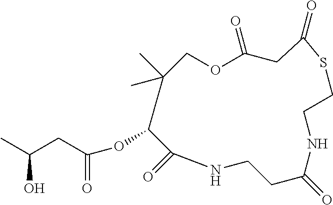 [SMILES: C[C@H](O)CC(=O)O[C@H]1C(=O)NCCC(=O)NCCSC(=O)CC(=O)OCC1(C)C] |
| 198 | 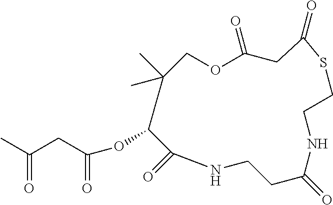 [SMILES: CC(=O)CC(=O)O[C@H]1C(=O)NCCC(=O)NCCSC(=O)CC(=O)OCC1(C)C] |
| 199 | 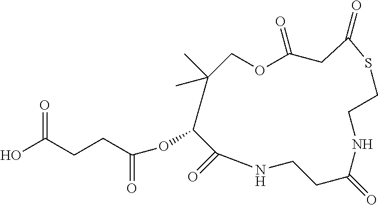 [SMILES: CC1(C)COC(=O)CC(=O)SCCNC(=O)CCNC(=O)[C@@H]1OC(=O)CCC(=O)O] |
| 200 | 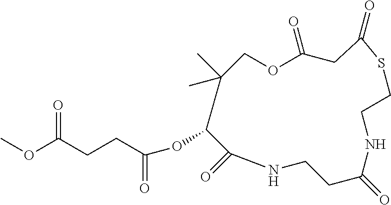 [SMILES: COC(=O)CCC(=O)O[C@H]1C(=O)NCCC(=O)NCCSC(=O)CC(=O)OCC1(C)C] |
| 201 | 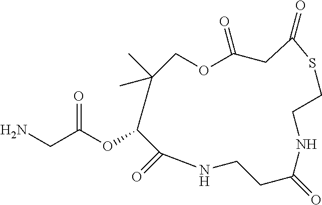 [SMILES: CC1(C)COC(=O)CC(=O)SCCNC(=O)CCNC(=O)[C@@H]1OC(=O)CN] |
| 202 | 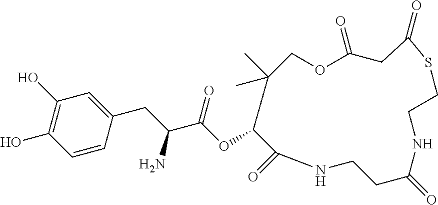 [SMILES: CC1(C)COC(=O)CC(=O)SCCNC(=O)CCNC(=O)[C@@H]1OC(=O)[C@@H](N)Cc1ccc(O)c(O)c1] |
| 203 | 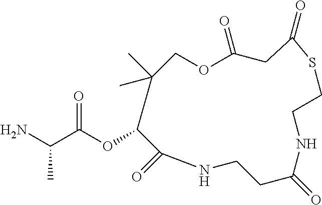 [SMILES: C[C@H](N)C(=O)O[C@H]1C(=O)NCCC(=O)NCCSC(=O)CC(=O)OCC1(C)C] |
| 204 | 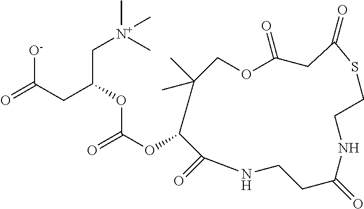 [SMILES: CC1(C)COC(=O)CC(=O)SCCNC(=O)CCNC(=O)[C@@H]1OC(=O)O[C@H](CC(=O)[O-])C[N+](C)(C)C] |
| 205 | 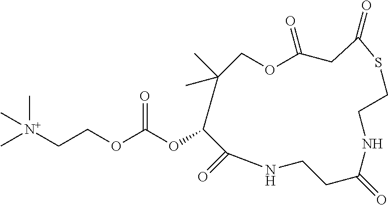 [SMILES: CC1(C)COC(=O)CC(=O)SCCNC(=O)CCNC(=O)[C@@H]1OC(=O)OCC[N+](C)(C)C] |
| 206 | 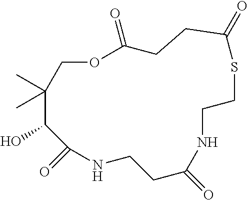 [SMILES: CC1(C)COC(=O)CCC(=O)SCCNC(=O)CCNC(=O)[C@@H]1O] |
| 207 | 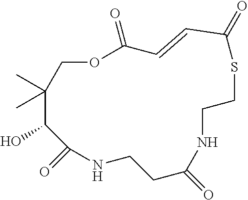 [SMILES: CC1(C)COC(=O)/C=C/C(=O)SCCNC(=O)CCNC(=O)[C@@H]1O] |
| 208 | 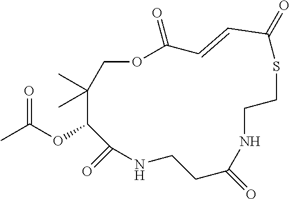 [SMILES: CC(=O)O[C@H]1C(=O)NCCC(=O)NCCSC(=O)/C=C/C(=O)OCC1(C)C] |
| 209 | 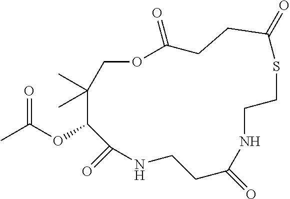 [SMILES: CC(=O)O[C@H]1C(=O)NCCC(=O)NCCSC(=O)CCC(=O)OCC1(C)C] |
| 210 | 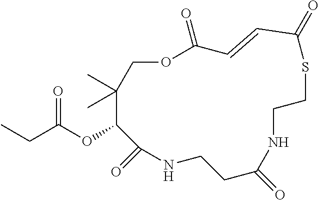 [SMILES: CCC(=O)O[C@H]1C(=O)NCCC(=O)NCCSC(=O)/C=C/C(=O)OCC1(C)C] |
| 211 | 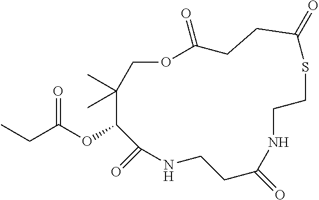 [SMILES: CCC(=O)O[C@H]1C(=O)NCCC(=O)NCCSC(=O)CCC(=O)OCC1(C)C] |
| 212 | 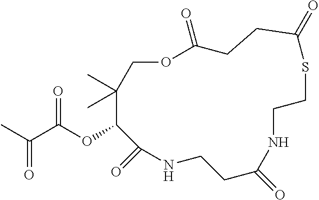 [SMILES: CC(=O)C(=O)O[C@H]1C(=O)NCCC(=O)NCCSC(=O)CCC(=O)OCC1(C)C] |
| 213 | 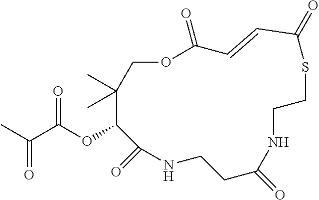 [SMILES: CC(=O)C(=O)O[C@H]1C(=O)NCCC(=O)NCCSC(=O)/C=C/C(=O)OCC1(C)C] |
| 214 | 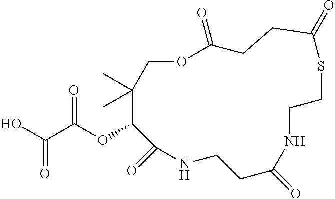 [SMILES: CC1(C)COC(=O)CCC(=O)SCCNC(=O)CCNC(=O)[C@@H]1OC(=O)C(=O)O] |
| 215 | 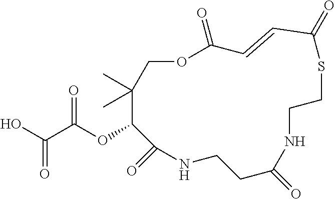 [SMILES: CC1(C)COC(=O)/C=C/C(=O)SCCNC(=O)CCNC(=O)[C@@H]1OC(=O)C(=O)O] |
| 216 | 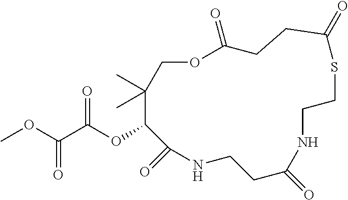 [SMILES: COC(=O)C(=O)O[C@H]1C(=O)NCCC(=O)NCCSC(=O)CCC(=O)OCC1(C)C] |
| 217 | 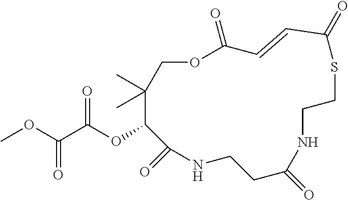 [SMILES: COC(=O)C(=O)O[C@H]1C(=O)NCCC(=O)NCCSC(=O)/C=C/C(=O)OCC1(C)C] |
| 218 | 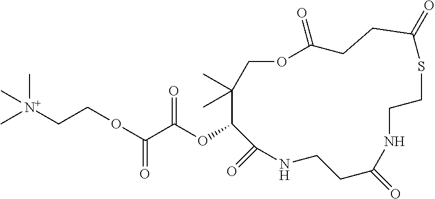 [SMILES: CC1(C)COC(=O)CCC(=O)SCCNC(=O)CCNC(=O)[C@@H]1OC(=O)C(=O)OCC[N+](C)(C)C] |
| 219 | 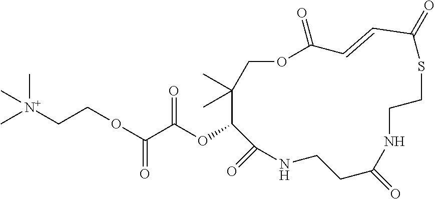 [SMILES: CC1(C)COC(=O)/C=C/C(=O)SCCNC(=O)CCNC(=O)[C@@H]1OC(=O)C(=O)OCC[N+](C)(C)C] |
| 220 | 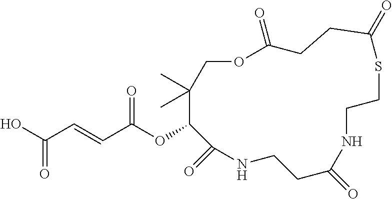 [SMILES: CC1(C)COC(=O)CCC(=O)SCCNC(=O)CCNC(=O)[C@@H]1OC(=O)/C=C/C(=O)O] |
| 221 | 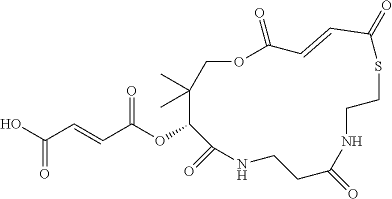 [SMILES: CC1(C)COC(=O)/C=C/C(=O)SCCNC(=O)CCNC(=O)[C@@H]1OC(=O)/C=C/C(=O)O] |
| 222 | 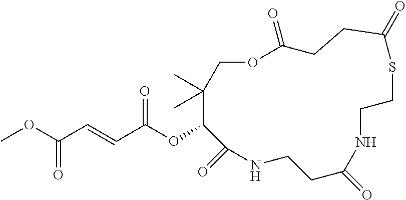 [SMILES: COC(=O)/C=C/C(=O)O[C@H]1C(=O)NCCC(=O)NCCSC(=O)CCC(=O)OCC1(C)C] |
| 223 | 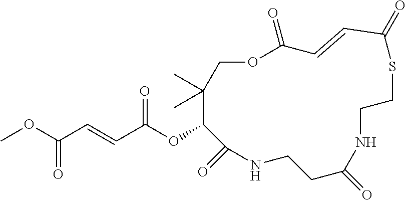 [SMILES: COC(=O)/C=C/C(=O)O[C@H]1C(=O)NCCC(=O)NCCSC(=O)/C=C/C(=O)OCC1(C)C] |
| 224 | 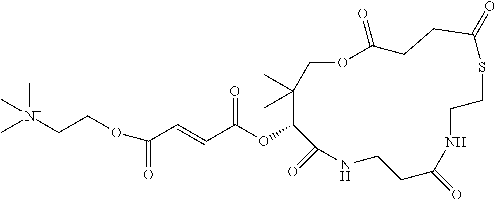 [SMILES: CC1(C)COC(=O)CCC(=O)SCCNC(=O)CCNC(=O)[C@@H]1OC(=O)/C=C/C(=O)OCC[N+](C)(C)C] |
| 225 | 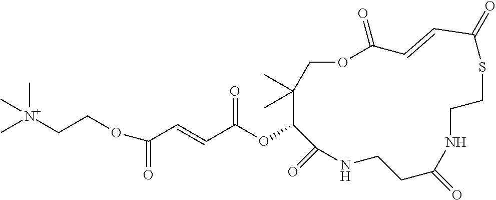 [SMILES: CC1(C)COC(=O)/C=C/C(=O)SCCNC(=O)CCNC(=O)[C@@H]1OC(=O)/C=C/C(=O)OCC[N+](C)(C)C] |
| 226 | 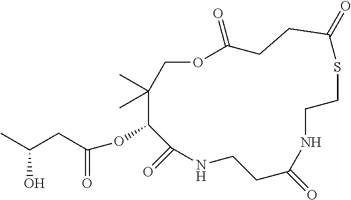 [SMILES: C[C@@H](O)CC(=O)O[C@H]1C(=O)NCCC(=O)NCCSC(=O)CCC(=O)OCC1(C)C] |
| 227 | 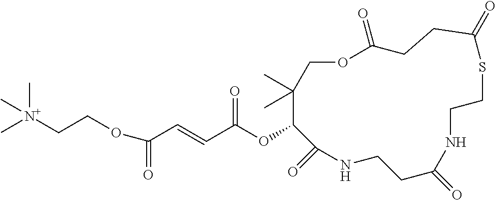 [SMILES: CC1(C)COC(=O)CCC(=O)SCCNC(=O)CCNC(=O)[C@@H]1OC(=O)/C=C/C(=O)OCC[N+](C)(C)C] |
| 228 | 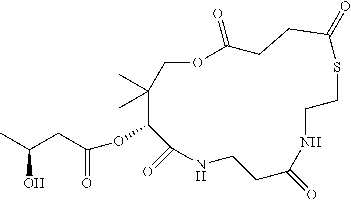 [SMILES: C[C@H](O)CC(=O)O[C@H]1C(=O)NCCC(=O)NCCSC(=O)CCC(=O)OCC1(C)C] |
| 229 | 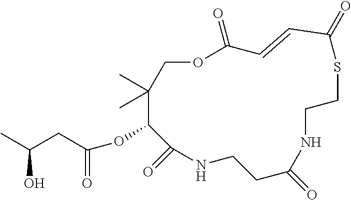 [SMILES: C[C@H](O)CC(=O)O[C@H]1C(=O)NCCC(=O)NCCSC(=O)/C=C/C(=O)OCC1(C)C] |
| 230 | 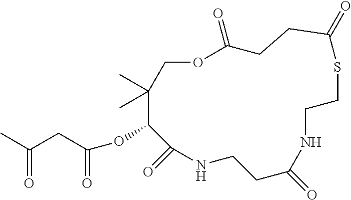 [SMILES: CC(=O)CC(=O)O[C@H]1C(=O)NCCC(=O)NCCSC(=O)CCC(=O)OCC1(C)C] |
| 231 | 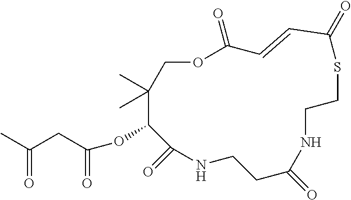 [SMILES: CC(=O)CC(=O)O[C@H]1C(=O)NCCC(=O)NCCSC(=O)/C=C/C(=O)OCC1(C)C] |
| 232 | 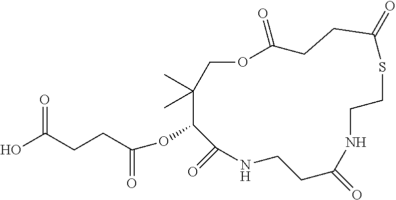 [SMILES: CC1(C)COC(=O)CCC(=O)SCCNC(=O)CCNC(=O)[C@@H]1OC(=O)CCC(=O)O] |
| 233 | 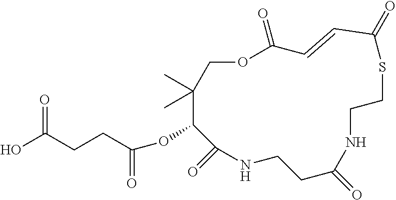 [SMILES: CC1(C)COC(=O)/C=C/C(=O)SCCNC(=O)CCNC(=O)[C@@H]1OC(=O)CCC(=O)O] |
| 234 | 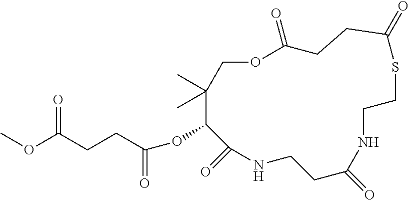 [SMILES: COC(=O)CCC(=O)O[C@H]1C(=O)NCCC(=O)NCCSC(=O)CCC(=O)OCC1(C)C] |
| 235 | 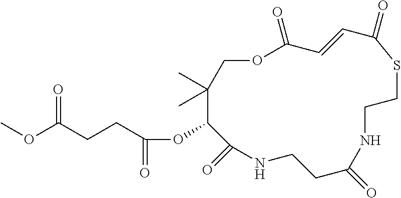 [SMILES: COC(=O)CCC(=O)O[C@H]1C(=O)NCCC(=O)NCCSC(=O)/C=C/C(=O)OCC1(C)C] |
| 236 | 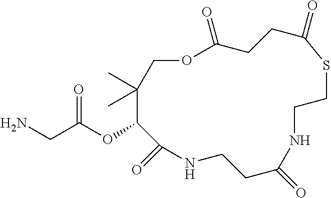 [SMILES: CC1(C)COC(=O)CCC(=O)SCCNC(=O)CCNC(=O)[C@@H]1OC(=O)CN] |
| 237 | 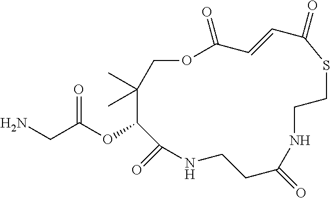 [SMILES: CC1(C)COC(=O)/C=C/C(=O)SCCNC(=O)CCNC(=O)[C@@H]1OC(=O)CN] |
| 238 | 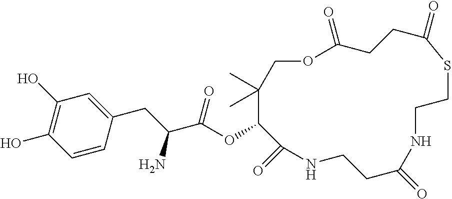 [SMILES: CC1(C)COC(=O)CCC(=O)SCCNC(=O)CCNC(=O)[C@@H]1OC(=O)[C@@H](N)Cc1ccc(O)c(O)c1] |
| 239 | 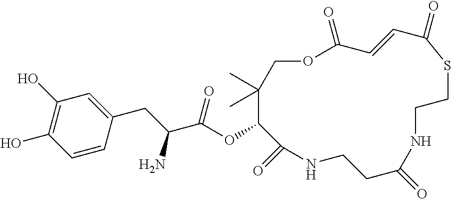 [SMILES: CC1(C)COC(=O)/C=C/C(=O)SCCNC(=O)CCNC(=O)[C@@H]1OC(=O)[C@@H](N)Cc1ccc(O)c(O)c1] |
| 240 | 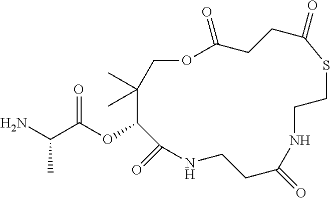 [SMILES: C[C@H](N)C(=O)O[C@H]1C(=O)NCCC(=O)NCCSC(=O)CCC(=O)OCC1(C)C] |
| 241 | 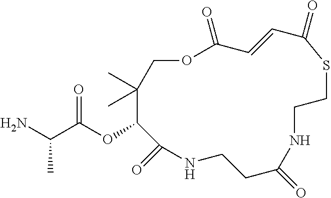 [SMILES: C[C@H](N)C(=O)O[C@H]1C(=O)NCCC(=O)NCCSC(=O)/C=C/C(=O)OCC1(C)C] |
| 242 | 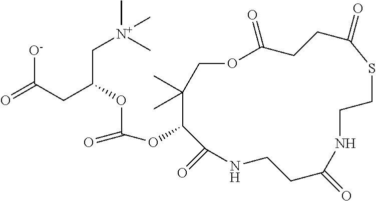 [SMILES: CC1(C)COC(=O)CCC(=O)SCCNC(=O)CCNC(=O)[C@@H]1OC(=O)O[C@H](CC(=O)[O-])C[N+](C)(C)C] |
| 243 | 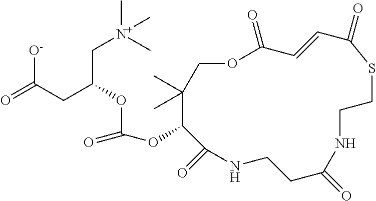 [SMILES: CC1(C)COC(=O)/C=C/C(=O)SCCNC(=O)CCNC(=O)[C@@H]1OC(=O)O[C@H](CC(=O)[O-])C[N+](C)(C)C] |
| 244 | 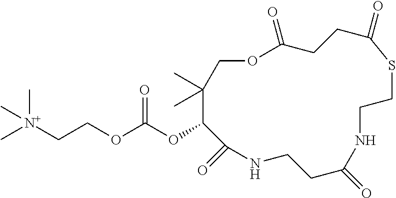 [SMILES: CC1(C)COC(=O)CCC(=O)SCCNC(=O)CCNC(=O)[C@@H]1OC(=O)OCC[N+](C)(C)C] |
| 245 | 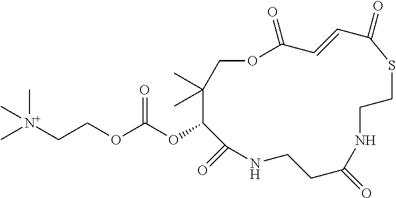 [SMILES: CC1(C)COC(=O)/C=C/C(=O)SCCNC(=O)CCNC(=O)[C@@H]1OC(=O)OCC[N+](C)(C)C] |
| 246 | 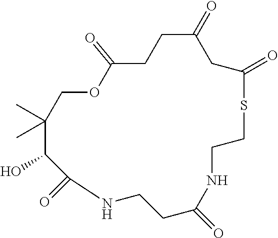 [SMILES: CC1(C)COC(=O)CCC(=O)CC(=O)SCCNC(=O)CCNC(=O)[C@@H]1O] |
| 247 | 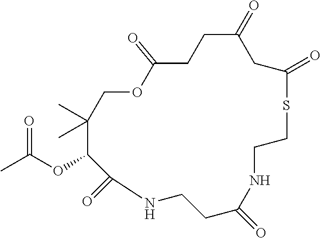 [SMILES: CC(=O)O[C@H]1C(=O)NCCC(=O)NCCSC(=O)CC(=O)CCC(=O)OCC1(C)C] |
| 248 | 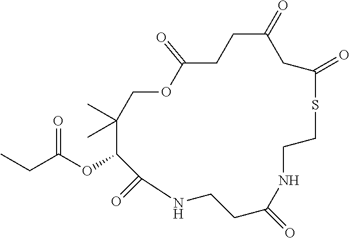 [SMILES: CCC(=O)O[C@H]1C(=O)NCCC(=O)NCCSC(=O)CC(=O)CCC(=O)OCC1(C)C] |
| 249 | 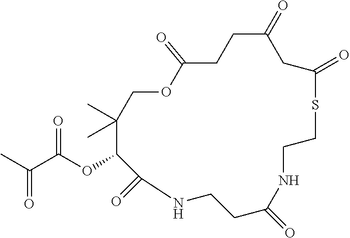 [SMILES: CC(=O)C(=O)O[C@H]1C(=O)NCCC(=O)NCCSC(=O)CC(=O)CCC(=O)OCC1(C)C] |
| 250 | 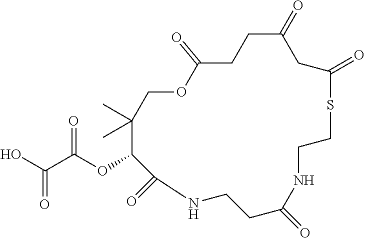 [SMILES: CC1(C)COC(=O)CCC(=O)CC(=O)SCCNC(=O)CCNC(=O)[C@@H]1OC(=O)C(=O)O] |
| 251 | 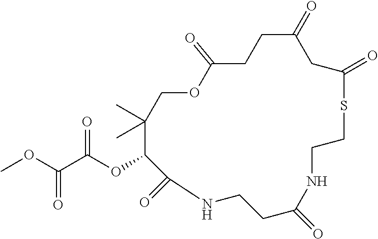 [SMILES: COC(=O)C(=O)O[C@H]1C(=O)NCCC(=O)NCCSC(=O)CC(=O)CCC(=O)OCC1(C)C] |
| 252 | 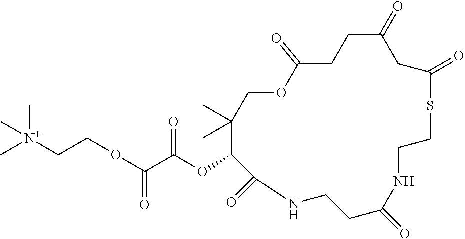 [SMILES: CC1(C)COC(=O)CCC(=O)CC(=O)SCCNC(=O)CCNC(=O)[C@@H]1OC(=O)C(=O)OCC[N+](C)(C)C] |
| 253 | 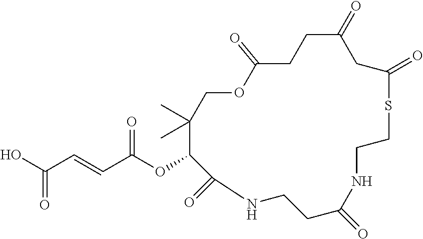 [SMILES: CC1(C)COC(=O)CCC(=O)CC(=O)SCCNC(=O)CCNC(=O)[C@@H]1OC(=O)/C=C/C(=O)O] |
| 254 | 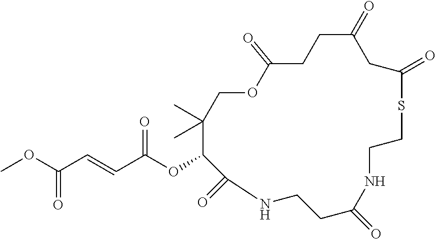 [SMILES: COC(=O)/C=C/C(=O)O[C@H]1C(=O)NCCC(=O)NCCSC(=O)CC(=O)CCC(=O)OCC1(C)C] |
| 255 | 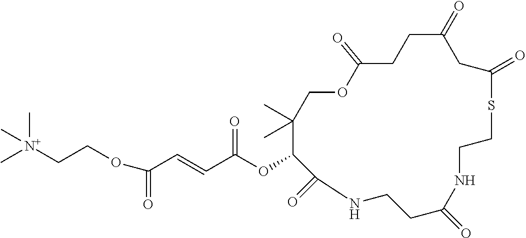 [SMILES: CC1(C)COC(=O)CCC(=O)CC(=O)SCCNC(=O)CCNC(=O)[C@@H]1OC(=O)/C=C/C(=O)OCC[N+](C)(C)C] |
| 256 | 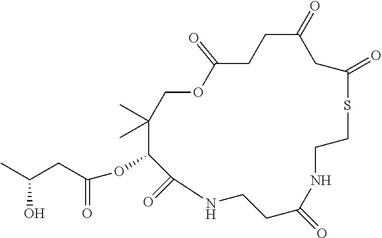 [SMILES: C[C@@H](O)CC(=O)O[C@H]1C(=O)NCCC(=O)NCCSC(=O)CC(=O)CCC(=O)OCC1(C)C] |
| 257 | 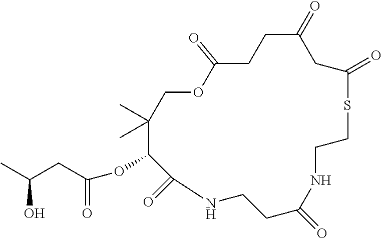 [SMILES: C[C@H](O)CC(=O)O[C@H]1C(=O)NCCC(=O)NCCSC(=O)CC(=O)CCC(=O)OCC1(C)C] |
| 258 | 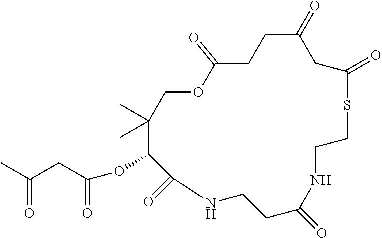 [SMILES: CC(=O)CC(=O)O[C@H]1C(=O)NCCC(=O)NCCSC(=O)CC(=O)CCC(=O)OCC1(C)C] |
| 259 | 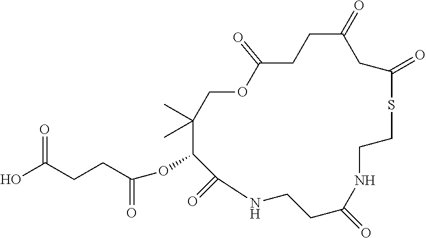 [SMILES: CC1(C)COC(=O)CCC(=O)CC(=O)SCCNC(=O)CCNC(=O)[C@@H]1OC(=O)CCC(=O)O] |
| 260 | 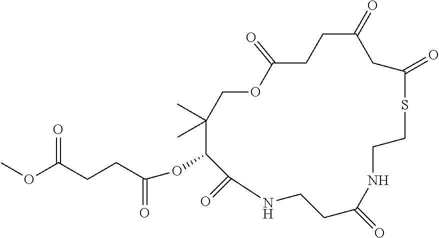 [SMILES: COC(=O)CCC(=O)O[C@H]1C(=O)NCCC(=O)NCCSC(=O)CC(=O)CCC(=O)OCC1(C)C] |
| 261 | 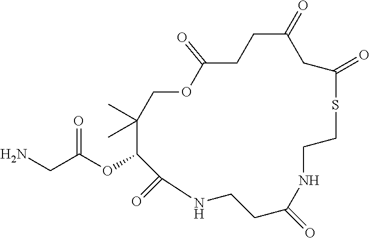 [SMILES: CC1(C)COC(=O)CCC(=O)CC(=O)SCCNC(=O)CCNC(=O)[C@@H]1OC(=O)CN] |
| 262 | 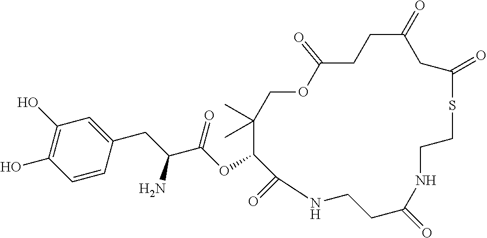 [SMILES: CC1(C)COC(=O)CCC(=O)CC(=O)SCCNC(=O)CCNC(=O)[C@@H]1OC(=O)[C@@H](N)Cc1ccc(O)c(O)c1] |
| 263 | 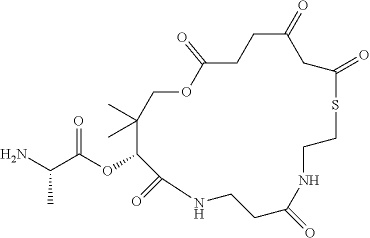 [SMILES: C[C@H](N)C(=O)O[C@H]1C(=O)NCCC(=O)NCCSC(=O)CC(=O)CCC(=O)OCC1(C)C] |
| 264 | 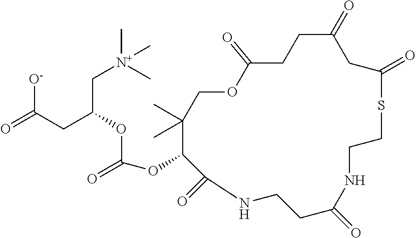 [SMILES: CC1(C)COC(=O)CCC(=O)CC(=O)SCCNC(=O)CCNC(=O)[C@@H]1OC(=O)O[C@H](CC(=O)[O-])C[N+](C)(C)C] |
| 265 | 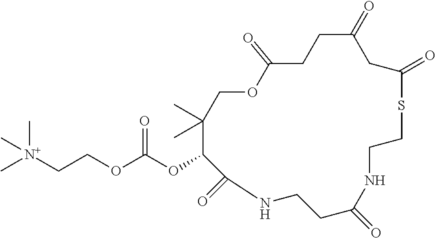 [SMILES: CC1(C)COC(=O)CCC(=O)CC(=O)SCCNC(=O)CCNC(=O)[C@@H]1OC(=O)OCC[N+](C)(C)C] |
| 266 | 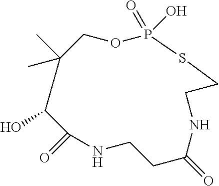 [SMILES: CC1(C)COP(=O)(O)SCCNC(=O)CCNC(=O)[C@@H]1O] |
| 267 | 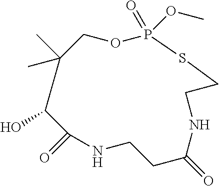 [SMILES: COP1(=O)OCC(C)(C)[C@@H](O)C(=O)NCCC(=O)NCCS1] |
| 268 | 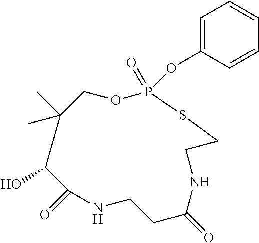 [SMILES: CC1(C)COP(=O)(Oc2ccccc2)SCCNC(=O)CCNC(=O)[C@@H]1O] |
| 269 | 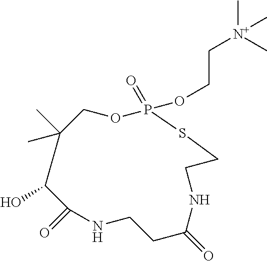 [SMILES: CC1(C)COP(=O)(OCC[N+](C)(C)C)SCCNC(=O)CCNC(=O)[C@@H]1O] |
| 270 | 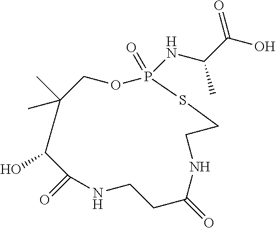 [SMILES: C[C@H](NP1(=O)OCC(C)(C)[C@@H](O)C(=O)NCCC(=O)NCCS1)C(=O)O] |
| 271 | 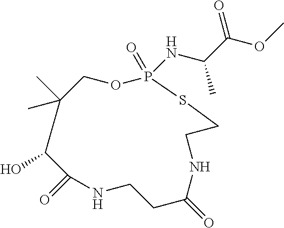 [SMILES: COC(=O)[C@H](C)NP1(=O)OCC(C)(C)[C@@H](O)C(=O)NCCC(=O)NCCS1] |
| 272 | 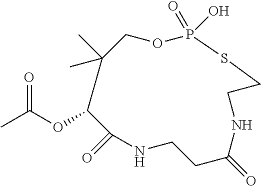 [SMILES: CC(=O)O[C@H]1C(=O)NCCC(=O)NCCSP(=O)(O)OCC1(C)C] |
| 273 | 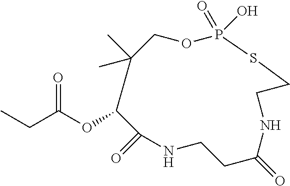 [SMILES: CCC(=O)O[C@H]1C(=O)NCCC(=O)NCCSP(=O)(O)OCC1(C)C] |
| 274 | 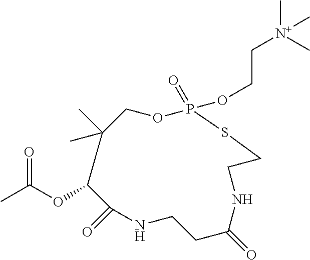 [SMILES: CC(=O)O[C@H]1C(=O)NCCC(=O)NCCSP(=O)(OCC[N+](C)(C)C)OCC1(C)C] |
| 275 | 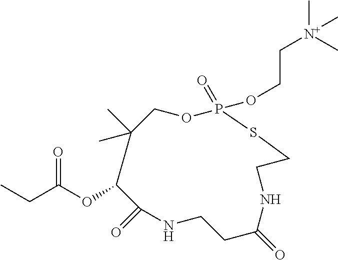 [SMILES: CCC(=O)O[C@H]1C(=O)NCCC(=O)NCCSP(=O)(OCC[N+](C)(C)C)OCC1(C)C] |
| 276 | 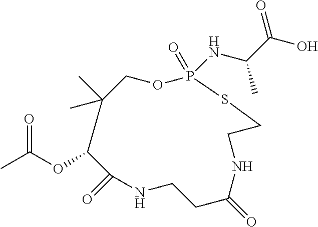 [SMILES: CC(=O)O[C@H]1C(=O)NCCC(=O)NCCSP(=O)(N[C@@H](C)C(=O)O)OCC1(C)C] |
| 277 | 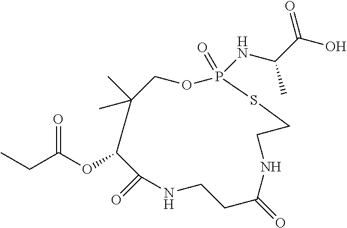 [SMILES: CCC(=O)O[C@H]1C(=O)NCCC(=O)NCCSP(=O)(N[C@@H](C)C(=O)O)OCC1(C)C] |
| 278 | 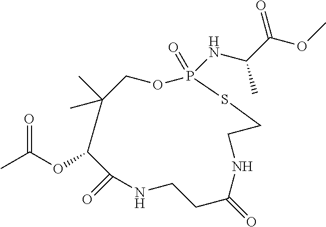 [SMILES: COC(=O)[C@H](C)NP1(=O)OCC(C)(C)[C@@H](OC(C)=O)C(=O)NCCC(=O)NCCS1] |
| 279 | 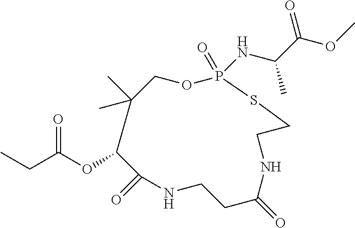 [SMILES: CCC(=O)O[C@H]1C(=O)NCCC(=O)NCCSP(=O)(N[C@@H](C)C(=O)OC)OCC1(C)C] |
| 280 | 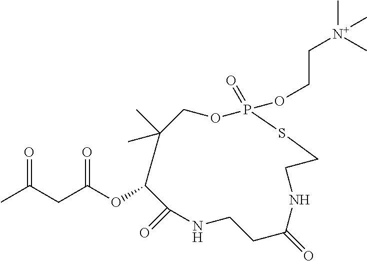 [SMILES: CC(=O)CC(=O)O[C@H]1C(=O)NCCC(=O)NCCSP(=O)(OCC[N+](C)(C)C)OCC1(C)C] |
| 281 | 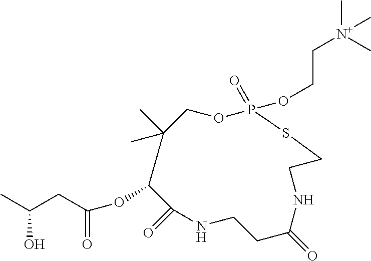 [SMILES: C[C@@H](O)CC(=O)O[C@H]1C(=O)NCCC(=O)NCCSP(=O)(OCC[N+](C)(C)C)OCC1(C)C] |
| 282 | 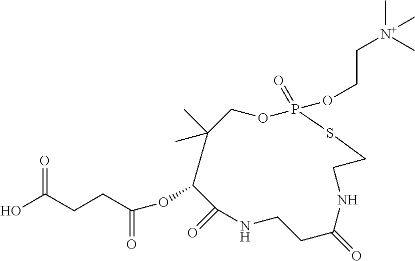 [SMILES: CC1(C)COP(=O)(OCC[N+](C)(C)C)SCCNC(=O)CCNC(=O)[C@@H]1OC(=O)CCC(=O)O] |
| 283 | 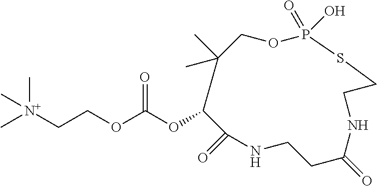 [SMILES: CC1(C)COP(=O)(O)SCCNC(=O)CCNC(=O)[C@@H]1OC(=O)OCC[N+](C)(C)C] |
| 284 | 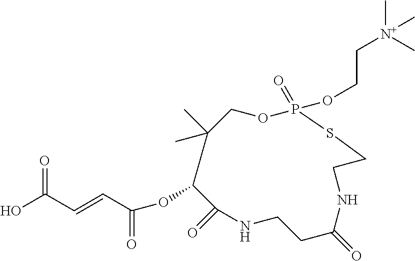 [SMILES: CC1(C)COP(=O)(OCC[N+](C)(C)C)SCCNC(=O)CCNC(=O)[C@@H]1OC(=O)/C=C/C(=O)O] |
| 285 | 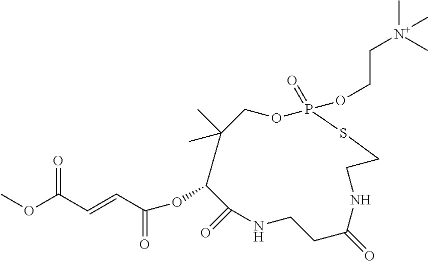 |
| 286 | 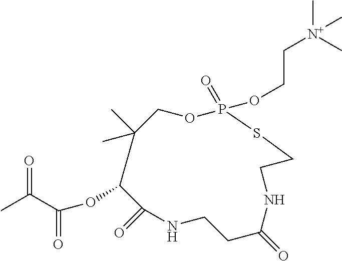 [SMILES: CC(=O)C(=O)O[C@H]1C(=O)NCCC(=O)NCCSP(=O)(OCC[N+](C)(C)C)OCC1(C)C] |
| 287 | 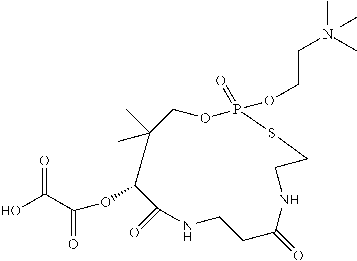 [SMILES: CC1(C)COP(=O)(OCC[N+](C)(C)C)SCCNC(=O)CCNC(=O)[C@@H]1OC(=O)C(=O)O] |
| 288 | 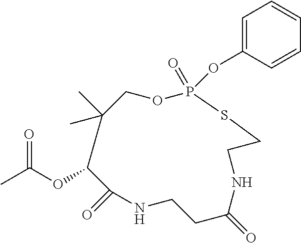 [SMILES: CC(=O)O[C@H]1C(=O)NCCC(=O)NCCSP(=O)(Oc2ccccc2)OCC1(C)C] |
| 289 | 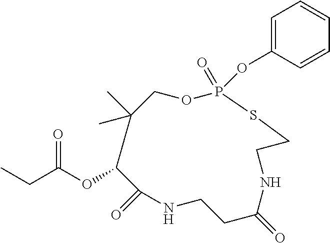 [SMILES: CCC(=O)O[C@H]1C(=O)NCCC(=O)NCCSP(=O)(Oc2ccccc2)OCC1(C)C] |
| 290 | 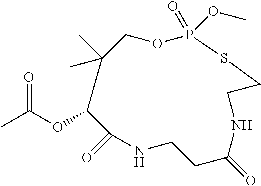 [SMILES: COP1(=O)OCC(C)(C)[C@@H](OC(C)=O)C(=O)NCCC(=O)NCCS1] |
| 291 | 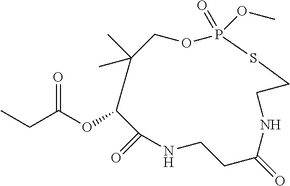 [SMILES: CCC(=O)O[C@H]1C(=O)NCCC(=O)NCCSP(=O)(OC)OCC1(C)C] |
| 292 | 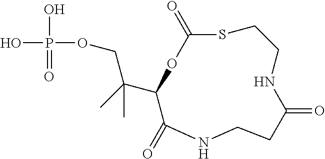 [SMILES: CC(C)(COP(=O)(O)O)[C@H]1OC(=O)SCCNC(=O)CCNC1=O] |
| 293 | 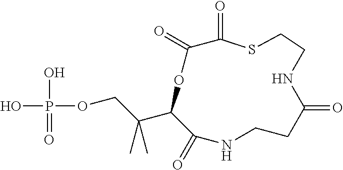 [SMILES: CC(C)(COP(=O)(O)O)[C@H]1OC(=O)C(=O)SCCNC(=O)CCNC1=O] |
| 294 | 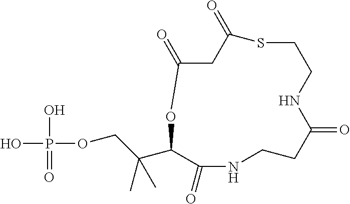 [SMILES: CC(C)(COP(=O)(O)O)[C@H]1OC(=O)CC(=O)SCCNC(=O)CCNC1=O] |
| 295 | 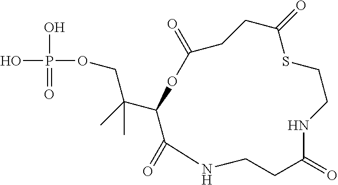 [SMILES: CC(C)(COP(=O)(O)O)[C@H]1OC(=O)CCC(=O)SCCNC(=O)CCNC1=O] |
| 296 | 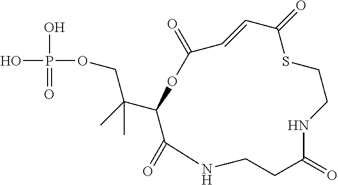 [SMILES: CC(C)(COP(=O)(O)O)[C@H]1OC(=O)/C=C/C(=O)SCCNC(=O)CCNC1=O] |
| 297 | 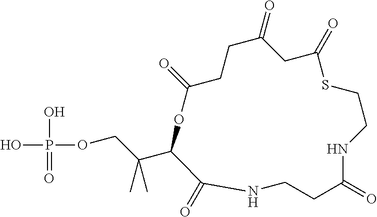 [SMILES: CC(C)(COP(=O)(O)O)[C@H]1OC(=O)CCC(=O)CC(=O)SCCNC(=O)CCNC1=O] |
| 298 | 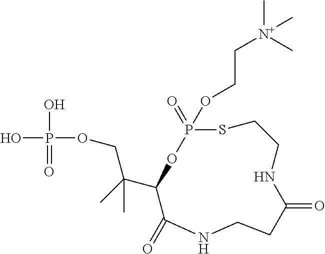 [SMILES: CC(C)(COP(=O)(O)O)[C@H]1OP(=O)(OCC[N+](C)(C)C)SCCNC(=O)CCNC1=O] |
| 299 | 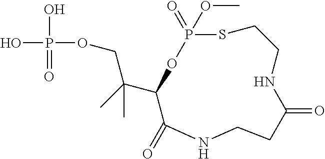 [SMILES: COP1(=O)O[C@H](C(C)(C)COP(=O)(O)O)C(=O)NCCC(=O)NCCS1] |
| 300 | 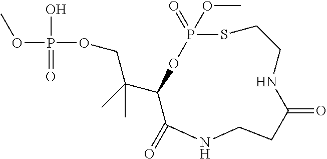 [SMILES: COP(=O)(O)OCC(C)(C)[C@H]1OP(=O)(OC)SCCNC(=O)CCNC1=O] |
| 301 | 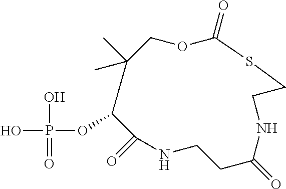 [SMILES: CC1(C)COC(=O)SCCNC(=O)CCNC(=O)[C@@H]1OP(=O)(O)O] |
| 302 | 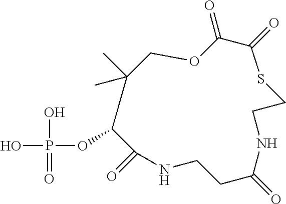 [SMILES: CC1(C)COC(=O)C(=O)SCCNC(=O)CCNC(=O)[C@@H]1OP(=O)(O)O] |
| 303 | 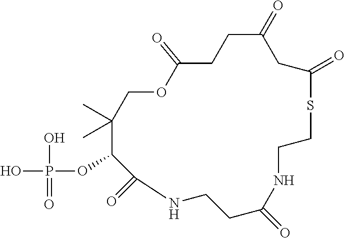 [SMILES: CC1(C)COC(=O)CCC(=O)CC(=O)SCCNC(=O)CCNC(=O)[C@@H]1OP(=O)(O)O] |
| 304 | 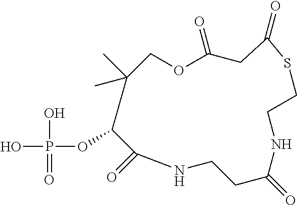 [SMILES: CC1(C)COC(=O)CC(=O)SCCNC(=O)CCNC(=O)[C@@H]1OP(=O)(O)O] |
| 305 | 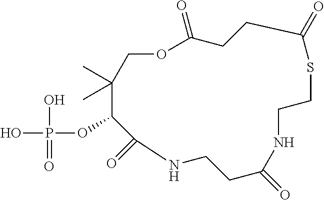 [SMILES: CC1(C)COC(=O)CCC(=O)SCCNC(=O)CCNC(=O)[C@@H]1OP(=O)(O)O] |
| 306 | 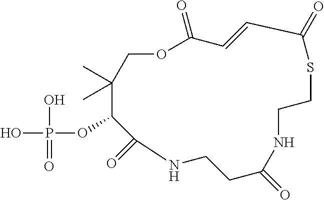 [SMILES: CC1(C)COC(=O)/C=C/C(=O)SCCNC(=O)CCNC(=O)[C@@H]1OP(=O)(O)O] |
| 307 | 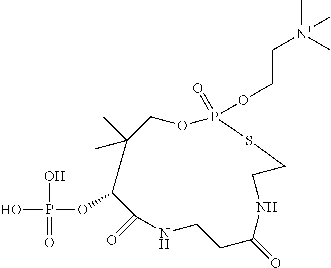 [SMILES: CC1(C)COP(=O)(OCC[N+](C)(C)C)SCCNC(=O)CCNC(=O)[C@@H]1OP(=O)(O)O] |
| 308 | 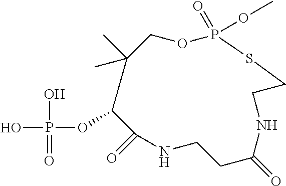 [SMILES: COP1(=O)OCC(C)(C)[C@@H](OP(=O)(O)O)C(=O)NCCC(=O)NCCS1] |
| 309 | 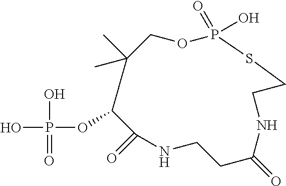 [SMILES: CC1(C)COP(=O)(O)SCCNC(=O)CCNC(=O)[C@@H]1OP(=O)(O)O] |

TABLE 3

| Compound No. | Structure |
|---|---|
| 310 | 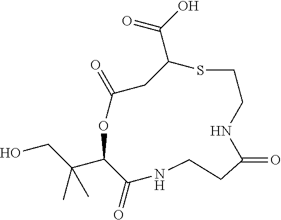 [SMILES: CC(C)(CO)[C@H]1OC(=O)CC(C(=O)O)SCCNC(=O)CCNC1=O] |
| 311 | 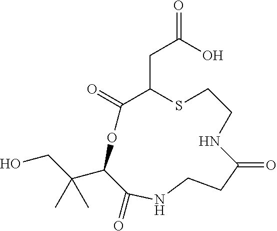 [SMILES: CC(C)(CO)[C@H]1OC(=O)C(CC(=O)O)SCCNC(=O)CCNC1=O] |
| 312 | 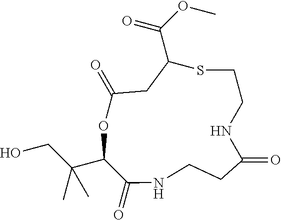 [SMILES: COC(=O)C1CC(=O)O[C@H](C(C)(C)CO)C(=O)NCCC(=O)NCCS1] |
| 313 | 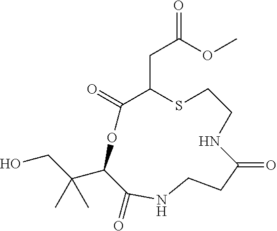 [SMILES: COC(=O)CC1SCCNC(=O)CCNC(=O)[C@@H](C(C)(C)CO)OC1=O] |
| 314 | 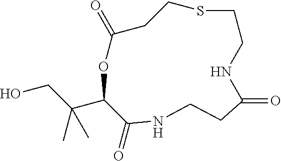 [SMILES: CC(C)(CO)[C@H]1OC(=O)CCSCCNC(=O)CCNC1=O] |
| 315 | 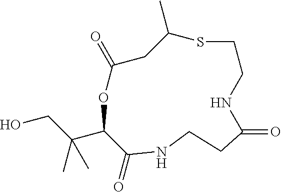 [SMILES: CC1CC(=O)O[C@H](C(C)(C)CO)C(=O)NCCC(=O)NCCS1] |
| 316 | 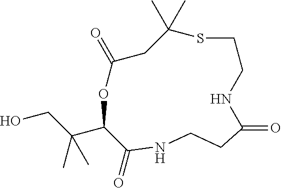 [SMILES: CC1(C)CC(=O)O[C@H](C(C)(C)CO)C(=O)NCCC(=O)NCCS1] |
| 317 | 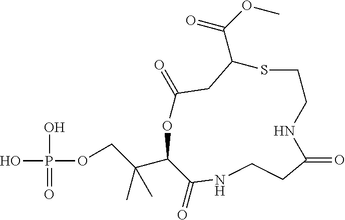 [SMILES: COC(=O)C1CC(=O)O[C@H](C(C)(C)COP(=O)(O)O)C(=O)NCCC(=O)NCCS1] |
| 318 | 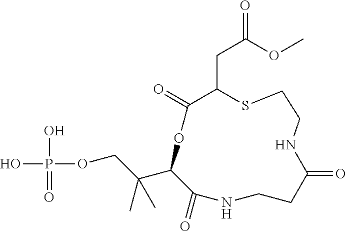 [SMILES: COC(=O)CC1SCCNC(=O)CCNC(=O)[C@@H](C(C)(C)COP(=O)(O)O)OC1=O] |
| 319 | 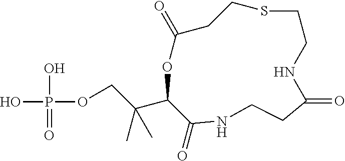 [SMILES: CC(C)(COP(=O)(O)O)[C@H]1OC(=O)CCSCCNC(=O)CCNC1=O] |
| 320 | 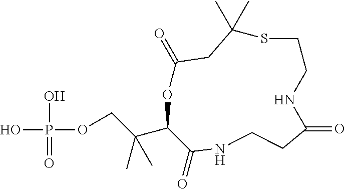 [SMILES: CC1(C)CC(=O)O[C@H](C(C)(C)COP(=O)(O)O)C(=O)NCCC(=O)NCCS1] |
| 321 | 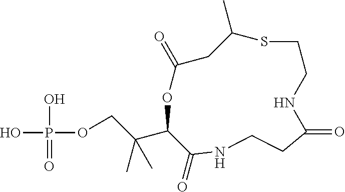 [SMILES: CC1CC(=O)O[C@H](C(C)(C)COP(=O)(O)O)C(=O)NCCC(=O)NCCS1] |
| 322 | 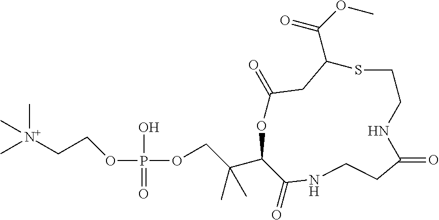 [SMILES: COC(=O)C1CC(=O)O[C@H](C(C)(C)COP(=O)(O)OCC[N+](C)(C)C)C(=O)NCCC(=O)NCCS1] |
| 323 | 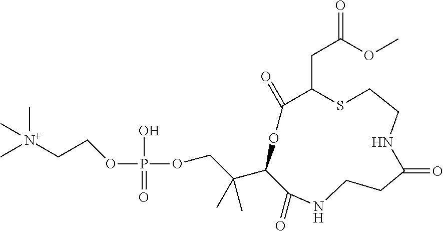 [SMILES: COC(=O)CC1SCCNC(=O)CCNC(=O)[C@@H](C(C)(C)COP(=O)(O)OCC[N+](C)(C)C)OC1=O] |
| 324 | 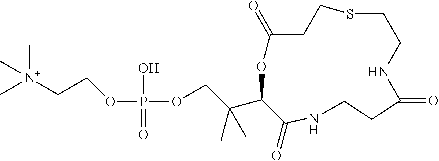 [SMILES: CC(C)(COP(=O)(O)OCC[N+](C)(C)C)[C@H]1OC(=O)CCSCCNC(=O)CCNC1=O] |
| 325 | 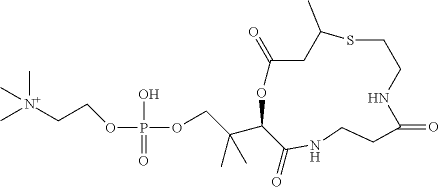 [SMILES: CC1CC(=O)O[C@H](C(C)(C)COP(=O)(O)OCC[N+](C)(C)C)C(=O)NCCC(=O)NCCS1] |
| 326 | 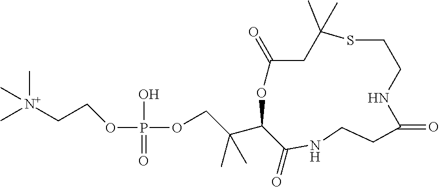 [SMILES: CC1(C)CC(=O)O[C@H](C(C)(C)COP(=O)(O)OCC[N+](C)(C)C)C(=O)NCCC(=O)NCCS1] |
| 327 | 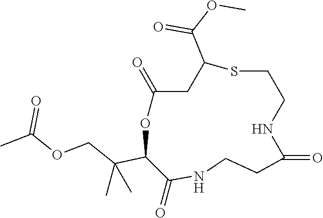 [SMILES: COC(=O)C1CC(=O)O[C@H](C(C)(C)COC(C)=O)C(=O)NCCC(=O)NCCS1] |
| 328 | 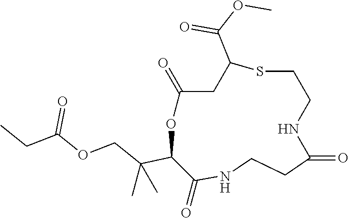 [SMILES: CCC(=O)OCC(C)(C)[C@H]1OC(=O)CC(C(=O)OC)SCCNC(=O)CCNC1=O] |
| 329 | 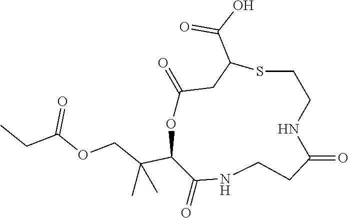 [SMILES: CCC(=O)OCC(C)(C)[C@H]1OC(=O)CC(C(=O)O)SCCNC(=O)CCNC1=O] |
| 330 | 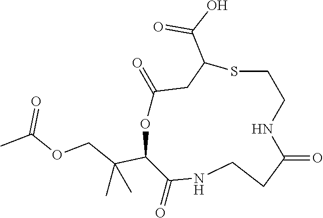 [SMILES: CC(=O)OCC(C)(C)[C@H]1OC(=O)CC(C(=O)O)SCCNC(=O)CCNC1=O] |
| 331 | 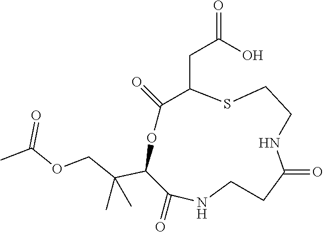 [SMILES: CC(=O)OCC(C)(C)[C@H]1OC(=O)C(CC(=O)O)SCCNC(=O)CCNC1=O] |
| 332 | 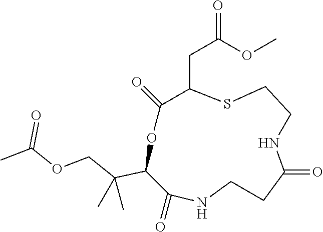 [SMILES: COC(=O)CC1SCCNC(=O)CCNC(=O)[C@@H](C(C)(C)COC(C)=O)OC1=O] |
| 333 | 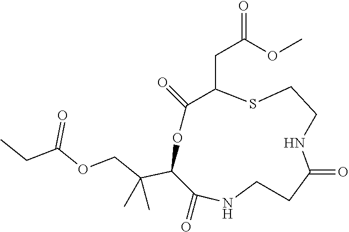 [SMILES: CCC(=O)OCC(C)(C)[C@H]1OC(=O)C(CC(=O)OC)SCCNC(=O)CCNC1=O] |
| 334 | 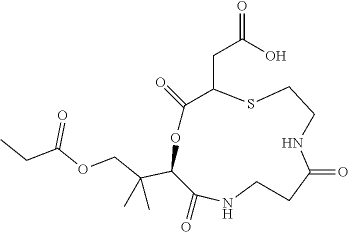 [SMILES: CCC(=O)OCC(C)(C)[C@H]1OC(=O)C(CC(=O)O)SCCNC(=O)CCNC1=O] |
| 335 | 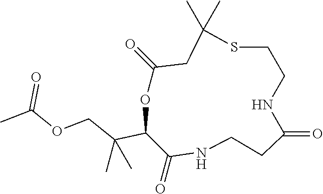 [SMILES: CC(=O)OCC(C)(C)[C@H]1OC(=O)CC(C)(C)SCCNC(=O)CCNC1=O] |
| 336 | 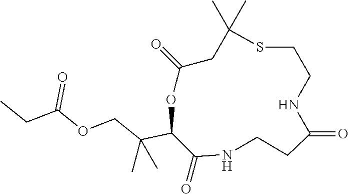 [SMILES: CCC(=O)OCC(C)(C)[C@H]1OC(=O)CC(C)(C)SCCNC(=O)CCNC1=O] |
| 337 | 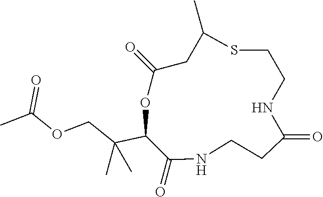 [SMILES: CC(=O)OCC(C)(C)[C@H]1OC(=O)CC(C)SCCNC(=O)CCNC1=O] |
| 338 | 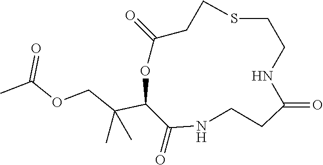 [SMILES: CC(=O)OCC(C)(C)[C@H]1OC(=O)CCSCCNC(=O)CCNC1=O] |
| 339 | 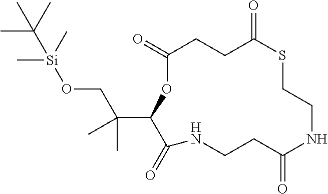 [SMILES: CC(C)(CO[Si](C)(C)C(C)(C)C)[C@H]1OC(=O)CCC(=O)SCCNC(=O)CCNC1=O] |
| 340 | 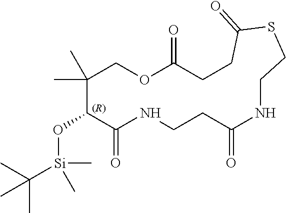 |

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and pharmaceutically acceptable salts and solvates thereof.

In some embodiments, the compound is an isotopic derivative of any one of the compounds described in Table 1.

It is understood that the isotopic derivative can be prepared using techniques known in the art. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

In some embodiments, the compound is a deuterium labeled compound of any one of the compounds described in Table 1 and pharmaceutically acceptable salts and solvates thereof.

In some embodiments, the compound is a deuterium labeled compound of any one of the compounds described in Table 1.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognised techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the invention or a pharmaceutically acceptable salt or solvate thereof that contains the aforementioned deuterium atom(s) is within the scope of the disclosure. Further, substitution with heavier deuterium (i.e., ²H) may afford certain therapeutic advantages resulting from greater metabolic stability, e.g., increased in vivo half-life or reduced dosage requirements.

It is to be understood that a compound of the present disclosure may be depicted in a neutral form, a cationic form (e.g., carrying one or more positive charges), an anionic form (e.g., carrying one or more negative charges), or a zwitterion form (e.g., carrying one or more positive charges and one or more negative charges), all of which are intended to be included in the scope of the present disclosure. For example, when a compound of the present disclosure is depicted in a neutral form, it should be understood that such depiction also refers to the various neutral forms, cationic forms, anionic forms, and zwitterion forms of the compound.

It is to be understood that the compounds of the present disclosure and any pharmaceutically acceptable salts and solvates thereof, comprise stereoisomers, mixtures of stereoisomers, polymorphs of all isomeric forms of said compounds.

As used herein, the term “pharmaceutically acceptable salt” refers to a derivative of the compound of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc. Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3. It is to be understood that all references to pharmaceutically acceptable salts include solvent addition forms (solvates) or crystal forms (polymorphs) as defined herein, of the same salt.

As used herein, the term “solvate” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413: Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cylcobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

It is also to be understood that certain compounds of the present disclosure may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of the present disclosure may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of the present disclosure may exist in a number of different tautomeric forms and references to compounds of the formula I include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

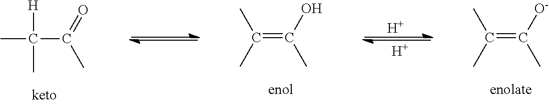

Compounds of the present disclosure containing an amine function may also form N-oxides. A reference herein to a compound of the Formula I that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of the present disclosure may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include in vivo cleavable ester derivatives that may be formed at a carboxy group or a hydroxy group in a compound of the the present disclosure and in vivo cleavable amide derivatives that may be formed at a carboxy group or an amino group in a compound of the present disclosure.

Accordingly, the present disclosure includes those compounds of the present disclosure as defined hereinbefore when made available by organic synthesis and when made available within the human or animal body by way of cleavage of a prodrug thereof. Accordingly, the present disclosure includes those compounds of the present disclosure that are produced by organic synthetic means and also such compounds that are produced in the human or animal body by way of metabolism of a precursor compound, that is a compound of the present disclosure may be a synthetically-produced compound or a metabolically-produced compound.

A suitable pharmaceutically acceptable prodrug of a compound of the present disclosure is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of the present disclosure that possesses a carboxy group is, for example, an in vivo cleavable ester thereof. An in vivo cleavable ester of a compound of the present disclosure containing a carboxy group is, for example, a pharmaceutically acceptable ester which is cleaved in the human or animal body to produce the parent acid. Suitable pharmaceutically acceptable esters for carboxy include C₁-C₆ alkyl esters such as methyl, ethyl and tert-butyl, C₁-C₆ alkoxymethyl esters such as methoxymethyl esters, C₁-C₆ alkanoyloxymethyl esters such as pivaloyloxymethyl esters, 3-phthalidyl esters, C₃-C₈ cycloalkylcarbonyloxy-C₁-C₆ alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁-C₆ alkoxycarbonyloxy-C1-6alkyl esters such as methoxycarbonyloxymethyl and 1-methoxycarbonyloxyethyl esters.

A suitable pharmaceutically acceptable prodrug of a compound of the present disclosure that possesses a hydroxy group is, for example, an in vivo cleavable ester or ether thereof. An in vivo cleavable ester or ether of a compound of the present disclosure containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of the present disclosure that possesses a carboxy group is, for example, an in vivo cleavable amide thereof, for example an amide formed with an amine such as ammonia, a C1-4 alkylamine such as methylamine, a (C₁-C₄ alkyl)2 amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of the present disclosure that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl,morpholinomethyl,piperazin-1-ylmethyl and 4-(C₁-C₄ alkyl)piperazin-1-ylmethyl.

The in vivo effects of a compound of the present disclosure may be exerted in part by one or more metabolites that are formed within the human or animal body after administration of a compound of the present disclosure. As stated hereinbefore, the in vivo effects of a compound of the present disclosure may also be exerted by way of metabolism of a precursor compound (a prodrug).

Though the present disclosure may relate to any compound or particular group of compounds defined herein by way of optional, preferred or suitable features or otherwise in terms of particular embodiments, the present disclosure may also relate to any compound or particular group of compounds that specifically excludes said optional, preferred or suitable features or particular embodiments. A feature of the disclosure concerns particular structural groups at R1, which is relevant to the scope of the claims, as defined herein. In some cases, specific groups define structures that are not relevant to the present invention and thus may be disclaimed. Such structures may be disclaimed where R1 corresponds to a phenyl directly substituted with at least 2 groups including: 1 halogen group and 1 methyl group; 2 or more halogen groups; or 2 methyl groups.

### Methods of Synthesis

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognised reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium on carbon.

The resultant compounds of Formula (I) can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

General routes for the preparation of a compound of the application are described in Schemes 1-3 herein.

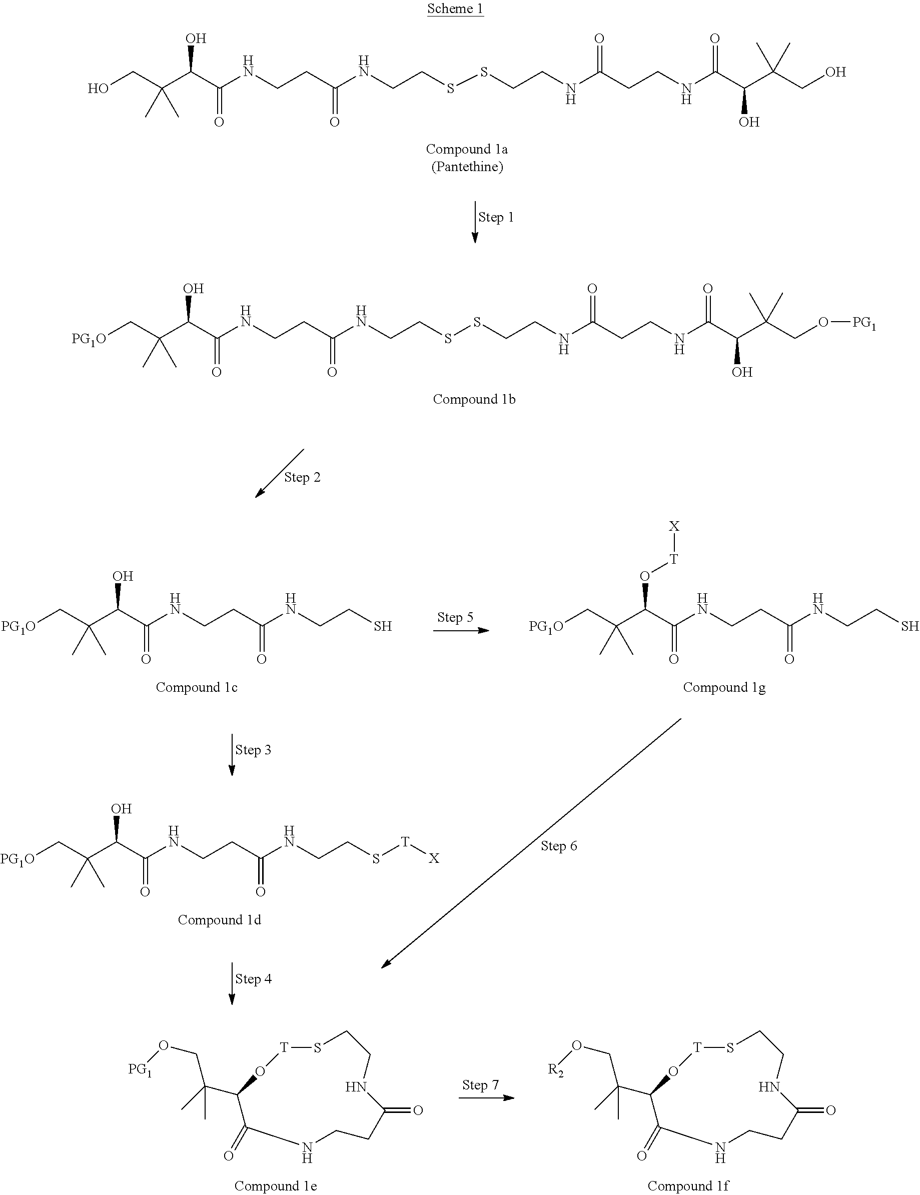

Compounds of the present disclosure are generally made by protection of the primary OH groups (into Compound 1b) of commercially-available pantethine (Compound 1a), followed by reduction of the disulfide Compound 1b with a suitable reducing agent to give the free thiol Compound 1c. Compound 1c can be reacted with a suitable electrophile (such as but not limited to phosphorousoxychloride (POCl3) and/or carbonyidiimidazole and/or oxalyl chloride and/or maleic anhydride and/or succinic anhydride) in the presence of a suitable base to give Compound 1d. Further reaction of Compound 1d with or without heating and in the presence of a suitable base would give the cyclized product Compound 1e. Removal of the alcohol protecting group PG1 would give the products where R₂=H. Further transformation of this alcohol with suitable electrophilic reagents and suitable bases would give Compounds if of the present disclosure (See Scheme 1).

Alternatively, Compound 1c can be reacted with a suitable electrophile in the presence of a suitable base to give Compound 1g. Further reaction of Compound 1g with or without heating and in the presence of a suitable base would give the cyclized product Compound 1e. ‘Removal of the alcohol protecting group PG1 would give the products where R₂=H. Further transformation of this alcohol with suitable electrophilic reagents and suitable bases would give Compounds if of the present disclosure (See Scheme 1).

Compounds of the present disclosure can also generally be made by sequential protection of the primary OH groups (into Compound 1b) of commercially-available pantethine (Compound 1a), followed by additional protection of the secondary OH groups on Compound 1b to give Compound 1h. Reduction of the disulfide Compound 1b with a suitable reducing agent would give the free thiol Compound 1i.

Deprotection of the -OPGI group with suitable deprotection conditions (such as but not limited to acid, base, or hydrogenation conditions) would give the free alcohol and free thiol Compound 1j. Compound 1j can be reacted with a suitable electrophile in the presence of a suitable base to give Compound 1k and/or Compound 1n. Further reaction of Compound 1k and/or Compound 1n with or without heating and in the presence of a suitable base would give the cyclized product Compound 1p (See Scheme 2).

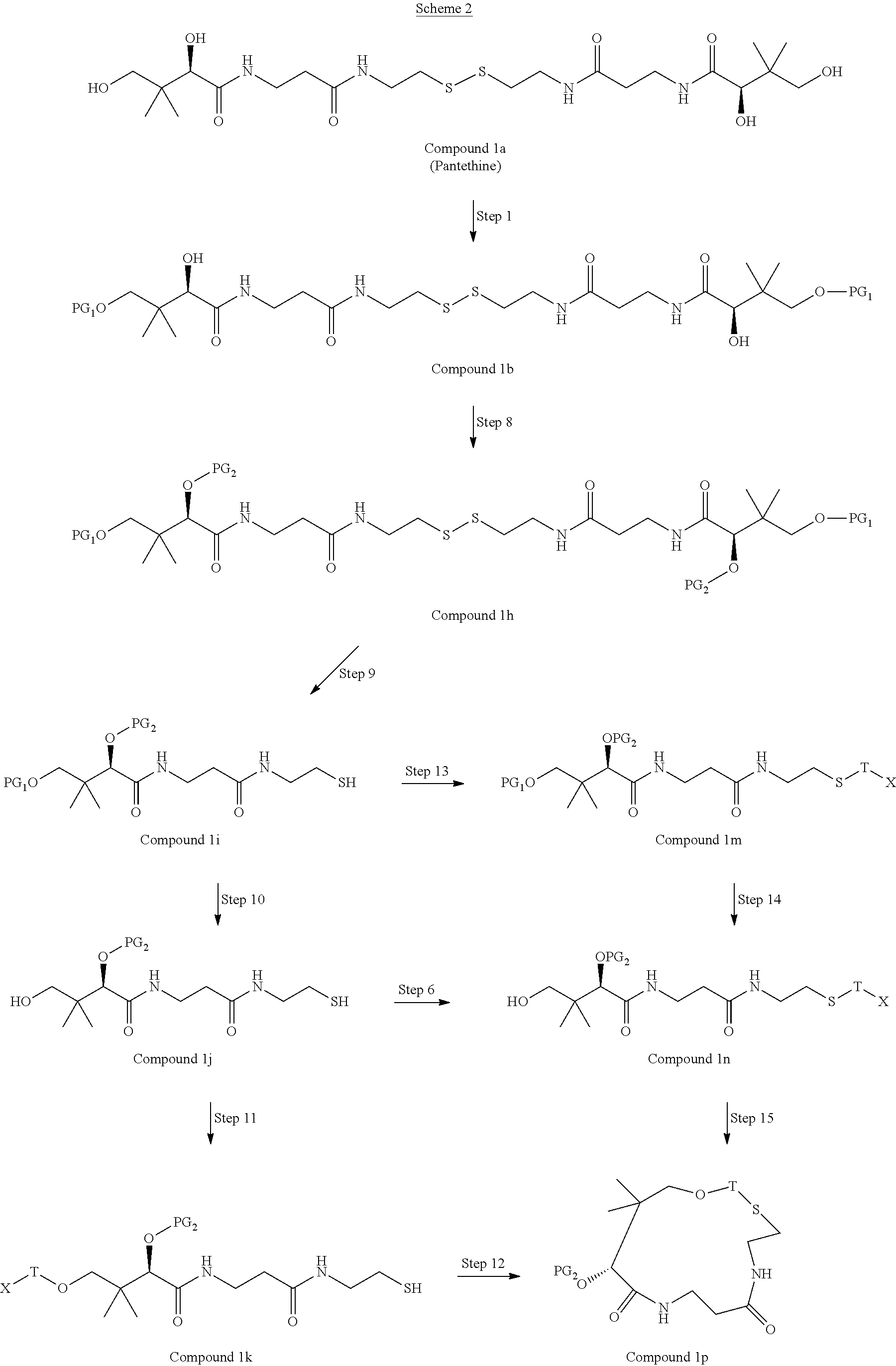

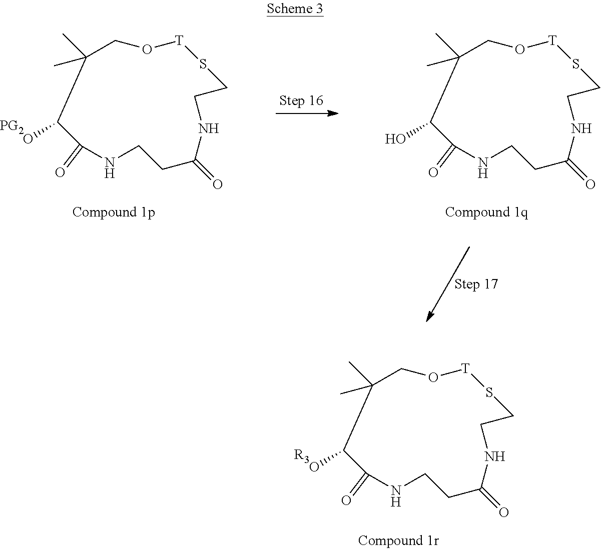

Removal of the alcohol protecting group PG2 would give the OH products of the present disclosure (Compounds 1q). Further transformation of this alcohol with suitable electrophilic reagents and suitable bases would give Compounds 1r of the present disclosure (See Scheme 3).

For some compounds of the present disclosure where the T linking moiety in Scheme 1 and/or Scheme 2 and/or Scheme 3 is —(P═O)(X)—, the X group can be displaced with a suitable nucleophile in the presence of a suitable base to produce Compounds of the present disclosure.

All of these transformations may be effectively conducted by one skilled in the art using suitable methods.

It should be understood that in the description and formulae shown above, the various groups are as defined herein, except where otherwise indicated. Furthermore, for synthetic purposes, the compounds in the Schemes are mere representatives with elected substituents to illustrate the general synthetic methodology of a compound disclosed herein.

### Biological Assays

Compounds and methods designed, selected and/or optimized as described above can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays are may be suitable for detecting the effect of the compounds of the present disclosure and detecting the effect of the methods of the present disclosure. These in vitro or in vivo biological assays can include, but are not limited to, enzymatic activity assays, electrophoretic mobility shift assays, reporter gene assays, in vitro cell viability assays, and the assays described herein.

### Pharmaceutical Compositions

In some aspects, the present disclosure provides a pharmaceutical composition comprising a compound of the present disclosure as an active ingredient.

In some embodiments, the pharmaceutical composition comprises a compound of the present disclosure, or a pharmaceutically acceptable salt or solvate thereof, and one or more pharmaceutically acceptable carriers or excipients.

In some embodiments, the pharmaceutical composition comprises a compound of any one of the Formulae disclosed herein.

In some embodiments, the pharmaceutical composition comprises a compound selected from Table 1.

It is to be understood that a pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., imprinting disorders, and the like) and the health of the patient should preferably be closely monitored during and for a reasonable period after treatment.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It may be especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

It is to be understood that the pharmaceutical compositions can be included in a container, pack, or dispenser together with instructions for administration.

### Methods of Use

In some aspects, the present disclosure provides methods comprising administering to a subject a therapeutically effective amount of at least one compound of the present disclosure, as described in full detail herein.

The present disclosure provides a method of activating or enhancing acetyl-CoA synthesis in a subject comprising administering to the subject a therapeutically effective amount of at least one compound of the present disclosure. The present disclosure provides at least one compound of the present disclosure for use in activating or enhancing acetyl-CoA synthesis in a subject, wherein the at least one compound of the present disclosure is for administration to the subject in at least one therapeutically effective amount. The present disclosure provides a use of at least one compound of the present disclosure for the manufacture of a medicament for activating or enhancing acetyl-CoA synthesis in a subject, wherein the at least one compound of the present disclosure is for administration to the subject in at least one therapeutically effective amount.

The present disclosure provides a method of increasing acetyl-CoA concentrations in a subject comprising administering to the subject a therapeutically effective amount of at least one compound of the present disclosure. The present disclosure provides at least one compound of the present disclosure for use in increasing acetyl-CoA concentrations in a subject, wherein the at least one compound of the present disclosure is for administration to the subject in at least one therapeutically effective amount. The present disclosure provides a use of at least one compound of the present disclosure for the manufacture of a medicament for increasing acetyl-CoA concentrations in a subject, wherein the at least one compound of the present disclosure is for administration to the subject in at least one therapeutically effective amount.

The present disclosure provides a method of treating a subject having a disease comprising administering to the subject a therapeutically effective at least one compound of the present disclosure. The present disclosure provides at least one compound of the present disclosure for use in treating a disease in a subject, wherein the at least one compound of the present disclosure is for administration to the subject in at least one therapeutically effective amount. The present disclosure provides a use of at least one compound of the present disclosure for the manufacture of a medicament for treating a disease in a subject, wherein the at least one compound of the present disclosure is for administration to the subject in at least one therapeutically effective amount.

The present disclosure provides a method of preventing a disease in a subject comprising administering to the subject a therapeutically effective amount of at least one compound of the present disclosure. The present disclosure provides at least one compound of the present disclosure for use in preventing a disease in a subject, wherein the at least one compound of the present disclosure is for administration to the subject in at least one therapeutically effective amount. The present disclosure provides a use of at least one compound of the present disclosure for the manufacture of a medicament for preventing a disease in a subject, wherein the at least one compound of the present disclosure is for administration to the subject in at least one therapeutically effective amount.

In some aspects, a disease can be a disease that is characterized by and/or associated with decreased concentrations of acetyl-CoA. Thus, the present disclosure provides a method of treating a subject having a disease characterized by and/or associated with decreased concentrations of acetyl-CoA comprising administering to the subject a therapeutically effective amount of at least one compound of the present disclosure. The present disclosure provides a method of preventing a disease characterized by and/or associated with decreased concentrations of acetyl-CoA in a subject comprising administering to the subject a therapeutically effective amount of at least one compound of the present disclosure.

In some aspects, a disease can be a disease that is characterized by and/or associated with the loss of or decrease in activity of short chain acyl-CoA dehydrogenase (also referred to as short chain 3-hydroxyacyl-CoA dehydrogenase). A disease can be characterized by and/or associated with short chain acyl-CoA dehydrogenase deficiency. Thus, the present disclosure provides a method of treating a subject having short chain acyl-CoA dehydrogenase deficiency comprising administering to the subject a therapeutically effective amount of at least one compound of the present disclosure. The present disclosure provides a method of preventing short chain acyl-CoA dehydrogenase deficiency in a subject comprising administering to the subject a therapeutically effective amount of at least one compound of the present disclosure.

A disease can be a disease that is characterized by and/or associated with lose of or decrease in activity of short chain acyl-CoA dehydrogenase such that the short chain acyl-CoA dehydrogenase activity in the subject having the disease is no more than 90%, or no more than 80%, or no more than 70%, or no more than 60%, or no more than 50%, or no more than 40%, or no more than 30%, or no more than 20%, or no more than 10% of the short chain acyl-CoA dehydrogenase activity in a subject not having the disease.

In some aspects, a disease can be a disease that is characterized by and/or associated with a loss of or decrease in activity of medium chain acyl-CoA dehydrogenase (also referred to as medium chain 3-hydroxyacyl-CoA dehydrogenase). A disease can be characterized by and/or associated with medium chain acyl-CoA dehydrogenase deficiency. Thus, the present disclosure provides a method of treating a subject having medium chain acyl-CoA dehydrogenase deficiency comprising administering to the subject a therapeutically effective amount of at least one compound of the present disclosure. The present disclosure provides a method of preventing medium chain acyl-CoA dehydrogenase deficiency in a subject comprising administering to the subject a therapeutically effective amount of at least one compound of the present disclosure.

A disease can be a disease that is characterized by and/or associated with lose of or decrease in activity of medium chain acyl-CoA dehydrogenase such that the medium chain acyl-CoA dehydrogenase activity in the subject having the disease is no more than 90%, or no more than 80%, or no more than 70%, or no more than 60%, or no more than 50%, or no more than 40%, or no more than 30%, or no more than 20%, or no more than 10% of the medium chain acyl-CoA dehydrogenase activity in a subject not having the disease.

In some aspects, a disease can be a disease that is characterized by and/or associated with a loss of or decrease in activity of long chain acyl-CoA dehydrogenase (also referred to as long chain 3-hydroxyacyl-CoA dehydrogenase). A disease can be characterized by and/or associated with long chain acyl-CoA dehydrogenase deficiency. Thus, the present disclosure provides a method of treating a subject having long chain acyl-CoA dehydrogenase deficiency comprising administering to the subject a therapeutically effective amount of at least one compound of the present disclosure. The present disclosure provides a method of preventing long chain acyl-CoA dehydrogenase deficiency in a subject comprising administering to the subject a therapeutically effective amount of at least one compound of the present disclosure.

A disease can be a disease that is characterized by and/or associated with lose of or decrease in activity of long chain acyl-CoA dehydrogenase such that the long chain acyl-CoA dehydrogenase activity in the subject having the disease is no more than 90%, or no more than 80%, or no more than 70%, or no more than 60%, or no more than 50%, or no more than 40%, or no more than 30%, or no more than 20%, or no more than 10% of the long chain acyl-CoA dehydrogenase activity in a subject not having the disease.

In some aspects, a disease can be a disease that is characterized by and/or associated with a loss of or decrease in activity of very long chain acyl-CoA dehydrogenase (also referred to as very long chain 3-hydroxyacyl-CoA dehydrogenase). A disease can be characterized by and/or associated with very long chain acyl-CoA dehydrogenase deficiency. Thus, the present disclosure provides a method of treating a subject having very long chain acyl-CoA dehydrogenase deficiency comprising administering to the subject a therapeutically effective amount of at least one compound of the present disclosure. The present disclosure provides a method of preventing very long chain acyl-CoA dehydrogenase deficiency in a subject comprising administering to the subject a therapeutically effective amount of at least one compound of the present disclosure.

A disease can be a disease that is characterized by and/or associated with lose of or decrease in activity of very long chain acyl-CoA dehydrogenase such that the very long chain acyl-CoA dehydrogenase activity in the subject having the disease is no more than 90%, or no more than 80%, or no more than 70%, or no more than 60%, or no more than 50%, or no more than 40%, or no more than 30%, or no more than 20%, or no more than 10% of the very long chain acyl-CoA dehydrogenase activity in a subject not having the disease.

In some aspects, a disease can be a disease that is characterized and/or associated with decreased concentrations of acetyl-CoA. Thus, the present disclosure provides a method of treating a subject having a disease characterized by and/or associated with decreased concentrations of acetyl-CoA comprising administering to the subject a therapeutically effective amount of at least one compound of the present disclosure. The present disclosure provides a method of preventing a disease characterized by and/or associated with decreased concentrations of acetyl-CoA in a subject comprising administering to the subject a therapeutically effective amount of at least one compound of the present disclosure.

In some aspects, a disease can be a disease that is characterized by and/or associated with a decrease in the concentration of acetyl-CoA, such that the concentration of acetyl-CoA in the subject having the disease is no more than 90%, or no more than 80%, or no more than 70%, or no more than 60%, or no more than 50%, or no more than 40%, or no more than 30%, or no more than 20%, or no more than 10% of the concentration of acetyl-CoA in a subject not having the disease.

In some aspects, a disease can be a disease that is characterized and/or associated with decreased concentrations of free CoA. Thus, the present disclosure provides a method of treating a subject having a disease characterized by and/or associated with decreased concentrations of free CoA comprising administering to the subject a therapeutically effective amount of at least one compound of the present disclosure. The present disclosure provides a method of preventing a disease characterized by and/or associated with decreased concentrations of free CoA in a subject comprising administering to the subject a therapeutically effective amount of at least one compound of the present disclosure. As used herein, free CoA is used in its broadest sense to refer to Coenzyme A with a free thiol group (CoA-SH).

In some aspects, a disease can be a disease that is characterized by and/or associated with a decrease in the concentration of free CoA, such that the concentration of free CoA in the subject having the disease is no more than 90%, or no more than 80%, or no more than 70%, or no more than 60%, or no more than 50%, or no more than 40%, or no more than 30%, or no more than 20%, or no more than 10% of the concentration of acetyl-CoA in a subject not having the disease.

In some aspects, a disease can be a disease that is characterized by and/or associated with an increase in at least one CoA species, including, but not limited to, acyl-CoA species. A disease can be a disease that is characterized and/or associated with an increase in at least one CoA species, including but not limited to, acyl-CoA species, such that the concentration of the at least one CoA species in the subject having the disease is at least about two times, or about three times, or about four times, or about five times, or about six times, or about seven times, or about eight times, or about nine times, or about ten times, or about 20 times, or about 30 times, or about 40 times, or about 50 times, or about 60 times, or about 70 times, or about 80 times, or about 90 times, or about 100 times, or about 1000 times the concentration of the at least one CoA species in a subject not having the disease. The increase in the at least one CoA species can cause a concomitant decrease in the concentration of free CoA and/or acetyl-CoA in the subject having the disease. The increase in the at least one CoA species can be caused by impaired fatty acid metabolism, impaired amino acid metabolism, impaired glucose metabolism or any combination thereof.

A disease can be a disease characterized by and/or associated with a disrupted balance between free CoA and acetyl-CoA.

A disease can be a CoA sequestration, toxicity or redistribution (CASTOR) disease. Thus, the present disclosure provides a method of treating a subject having a CASTOR disease comprising administering to the subject a therapeutically effective amount of at least one compound of the present disclosure. The present disclosure provides a method of preventing a CASTOR disease in a subject comprising administering to the subject a therapeutically effective amount of at least one compound of the present disclosure.

In some aspects, a disease can be a disease that is characterized by and/or associated with insufficient pantothenate kinase activity. A disease can be a disease that is characterized by and/or associated with an inhibition of one or more pantothenate kinases (e.g., wild type pantothenate kinases). The inhibition of one or more pantothenate kinases can be caused by the over-accumulation of one or more CoA species, including, but not limited to, acyl-CoA species.

In some aspects, a disease can be a disease that is characterized by and/or associated with impaired or inhibited degradation of one or more acyl-CoA species. Thus, the present disclosure provides a method of treating a subject having a disease characterized by and/or associated with impaired or inhibited degradation of one or more acyl-CoA species comprising administering to the subject a therapeutically effective amount of at least one compound of the present disclosure. The present disclosure provides a method of preventing a disease characterized by and/or associated with impaired or inhibited degradation of one or more acyl-CoA species in a subject comprising administering to the subject a therapeutically effective amount of at least one compound of the present disclosure.

In some aspects, a disease can be a disease that is characterized by and/or associated with accumulation of one or more fatty acids. Thus, the present disclosure provides a method of treating a subject having a disease characterized by and/or associated with accumulation of one or more fatty acids comprising administering to the subject a therapeutically effective amount of at least one compound of the present disclosure. The present disclosure provides a method of preventing a disease characterized by and/or associated with accumulation of one or more fatty acids in a subject comprising administering to the subject a therapeutically effective amount of at least one compound of the present disclosure.

In some aspects, a disease can be a disease that is characterized by and/or associated with impaired, inhibited and/or decreased degradation of one or more fatty acids. Thus, the present disclosure provides a method of treating a subject having a disease characterized by and/or associated with impaired, inhibited and/or decreased degradation of one or more fatty acids comprising administering to the subject a therapeutically effective amount of at least one compound of the present disclosure. The present disclosure provides a method of preventing a disease characterized by and/or associated with impaired, inhibited and/or decreased degradation of one or more fatty acids in a subject comprising administering to the subject a therapeutically effective amount of at least one compound of the present disclosure.

In some aspects, a disease can be a disease that is characterized by and/or associated with abnormal CoA homeostasis. A disease can be a disease that is characterized by and/or associated with abnormal acetyl-CoA homeostasis. A disease can be a disease that is characterized by and/or associated with abnormal acyl-CoA homeostasis. A disease can be a disease that is characterized by and/or associated with abnormal succinyl-CoA homeostasis.

In some aspects, a disease can be a disease selected from the group comprising medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type I, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, 3-methylglutaconic aciduria, PLA2G6-associated neurodegeneration, glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency/Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-CoA:amino acid N-acyltransferase deficiency, bile acid-CoA ligase deficiency, holocarboxylase synthetase deficiency, Succinate dehydrogenase deficiency, α-Ketoglutarate dehydrogenase deficiency, deficiency of CoA synthase enzyme complex (CoASY), glutaric acidemia type II/multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, Systemic primary carnitine deficiency, carnitine: acylcarnitine translocase deficiency I and II, acetyl-CoA carboxylase deficiency, Malonyl-CoA decarboxylase deficiency, Mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency), acyl-CoA oxidase deficiency, alpha-methylacyl-CoA racemase (AMACR) deficiency, sterol carrier protein x deficiency, 2,4-dienoyl-CoA reductase deficiency, Cytosolic acetoacetyl-CoA thiolase deficiency, Cytosolic HMG-CoA synthase deficiency, lecithin cholesterol acyltransferase deficiency, choline acetyl transferase deficiency, Congenital myasthenic syndrome, pyruvate dehydrogenase deficiency, phosphoenolpyruvate carboxykinase deficiency, pyruvate carboxylase deficiency, serine palmiotyl-CoA transferase deficiency/Hereditary sensory and autonomic neuropathy type I, and ethylmalonic encephalopathy, medium-chain acyl-CoA dehydrogenase deficiency, biotinidase deficiency, isovaleric acidemia, very long-chain acyl-CoA dehydrogenase deficiency, long-chain L-3-OH acyl-CoA dehydrogenase deficiency, glutaric acidemia type T, 3-hydroxy-3-methylglutaric acidemia, trifunctional protein deficiency, multiple carboxylase deficiency, methylmalonic acidemia (methylmalonyl-CoA mutase deficiency), 3-methylcrotonyl-CoA carboxylase deficiency, methylmalonic acidemia (Cbl A,B), propionic acidemia, carnitine uptake defect, beta-ketothiolase deficiency, short-chain acyl-CoA dehydrogenase deficiency, glutaric acidemia type II, medium/short-chain L-3-OH acyl-CoA dehydrogenase deficiency, medium-chain ketoacyl-CoA thiolase deficiency, carnitine palmitoyltransferase II deficiency, D-bifunctional protein deficiency methylmalonic acidemia (Cbl C,D), malonic acidemia, carnitine: acylcarnitine translocase deficiency, isobutyryl-CoA dehydrogenase deficiency, 2-methyl 3-hydroxybutyric aciduria, dienoyl-CoA reductase deficiency, 3-methylglutaconic aciduria, PLA2G6-associated neurodegeneration, glycine N-acyltransferase deficiency, 2-methylbutyryl-CoA-dehydrogenase-deficiency, mitochondrial acetoacetyl-CoA thiolase deficiency, dihydrolipoamide dehydrogenase deficiency/Branched chain alpha-ketoacid dehydrogenase (BCKDH) deficiency, 3-methylglutaconyl-CoA hydratase deficiency, 3-hydroxyisobutyrate dehydrogenase deficiency, 3-hydroxy-isobutyryl-CoA hydrolase deficiency, isobutyryl-CoA dehydrogenase deficiency, methylmalonate semialdehyde dehydrogenase deficiency, bile acid-CoA:amino acid N-acyltransferase deficiency, bile acid-CoA ligase deficiency, holocarboxylase synthetase deficiency, Succinate dehydrogenase deficiency, α-Ketoglutarate dehydrogenase deficiency, CoASY, glutaric acidemia type II/multiple acyl-CoA dehydrogenase deficiency, long chain 3-ketoacyl-CoA thiolase, D-3-hydroxyacyl-CoA dehydrogenase deficiency (part of DBD), acyl-CoA dehydrogenase 9 deficiency, Systemic primary carnitine deficiency, carnitine: acylcarnitine translocase deficiency I and II, acetyl-CoA carboxylase deficiency, Malonyl-CoA decarboxylase deficiency, Mitochondrial HMG-CoA synthase deficiency, succinyl-CoA:3-ketoacid CoA transferase deficiency, phytanoyl-CoA hydroxylase deficiency/Refsum disease, D-bifunctional protein deficiency (2-enoyl-CoA-hydratase and D-3-hydroxyacyl-CoA-dehydrogenase deficiency), acyl-CoA oxidase deficiency, alpha-methylacyl-CoA racemase (AMACR) deficiency, sterol carrier protein x deficiency, 2,4-dienoyl-CoA reductase deficiency, Cytosolic acetoacetyl-CoA thiolase deficiency, Cytosolic HMG-CoA synthase deficiency, lecithin cholesterol acyltransferase deficiency, choline acetyl transferase deficiency/Congenital myasthenic syndrome, pyruvate dehydrogenase deficiency, phosphoenolpyruvate carboxykinase deficiency, pyruvate carboxylase deficiency, serine palmitoyl-CoA transferase deficiency, Hereditary sensory and autonomic neuropathy type I, ethylmalonic encephalopathy, Glutaric acidemia type 1, methylmalonic academia, propionyl-CoA carboxylase deficiency, propionic academia, 3-methylcrotonyl carboxylase deficiency, Reye syndrome and isovaleryl-CoA dehydrogenase deficiency.

In some aspects, a disease can be a disease that is characterized by and/or associated with an increase of reactive oxygen species (ROS). A disease can be a disease that is characterized and/or associated with an increase of reactive oxygen species (ROS) such that the concentration of ROS in the subject having the disease is at least about two times, or about three times, or about four times, or about five times, or about six times, or about seven times, or about eight times, or about nine times, or about ten times, or about 20 times, or about 30 times, or about 40 times, or about 50 times, or about 60 times, or about 70 times, or about 80 times, or about 90 times, or about 100 times, or about 1000 times the concentration of ROS in a subject not having the disease.

In some aspects, a disease can be a disease that is characterized by and/or associated with a decrease in fatty acid metabolism. A disease can be a disease that is characterized by and/or associated with a decrease in fatty acid metabolism such that the fatty acid metabolism activity in the subject having the disease is no more than 90%, or no more than 80%, or no more than 70%, or no more than 60%, or no more than 50%, or no more than 40%, or no more than 30%, or no more than 20%, or no more than 10% of the fatty acid metabolism activity in a subject not having the disease.

In some aspects, a disease can be a disease that is characterized by and/or associated with a decrease in amino acid metabolism. A disease can be a disease that is characterized by and/or associated with a decrease in amino acid metabolism such that the amino acid metabolism activity in the subject having the disease is no more than 90%, or no more than 80%, or no more than 70%, or no more than 60%, or no more than 50%, or no more than 40%, or no more than 30%, or no more than 20%, or no more than 10% of the amino acid metabolism activity in a subject not having the disease.

The present disclosure provides a method of increasing Acetyl-CoA biosynthesis in a subject comprising administering to the subject a therapeutically effective amount at least one compound of the present disclosure.

An increase in acetyl-CoA biosynthesis can be about a 10%, or about a 20%, or about a 30%, or about a 40%, or about a 50%, or about a 60%, or about a 70%, or about an 80%, or about a 90%, or about a 100%, or about a 110%, or about a 120%, or about a 130%, or about a 140%, or about a 150%, or about a 160%, or about a 170%, or about a 180%, or about a 190%, or about a 200%, or about a 250%, or about a 300%, or about a 350%, or about a 400%, or about a 450%, or about a 500%, or about a 600%, or about a 700%, or about an 800%, or about a 900%, or about a 1000% increase in acetyl-CoA biosynthesis.

The present disclosure provides a method of decreasing degradation of CoA in a subject comprising administering to the subject a therapeutically effective amount of at least one compound of the present disclosure. The decreased degradation of CoA can prolong the availability and utilization of CoA.

A decrease in degradation of CoA can be about a 1%, or about a 2%, or about a 3%, or about a 4%, or about a 5%, or about a 6%, or about a 7%, or about an 8%, or about a 9%, or about a 10%, or about a 15%, or about a 20%, or about a 25%, or about a 30%, or about a 35%, or about a 40%, or about a 45%, or about a 50%, or about a 55%, or about a 60%, or about a 65%, or about a 70%, or about a 75%, or about an 80%, or about a 85%, or about a 90%, or about a 95% decrease in degradation of CoA.

The present disclosure provides a method of increasing the half-life of CoA in a subject comprising administering to the subject a therapeutically effective amount of at least one compound of the present disclosure.

An increase in the half-life of CoA can be about a 10%, or about a 20%, or about a 30%, or about a 40%, or about a 50%, or about a 60%, or about a 70%, or about an 80%, or about a 90%, or about a 100%, or about a 110%, or about a 120%, or about a 130%, or about a 140%, or about a 150%, or about a 160%, or about a 170%, or about a 180%, or about a 190%, or about a 200%, or about a 250%, or about a 300%, or about a 3 50%, or about a 400%, or about a 4 50%, or about a 500%, or about a 600%, or about a 700%, or about an 800%, or about a 900%, or about a 1000% increase in the half-life of CoA.

The present disclosure provides a method of prolonging the availability of CoA in a subject comprising administering to the subject a therapeutically effective amount of at least one compound of the present disclosure.

The present disclosure provides a method of prolonging the utilization of CoA in a subject comprising administering to the subject a therapeutically effective amount of at least one compound of the present disclosure.

The present disclosure provides a method of delivering an acyl moiety into the mitochondrial matrix of a mitochondrion of a subject comprising administering to the subject a therapeutically effective amount of at least one compound of the present disclosure.

The present disclosure provides a method of delivering a cargo molecule to a particular tissue, cell, or organelle in a subject comprising: providing at least one compound of the present disclosure, administering to the subject a therapeutically effective amount of the at least one compound of the present disclosure.

The present disclosure provides a method of decreasing the concentration of reactive oxygen species (ROS) in a subject comprising administering to the subject a therapeutically effective amount of at least one compound of the present disclosure.

A decrease in the concentration of ROS can be about a 1%, or about a 2%, or about a 3%, or about a 4%, or about a 5%, or about a 6%, or about a 7%, or about an 8%, or about a 9%, or about a 10%, or about a 15%, or about a 20%, or about a 25%, or about a 30%, or about a 35%, or about a 40%, or about a 45%, or about a 50%, or about a 55%, or about a 60%, or about a 65%, or about a 70%, or about a 75%, or about an 80%, or about a 85%, or about a 90%, or about a 95% decrease in the concentration of ROS.

The present disclosure provides a method of decreasing the concentration of an at least one acyl-CoA species in a subject comprising administering to the subject a therapeutically effective amount at least one compound of the present disclosure.

A decrease in the concentration of an at least one acyl-CoA species can be about a 1%, or about a 2%, or about a 3%, or about a 4%, or about a 5%, or about a 6%, or about a 7%, or about an 8%, or about a 9%, or about a 10%, or about a 15%, or about a 20%, or about a 25%, or about a 30%, or about a 35%, or about a 40%, or about a 45%, or about a 50%, or about a 55%, or about a 60%, or about a 65%, or about a 70%, or about a 75%, or about an 80%, or about a 85%, or about a 90%, or about a 95% decrease in the concentration of the at least one acyl-CoA species.

The present disclosure provides a method of increasing the fatty acid metabolism in a subject comprising administering to the subject a therapeutically effective amount of at least one compound of the present disclosure.

An increase in fatty acid metabolism can be about a 10%, or about a 20%, or about a 30%, or about a 40%, or about a 50%, or about a 60%, or about a 70%, or about an 80%, or about a 90%, or about a 100%, or about a 110%, or about a 120%, or about a 130%, or about a 140%, or about a 150%, or about a 160%, or about a 170%, or about a 180%, or about a 190%, or about a 200%, or about a 250%, or about a 300%, or about a 350%, or about a 400%, or about a 450%, or about a 500%, or about a 600%, or about a 700%, or about an 800%, or about a 900%, or about a 1000% increase in fatty acid metabolism.

The present disclosure provides a method of increasing the amino acid metabolism in a subject comprising administering to the subject a therapeutically effective amount of at least one compound of the present disclosure.

An increase in amino acid metabolism can be about a 10%, or about a 20%, or about a 30%, or about a 40%, or about a 50%, or about a 60%, or about a 70%, or about an 80%, or about a 90%, or about a 100%, or about a 110%, or about a 120%, or about a 130%, or about a 140%, or about a 150%, or about a 160%, or about a 170%, or about a 180%, or about a 190%, or about a 200%, or about a 250%, or about a 300%, or about a 350%, or about a 400%, or about a 450%, or about a 500%, or about a 600%, or about a 700%, or about an 800%, or about a 900%, or about a 1000% increase in amino acid metabolism.

The present disclosure provides a method of increasing mitochondrial respiration in a subject comprising administering to the subject a therapeutically effective amount of at least one compound of the present disclosure.

An increase in mitochondrial respiration can be about a 10%, or about a 20%, or about a 30%, or about a 40%, or about a 50%, or about a 60%, or about a 70%, or about an 80%, or about a 90%, or about a 100%, or about a 110%, or about a 120%, or about a 130%, or about a 140%, or about a 150%, or about a 160%, or about a 170%, or about a 180%, or about a 190%, or about a 200%, or about a 250%, or about a 300%, or about a 350%, or about a 400%, or about a 450%, or about a 500%, or about a 600%, or about a 700%, or about an 800%, or about a 900%, or about a 1000% increase mitochondrial respiration.

As used herein, the terms “mitochondrial respiration” and “oxidative phosphorylation” are used interchangeably in their broadest sense to refer to the set of metabolic reactions and process requiring oxygen that takes place in mitochondria to convert the energy stored in macronutrients to ATP.

The present disclosure provides a method of increasing ATP concentration in a subject comprising administering to the subject therapeutically effective amount of at least one compound of the present disclosure.

An increase in ATP concentration can be about a 10%, or about a 20%, or about a 30%, or about a 40%, or about a 50%, or about a 60%, or about a 70%, or about an 80%, or about a 90%, or about a 100%, or about a 110%, or about a 120%, or about a 130%, or about a 140%, or about a 150%, or about a 160%, or about a 170%, or about a 180%, or about a 190%, or about a 200%, or about a 250%, or about a 300%, or about a 350%, or about a 400%, or about a 450%, or about a 500%, or about a 600%, or about a 700%, or about an 800%, or about a 900%, or about a 1000% increase mitochondrial respiration.

### Methods of Use—Cancer

The fundamental patterns of epigenetic components, such as histone modifications, are frequently altered in tumor cells. Epigenetic re-programming has evolved as a means to provide cancer cells a survival advantage by altering the expression of genes associated with key cell regulating effects and suppressing immune response to the altered cell. HDACs are involved in modulating most key cellular processes, including transcriptional regulation, apoptosis, DNA damage repair, cell cycle control, autophagy, metabolism, senescence and chaperone function. Because HDACs have been found to function incorrectly or have aberrant expression in cancer, resulting in abnormal acetylation patterns, various histone deacetylase inhibitors (HDACis) have been investigated to act as cancer chemotherapeutics.

HDACis are a class of epigenetic-modifying drugs that dose-dependently inhibit HDACs and induce acetylation of histone and non-histone proteins, resulting in a variety of effects on cell proliferation, differentiation, anti-inflammation, and anti-apoptosis. Changes in cell differentiation are often the cause for tumor progression and acquired resistance to anti-cancer treatment. Four HDACis have FDA approval to treat hematologic cancers and several more are in various stages of development to treat a wide range of hematologic and solid cancers. BELEODAQ®, FARYDAK® and ZOLINZA® are all pan inhibitors (Classes I, II, and IV) while ISTODAX® is a more specific inhibitor (Class I). HDACis have shown benefits in cancer therapy by induction of tumor cell apoptosis, cell cycle arrest, differentiation and senescence, by enhancing the body's own immune response against the cancer, by inhibition of angiogenesis, and through augmentation of the apoptotic effects of other anti-cancer agents. The sensitivity of tumor cells and relative resistance of normal cells to HDACi may reflect the multiple defects that make cancer cells less likely than normal cells to compensate for inhibition of one or more prosurvival factors or activation of a pro-death pathway.

In some aspects, the present disclosure provides a method of treating a subjecting having a cancer comprising administering to the subject a therapeutically effective amount of at least one compound of the present disclosure. The present disclosure provides at least one compound of the present disclosure for use in treating a cancer in a subject, wherein the at least one compound of the present disclosure is for administration to the subject in at least one therapeutically effective amount. The present disclosure provides a use of at least one compound of the present disclosure for the manufacture of a medicament for treating a cancer in a subject, wherein the at least one compound of the present disclosure is for administration to the subject in at least one therapeutically effective amount.

The present disclosure provides a method of preventing a cancer in a subject comprising administering to the subject a therapeutically effective amount of at least one compound of the present disclosure. The present disclosure provides at least one compound of the present disclosure for use in preventing a cancer in a subject, wherein the at least one compound of the present disclosure is for administration to the subject in at least one therapeutically effective amount. The present disclosure provides a use of at least one compound of the present disclosure for the manufacture of a medicament for preventing a cancer in a subject, wherein the at least one compound of the present disclosure is for administration to the subject in at least one therapeutically effective amount.

In some aspects, the present disclosure provides a method of reducing the size of a tumor comprising administering to the subject a therapeutically effective amount of at least one compound of the present disclosure.

The present disclosure provides a method of inducing tumor cell apoptosis in a subject comprising administering to the subject a therapeutically effective amount of at least one compound of the present disclosure.

The present disclosure provides a method of inducing cell cycle arrest in a tumor cell in a subject comprising administering to the subject a therapeutically effective amount of at least one compound of the present disclosure.

The present disclosure provides a method of inducing differentiation of a cell in a subject comprising administering to the subject a therapeutically effective amount of at least one compound of the present disclosure. The present disclosure provides a method of inducing senescence in a cell in a subject comprising administering to the subject a therapeutically effective amount of at least one compound of the present disclosure. A cell can be a cancerous cell.

The present disclosure provides a method of enhancing an immune response against cancer in a subject comprising administering to the subject a therapeutically effective amount of at least one compound of the present disclosure.

The present disclosure provides a method of inhibiting angiogenesis in a subject comprising administering to the subject a therapeutically effective amount of at least one compound of the present disclosure.

The present disclosure provides a method of enhancing the apoptotic effect of an anti-cancer agent comprising administering to a subject a combination of a therapeutically effective amount of the anti-cancer agent and a therapeutically effective amount of at least one compound of the present disclosure.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, leukemia and germ cell tumors. More particular examples of such cancers include adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer, cervical cancer, Acute lymphoblastic leukemia (ALL), Acute myeloid leukemia (AML), Adrenal gland tumors, Anal cancer, Bile duct cancer, Bladder cancer, Bone cancer, Bowel cancer, Brain tumors, Breast cancer, Cancer of unknown primary (CUP), Cancer spread to bone, Cancer spread to brain, Cancer spread to liver, Cancer spread to lung, Carcinoid, Cervical cancer, Children's cancers, Chronic lymphocytic leukemia (CLL), Chronic myeloid leukemia (CML), Colorectal cancer, Ear cancer, Endometrial cancer, Eye cancer, Follicular dendritic cell sarcoma, Gallbladder cancer, Gastric cancer, Gastro esophageal junction cancers, Germ cell tumors, Gestational trophoblastic disease (GTD), Hairy cell leukemia, Head and neck cancer, Hodgkin lymphoma, Kaposi's sarcoma, Kidney cancer, Laryngeal cancer, Leukemia, Linitis plastica of the stomach, Liver cancer, Lung cancer, Lymphoma, Malignant schwannoma, Mediastinal germ cell tumors, Melanoma skin cancer, Men's cancer, Merkel cell skin cancer, Mesothelioma, Molar pregnancy, Mouth and oropharyngeal cancer, Myeloma, Nasal and paranasal sinus cancer, Nasopharyngeal cancer, Neuroblastoma, Neuroendocrine tumors, Non-Hodgkin lymphoma (NHL), Esophageal cancer, Ovarian cancer, Pancreatic cancer, Penile cancer, Persistent trophoblastic disease and choriocarcinoma, Phaeochromocytoma, Prostate cancer, Pseudomyxoma peritonei, Rectal cancer, Retinoblastoma, Salivary gland cancer, Secondary cancer, Signet cell cancer, Skin cancer, Small bowel cancer, Soft tissue sarcoma, Stomach cancer, T cell childhood non Hodgkin lymphoma (NHL), Testicular cancer, Thymus gland cancer, Thyroid cancer, Tongue cancer, Tonsil cancer, Tumors of the adrenal gland, Uterine cancer, Vaginal cancer, Vulval cancer, Wilms' tumor, Womb cancer and gynaecological cancer. Examples of cancer also include, but are not limited to, Hematologic malignancies, Lymphoma, Cutaneous T-cell lymphoma, Peripheral T-cell lymphoma, Hodgkin's lymphoma, Non-Hodgkin's lymphoma, Multiple myeloma, Chronic lymphocytic leukemia, chronic myeloid leukaemia, acute myeloid leukaemia, Myelodysplastic syndromes, Myelofibrosis, Biliary tract cancer, Hepatocellular cancer, Colorectal cancer, Breast cancer, Lung cancer, Non-small cell lung cancer, Ovarian cancer, Thyroid Carcinoma, Renal Cell Carcinoma, Pancreatic cancer, Bladder cancer, skin cancer, malignant melanoma, merkel cell carcinoma, Uveal Melanoma or Glioblastoma multiforme.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

An anti-cancer agent can comprise, but is not limited to, 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abemaciclib, Abiraterone acetate, Abraxane, Accutane, Actinomycin-D, Adcetris, Ado-Trastuzumab Emtansine, Adriamycin, Adrucil, Afatinib, Afinitor, Agrylin, Ala-Cort, Aldesleukin, Alemtuzumab, Alecensa, Alectinib, Alimta, Alitretinoin, Alkaban-AQ, Alkeran, All-transretinoic Acid, Alpha Interferon, Altretamine, Alunbrig, Amethopterin, Amifostine, Aminoglutethimide, Anagrelide, Anandron, Anastrozole, Apalutamide, Arabinosylcytosine, Ara-C, Aranesp, Aredia, Arimidex, Aromasin, Arranon, Arsenic Trioxide, Arzerra, Asparaginase, Atezolizumab, Atra, Avastin, Avelumab, Axicabtagene Ciloleucel, Axitinib, Azacitidine, Bavencio, Bcg, Beleodaq, Belinostat, Bendamustine, Bendeka, Besponsa, Bevacizumab, Bexarotene, Bexxar, Bicalutamide, Bicnu, Blenoxane, Bleomycin, Blinatumomab, Blincyto, Bortezomib, Bosulif, Bosutinib, Brentuximab Vedotin, Brigatinib, Busulfan, Busulfex, C225, Cabazitaxel, Cabozantinib, Calcium Leucovorin, Campath, Camptosar, Camptothecin-11, Capecitabine, Caprelsa, Carac, Carboplatin, Carfilzomib, Carmustine, Carmustine Wafer, Casodex, CCI-779, Ccnu, Cddp, Ceenu, Ceritinib, Cerubidine, Cetuximab, Chlorambucil, Cisplatin, Citrovorum Factor, Cladribine, Clofarabine, Clolar, Cobimetinib, Cometriq, Cortisone, Cosmegen, Cotellic, Cpt-11, Crizotinib, Cyclophosphamide, Cyramza, Cytadren, Cytarabine, Cytarabine Liposomal, Cytosar-U, Cytoxan, Dabrafenib, Dacarbazine, Dacogen, Dactinomycin, Daratumumab, Darbepoetin Alfa, Darzalex, Dasatinib, Daunomycin, Daunorubicin, Daunorubicin Cytarabine (Liposomal), daunorubicin-hydrochloride, Daunorubicin Liposomal, DaunoXome, Decadron, Decitabine, Degarelix, Delta-Cortef, Deltasone, Denileukin Diftitox, Denosumab, DepoCyt, Dexamethasone, Dexamethasone Acetate, Dexamethasone Sodium Phosphate, Dexasone, Dexrazoxane, Dhad, Dic, Diodex, Docetaxel, Doxil, Doxorubicin, Doxorubicin Liposomal, Droxia, DTIC, Dtic-Dome, Duralone, Durvalumab, Eculizumab, Efudex, Ellence, Elotuzumab, Eloxatin, Elspar, Eltrombopag, Emcyt, Empliciti, Enasidenib, Enzalutamide, Epirubicin, Epoetin Alfa, Erbitux, Eribulin, Erivedge, Erleada, Erlotinib, Erwinia L-asparaginase, Estramustine, Ethyol, Etopophos, Etoposide, Etoposide Phosphate, Eulexin, Everolimus, Evista, Exemestane, Fareston, Farydak, Faslodex, Femara, Filgrastim, Firmagon, Floxuridine, Fludara, Fludarabine, Fluoroplex, Fluorouracil, Fluorouracil (cream), Fluoxymesterone, Flutamide, Folinic Acid, Folotyn, Fudr, Fulvestrant, G-Csf, Gazyva, Gefitinib, Gemcitabine, Gemtuzumab ozogamicin, Gemzar, Gilotrif, Gleevec, Gleostine, Gliadel Wafer, Gm-Csf, Goserelin, Granix, Granulocyte—Colony Stimulating Factor, Granulocyte Macrophage Colony Stimulating Factor, Halaven, Halotestin, Herceptin, Hexadrol, Hexalen, Hexamethylmelamine, Hmm, Hycamtin, Hydrea, Hydrocort Acetate, Hydrocortisone, Hydrocortisone Sodium Phosphate, Hydrocortisone Sodium Succinate, Hydrocortone Phosphate, Hydroxyurea, Ibrance, Ibritumomab, Ibritumomab Tiuxetan, Ibrutinib, Iclusig, Idamycin, Idarubicin, Idelalisib, Idhifa, Ifex, IFN-alpha, Ifosfamide, IL-11, IL-2, Imbruvica, Imatinib Mesylate, Imfinzi, Imidazole Carboxamide, Imlygic, Inlyta, Inotuzumab Ozogamicin, Interferon-Alfa, Interferon Alfa-2b (PEG Conjugate), Interleukin-2, Interleukin-11, Intron A (interferon alfa-2b), Ipilimumab, Iressa, Irinotecan, Irinotecan (Liposomal), Isotretinoin, Istodax, Ixabepilone, Ixazomib, Ixempra, Jakafi, Jevtana, Kadcyla, Keytruda, Kidrolase, Kisqali, Kymriah, Kyprolis, Lanacort, Lanreotide, Lapatinib, Lartruvo, L-Asparaginase, Lbrance, Lcr, Lenalidomide, Lenvatinib, Lenvima, Letrozole, Leucovorin, Leukeran, Leukine, Leuprolide, Leurocristine, Leustatin, Liposomal Ara-C, Liquid Pred, Lomustine, Lonsurf, L-PAM, L-Sarcolysin, Lupron, Lupron Depot, Lynparza, Marqibo, Matulane, Maxidex, Mechlorethamine, Mechlorethamine Hydrochloride, Medralone, Medrol, Megace, Megestrol, Megestrol Acetate, Mekinist, Mercaptopurine, Mesna, Mesnex, Methotrexate, Methotrexate Sodium, Methylprednisolone, Meticorten, Midostaurin, Mitomycin, Mitomycin-C, Mitoxantrone, M-Prednisol, MTC, MTX, Mustargen, Mustine, Mutamycin, Myleran, Mylocel, Mylotarg, Navelbine, Necitumumab, Nelarabine, Neosar, Neratinib, Nerlynx, Neulasta, Neumega, Neupogen, Nexavar, Nilandron, Nilotinib, Nilutamide, Ninlaro, Nipent, Niraparib, Nitrogen Mustard, Nivolumab, Nolvadex, Novantrone, Nplate, Obinutuzumab, Octreotide, Octreotide Acetate, Odomzo, Ofatumumab, Olaparib, Olaratumab, Omacetaxine, Oncospar, Oncovin, Onivyde, Ontak, Onxal, Opdivo, Oprelvekin, Orapred, Orasone, Osimertinib, Otrexup, Oxaliplatin, Paclitaxel, Paclitaxel Protein-bound, Palbociclib, Pamidronate, Panitumumab, Panobinostat, Panretin, Paraplatin, Pazopanib, Pediapred, Peg Interferon, Pegaspargase, Pegfilgrastim, Peg-Intron, PEG-L-asparaginase, Pembrolizumab, Pemetrexed, Pentostatin, Perjeta, Pertuzumab, Phenylalanine Mustard, Platinol, Platinol-AQ, Pomalidomide, Pomalyst, Ponatinib, Portrazza, Pralatrexate, Prednisolone, Prednisone, Prelone, Procarbazine, Procrit, Proleukin, Prolia, Prolifeprospan 20 with Carmustine Implant, Promacta, Provenge, Purinethol, Radium 223 Dichloride, Raloxifene, Ramucirumab, Rasuvo, Regorafenib, Revlimid, Rheumatrex, Ribociclib, Rituxan, Rituxan Hycela, Rituximab, Rituximab Hyalurodinase, Roferon-A (Interferon Alfa-2a), Romidepsin, Romiplostim, Rubex, Rubidomycin Hydrochloride, Rubraca, Rucaparib, Ruxolitinib, Rydapt, Sandostatin, Sandostatin LAR, Sargramostim, Siltuximab, Sipuleucel-T, Soliris, Solu-Cortef, Solu-Medrol, Somatuline, Sonidegib, Sorafenib, Sprycel, Sti-571, Stivarga, Streptozocin, SU11248, Sunitinib, Sutent, Sylvant, Synribo, Tafinlar, Tagrisso, Talimogene Laherparepvec, Tamoxifen, Tarceva, Targretin, Tasigna, Taxol, Taxotere, Tecentriq, Temodar, Temozolomide, Temsirolimus, Teniposide, Tespa, Thalidomide, Thalomid, TheraCys, Thioguanine, Thioguanine Tabloid, Thiophosphoamide, Thioplex, Thiotepa, Tice, Tisagenlecleucel, Toposar, Topotecan, Toremifene, Torisel, Tositumomab, Trabectedin, Trametinib, Trastuzumab, Treanda, Trelstar, Tretinoin, Trexall, Trifluridine/Tipiricil, Triptorelin pamoate, Trisenox, Tspa, T-VEC, Tykerb, Valrubicin, Valstar, Vandetanib, VCR, Vectibix, Velban, Velcade, Vemurafenib, Venclexta, Venetoclax, VePesid, Verzenio, Vesanoid, Viadur, Vidaza, Vinblastine, Vinblastine Sulfate, Vincasar Pfs, Vincristine, Vincristine Liposomal, Vinorelbine, Vinorelbine Tartrate, Vismodegib, Vlb, VM-26, Vorinostat, Votrient, VP-16, Vumon, Vyxeos, Xalkori Capsules, Xeloda, Xgeva, Xofigo, Xtandi, Yervoy, Yescarta, Yondelis, Zaltrap, Zanosar, Zarxio, Zejula, Zelboraf, Zevalin, Zinecard, Ziv-aflibercept, Zoladex, Zoledronic Acid, Zolinza, Zometa, Zydelig, Zykadia, Zytiga, or any combination thereof.

### Methods of Use—Post Translational Modifications

Protein acetylation, in which the acetyl group from acetyl-CoA is transferred to a specific site on a polypeptide chain, is an important post-translational modification that enables the cell to react specifically and rapidly to internal and external perturbations. Acetyl-CoA mediated acetylation of proteins can alter the functional profile of a specific protein by influencing its catalytic activity, its capacity to interact with other molecules (including other proteins), its subcellular localization, and/or its stability. Acetylation and deacetylation occurs on histones and nonhistone proteins within the nucleus, cytoplasm, and mitochondria by a complex interaction between histone deacetylases (HDACs), histone acetyltransferases (HATs), lysine acetyltransferases (KATs), and non-enzymatic acetylation. The 18 identified mammalian HDACs are divided into four classes with Class I, II and IV primarily distributed in the nucleus and cytoplasm whereas Class I (sirtuins) are additionally located in mitochondria.

Histone acetylation and deacetylation can regulate chromosome assembly, gene transcription, and posttranslational modification. Acetylation is almost invariably associated with activation of transcription. Many nonhistone proteins have been identified that are substrates for one or another of the HDACs and these substrates include proteins that have regulatory roles in cell proliferation, cell migration, and cell death.

The present disclosure provides a method of increasing the post-translational modification of proteins in a subject comprising administering to the subject a therapeutically effective amount of at least one compound of the present disclosure.

An increase in post-translational modification of proteins can be about a 10%, or about a 20%, or about a 30%, or about a 40%, or about a 50%, or about a 60%, or about a 70%, or about an 80%, or about a 90%, or about a 100%, or about a 110%, or about a 120%, or about a 130%, or about a 140%, or about a 150%, or about a 160%, or about a 170%, or about a 180%, or about a 190%, or about a 200%, or about a 250%, or about a 300%, or about a 350%, or about a 400%, or about a 450%, or about a 500%, or about a 600%, or about a 700%, or about an 800%, or about a 900%, or about a 1000% increase in post-translational modification of proteins.

Post-translational modification of proteins includes but is not limited to acetylation, N-terminal acetylation, lysine acetylation, acylation, O-acylation, N-acylation, S-acylation, Myristoylation, palmitoylation, isoprenylation, prenylation, farnesylation geranilgeranilatyon, glycosylphosphatidylinositol (GPI) anchor formation, lipoylation, flavin moiety (FMN or FAD) attachment, heme C attachment, phosphopantetheinylation, retinylidene Schiff base formation, diphthamide formation, ethanolamine phosphoglycerol attachment, hypusine formation, beta-Lysine addition, formylation, alkylation, methylation, amidation at C-terminus, amide bond formation, amino acid addition, arginylation, polyglutamylation, polyglycylation, butyrylation, gamma-carboxylation, glycosylation, polysialylation, malonylation, hydroxylation, iodination, nucleotide addition, phosphate ester formation, phosphoramidate formation, phosphorylation, adenylylation, uridylylation, propionylation, pyroglutamate formation, S-glutathionylation, S-nitrosylation, S-sulfenylation (S-sulphenylation), S-sulfinylation, S-sulfonylation, succinylation, sulfation, or any combination thereof. In some preferred aspects, post-translational modification of proteins includes but is not limited to acetylation of histones, acetylation of tubulin, or any combination thereof. Post-translational modification of proteins also includes, but is not limited to the modification of lysine by an acyl group, including, but not limited to, a formyl group, a acetyl group, a propionyl group, a butyryl group, a crotonyl group, a malonyl group, a succinyl group, a glutaryl group, a myristoyl group or any combination thereof.

The present disclosure provides a method of increasing acetylation of proteins in a subject comprising administering to the subject a therapeutically effective amount of at least one compound of the present disclosure. The present disclosure provides a method of increasing acetylation of histones in a subject comprising administering to the subject a therapeutically effective amount of at least one compound of the present disclosure. The present disclosure provides a method of increasing acetylation of tubulin in a subject comprising administering to the subject a therapeutically effective amount of at least one compound of the present disclosure.

An increase in acetylation can be about a 10%, or about a 20%, or about a 30%, or about a 40%, or about a 50%, or about a 60%, or about a 70%, or about an 80%, or about a 90%, or about a 100%, or about a 110%, or about a 120%, or about a 130%, or about a 140%, or about a 150%, or about a 160%, or about a 170%, or about a 180%, or about a 190%, or about a 200%, or about a 250%, or about a 300%, or about a 350%, or about a 400%, or about a 450%, or about a 500%, or about a 600%, or about a 700%, or about an 800%, or about a 900%, or about a 1000% increase in acetylation.

An increase in acetylation can be an increase in acetylation by at least one HAT. An increase in acetylation can be an increase in acetylation by a non-enzymatic acetylation mechanism.

Acetylation of histones can include, but is not limited to, acetylation at Lysine 5 of H2A, at Lysine 9 of H2A, at lysine 2 of H2B, at Lysine 5 of H2B, Lysine 12 of H2B, Lysine 15 of H2B, Lysine 20 of H2B, Lysine 9 of H3, Lysine 14 of H3, Lysine 18 of H3, Lysine 23 of H3, Lysine 27 of H3, Lysine 36 of H3, Lysine 56 of H3, Lysine 5 of H4, Lysine 8 of H4, Lysine 12 of H4, Lysine 16 of H4 or any combination thereof. Acetylation of tubulin can include, but is not limited to, acetylation at Lysine 40 of α-tubulin.

In some aspects, a disease can be a disease characterized by and/or associated with decreased post-translational modification (for example, but not limited to, hypo-acetylation). The present disclosure provides a method of restoring reduced post-translational modification by about 5%, or about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100% back towards normality comprising administering to the subject a therapeutically effective amount of at least one compound of the present disclosure.

In some aspects, the present disclosure provides a method of restoring acetylation of proteins from a hypo-acetylated state comprising administering to the subject a therapeutically effective amount of at least one compound of the present disclosure.

The present disclosure provides a method of increasing crotonylation of proteins in a subject comprising administering to the subject a therapeutically effective amount of at least one compound of the present disclosure. The present disclosure provides a method of increasing crotonylation of histones in a subject comprising administering to the subject a therapeutically effective amount of at least one compound of the present disclosure.

An increase in crotonylation can be about a 10%, or about a 20%, or about a 30%, or about a 40%, or about a 50%, or about a 60%, or about a 70%, or about an 80%, or about a 90%, or about a 100%, or about a 110%, or about a 120%, or about a 130%, or about a 140%, or about a 150%, or about a 160%, or about a 170%, or about a 180%, or about a 190%, or about a 200%, or about a 250%, or about a 300%, or about a 350%, or about a 400%, or about a 450%, or about a 500%, or about a 600%, or about a 700%, or about an 800%, or about a 900%, or about a 1000% increase in crotonylation.

Acute kidney injury (AKI) is a potentially lethal condition for which no therapy is available beyond replacement of renal function. Post-translational histone modifications modulate gene expression and kidney injury. Histone crotonylation is a post-translational modification and is physiologically significant and functionally distinct from or redundant to histone acetylation. Histone crotonylation exhibits a crucial role in a wide range of biological processes and may be critically implicated in the pathogenesis of diseases. Enrichment of histone crotonylation is observed at the genes encoding the mitochondrial biogenesis regulator PGC-1α and the sirtuin-3 decrotonylase in AKI kidney tissue. Addition of crotonate increases the expression of PGC-1α and sirtuin-3, and decreases CCL2 expression in cultured tubular cells and healthy kidneys. Systemic crotonate administration protected from experimental AKI, preventing the decrease in renal function and in kidney PGC-1α and sirtuin-3 levels as well as the increase in CCL2 expression. Increasing histone crotonylation has a beneficial effect on AKI and indicates the strong in vivo potential of the therapeutic manipulation of histone crotonylation in a disease state.

### Methods of Use—Neurological Diseases and Disorders

The present disclosure provides a method of treating a subject having a neurodegenerative disease comprising administering to the subject a therapeutically effective amount of at least one compound of the present disclosure. The present disclosure provides at least one compound of the present disclosure for use in treating a neurodegenerative disease in a subject, wherein the at least one compound of the present disclosure is for administration to the subject in at least one therapeutically effective amount. The present disclosure provides a use of at least one compound of the present disclosure precursor for the manufacture of a medicament for treating a neurodegenerative disease in a subject, wherein the at least one compound of the present disclosure is for administration to the subject in at least one therapeutically effective amount.

The present disclosure provides a method of preventing a neurodegenerative disease in a subject comprising administering to the subject a therapeutically effective amount of at least one compound of the present disclosure. The present disclosure provides at least one compound of the present disclosure for use in preventing a neurodegenerative disease in a subject, wherein the at least one compound of the present disclosure is for administration to the subject in at least one therapeutically effective amount. The present disclosure provides a use of at least one compound of the present disclosure precursor for the manufacture of a medicament for preventing a neurodegenerative disease in a subject, wherein the at least one compound of the present disclosure is for administration to the subject in at least one therapeutically effective amount.

Neurodegenerative diseases include, but are not limited to, Alzheimer's disease, dementia, Parkinson's disease, Parkinson's disease-related disorders, Prion diseases, motor neuron diseases, Huntington's disease, Spinocerebellar ataxia, Spinal muscular atrophy, Amyotrophic lateral sclerosis (ALS), Batten disease, Argyrophilic grain disease, tauopathy, Pick's disease, FTD with parkinsonism linked to chromosome 17 (FTDP-17), Dementia lacking distinctive histology, progressive supranuclear palsy (PSP), corticobasal degeneration, multiple system atrophy, ataxias, familial British dementia, Dementia with Lewy Bodies (DLB), fronto-temporal degeneration (FTD), fronto-temporal dementia, primary progressive aphasia, and semeantic dementia.

Alzheimer's disease (AD) is an irreversible, progressive brain disorder that slowly destroys memory and thinking skills leading to dementia. Damage to the brain starts a decade or more before memory and other cognitive problems appear. Toxic changes, including abnormal deposits of proteins forming extracellular amyloid-β (Aβ) plaques and intra-neuronal neurofibrillary tau protein type degenerative tangles, initially occur in the hippocampus, the part of the brain essential in forming memories. By the final stage of AD, damage becomes widespread, and the entire brain will have shrunken significantly. The “amyloid hypothesis” which maintains that the accumulation of Aβ is the primary driver of AD-related pathogenesis, including neurofibrillary tangle formation, synapse loss, and neuronal cell death remains as the predominant thinking for the root cause of the disease. Implicit in the amyloid hypothesis is that the Aβ peptide harbors neurotoxic properties and one hypothesis proposes that proinflammatory molecules, such as cytokines, in the AD brain produced principally by activated microglia clustered around senile plaques are responsible (Bamberger 2001). Growing evidence indicates that mitochondrial dysfunction is an early event during the progression of AD and one of the key intracellular mechanisms associated with the pathogenesis of this disease. Aβ accumulates in synapses and synaptic mitochondria, leading to abnormal mitochondrial dynamics and synaptic degeneration in AD neurons. However, the precise mechanism by which Aβ exerts these putative toxic effects on neurons remains unclear.

FDA approved cholinesterase inhibitors drugs directly increase synaptic acetylcholine while FDA approved Namenda is a NMDA antagonist. These drugs are used separately and in combination and may help reduce symptoms but they don't change the underlying disease process, are only effective for a subset of patients, and usually help for only a limited amount of time.

While defective cholinergic pathways may not be the root cause of AD, they do play a major role in the symptomology of the disease and changes have been observed early in course of the disease. Brain neurons, to support their neurotransmitter functions, require a much higher supply of glucose than quiescent cells. Glucose-derived pyruvate is a principal source of acetyl-CoA in all brain cells, through the pyruvate dehydrogenase complex (PDHC) reaction. Decreased PDHC activity and other enzymes of TCA cycle (e.g. α-ketoglutarate dehydrogenase complex (KGDHC)) have been reported in postmortem studies of AD brains yielding depression of acetyl-CoA synthesis. This attenuates metabolic flux through the TCA cycle, yielding energy deficits and inhibition of diverse synthetic acetylation reactions throughout the neuron which may directly affect acetylcholine synthesis, histone and nonhistone acetylations, and gene expression.

Epigenetic mechanisms including histone acetylation may also be involved in the pathology of AD. Evidence in rodents indicates that histone acetylation plays a role in rescuing learning and memory impairment. Studies have shown that histone acetylation is reduced in various neurodegenerative disorders, such as AD. In AD animal models, HDACis have shown some promise by showing improvement in learning and memory deficits by promoting neural stem cell generation and synaptic development and by increasing hippocampal nerve growth factor in transgenic AD mice, correlating with cognitive improvement. In addition, HDACis have been shown to lower levels of Aβ, and to improve learning and memory and ameliorate clinical symptoms in AD mice. Another HDAC inhibitor has demonstrated suppression of Aβ neurotoxicity by inhibiting microglial-mediated neuroinflammation.

Mitochondria are the energy-generating system of the cell all of which is necessary to fuel the numerous normal cell functions but also needed to protect the cell against the harmful inflammatory and oxidative stresses of the external environment and needed to remove toxic by-products that form in deteriorating cells. Without being bound by theory, the present disclosure is based on, inter alia, the discovery that by improving mitochondrial function, it the high energy requiring neurons, especially cholinergic ones, function better overall and are better able to provide sufficient amounts of acetylcholine. Preserving a proper supply of acetyl-CoA in the diseased brain attenuates the high susceptibility of cholinergic neurons to AD. For example, the FDA approved cholinesterase inhibitors improve symptoms in AD for some period of time so preserving acetylcholine levels is beneficial. Eventually these drugs lose their effectiveness as the neurons die.

In some aspects, the present disclosure provides a method of treating a subject having Alzheimer's disease comprising administering to the subject a therapeutically effective amount of at least one compound of the present disclosure. The present disclosure provides a method of preventing Alzheimer's disease in a subject comprising administering to the subject a therapeutically effective amount of at least one compound of the present disclosure.

The present disclosure provides at least one compound of the present disclosure for use in treating Alzheimer's disease in a subject, wherein the at least one compound of the present disclosure is for administration to the subject in at least one therapeutically effective amount. The present disclosure provides a use of at least one compound of the present disclosure for the manufacture of a medicament for treating Alzheimer's disease in a subject, wherein the at least one compound of the present disclosure is for administration to the subject in at least one therapeutically effective amount.

The present disclosure provides a method of improving mitochondrial health in a subject comprising administering to the subject a therapeutically effective amount of at least one compound of the present disclosure.

The present disclosure provides a method of reducing neuroinflammation in a subject comprising administering to the subject a therapeutically effective amount of at least one compound of the present disclosure.

The present disclosure provides a method of improving neuronal function comprising administering to the subject a therapeutically effective amount of at least one compound of the present disclosure.

The present disclosure provides a method of improving neuronal survival comprising administering to the subject a therapeutically effective amount of at least one compound of the present disclosure.

The present disclosure provides a method of inhibiting microglial-mediated neuroinflammation comprising administering to the subject a therapeutically effective amount of at least one compound of the present disclosure.

Huntington's disease (HD) is a progressive neurological disorder for which there are no disease-modifying treatments. HD is caused by a mutation encoding an abnormal expansion of trinucleotide (CAG)-encoded polyglutamine repeats in a protein called huntingtin (htt). The pathogenic mechanism(s) by which mutant htt (mhtt) causes neuronal dysfunction and death still remains unclear. However, increasing evidence suggests that mhtt disrupts the normal transcriptional regulation of susceptible neurons. One of the proposed causes of this dysregulation is defective neuronal histone acetylation.

The transcriptional activation and repression regulated by chromatin acetylation has been found to be impaired in HD pathology and a clear link correlating mhtt interaction with various HDACs has been established. For example it has been observed that inhibiting HDAC1 increases acetylated forms of mhtt and improved mhtt clearance from the cell. HDAC3 has been reported to be selectively toxic to neurons. It has been demonstrated that normal htt interacts with HDAC3 and protects neurons through its sequestration. In HD it has been shown that the mhtt interacts poorly with HDAC3, and hence de-repressing its neurotoxic activity and mhtt neurotoxicity was inhibited by the knock-down of HDAC3 and markedly reduced in HDAC3-deficient neurons. HDAC4 is traditionally associated with roles in transcription repression and recent findings have increasingly described a widespread peripheral organ pathology in HD, such as skeletal muscles atrophy and heart failure often associated with an increased HDAC4 expression. Interestingly, in addition to these, elevated HDAC4 levels have been shown in post mortem HD brains. It has been well demonstrated that HDAC4 genetic knockdown ameliorates the HD phenotype in mouse models. and reduction of HDAC4 levels delayed cytoplasmic aggregate formation indifferent brain regions and rescued cortico-striatal neuronal synaptic function in HD mouse models accompanied by an improvement in motor co-ordination, neurological phenotypes and increased lifespan. HDAC6, Sirtuin1 and Sirtuin2 inhibition have also been linked to diminished mhtt toxicity. Further studies carried out in cell culture, yeast, Drosophila and rodent model(s) have indicated that HDAC inhibitors (HDACis) might provide useful class of therapeutic agents for HD. Clinical trials have also reported the beneficial effects of HDACis in patients suffering from HD.

In some aspects, the present disclosure provides a method of treating a subject having Huntington's disease comprising administering to the subject a therapeutically effective amount of at least one compound of the present disclosure. The present disclosure provides a method of preventing Huntington's disease in a subject comprising administering to the subject a therapeutically effective amount of at least one compound of the present disclosure.

The present disclosure provides at least one compound of the present disclosure for use in treating Huntington's disease in a subject, wherein the at least one compound of the present disclosure is for administration to the subject in at least one therapeutically effective amount. The present disclosure provides a use of at least one compound of the present disclosure for the manufacture of a medicament for treating Huntington's disease in a subject, wherein the at least one compound of the present disclosure is for administration to the subject in at least one therapeutically effective amount.

Ataxias are a heterogeneous group of disorders characterized by loss of coordination due to the degeneration of the neuronal networks closely linked to cerebellar function.

Friedreich's Ataxia is the most prevalent form of hereditary ataxia and is caused by downregulation of the FXN gene, which encodes frataxin, a mitochondrial protein involved in many cellular functions. In addition to homozygous mutation consisting of a GAA repeat impeding the progress of RNA polymerase, FXN silencing has also been shown to be caused by histone hypoacetylation, which inhibits access of transcription factors to the FXN gene. Several studies have shown that HDAC inhibitors were able to reverse the FXN silencing and restore frataxin levels in both patient neurons and mouse models.

The spinocerebellar ataxia type 3 (SCA-3), also named Machado-Joseph disease is caused by mutation of ATXN3 gene, which encodes ataxin-3. The mutated protein can interact with and impair neuroprotective transcription factors and histone acetyltransferase activity, resulting in histone hypoacetylation and transcriptional defects. Literature suggests that HDAC inhibitor could prevent ataxin-3-Q79-induced hypoacetylation of H3 and H4 histones associated with proximal promoters of downregulated genes in the cerebella of SCA3 transgenic mice; in this way, HDAC inhibitors could recover normal gene expression Spinocerebellar ataxia type 1.

Spinocerebellar ataxia type 1 (SCA-1) is a dominantly inherited neurodegenerative disorder caused by mutations in ATXN1. ATXN1 normally binds HDAC3, a class I HDAC, but in its mutated form it no longer inhibits the HDAC3, thereby resulting in repressed gene transcription through a decrease in histone acetylation at the promoters of genes.

Spinocerebellar ataxia type 7 (SCA-7) presents with autosomal-dominant cerebellar ataxia, representing the only SCA that affects the retina. The SCA7 gene product, ataxin-7, is an integral component of the mammalian SAGA-like complexes, a transcriptional coactivator complex that has histone acetyltransferase activity. In the murine model of SCA7 the ataxin-7 mutation leads to reduced levels of acetylated H3 on promoter/enhancer regions of photoreceptor genes, and thereby contributing to the transcriptional alterations observed in SCA7 retinal degeneration. This phenomenon occurs concomitantly with onset of retinal degeneration. Concerning cerebellar degeneration, a cultured SCA7 human astrocyte model has been used to study the effects of treatment with trichostatin A, but not other HDAC inhibitors, which partially restored RELN transcription.

The present disclosure provides a method of treating a subject having an ataxia disease comprising administering to the subject a therapeutically effective amount of at least one compound of the present disclosure. The present disclosure provides a method of preventing an ataxia in a subject comprising administering to the subject a therapeutically effective amount of at least one compound of the present disclosure. In some aspects, the ataxia can be, but is not limited to, Friedreich's Ataxia, spinocerebellar ataxia type 3 (SCA-3), Spinocerebellar ataxia type 1 (SCA-1) or Spinocerebellar ataxia type 7 (SCA-7).

Multiple sclerosis is a debilitating neurological pathology in which an abnormal response of the body's immune system is directed against the central nervous system, causing inflammation that damages myelin as well as the nerve fibers themselves, and the specialized cells that make myelin. Tecfidera (dimethyl fumarate), an FDA approved drug for treatment of psoriasis and multiple sclerosis has been known to have anti-oxidant properties through its activation a protein called Nrf2, however its anti-inflammatory mode of action has not been well understood until recently, when the direct molecular target of DMF has been identified confirming the mechanism how DMF is able to inhibit several pathways linked to a set of proteins called toll-like receptors (TLRs), which play a key role in innate immune system responses and cytokine production. It been well established that acylation, and in particular acetylation, determines the Toll-like receptor (TLR)-dependent regulation of pro-inflammatory Cytokines, including directly as well as indirectly through related regulatory and signaling pathways such as acetylation of mitogen-activated protein kinase phosphatase-1, which inhibits the Toll-like receptor signaling, reducing inflammation.

The present disclosure provides a method of treating a subject having multiple sclerosis comprising administering to the subject a therapeutically effective amount of at least one compound of the present disclosure. The present disclosure provides at least one compound of the present disclosure for use in treating multiple sclerosis in a subject, wherein the at least one compound of the present disclosure is for administration to the subject in at least one therapeutically effective amount. The present disclosure provides a use of at least one compound of the present disclosure for the manufacture of a medicament for treating multiple sclerosis in a subject, wherein the at least one compound of the present disclosure is for administration to the subject in at least one therapeutically effective amount.

Epilepsy is a neurological disorder in which brain activity becomes abnormal, causing seizures or periods of unusual behavior, sensations, and sometimes loss of awareness. HDAC inhibitor valproic acid has been used as an anticonvulsant and mood-stabilizer drugs in the treatment of epilepsy and bipolar disorder as well as major depression and Schizophrenia without much knowledge of mode of action. Additionally, stringent ketogenic diet has been shown to be very positive for patients with epilepsy and although the exact mechanisms of the diet are unknown, ketone bodies have been hypothesized to contribute to the anticonvulsant and antiepileptic effects and provide an efficient source of Acetyl-CoA for the neural cells. A role for cytosolic Acyl-CoA thioester hydrolase (ACOT) in neurological function was recently suggested by the discovery of low to absent levels of an isoform of ACOT7 in the hippocampus of patients with mesial temporal lobe epilepsy. A very characteristic phenotype of epilepsy with mild intellectual disability, and abnormal behavior was demonstrated also in ACOT7 N−/− mouse model. Cytosolic Acyl-CoA thioester hydrolases are necessary to release CoA from cytosolic Acyl-CoA and allow carboxylic acids to be transported to mitochondria for further metabolism. In epilepsy patients with aberrant ACOT7 levels the cytosolic Acyl-CoAs cannot be processed efficiently enough and thus are sequestering the free CoA.

The present disclosure provides a method of treating a subject having epilepsy comprising administering to the subject a therapeutically effective amount of at least one compound of the present disclosure. The present disclosure provides at least one compound of the present disclosure for use in treating epilepsy in a subject, wherein the at least one compound of the present disclosure is for administration to the subject in at least one therapeutically effective amount. The present disclosure provides a use of at least one compound of the present disclosure for the manufacture of a medicament for treating epilepsy in a subject, wherein the at least one compound of the present disclosure is for administration to the subject in at least one therapeutically effective amount.

Schizophrenia is a complex disorder that is influenced by both genes and environment and can result in presenting an aberrant epigenetic mechanism. The hallmark of these epigenetic mechanisms is monitored through the altered state of histone modifications and other post-translational modifications and miRNAs. The dynamic nature and reversibility of the epigenetic marks raise the possibility that the epigenetic defects can be corrected by therapeutic interventions addressing these epigenetic aberrations. Several lines of evidence suggest that histone modifications in the candidate genes of schizophrenia specific loci may contribute to the pathogenesis of prefrontal dysfunction. Histone H3K9K14 levels were shown to be hypoacetylated at the promoter regions of GAD67, HTR2C, TOMM70A and PPM1E genes in young subjects with schizophrenia. Microarray analysis of a postmortem brain collection of 19 subjects with schizophrenia compared with 25 controls revealed significantly increased expression of the class I histone deacetylase, in prefrontal cortex (on average 30-50%). Recent findings in preclinical model systems corroborate that epigenetic modulation might emerge as a promising target for the treatment of cognitive disorders.

The present disclosure provides a method of treating a subject having schizophrenia comprising administering to the subject a therapeutically effective amount of at least one compound of the present disclosure. The present disclosure provides at least one compound of the present disclosure for use in treating schizophrenia in a subject, wherein the at least one compound of the present disclosure is for administration to the subject in at least one therapeutically effective amount. The present disclosure provides a use of at least one compound of the present disclosure for the manufacture of a medicament for treating schizophrenia in a subject, wherein the at least one compound of the present disclosure is for administration to the subject in at least one therapeutically effective amount.

### Methods of Use—Nonalcoholic Steatohepatitis

Nonalcoholic Steatohepatitis (NASH) is the advanced form of nonalcoholic fatty liver disease (NAFLD) and is defined histologically by the presence of hepatic fat (steatosis) with inflammation and hepatocellular ballooning. Accumulation of fat within the hepatocytes when import or synthesis of fat exceeds its export or degradation. NASH is a progressive disease that can lead to further liver injury, advanced fibrosis, cirrhosis, and hepatocellular carcinoma. A cascade of events occurs in these lipotoxic hepatocytes, including activation of immune mediators and inflammation, hepatic cell damage/death with matrix remodeling via fibrogenesis and fibrinolysis, angiogenesis, and mobilization of liver progenitor cells. Moreover, mitochondrial dysfunction appears to be a key component of the progressing disease, including inappropriate fatty acid oxidation, oxidative stress, and impaired energy production. There are no approved therapies for NASH but there has been an increasing focus on modulating the mediators of these pathways as the therapeutic target.

A central feature of NASH is the aberrant regulation of lipids within hepatocytes. Increased lipogenesis, impaired fatty acid oxidation, and the generation of biologically active fatty acid signaling molecules are factors in NASH pathogenesis leading to lipotoxicity including metabolic and oxidative stress in the liver cells and lead to increased synthesis and deposition of triglycerides. Increased malonyl-CoA, which inhibits carnitine-palmitoyl transferase, inhibited fatty acid oxidation. The critical role of beta oxidation and ketogenesis in prevention of steatohepatitis is further demonstrated by a murine model of mitochondrial 3-hydroxymethylglutaryl CoA synthase (HMGCS2)-deficiency. When fed a high-fat diet, these mice suffer from defective Krebs cycle and gluconeogenesis caused by CoA sequestration and develop severe hepatocyte injury and inflammation. Gluconeogenesis and Krebs cycle are normalized upon supplementation of CoA precursors pantothenic acid and cysteine. This demonstrates the role of CoA homeostasis in NAFLD.

The present disclosure provides a method of treating nonalcoholic steatohepatitis in a subject comprising administering to the subject a therapeutically effective amount of at least one compound of the present disclosure The present disclosure provides a method of preventing nonalcoholic steatohepatitis in a subject comprising administering to the subject a therapeutically effective amount of at least one compound of the present disclosure.

The present disclosure provides a method of treating nonalcoholic fatty liver disease in a subject comprising administering to the subject a therapeutically effective amount of at least one compound of the present disclosure. The present disclosure provides a method of preventing nonalcoholic fatty liver disease in a subject comprising administering to the subject a therapeutically effective amount of at least one compound of the present disclosure.

The present disclosure provides a method of preventing an inappropriate shift to fatty acid biosynthesis in a subject comprising administering to the subject a therapeutically effective amount of at least one compound of the present disclosure.

The present disclosure provides a method of re-establishing CoA homeostasis in a subject comprising administering to the subject a therapeutically effective amount of at least one compound of the present disclosure.

### Methods of Use—Inflammatory Diseases

In multiple cardiac models of heart failure and ischemic-reperfusion injury, the use of an HDACis, which increase the acetylation of histones and nonhistone proteins, protected cardiac tissue and function by inhibition of pathological remodeling through autophagy which serves to protect cardiomyocytes during ischemia by resupplying energy and by reducing inflammation, oxidative stress and fibrosis. In murine models of colitis or chronic intestinal inflammation, HDACis were found to reduce inflammation and tissue damage by acetylation of transcription factors, increased mononuclear apoptosis, reduction of proinflammatory cytokine release, and increase in the number and activity of Regulatory T cells (Tregs). Tregs act as the nucleus in enforcing immune tolerance and also function to preserve intestinal homeostasis and participate in tissue repair. Immuron's oral IMM-124E approach to treating NASH focuses on stimulating Tregs. HDAC inhibition was found to attenuate inflammatory changes in a dextran sulfate sodium-induced colitis mouse model by suppressing local secretion of pro-inflammatory cytokines and chemokines and also by suppressing mobilization and accumulation of inflammatory cells.

The present disclosure provides a method of treating an inflammatory disease in a subject comprising administering to the subject a therapeutically effective amount of at least one compound of the present disclosure. The present disclosure provides at least one compound of the present disclosure for use in treating an inflammatory disease in a subject, wherein the compound is for administration to the subject in at least one therapeutically effective amount. The present disclosure provides a use of at least one compound of the present disclosure for the manufacture of a medicament for treating an inflammatory disease in a subject, wherein the compound is for administration to the subject in at least one therapeutically effective amount.

The present disclosure provides a method of preventing an inflammatory disease in a subject comprising administering to the subject a therapeutically effective amount of at least one compound of the present disclosure. The present disclosure provides at least one compound of the present disclosure for use in preventing an inflammatory disease in a subject, wherein the compound is for administration to the subject in at least one therapeutically effective amount. The present disclosure provides a use of at least one compound of the present disclosure for the manufacture of a medicament for preventing an inflammatory disease in a subject, wherein the compound is for administration to the subject in at least one therapeutically effective amount.

Inflammatory diseases can include, but are not limited to, arthritis, inflammatory bowel disease, hypertension, septic shock, colitis and graft-versus-host-disease (GVHD).

The present disclosure provides a method of reducing inflammation in a subject comprising administering to the subject a therapeutically effective amount of at least one compound of the present disclosure.

A reduction in inflammation can be about a 1%, or about a 2%, or about a 3%, or about a 4%, or about a 5%, or about a 6%, or about a 7%, or about an 8%, or about a 9%, or about a 10%, or about a 15%, or about a 20%, or about a 25%, or about a 30%, or about a 35%, or about a 40%, or about a 45%, or about a 50%, or about a 55%, or about a 60%, or about a 65%, or about a 70%, or about a 75%, or about an 80%, or about a 85%, or about a 90%, or about a 95%, or about a 99%, or about a 99.5% or about a 100% reduction in inflammation.

The present disclosure provides a method of reducing fibrosis in a subject comprising administering to the subject a therapeutically effective amount of at least one compound of the present disclosure.

A reduction in fibrosis can be about a 1%, or about a 2%, or about a 3%, or about a 4%, or about a 5%, or about a 6%, or about a 7%, or about an 8%, or about a 9%, or about a 10%, or about a 15%, or about a 20%, or about a 25%, or about a 30%, or about a 35%, or about a 40%, or about a 45%, or about a 50%, or about a 55%, or about a 60%, or about a 65%, or about a 70%, or about a 75%, or about an 80%, or about a 85%, or about a 90%, or about a 95%, or about a 99%, or about a 99.5% or about a 100% reduction in fibrosis.

The present disclosure provides a method of stimulating the activity of Regulatory T cells in a subject comprising administering to the subject a therapeutically effective amount of at least one compound of the present disclosure.

Stimulating activity of regulatory T cells can comprise an increase in the activity of regulatory T cells. An increase in activity of regulatory T cells can be about a 10%, or about a 20%, or about a 30%, or about a 40%, or about a 50%, or about a 60%, or about a 70%, or about an 80%, or about a 90%, or about a 100%, or about a 110%, or about a 120%, or about a 130%, or about a 140%, or about a 150%, or about a 160%, or about a 170%, or about a 180%, or about a 190%, or about a 200%, or about a 250%, or about a 300%, or about a 350%, or about a 400%, or about a 450%, or about a 500%, or about a 600%, or about a 700%, or about an 800%, or about a 900%, or about a 1000% increase in the activity of regulatory T cells.

### Methods of Use—Inflammatory Bowel Diseases

Inflammatory bowel diseases (IBD) are chronic inflammatory disorders of the intestinal tract and comprise Crohn's disease (CD), ulcerative colitis (UC) and colitis of uncertain type/aetiology. The etiology of IBD remains unknown and disease pathogenesis not fully understood but it appears that genetic, environmental, microbiological and immunological factors drive uncontrolled intestinal inflammatory activation leading to cycles of tissue damage and repair. Although the etiology of IBD is largely unknown, epigenetics is considered an important factor in IBD onset and pathogenesis. Epigenetic alterations such as differential patterns of histone acetylation are found in both biopsies from IBD patients and mouse models of colitis.

Crohn's disease is an inflammatory bowel disease that can involve different areas of the digestive tract and often spreads deep into the layers of affected bowel tissue. Crohn's disease can be both painful and debilitating, and sometimes may lead to life-threatening complications. Active disease usually presents with diarrhea, often bloody, fever, and pain. The inflammation may also present in the skin, eyes, joints or liver. A long-term complication of the chronic inflammation in Crohn's is the development of colorectal cancer and the risk increases significantly with duration as well as with extension of disease. There is no cure for Crohn's disease and there is no one treatment that works for everyone but the goal of medical treatment is to reduce the inflammation. A number of anti-inflammatory and immune suppressor drugs are utilized and up to 50% of patients will require at least one surgery to remove damaged bowel.

In murine models of colitis or chronic intestinal inflammation, HDACis were found to reduce inflammation and tissue damage by increasing the expression of human B-defensin-2 (peptide that protects intestinal mucosa against bacterial invasion as part of the innate defense system toward a proinflammatory response), acetylation of transcription factors, increased mononuclear apoptosis, reduction of proinflammatory cytokine release, and increase in the number and activity of Regulatory T cells. Moreover, HDACis have been found to decrease tumor number and size in models of inflammation-driven tumorigenesis suggesting that in addition to having antiproliferative effects, their antiinflammatory effects and, as a consequence, mucosal healing may contribute to preventing colorectal cancer. Tregs act as the nucleus in enforcing immune tolerance and also function to preserve intestinal homeostasis and participate in tissue repair. Immuron's oral IMM-124E approach to treating colitis focuses on stimulating Tregs and they have reported alleviation of bowel inflammation in murine models. HDAC inhibition was found to attenuate inflammatory changes in a dextran sulfate sodium-induced colitis mouse model by suppressing local secretion of pro-inflammatory cytokines and chemokines and also by suppressing mobilization and accumulation of inflammatory cells.

Ulcerative colitis (UC) is an inflammatory bowel disease that causes long-lasting inflammation and ulcers in the innermost lining of the colon and rectum. UC can be debilitating and the main symptom is usually bloody diarrhea, sometimes with pus, and other problems include crampy abdominal pain, fever, urgency to defecate, and sometime perforation of the colon. The inflammation may also present in the eyes and joints as pain or as canker sores or result in bone loss. UC does increase the risk of colon cancer. Diet and a number of anti-inflammatory and immune suppressor drugs are utilized for treatment but if these treatments don't work or if the disease is severe, a colectomy may be needed.

The present disclosure provides a method of treating Crohn's disease in a subject comprising administering to the subject a therapeutically effective amount of at least one compound of the present disclosure. The present disclosure provides a method of preventing Crohn's disease in a subject comprising administering to the subject a therapeutically effective amount of at least one compound of the present disclosure.

The present disclosure provides a method of treating colitis in a subject comprising administering to the subject a therapeutically effective amount of at least one compound of the present disclosure. The present disclosure provides a method of preventing colitis in a subject comprising administering to the subject a therapeutically effective amount of at least one compound of the present disclosure.

The present disclosure provides a method of treating chronic intestinal inflammation in a subject comprising administering to the subject a therapeutically effective amount of at least one compound of the present disclosure. The present disclosure provides a method of preventing chronic intestinal inflammation in a subject comprising administering to the subject a therapeutically effective amount of at least one compound of the present disclosure.

### Methods of Use—HIV

Combinations of anti-HIV drugs can effectively suppress virus replication but infected individuals possess a reservoir of latent HIV-1. Upon cessation of drugs, viruses in this reservoir reactivate and re-kindle infection. HIV-1 persistence in long-lived cellular reservoirs remains a major barrier to a cure. Patients have to remain on anti-HIV drugs the rest of their lives and there is a strong incentive to be able to either reduce or stop these drugs given the long-term side-effects and burden of taking these drugs. A strategy is being explored to reactivate latent HIV without inducing global T cell activation whereupon a patient's immune system can potentially eradicate the virus. HDACis have been found to reactivate these latently infected cells in nonclinical models and in initial human studies. However, HDACis do not have the ability to completely rid the body of latently infected cells and this approach may need to be combined with an immune modulator, such as IFN-alpha2a, to significantly affect the latent HIV reservoir.

Without being bound by theory, an increase in the acetylation of histones and nonhistone proteins through HATs and non-enzymatic acetylation could stimulate HIV-1 latency reduction or eradication by reactivating latent HIV without inducing global T cell activation. This reactivation would make the HIV infected cells visible to the immune system; the immune response (native plus addition of an immune modulator such as IFN-alpha2a) and antiretroviral cocktail would then be able to attack and eliminate the infected cells.

The present disclosure provides a method of treating HIV in a subject comprising administering to the subject a therapeutically effective amount of at least one compound of the present disclosure.

The present disclosure provides a method of treating HIV in a subject comprising administering to the subject a combination of a therapeutically effective amount of at least one compound of the present disclosure and a therapeutically effective amount of an immune modulator compound.

An immune modulator compound can include, but is not limited to, IFN-alpha 2A or an antiretroviral cocktail.

The present disclosure provides a method of treating HIV in a subject comprising administering to the subject a combination of a therapeutically effective amount of at least one compound of the present disclosure and a therapeutically effective amount of an anti-HIV agent.

Anti-HIV agents include, but are not limited to, abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, saquinavir, tipranavir, enfuvirtide, maraviroc, dolutegravir, raltegravir, ibalizumab, cobicistat, abacavir/lamivudine combination, abacavir/dolutegravir/lamivudine combination, abacavir/lamivudine/zidovudine combination, atazanavir/cobicistat combination, bictegravir/emtricitabine/tenofovir alafenamide combination, darunavir/cobicistat combination, darunavir/cobicistat/emtricitabine/tenofovir alafenamide combination, dolutegravir/rilpivirine combination, doravirine/lamivudine/tenofovir disoproxil fumarate combination, efavirenz/emtricitabine/tenofovir disoproxil fumarate combination, efavirenz/lamivudine/tenofovir disoproxil fumarate combination, elvitegravir/cobicistat/emtricitabine/tenofovir alafenamide fumarate combination, elvitegravir/cobicistat/emtricitabine/tenofovir disoproxil fumarate combination, emtricitabine/rilpivirine/tenofovir alafenamide combination, emtricitabine/rilpivirine/tenofovir disoproxil fumarate combination, emtricitabine/tenofovir alafenamide combination, emtricitabine/tenofovir disoproxil fumarate combination, lamivudine/tenofovir disoproxil fumarate combination, lamivudine/zidovudine combination, lopinavir/ritonavir combination or any combination thereof.

The present disclosure provides a method of reactivating latent HIV in a subject comprising administering to the subject a therapeutically effective amount of at least one compound of the present disclosure. The present disclosure provides a method of reactivating latent HIV without inducing global T cell activation in a subject comprising administering to the subject a therapeutically effective amount of at least one compound of the present disclosure.

### Methods of Use—Acute Coronary Syndrome

Acute Coronary Syndrome (ACS) is a group of conditions including unstable angina and myocardial infarctions (MI) with or without an observed ST elevation with atherosclerosis being the primary cause. Acute therapy involves interventional and/or medical therapy (anti-thrombotic, anticoagulant, anti-ischemic, anti-lipid). Secondary prevention treatment post ACS includes lifestyle changes, medical treatment to control risk factors and continued anti-thrombotic therapy. Despite SOC, there remains a significant risk of reinfarction, ischemic stroke, and death (up to 18% in the first year post ACS).

Studies have shown that acetylation level through HDACs is associated with cardiovascular disease, such as hypertension, diabetic cardiomyopathy, coronary artery disease, arrhythmia, and heart failure. Moreover, HDACs appear to be closely linked with in the progression of atherosclerosis and HDAC inhibitors successfully prevent the progression of atherosclerosis. Positive effects of pan-selective HDAC inhibitors, which increase the acetylation of histones and nonhistone proteins, in rodent models of heart failure have been reviewed extensively. Importantly, HDAC inhibition is capable of regressing established cardiac hypertrophy and systolic dysfunction in mice subjected to aortic constriction. In a rabbit ischemic-reperfusion injury, the use of an HDACi protected cardiac tissue and function by inhibition of pathological remodeling through autophagy which serves to protect cardiomyocytes during ischemia by resupplying energy and by reducing inflammation, oxidative stress and fibrosis.

The present disclosure provides a method of treating Acute Coronary Syndrome in a subject comprising administering to the subject a therapeutically effective amount of at least one compound of the present disclosure. The present disclosure provides a method of reducing damage to cardiac cells in a subject having acute coronary syndrome comprising administering to the subject a therapeutically effective amount of at least one compound of the present disclosure. The present disclosure provides a method of reducing damage imparted by ischemia, inflammation, fibrotic remodeling or any combination thereof in a subject comprising administering to the subject a therapeutically effective amount of at least one compound of the present disclosure.

Acute Coronary Syndrome can include, but is not limited to, a heart attack, an unstable angina, ST elevation myocardial infarction, non ST elevation myocardial infarction or any combination thereof.

The present disclosure provides a method of preventing reinfarction in a subject comprising administering to the subject a therapeutically effective amount of at least one compound of the present disclosure. The present disclosure provides a method of preventing ischemic stroke in a subject comprising administering to the subject a therapeutically effective amount of at least one compound of the present disclosure.

The present disclosure provides a method of increasing the survival of cardiac cells in a subject comprising administering to the subject a therapeutically effective amount of at least one compound of the present disclosure.

An increase in survival of cardiac cells can be about a 10%, or about a 20%, or about a 30%, or about a 40%, or about a 50%, or about a 60%, or about a 70%, or about an 80%, or about a 90%, or about a 100%, or about a 110%, or about a 120%, or about a 130%, or about a 140%, or about a 150%, or about a 160%, or about a 170%, or about a 180%, or about a 190%, or about a 200%, or about a 250%, or about a 300%, or about a 350%, or about a 400%, or about a 450%, or about a 500%, or about a 600%, or about a 700%, or about an 800%, or about a 900%, or about a 1000% increase in the survival of cardiac cells.

### Methods of Use—Depression

Major depressive disorder is a chronic, remitting syndrome involving widely distributed circuits in the brain. Stable alterations in gene expression that contribute to structural and functional changes in multiple brain regions are implicated in the heterogeneity and pathogenesis of the illness. Epigenetic events that alter chromatin structure to regulate programs of gene expression have been associated with depression-related behavior, antidepressant action, and resistance to depression or ‘resilience’ in animal models, with increasing evidence for similar mechanisms occurring in postmortem brains of depressed humans.

The role of epigenetics and more specifically histone acetylation in depression comes primarily from chronic stress derived animal models. Certain behavioral alterations induced by chronic stress are long-lasting and can be effectively reversed by a chronic treatment antidepressant regimen that could be considered comparable with that used in depressed patients. Chronic stress paradigms involve prolonged exposure to either physical stressors or bouts of social subordination that produce anhedonia-like symptoms, characterized by a decrease in reward-related behaviors such as preferences for sucrose or high fat diets and social interaction. The potential importance of histone acetylation in depression was initially suggested by observations that HDAC inhibition alone, or in combination with, antidepressant treatment ameliorated depression-like behaviors in rodents. Changes in brain-derived neurotrophic factor (BDNF) and nerve growth factor (VGF) in the prefrontal cortex, hippocampus, and nucleus accumbens have been implicated in depressed humans and/or following chronic stress in rodent models and can be reversed by chronic treatment with antidepressants (Sun 2013). Histone acetylation has been found to be persistently increased in the nucleus accumbens (NAc; and HDAC2 reduced) in a chronic social defeat stress animal model. These changes were also observed in the NAc of depression patients in postmortem examination. Similarly, a large body of literature has suggested that histone acetylation in the hippocampus has an overall adaptive role in stress and antidepressant responses.

The present disclosure provides a method of treating major depressive disorder in a subject comprising administering to the subject a therapeutically effective amount of at least one compound of the present disclosure. The present disclosure provides a method of preventing major depressive disorder in a subject comprising administering to the subject a therapeutically effective amount of at least one compound of the present disclosure.

The present disclosure provides a method of reversing acetylation patterns induced by major depressive disorder in a subject comprising administering to the subject a therapeutically effective amount of at least one compound of the present disclosure.

The present disclosure provides a method of augmenting the therapeutic effect of an anti-depressant compound in a subject comprising administering to the subject a combination of a therapeutically effective amount of the anti-depressant compound and a therapeutically effective amount of at least one compound of the present disclosure.

Anti-depressant compounds can include, part are not limited to, selective serotonin reuptake inhibitors, serotonin-norepinephrine reuptake inhibitors, serotonin modulators and stimulators, serotonin antagonists and reuptake inhibitors, norepinephrine reuptake inhibitors, norepinephrine-dopamine reuptake inhibitors, tricyclic antidepressants, tetracyclic antidepressants, monoamine oxidase inhibitors and atypical antipsychotics. Anti-depressant compounds can include, part are not limited to, Citalopram, Escitalopram, Paroxetine, Fluoxetine, Fluvoxamine, Sertraline, Indalpine, zimelidine, Desvenlafaxine, Duloxetine, Levomilnacipran, Milnacipran, Venlafaxine, Vilazodone, Vortioxetine, Nefazodone, Trazodone, Etoperidone, Reboxetine, Teniloxazine, Viloxazine, reboxetine, Atomoxetine, Bupropion, Amineptine, Methylphenidate, Lisdexamfetamine, Amitriptyline, Amitriptylinoxide, Clomipramine, Desipramine, Dibenzepin, Dimetacrine, Dosulepin, Doxepin, Imipramine, Lofepramine, Melitracen, Nitroxazepine, Nortriptyline, Opipramol, Pipofezine, Protriptyline, Trimipramine, Butriptyline, demexiptiline, fluacizine, imipraminoxide, iprindole, metapramine, propizepine, quinupramine, Tiazesim, tofenacin, Amineptine, tianeptine, Amoxapine, Maprotiline, Mianserin, Mirtazapine, Setiptiline, Isocarboxazid, Phenelzine, Tranylcypromine, benmoxin, iproclozide, iproniazid, mebanazine, nialamide, octamoxin, pheniprazine, phenoxypropazine, pivhydrazine, safrazine, Selegiline, Caroxazone, Metralindole, Moclobemide, Pirlindole, Toloxatone, Eprobemide, minaprine, Bifemelane, Amisulpride, Lurasidone, Quetiapine, Agomelatine, Ketamine, Tandospirone, Tianeptine, α-Methyltryptamine, Etryptamine, Indeloxazine, Medifoxamine, Oxaflozane, Pivagabine, Ademetionine, Hypericum perforatum, Oxitriptan, Rubidium chloride, Tryptophan, Aripiprazole, Brexpiprazole, Lurasidone, Olanzapine, Quetiapine, Risperidone, Buspirone, Lithium, Thyroxine, Triiodothyronine, Pindolol, Amitriptyline/perphenazine, Flupentixot/melitracen, Olanzapine/fluoxetine, Tranylcypromine/trifluoperazine or any combination thereof

### Methods of Use—Autoimmune Disease

Systemic autoimmune rheumatic diseases such as rheumatic arthritis (RA), juvenile idiopathic arthritis, and systemic lupus erythematosus (SLE) are characterized by chronic inflammation and pain, which consequently leads to tissue destruction and reduction of patients' mobility. Immune cells play a key role in inflammation due to involvement in initiation and maintenance of the chronic inflammatory stages and epigenetic mechanisms can mediate the development of chronic inflammation. Rheumatoid arthritis (RA) and juvenile idiopathic arthritis (JIA) are autoimmune diseases characterized by chronic joint inflammation with pain and swelling, joint destruction and disability. Activation of nonspecific innate immunity, results in persistent chronic inflammation orchestrated by uncontrolled production of many proinflammatory mediators, such as cytokines, chemokines and other soluble factors, becoming a loop of self-reverberating inflammation that becomes independent of the original trigger. Cytokines such as tumor necrosis factor (TNF) and interleukin (IL)-1β produced by macrophages and lymphocytes infiltrating the synovial tissue lead to the abnormal activation of fibroblast-like synoviocytes (FLS), which in turn causes bone and cartilage deterioration. Inhibition of HDAC activity can contribute to the immunopathology of RA and JIA via epigenetic mechanisms. When comparing healthy individuals and RA disease controls, synovial tissue displays a marked reduction in total HDAC activity and HDAC1 and HDAC2 protein expression, particularly in synovial macrophages. The use of pan-HDACis reduce cytokine production in in fibroblast-like synoviocytes and in immune cells from patients with RA, display antiarthritic properties in vivo and demonstrated primary clinical efficacy in the treatment of rheumatic diseases. This demonstrates that protein acetylation plays a role in treating rheumatic diseases.

The present disclosure provides a method of treating rheumatic disease including rheumatoid arthritis and juvenile idiopathic arthritis in a subject comprising administering to the subject a therapeutically effective amount of at least one compound of the present disclosure. The present disclosure provides a method of increasing crotonylation of histones in a subject comprising administering to the subject a therapeutically effective amount of an acetyl-CoA precursor.

Systemic lupus erythematosus (SLE) is a systemic autoimmune disease characterized by the activation of autoreactive T and B cells. SLE can affect many parts of the body, including the joints, skin, kidneys, heart, lungs, blood vessels and brain but some of the most common symptoms include extreme fatigue, painful or swollen joints, fever, photosensitivity, hair loss, skin rashes (specifically the characteristic red butterfly or malar rash across the nose and cheeks), and renal impairment. SLE treatment consists primarily of immunosuppressive drugs. HDAC expression and activity is found to be upregulated in murine models of disease (Regina) and HDACis can reduce disease in lupus-prone mice (Reilly 2004 and 2011).

The present disclosure provides a method of treating SLE in a subject comprising administering to the subject a therapeutically effective amount of at least one compound of the present disclosure.

### Definitions

Unless otherwise stated, the following terms used in the specification and claims have the following meanings set out below.

As use herein, the phrase “compound of the present disclosure” refers to those compounds which are disclosed herein generically, sub-generically, and specifically (i.e., at species level).

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, adamantly, hexahydroindacenyl. It is understood that for polycyclic (e.g., fused, bridged, or spiro rings) system, only one of the rings therein needs to be non-aromatic. For example, the cycloalkyl may be hexahydroindacenyl.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic non-aromatic rings, only one of the rings needs to be non-aromatic (e.g., 1,2,3,4-tetrahydronaphthalenyl or 2,3-dihydroindole).

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. In some embodiments, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include, but are not limited to, pyrrole, furan, thiophene, thiazole, isothiazole, imidazole, triazole, tetrazole, pyrazole, oxazole, isoxazole, pyridine, pyrazine, pyridazine, pyrimidine, benzoxazole, benzodioxazole, benzothiazole, benzoimidazole, benzothiophene, quinoline, isoquinoline, naphthrydine, indole, benzofuran, purine, benzofuran, deazapurine, and indolizine.

As used herein, the term “optionally substituted”, unless specified otherwise, refers to being unsubstituted or having designated substituents replacing one or more hydrogen atoms on one or more designated atoms of the referred moiety. Suitable substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

As used herein, the term “halo” or “halogen” refers to fluoro, chloro, bromo and iodo.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

The terms “effective amount” and “therapeutically effective amount” of an agent or compound are used in the broadest sense to refer to a nontoxic but sufficient amount of an active agent or compound to provide the desired effect or benefit.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

Organelles can include, but are not limited to, lysosomes, the endoplasmic reticulum, endosomes, the nucleus, mitochondria, the golgi apparatus, the vacuole and peroxisomes. The phrase “particular organelle” is also used to refer to specific substructures within an organelle, such as, but not limited to, intermembrane space of mitochondria, the cristae of mitochondria, the matrix of mitochondria, the perinuclear space of the nucleus, the rough endoplasmic reticulum, the smooth endoplasmic reticulum, the cis golgi and the trans golgi.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is an imprinting disorder. It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration. Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human. A subject in need thereof can be one who has been previously diagnosed or identified as having an imprinting disorder. A subject in need thereof can also be one who has (e.g., is suffering from) an imprinting disorder. Alternatively, a subject in need thereof can be one who has an increased risk of developing such disorder relative to the population at large (i.e., a subject who is predisposed to developing such disorder relative to the population at large). A subject in need thereof can have a refractory or resistant imprinting disorder (i.e., an imprinting disorder that doesn't respond or hasn't yet responded to treatment). The subject may be resistant at start of treatment or may become resistant during treatment. In some embodiments, the subject in need thereof received and failed all known effective therapies for an imprinting disorder. In some embodiments, the subject in need thereof received at least one prior therapy. In a preferred embodiment, the subject has an imprinting disorder.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

As used herein, the term “preventing,” “prevent,” or “protecting against” describes reducing or eliminating the onset of the symptoms or complications of such disease, condition or disorder.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is understood that, when a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

It is understood that, when any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that, for the compounds of the present disclosure being capable of further forming salts, all of these forms are also contemplated within the scope of the claimed disclosure.

It is to be understood that the compounds of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

It is to be understood that the compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognise the advantages of certain routes of administration.

It is to be understood that dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

It is to be understood that a pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., imprinting disorders, and the like) and the health of the patient should preferably be closely monitored during and for a reasonable period after treatment.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It may be especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

It is to be understood that the pharmaceutical compositions can be included in a container, pack, or dispenser together with instructions for administration.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1: In Vitro Biology Experimental Procedures A for Testing Compounds of the Present Disclosure

Efficacy of compounds of the present disclosure can be assessed via similar procedures as those described in examples 1-9 by one skilled in the art. Compounds of the present disclosure can be dosed in cells (included but not limited to cell lines, patient derived cells, iPSC of any kind, EC and tissue organoids) with metabolic impartments (including but not limited to impaired amino acid metabolism, impaired fatty acid metabolism, impaired TCA cycle, impaired glucose metabolism, impaired metabolic respiration, impaired carbohydrate metabolism, impartments of organic acid metabolism and the like) by incubating several concentrations of compounds of the present disclosure, either alone or in combination (with other small molecule drugs, biologic drugs, adjuvant therapies) in a suitable vehicle formulation (such as but not limited to saline, HPMC, PEG400, HPBCD and the like) over a period of minutes, hours up to several days. Following incubations, cells (including supernatants) can be assayed in multiple ways (as indicated in Biology Experimental 1-8) including but not limited to bioanalytical, biochemical, biomarker, functional. One can analyze tissues for CoA and Acyl-CoA species (such as but not limited to Acetyl-CoA, Succinyl-CoA, Malonyl-CoA, TCA cycle intermediates and the like), Acyl-Carnitines, Carnitine and AcylCarnitine Transport and transporters, ketone bodies, Organic Acids, and other metabolites consistent with the biochemical and metabolic pathways, utilizing analytical methods including but not limited to HPLC, MS, LCMS, MRI, western blot, ELISA, PCR, Reactive Oxygen Species, tubulin acetylation and other Post Translation Modifications, Next Generation Sequence, enzyme processing, enzyme inhibition, complex formation and the like. One can measure functional aspects and changes in functional readouts such as Mitochondrial Bioenergetics (including but not limited to OCR, ECAR, Complex formation, ATP production), mitochondrial membrane potential, mitochondrial morphology and/or architectural changes (including but not limited to fusion, fission, membrane structure and morphology), Patch-clamp electrophysiology. One can measure metabolomic changes and improvements in metabolic flux and TCA function.

### Example 2: Isolation and Purification of Acyl-Coenzyme A Esters (Including Acetyl-CoA)

### Sample Preparation for Acyl-CoA Profiling (In Vivo)

Animals could be killed by exposure to CO2 followed by cervical dislocation. The liver was rapidly excised, frozen in liquid nitrogen and then powdered under liquid nitrogen. For each analysis, precisely-measured amounts (between 0.1 to 0.2 g) of powdered tissue were spiked to a final concentration of 20 ppm in a final volume of 100 mL with the [D3]acetyl-CoA standard, then homogenized in 2 mL ice-cold 10% trichloroacetic acid with 2 mM DTT using a Polytron (Kinematica Tnc, Bohemia, NY). The tubes were vortexed for 5 sec and centrifuged at 4 uC for 5 min at 13,000 g. The supernatants were then applied to a 3 cc Oasis HLB solid-phase extraction column (Waters, Milford, MA, USA) preconditioned with 2 mL of methanol and 2 mL of water. The column was then washed with 2 mL of 2 mM DTT in water and eluted with 2 mL of 2 mM DTT in methanol. The eluate was evaporated under a stream of nitrogen, reconstituted in 100 mL of 2 mM DTT in water. 20 mL served for high performance liquid chromatography coupled to tandem mass spectrometry (HPLC/MS/MS) analysis.

### HPLC/MS/MS Assay of Short Chain Acyl-CoAs

The HPLC/MS/MS system consists of a 2795 Waters HPLC coupled to a Micromass Quattro Premier XE (Milford, MA, USA). The column was a 15063 mm Gemini-NX C18 (5 microns) from Phenomenex (Torrance, CA). Eluent A was 2 mM ammonium acetate in water and eluent B was 2 mM ammonium acetate in acetonitrile. The gradient was 100% A for 5 min, going to 50% B after 30 min, then to 100% B after 31 min, maintained at this composition until 36 min, returning to the initial composition at 37 min and stabilized until 42 min. Flow rate was 0.4 mL/min. The MS was operated in negative ionization electrospray with the following settings: desolvation gas 100 L/Hr; cone gas 10 L/Hr; capillary voltage 2.5 kV; source temperature 120 uC; and cone voltage 20 V. The mass spectrometric data were obtained in multiple reaction monitoring acquisition mode for nine short chain acyl-CoA species using the following transitions (m/z) and collision energies: free CoA (382.5.685.9, 17 V), succinyl-CoA (432.5.685.7, 15 V), isovaleryl-CoA (424.5.769.9, 18 V), HMG-CoA (454.5.382.5, 15 V), acetoacetyl-CoA (424.6.382.4, 11 V), butyryl-CoA (417.7.755.7, 17 V), methylcrotonyl-CoA (423.7.685.7, 20 V), acetyl-CoA (403.6.728, 15 V) and the internal standard [D3]acetyl-CoA (404.6.730.9, 15 V). The parent and daughter ions and the collision energy used for each acyl-CoA multiple reaction monitoring were determined using pure samples. Standard curves were constructed for each acyl-CoA using pure molecules. Standard curves were spiked with the internal standard [D3]acetyl-CoA to compare the relative response factor between each molecule and the standard for the quantification of those short chain acyl-CoAs in the mouse liver sample.

### MS Determination of Unidentified Acyl-CoAs

To identify unknown acyl-CoA species, analyses were performed on a 6224 TOF MS coupled to a 1260 Infinity HPLC system, both from Agilent Technologies Inc. Ionization was performed in negative mode on a dual spray ESI source and mass spectra were acquired from m/z 100 to 3200. Samples were diluted to 50 mL, then 2 mL aliquots were injected into the LC-MS system. The chromatographic column was an XBridge C18, 3.5 mm, 4.6650 mm from Waters. Elution was performed under a two step gradient using acetonitrile and 10 mM ammonium acetate as mobile phases. Deprotonated species were taken into account for accurate mass calculation.

In a 12×75-mm glass tube was placed powdered rat liver (20-26 mg) and radiolabeled acyl-coenzyme A standards ranging 44,440-55,000 dpm and 0.35-0.46 nmol. These amounts of added radiolabeled acyl-coenzyme A esters are in the concentration ranges reported in the literature.

Next, 1.5 ml of acetonitrile/isopropanol (3+1, v+v) was added and a 30-s homogenization was performed using an OMNI 2000 tissue homogenizer followed by addition of 0.5 ml of 0.1 M KH2PO4 (pH 6.7) and a second 30-s homogenization. The resulting homogenate was vortex-mixed (5 s), and two 200-ll aliquots were transferred to scintillation vials for radioactivity determination (100% recovery). The remainder was transferred to a microcentrifuge tube and centrifuged for 5 min at 16,000 g. Two 200-ll aliquots were removed from the supernatant for determination of recovery by radioactivity counting, and 1 ml of the remaining supernatant was transferred to a 12×75-mm glass tube and acidified by adding 0.25 ml of glacial acetic acid and vortex-mixing. The SPE column was conditioned with 1 ml of acetonitrile/isopropanol/water/acetic acid (9+3+4+4, v+v+v+v). This solution ensures protonation of the pyridyl functional group, so that it will function as an anion-exchanger. Following application and flowthrough of the supernatant (collected in 625-ll aliquots), the SPE column was washed with 1 ml of acetonitrile/isopropanol/water/acetic acid (9+3+4+4, v+v+v+v) to remove unretained species (collected in 500-ll aliquots). Acyl-coenzyme A esters were then eluted with 2 ml of methanol/250 mM ammonium formate (4+1, v+v; collected in 500-ll aliquots). This eluent has a pH of 7, which neutralizes the pyridyl functional group. All aliquots had their radioactivity content determined by liquid scintillation counting. This was performed, following the addition of 4 ml/vial of Ultima Gold scintillation cocktail (Perkin Elmer, Waltham, MA), using an LS 6500 scintillation counter (Beckman Coulter, Fullerton, CA). Recoveries were calculated from the determined radioactivity using correction factors for the percentage of the volume that was counted.

### Example 3: Determination of Reactive Oxygen Species (ROS)

Human neurons were incubated with Alexa Fluor 647 mouse anti-human CD56 (anti-NCAM, BD Biosciences, diluted 1:40) for 1 h, with 20 μM of 2′,7′-dichlorodihydrofluorescein diacetate (H₂DCFDA; Molecular Probes) for 15 min, and with 2 μg/ml of Hoechst 33342 for 2 min. All incubations were performed at 37° C. The cells were washed and randomly analyzed using an IN Cell Analyzer 1000 system (GE Healthcare). The fluorescence of DCF from NCAM-positive cells was collected to compare the relative ROS contents. The quantification of the signal was performed using the NIH image software, ImageJ. A minimum of 100 neurons for each patient and control was analyzed in at least three independent experiments for each sample.

### Example 4: Determination of Mitochondrial Membrane Potential

Human neurons were incubated with Alexa Fluor 488 mouse anti-human CD 56 (anti-NCAM; BD Biosciences) for 1 h, with 20 nM of TMRM (Molecular Probes) for 15 min, and with 2 μg/ml of Hoechst 33342 for 2 min. All of these incubations were performed at 37° C. The cells were washed and randomly analyzed by IN Cell Analyzer 1000 system (GE Healthcare). The fluorescence of TMRM from NCAM-positive cells was collected to compare the relative mitochondrial membrane potential. A minimum of 100 neurons for each patient and control was analyzed in at least three independent experiments for each sample.

### Example 5: Patch-Clamp Elecrophysiology (iPSC Neuronal)

Co-culture experiments of 6×10⁴ cells (half GFP controls and half tdTomato patients) were seeded on Matrigel-coated covers. After 5 days, 2×10⁴ cortical mice neurons were added to improve differentiation and electrophysiological activity. Individual slides containing co-cultured PKAN and control neurons were transferred in a recording chamber mounted on the stage of an upright BX51WI microscope (Olympus, Japan) equipped with differential interference contrast optics (DIC) and an optical filter set for the detection of GFP and tdTomato fluorescence (Semrock, Rochester, NY, USA). Cells were perfused with artificial cerebrospinal fluid (ACSF) containing (in mM): 125 NaCl, 3.5 KCl, 1.25 NaH₂PO₄, 2 CaCl₂), 25 NaHCO₃, 1 MgCl₂, and 11 D-glucose, saturated with 95% O₂ and 5% CO₂ (pH 7.3). The ACSF was continuously flowing at a rate of 2-3 ml/min at room temperature. Whole-cell patch-clamp recordings were performed using glass pipettes filled with a solution containing the following (in mM): 10 NaCl, 124 KH₂PO₄, 10 HEPES, 0.5 EGTA, 2 MgCl₂, 2 Na₂-ATP, 0.02 Na-GTP, (pH 7.2, adjusted with KOH; tip resistance: 4-6 MΩ). All recordings were performed using a MultiClamp 700B amplifier interfaced with a PC through a Digidata 1440A (Molecular Devices). Data were acquired using pClamp10 software (Molecular Devices) and analyzed with GraphPad Prism 5 and SigmaStat 3.5 (Systat Software Inc.). Voltage- and current-clamp traces were sampled at a frequency of 10 kHz and low-pass filtered at 2 kHz. The input resistance (Rᵢₙ) was calculated by dividing the steady-state voltage response to a negative current step (−10 to −50 pA, 1 s) by the amplitude of the injected current. Labeled GFP or tdTomato neurons were randomly chosen for measurement, and no blind experiments were done for electrophysiology studies.

### Example 6: Determination of Respiratory Activity (Basal, ATP Production-Linked, Maximal, and Proton Leak-Linked Oxygen Consumption Rate)

Oxygen consumption rate (OCR) was measured in PKAN and control neurons with a XF96 Extracellular Flux Analyzer (Seahorse Bioscience, Billerica, MA, USA). Each control and PKAN NPC was seeded on a XF 96-well cell culture microplate (Seahorse Bioscience) at a density of 15-20×10³ cells/well and differentiated as previously described. After replacing the growth medium with 180 μl of bicarbonate-free DMEM pre-warmed at 37° C., cells were incubated at 37° C. without CO₂ for 1 h before starting the assay procedure. Then, baseline measurements of OCR, after addition of 1 μM oligomycin and of 2.1 μM carbonyl cyanide 4-(trifluoromethoxy) phenylhydrazone (FCCP), were measured using an already established protocol (Invernizzi et al, 2012). Data were expressed as pmol of O₂ per minute and normalized by cell number measured by the CyQUANT Cell proliferation kit (Invitrogen), which is based on a fluorochrome binding to nucleic acids. Fluorescence was measured in a microplate luminometer with excitation wavelength at 485±10 nm and emission detection wavelength at 530±12.5 nm.

### Example 7: Western Blot Analysis of Tubulin Acetylation in Livers from Mice

Livers were homogenized on ice with a glass-glass potter and lysed using RIPA buffer (50 mM Tris pH 8, 150 mM NaCl, 1% NP40, 0.5% Na-deoxycholate, 0.1% SDS, 5 mM EDTA pH 8) with addition of protease inhibitor cocktail (Roche). Proteins were quantified by BioRad protein assay according to manufacturer instructions. Equal amounts of proteins (20 μg) were resolved on a 12% SDS-polyacrilamide gel and electroblotted onto nitrocellulose membrane. Filters were incubated with mouse monoclonal anti-acetylated tubulin antibody (clone 6-11B-1, Sigma). Equal loading was verified using a mouse monoclonal anti-GAPDH antibody (clone 6C5, Millipore). Peroxidase-conjugated secondary antibodies (Amersham) were visualized using the ECL method with autoradiography film.

### Example 8: Mitochondrial Protein Acetylation: Lysine Acetylation on Proteins

### Acetyl Lysine Analysis in Human Fibroblasts

Human dermal fibroblasts were routinely cultured in DMEM supplemented with 10% (v/v) fetal calf serum, 2 mm glutamine and 1% (v/v/v) pen/strep/fungizone. For acetyl lysine analysis we incubated cells either in serum-free Eagle's minimal essential medium (MEM) supplemented with 400 μml-carnitine and 120 μm palmitate for 96 h [a metabolic condition characterized by high fatty acid turnover] or in DMEM. After exposure, the cell pellet was resuspended in 50 mm NH₄CO₃ buffer containing deacetylase inhibitors (1 μm Trichostatin A and 10 mm nicotinamide) followed by sonication at 40 J/Ws. To digest the protein into amino acids, samples were incubated with pronase at a protein to pronase ratio of 10:1, in 50 mm NH₄CO₃ for 4 h at 37° C. The reaction was stopped with 5 volumes of acetonitrile, 10 μl 2.5 mm D4-labeled 1-lysine internal standard (DLM-2640, Cambridge Isotopes Laboratories) and 10 μl 10 μm D8-labelled acetyl lysine internal standard (D-6690, CDN Isotopes). The samples were briefly vortexed and centrifuged at 14 000 rpm, 4° C. 10 for 10 min followed by solvent evaporation at 40° C. under a gentle stream of nitrogen. Samples were then taken up in 0.01% heptafluorobutyric acid and analyzed with LC-MS/MS.

### Acetyl-Lysine Measurement Using LC-MSMS

Ten microliters of the sample extract was injected onto a BEH C18 column (2.1×100 mm, 1.7 μm, Waters Corp. Milford MA) using a UPLC system consisting of an Acquity solvent manager with degasser and an Acquity Sample Manager with column oven (Waters Corp.). The system was controlled by MassLynx 4.1 software. The flow rate was set to 500 μl/min. Elution solvent A consisted of 0.1% heptafluorobutyric acid and solvent B was 80% acetonitrile. The chromatographic conditions were as follows: 0-2 min 1000% A, 2-5 min to 50% B, 5-6 min to 1000% B, at 6.1 min back to 1000% A and equilibration time with 100% A was 3 min. Separation was performed at 50° C. The Quattro Premier XE triple-quadrupole mass spectrometer (Waters Corp.) was used in the positive electrospray ionization (ESI) mode. Nitrogen was used as nebulizing gas and argon was used as collision gas at a pressure of 2.5e-3 mbar. The capillary voltage was 3.0 kV, the source temperature was 120° C. and desolvation temperature was 300° C. Cone gas flow was 50 l/h and desolvation gas flow was 900 l/h. All components were measured by using multiple reaction monitoring (MRM) in the positive ionization mode, using the transitions: m/z 147.0>84.1 for lysine, 151.0>88.1 for lysine-²H₄ (internal standard), 189.2>84.1 for N-acetyl lysine and 197.2>91.1 for N-acetyl lysine-²H₈ (internal standard) with optimal collision energy of 20 eV for lysine and 30 eV for N-acetyl lysine.

### Example 9: General In Vivo Biology Experimental Procedures for Testing Compounds

Administration of compounds of the present disclosure to animals generated in Biology Experimentals 10-B10 or other models of metabolic diseases (including but not limited to impaired amino acid metabolism models, impaired fatty acid metabolism models, impaired TCA cycle models, impaired glucose metabolism models, impaired metabolic respiration models, organ transplant models, impaired carbohydrate metabolism models, models of disorders of organic acid metabolism and the like) or other models of post-translational modification (including but not limited to impaired histone prenylation (such as Acetylation) models, impaired tubulin prenylation (such as Acetylation) models and the like), by dosing (either orally, ip, sc, iv or other route of administration) and either alone or in combination with another compound or another agent (such as but not limited to other small molecule drugs, biologic drugs, adjuvant therapies, gene therapies and the like) in a suitable vehicle formulation (such as but not limited to saline, HPMC, PEG400, HPBCD and the like) over a period of minutes to days (up to several months), would demonstrate benefit. Following dosing, animals can be sacrificed and tissues and organs collected (such as but not limited to blood, plasma, serum, CSF, liver, brain, heart, kidney, lungs, skin, muscle). These animals and tissue samples can be analyzed in multiple ways, including but not limited to clinical signs, bioanalytical, biochemical, biomarker, functional, behavioral, movement, cognitive and metabolic measures of efficacy. One can analyze tissues for CoA and Acyl-CoA species (such as but not limited to Acetyl-CoA, Succinyl-CoA, Malonyl-CoA, TCA cycle intermediates and the like), Acyl-Carnitines, Carnitine and AcylCarnitine Transport and transporters, ketone bodies, Organic Acids, and other metabolites consistent with the biochemical and metabolic pathways, utilizing analytical methods including but not limited to HPLC, MS, LCMS, MRI, CAT scan, PET scan, western blot, ELISA, PCR, enzyme processing, enzyme inhibition, complex formation and the like. One can measure functional aspects and changes in functional readouts such as Mitochondrial Bioenergetics (including but not limited to OCR, ECAR, Complex formation, ATP production), mitochondrial morphology and/or architectural changes (including but not limited to fusion, fission, membrane structure and morphology). One can measure prolongation of life in these animal models, temperature changes, mobility (including but not limited to walking, running, open field test, maze, treadmill), motor coordination (such as but not limited to Rotarod test), strength, and other functional measures of movement and cognition following treatment of Compounds. One can measure metabolomic changes and improvements in metabolic and TCA function.

### Example 10: Generation of a Hypomorphic Model of Propionic Acidemia (hPCCA Hypomorph Mice)

Segments of human PCCA cDNA with mutations leading to A75P or A138T defects were synthesized by GenScript USA (Piscataway, NJ). These were used to replace wild-type Pcca in plasmid pShuttleCMV-FL-hPCCA-IRES-hrGFP. These mutant PCCA cDNAs were transferred to pCALL2-Δ-LoxP to generate plasmids pCALL2-Δ-LoxP-hPCCA-A75P and pCALL2-Δ-LoxP-hPCCA-A138T in which hPCCA is followed by an IRES-EGFP element to allow screening for transgenics. The pCALL2-Δ-LoxP plasmids were digested with BamHI and BsaWI and this transgene fragment was microinjected into the fertilized eggs of FVB mice. Founder mice were screened for GFP expression and by PCR using primers specific for the transgene cassette (F: CGGATTACGCGTAGCATGGTGAGCAA R: GCCTAAACGCGTTTACTTGTACAGCT). Positive mice were then crossed to Pcca+/− mice. All resulting progeny were screened using primers specific for the endogenous mPCCA gene, neomycin resistance gene (neo) and the transgene cassette described previously.

### Example 11: Production of Liver-Specific HL-Deficient Mice

Construction of the gene targeting vector and targeting in embryonal stem cells are described in Supplemental Information. Targeted embryonal stem cell clones were microinjected into C57BL/6J blastocysts and transferred to pseudopregnant recipients. We obtained 4 chimeras from one clone and 6 from the other. Chimeras were bred to C57BL/6J mice. Agouti offspring were genotyped to identify heterozygotes (HL+/L). In order to obtain the excision in liver of HL exon 2, which is catalytically essential [16], HL heterozygotes (HL+/L) were bred to Alb-Cre mice (B6.Cg-Tg (Alb-cre) 21 Mgn/J, 003574. Alb-Cre mice express Cre recombinase from the hepatocyte-specific albumin promoter. HL+/LCre+ mice were crossed to obtain Cre transgenic HLL/L homozygotes (HLL/LCre+; henceforth designated HL liver knockout (HLLKO) mice).

### Example 12: Generation of Mut^{ki/ki} and Mut^{ko/ki} Mouse Models, which Survive Long Term

The generation of mice carrying the Mut-p.Met698Lys mutation was performed by Polygene (Rümlang, Switzerland) using embryonic stem cell targeting. To generate Mut^{ko/ki} mice, female Mut^{ko/wt} (Peters H, 2003) were crossed to Mut^{ki/ki} males. Mouse genotyping was performed on genomic DNA from ear punch biopsies using the primers 5′-GTGGGTGTCAGCACACTTG-3′ (forward) and 5′-CGTATGACTGGGATGCCT-3′ (reverse) for the ki allele and 5′-ACAACTCCTTGTGTAGGTC-3′ (forward) and 5′-CCTTTAGGATGTCATTCTG-3′ (reverse) for the ko allele.

### Example 13: Generation of PDC-Deficient Mice

Generation of a mouse colony harboring a silent mutation in the Pdha1 gene (two loxP sites into intron sequences flanking exon 8; referred to as the Pdha1flox8 allele). These mice were maintained on a standard rodent laboratory diet and water ad libitum. To initiate deletion of exon 8 in vivo in all tissues of the progeny, homozygous floxed females (genotypes: Pdha1flox8/Pdha1-flox8) were bred with homozygous males from an EIIa-Cre transgenic mouse line (genotype: Pdha1wt/Y; Creall+; referred to as Cre transgenic males) to generate experimental heterozygous female progeny (referred to as PDC-deficient females with the genotype: Pdha1 wt/PDHa1Dex8, Creall+). The transgenic Creall+mouse strain was homozygous for an autosomally integrated Cre transgene under the control of the adenovirus EIIa promoter that targets expression of Cre recombinase beginning on embryonic day 1. To generate control female progeny (referred to as controls) wild-type males (without carrying a Cre transgene), were bred with homozygous Pdha1 flox8 females.

### Example 14: Generation of Long-Chain Acyl-CoA Dehydrogenase-Deficient Mice (LCADD-Mice)

The targeting vector pAcadl^{tm1Uab} was constructed by using a 7.5-kb Acadl (Nod/HindIII) fragment of 129/SvJ DNA and a neo^{r} cassette derived from PGKneobpA, under the control of the phosphoglycerate kinase gene promoter and a bovine poly(A) signal and subcloned into pGEM-11zf(+) (Promega). An 821-bp deletion of the Acadl sequence, spanning exon 3 with flanking intron sequence, was created in the vector before electroporation and served as the site of linearization. Repair of this deletion on homologous recombination via the double-stranded-break repair model (Scostak J W, 1983) served as the basis for screening ES cell colonies for correct targeting by Southern blot analysis. Duplication of exon 3 can occur only on homologous recombination. Linearized vector was electroporated into TC-1 ES cells derived from 129/SvEvTacfBR (129) mice, and G418-resistant clones were analyzed by using Southern blot analysis. Correctly targeted clones were microinjected into C57BL/6J (B6) blastocysts to generate chimeras that were backcrossed to C57BL/6NTacfBR mice (Taconic). All mice analyzed in these studies were generation 2-3 with B6,129-Acadl^{tm1Uab/tm1Uab} (LCAD−/−) or B6,129-Acadl^{+/+} (normal control) genotypes from intercrosses of B6,129-Acadl^{tm1Uab/+} (LCAD−/+) mice. Genotypes were determined by using Southern blot analysis. Mice were negative for murine pathogens based on a panel of 10 virus serologies, aerobic bacterial cultures of nasopharynx and cecum, endo- and ectoparasite exams, and histopathology of all major organs.

### Example 15: Generation of Glutaryl-CoA Dehydrogenase-Deficient Mice

A line of Gcdh^{-/-} mice [Gcdh^{tm1Dmk (-/-)}] was generated via homologous insertion of a gene targeting vector which resulted in a deletion of the first 7 exons of the Gcdh gene, and the insertion of a β-galactosidase reporter gene (nlacF) controlled by Gcdh chromosomal regulatory elements. Homologous insertion of the targeting vector was identified by PCR analysis of both the 5′ and 3′ ends of the locus. Enzymatic assay of glutaryl-CoA dehydrogenase activity from samples of liver confirmed a complete loss of activity in Gcdh^{-/-} animals (not shown). Genotype analysis of the progeny of heterozygote-by-heterozygote matings (Gcdh^{+/-}×Gcdh^{+/-}) showed the expected Mendelian segregation ratio, indicating that Gcdh^{-/-} animals have normal fetal and post-natal viability. There was no effect of genotype on birth weight, neonatal growth or final adult weight.

### Example 16: Generation of Carnitine Palmitoyltransferase 1a (Liver Isoform) Deficiency Model

### Construction of Targeting Vector and Gene Targeting in ES Cells

The Cpt-1a targeting vector was constructed from genomic DNA fragments derived from a mouse 129X1/SvJ genomic P1 clone, PV1. The P1 clone was identified by screening a mouse 129X1/SvJ strain genomic library by PCR Exons 11-18 were deleted by a replacement gene targeting strategy by gene transfer into ES cells. The targeted ES cells were used to generate mice with a null allele (Cpt-1a^{tm1Uab}). ES cells (TC-1) were originally derived from 129S6/SvEv mice. Screening for recombinant ES cell clones was done by G418 selection (350 μg/ml) for 7 days. Surviving colonies were picked and expanded for Southern blot analysis.

### Mice

Chimeric mice were produced by microinjection of gene targeted ES cells into C57BL/6NTac (B6) embryos. The chimeric founders were bred to 129S6/SvEvTac (129) or B6 for perpetuation of mice used in these studies. All three genotypes (wild-type, heterozygous mutants and homozygous mutants) on both B6;129 and 129 backgrounds were produced for these studies.

### Example 17: Generation of Carnitine Palmitoyltransferase 1b (Muscle Isoform) Deficiency Model

The mutant mouse line had been generated previously using a targeted mutagenesis strategy by replacing a segment of 1468 bp (exons 1-3) in mouse Cpt-1b with a 3 kb neo-tk cassette in the C57BL/6J×129X1/SvJ ES cells. Mice in the current study were the second generation from 3 male founders, which were offspring from a male chimera and C57BL/6J (B6J) females. Mice were fasted for ˜18 h and euthanized with CO₂ before collecting blood for biochemical markers. The mice were also fasted for ˜18 h prior to cold tolerance testing. Alternatively, the mice used to measure mRNA expression and for collecting tissue for activity assays were not fasted before being euthanized with CO₂ inhalation. Also, two different mating pair arrangements were setup to obtain fetal tissue for genotyping and to isolate the corresponding placenta for RNA preparation. One strategy included male CPT-1b+/+ mice mated with female CPT-1b+/− mice; the other included male CPT-1b+/− mice mated with female CPT-1b+/+ mice. At embryonic day 12-14, pregnant females were sacrificed.

### Example 18: Generation of Medium-Chain Acyl-CoA Delhydrogenase Deficiency Model

MCADD insertion vector (MCAD IV2) was designed to undergo gap repair of the 1.3-kb deleted region upon homologous recombination in 129P2 (129P2/OlaHsd) ES cells E14-1. Correct targeting of the MCAD locus resulted in a duplication of exons 8, 9, and 10 and integration of flanking plasmid and Neo sequences. The insertion vector was designed to duplicate exon 8, 9, and 10 at the MCAD locus. Translation of the duplicated exon 8 region results in the formation of premature stop codons resulting in truncation of the MCAD monomer.

Specifically, the first premature stop codon arises after translation of only seven amino acids from the duplicated exon 8. The resulting MCAD monomer is missing the C-terminal domain α-helixes that are responsible for making intersubunit contacts to generate the functional MCAD homotetramer.

ES cell clones were screened by PCR and confirmed by Southern blot analysis. Southern blot analysis used an exon 10 probe (probe A), not present in the targeting vector, hybridized to a 13.2-kb band in addition to the 3.1-kb endogenous band indicating targeted insertion of the vector at the Acadm locus. Correctly targeted ES cell clones were microinjected into B6 (C57BL/6NTac) blastocysts to generate chimeric mice. Chimeric mice were backcrossed to both 129P2 and B6 inbred mice to produce MCAD^{+/-} and eventually MCAD^{-/-} mice on a B6/129 mixed background. The studies described here were conducted exclusively on the B6/129 mixed background compared with littermate controls or B6/129 control groups maintained by intercrosses as were the mutants. Perpetuating this mutation as a congenic mutant line on the 129P2 background proved impractical. The 129P2 mice were poor breeders as wild-types, and when introduced, the Acadm mutation was nearly lost on this background because of the high rate of neonatal death. Because of the molecular structure of the targeted allele, it proved virtually impossible to distinguish all three potential genotypes. One could clearly detect the presence or absence of the targeted allele, however, whether a particular mouse was MCAD^{-/-} or MCAD^{-/-} could not be determined by Southern blot or PCR of genomic DNA. Ultimately MCAD^{-/-} mice were ascertained by immunoblot analysis of offspring with subsequent perpetuation of MCAD_{-/-} and MCAD^{+/+} mice as separate groups.

### Example 19. Effect of Compounds on Mitochondrial Respiration

The effect of the compounds of the present disclosure on mitochondrial respiration was measured with a XFe96 Extracellular Flux Analyzer (Seahorse Bioscience, Agilent Technologies) and Oxygen consumption Rate (OCR) and Extracellular acidification rate (ECAR) determined.

Cell culture and treatments. Primary adherent fibroblasts were cultured in minimum essential medium (MEM) (Gibco, 25030081) supplemented with 2 mM L-Glutamine (Gibco, 25030081), 15% fetal bovine serum (FBS) (Gibco, 26400044) and 1% penicillin/streptomycin (Gibco, 5140122) at 37° C. and 5% CO₂. Cells were collected for either passaging or experiment at ˜70-80% confluence. Cells were obtained by trypsinization and seeded at 10,000 cells/well in cell culture microplates (Seahorse Bioscience, 101085-004) and allowed to adhere for 48 hours in culture media.

By profiling different primary cells and/or optimizing their culture media or environmental components one can select cell lines and/or conditions that are appropriate for the biological or disease model. Cells which can be profiled include: Propionic Acidemia (PA) (GM00371, GM03590 Coriell Institute for Medical Research, Tsi 6337, Tsi 4626, Tsi 3618 Trans-Hit Bio), Methylmalonic Acidemia (MMA) (GM01673, Coriell Institute for Medical Research, Tsi 5224 Trans-Hit Bio), Branched chain ketoacid dehydrogenase kinase (BCKDK) (GM00612, GM00649 Coriell Institute for Medical Research), Subnormal activation of pyruvate dehydrogenase complex (PDH) (GM01503 Coriell Institute for Medical Research), Very long-chain acyl-CoA dehydrogenase (VLCAD) (GM17475), Leigh Syndrome (LS) (GM03672, GM13411 Coriell Institute for Medical Research), Pyruvate Carboxylase Deficiency (PC) (GM00444 Coriell Institute for Medical Research), Glutaric Acidemia-I (GA) (GM10653), Friedreich's Ataxia (FXN) (GM04078 Coriell Institute for Medical Research, Huntington's disease (HD) (GM21756 Coriell Institute for Medical Research), Ornithine Transcarbamylase Deficiency (OTC) (GM12756 Coriell Institute for Medical Research), Kearns-Sayre Syndrome (KSS) (GM06225 Coriell Institute for Medical Research).

In the present example, 1 hour before the assay, the cells were washed with freshly prepared unbuffered serum free-Seahorse XF Assay medium (Seahorse Bioscience, North America, USA, 103575-100) with the appropriate supplements for the different primary cells (10 mM glucose, 2 mM L-glutamine, 1 mM pyruvate; 1 mM glucose, 2 mM L-glutamine, 1 mM pyruvate; 10 mM glucose; 5 mM glucose; 1 mM glucose).

Determining the optimized cell density and concentrations for stress test, such as FCCP, were achieved through methods well known to those of skill in the art. After baseline measurements of OCR, cells were challenged with compounds of the present disclosure at different concentrations (10 to 50 μM) or vehicle (DMSO, 0.1%) and a post-compound baseline was recorded. OCR was measured after sequentially adding to each well 1.5 μg/ml oligomycin (inhibitor of ATP synthase Sigma-Aldrich, 753531), then maximal OCR was determined with carbonyl cyanide 4-(trifluoromethoxy) phenylhydrazone (FCCP, Sigma-Aldrich, C2920), (uncoupler of oxidative phosphorylation) and 1.0 μM of rotenone (Sigma-Aldrich, R8875) plus antimycin A (Sigma-Aldrich, A8674) (inhibitors of mitochondrial complex I and III) for determination of rotenone-antimycin insensitive respiration together with Hoechst for nuclear staining in situ when normalized to cell counts. After the analysis and nuclear staining, the XFp microplate was transferred to the Cytation 5, and the nuclear images were captured, the individual nuclei were identified and counted by BioTek Gen5 software. Data were expressed as pmol of O₂ per minute and normalized by cell number using Agilent's Seahorse Cell Analysis software, and baselined to pre-compound addition. Extracellular acidification rate (ECAR) was optionally measured on the Seahorse XFe96 analyser simultaneously with the OCR measurements in the same wells.

Assay conditions were as stated below: each cycle consists of a 3 min mix, and a 3 min measure step.

| Condition | Cycles | | Time |
|---|---|---|---|
| Baseline | 3 | cycles | 24 min |
| Compound Injection | 3 | cycles | 24 min |
| Oligomycin Injection | 3 | cycles | 24 min |
| FCCP injection | 20/40 | cycles | 160 min |
| Antimycin | 3 | cycles | 18 min |

A/Rotenone/Hoechst

### Analysis.

OCR and ECAR values were expressed relative to vehicle. Several parameters can be evaluated from OCR and ECAR measurements as follows:
Mitochondrial Basal OCR (corresponds to baseline OCR minus rotenone/antimycin-insensitive OCR).
ATP-linked OCR (corresponds to basal OCR minus oligomycin-insensitive OCR).
Proton leak-linked OCR (corresponds to oligomycin-insensitive OCR minus rotenone/antimycin-insensitive OCR).
Maximal OCR (corresponds to FCCP-induced OCR minus rotenone/antimycin-insensitive OCR).
Spare respiratory capacity measured as the difference between Maximal and Basal OCR.
Non-mitochondrial OCR (corresponds to rotenone/antimycin-insensitive OCR).
Maximal OCR Area Under the Curve (AUC) (corresponds to AUC from the first measurement after FCCP injection to the last FCCP measurement minus non-mitochondrial respiration).
Spare Capacity AUC (corresponds to AUC of the first measurement after FCCP injection to the last FCCP).
AUC ECAR (between post-oligomycin injection and pre-FCCP injection).

### Example 10: Measurement of ATP

Primary adherent fibroblasts were cultured in minimum essential medium (MEM) (Gibco, 25030081) supplemented with 2 mM L-Glutamine (Gibco, 25030081), 15% fetal bovine serum (FBS) (Gibco, 26400044) and 1% penicillin/streptomycin (Gibco, 5140122) at 37° C. and 5% CO₂. Cells were collected for either passaging or experiment at ˜70-80% confluence. Cells were obtained by trypsinization and seeded in cell culture microplates either white plate (Thermo Fisher Scientific, 152028) for luminescent measurement or black plate (Thermo Fisher Scientific, 165305) for fluorescent based read out for different assays listed below. Cells were obtained by trypsinization and 5000K cells were seeded and allowed to adhere for 16-18 hours to have confluency around 70-80% in the cell well with culture media. After 24 hours prior to measurements (37° C., 5% CO₂) media was changed to Dulbecco's Modified Eagle Medium (DMEM, Agilent Seahorse cat #103575-100) with the appropriate supplements (1 mM glucose, 2 mM L-glutamine, 1 mM pyruvate, 10% FBS).

Assay was performed according to manufacturer instructions (ATPlite Assay Perkin Elmer, 6016941). 50 μl of cell lysis buffer was added to each well with cells in 100 μl of media and incubated for 5 mins at RT in an orbital shaker at 700 rpm to lyse the cells and stabilize the ATP. After, 50 μL luciferase-based reagent was added to the wells and incubated for 5 mins at RT in an orbital shaker at 700 rpm. The amount of signal was directly proportional to the ATP content. When normalised to cell count, a total dead cell count was performed as described in Example 22 and only alive cells were used for analysis.

### Example 21: Measurement of Total GSH and GSSG Dimer

Primary adherent fibroblasts were cultured in minimum essential medium (MEM) (Gibco, 25030081) supplemented with 2 mM L-Glutamine (Gibco, 25030081), 15% fetal bovine serum (FBS) (Gibco, 26400044) and 1% penicillin/streptomycin (Gibco, 5140122) at 37° C. and 5% CO₂. Cells were collected for either passaging or experiment at ˜70-80% confluence. Cells were obtained by trypsinization and seeded in a white cell culture microplates (Thermo Fisher Scientific, 152028) for luminescent measurement. Cells were obtained by trypsinization and 7000K cells were seeded and allowed to adhere for 16-18 hours to have confluency around 70-80% in the cell well with culture media. After 24 hours prior to measurements (37° C, 5% CO₂) media was changed to Dulbecco's Modified Eagle Medium (DMEM, Agilent Seahorse cat #103575-100) with the appropriate supplements (1 mM glucose, 2 mM L-glutamine, 1 mM pyruvate, 10% FBS).

Assay was performed according to manufacturer instructions (Promega, V6612). At the end of appropriate time point, the assay requires one plate for total measurement of GSH and one plate for GSSG. For every assay a 11-point standard curve is prepared ranging from 8 μM to 0.013 μM. Media is removed completely. For total GSH measurements, 50 μl/well of total glutathione lysis reagent were added for GSH identification and 50 μl/well of oxidized glutathione lysis reagent were added to all wells for GSSG identification (5 minute, shaking condition). Next, 50 μl/well of Luciferin Generation Reagent were added to all wells and allowed to incubate at RT for 30 minutes under shaking conditions. Finally, 100 μl/well of Luciferin Detection reagent were added and incubated for 15 minutes before the chemiluminescence was detected. Free GSH/GSSG ratio was calculated as (Total GSH-GSSH)/(GSSG/2). When normalised to cell count, a total dead cell count was performed as described in Example 22 and only alive cells were used for analysis.

### Example 22: Measurement of Total and Dead Cell Count

For various plate based assays, a total dead cell count was performed and only alive cells were used for analysis. Assay was performed according to manufacturer instructions (EarlyTox Cell Integrity Kit, Molecular Devices, R8214). Media was gently removed and 100 μl per well of total live red dye and dead green dye (1:2000) were added and further incubated for 15-30 minutes at 37° C. and 5% CO₂.

The reactive Live Red Dye is cell permeant and stained both live and dead cells resulting in the total cell count measurements (Excitation: 622 nm/Emission: 645 nm). In contrast, the reactive Dead Green Dye is cell impermeant and stained only cells with damaged outer membranes i.e. dead cells (Excitation: 503 nm/Emission: 526 nm/Em: 713 nm). Alive cells were calculated as (Total cells minus dead cells).

### Example 23: M1 Differentiation Assay

Monocytes were isolated by positive isolation with CD14+ microbeads (Miltenyi, 130-050-201). Monocytes were 99% viable and were 96% purity as analyzed by FACS and CD14+ (BD, 563561). 100,000 monocytes along with compounds at the dose of 10 and 50 μM were allowed to differentiate to macrophages with 10 ng/ml GMCSF (R&D, 15-GM-050/CF) in RPMI complete media (Invitrogen, 22400089) with 15% FBS (Hyclone SV30087.03) and 1% Penicillin-Streptomycin (Gibco, 15140-122). On day 2 and 4 half the media was refreshed with fresh GM-CSF and compounds at 10 and 50 μM dose. On day 6 the macrophages were matured with GMCSF, IFNγ (R&D 285-IF-100/CF) and LPS (Sigma, L6143) in the presence of 10 μM and 50 μM of compounds. After 24 h, supernatant was collected for the measurement of TNFα (DKW, 1117202) IL6 (DKW, 1110602), IL10 (DKW, 1110003) by ELISA. Macrophages were detached gently on ice with EDTA (Invitrogen, 15575-038) and analyzed by FACS (BD LSR Fortessa, 853492). Cells were stained for live/dead dye, surface and intracellular markers with fixation/permeabilization solution (BD, 554714) and ALIVE/DEAD™ Fixable Near-IR Dead Cell Stain Kit (BD, L34976), mouse anti human CD86 APC (BD, 555660), mouse anti-human CD163 PE (BD, 556018), mouse anti-human CD68 FITC (BD, 562117) or isotype controls anti-mouse IgG1 κ PE (BD, 559320) and anti-mouse IgG1 κ APC (BD, Anti mouse IgG1 κ APC (BD, 55571). Mature M1 macrophages were defined as CD86+CD68+CD163− and increase in TNFα, IL6, and decrease in IL-10.

### Example 24: Effect of Compounds on Mitocondrialfusion and Networking

Cells were seeded in 96-well plates (density 3000 cells/well) in culture minimum MEM (GIBCO, 10370-021) supplemented with 2 mM L-Glutamine (Thermo Fisher Scientific), 15% FBS (Thermo Fisher Scientific 26400044) and 1% penicillin/streptomycin. Cells were incubated for 48 hours (37° C, 5% CO2), then treated with 10 μM compounds from the present disclosure, 1% DMSO (vehicle) or 5 μM FCCP (control) for 2 hours in fasted conditional medium: Agilent XF DMEM (Agilent 103575-100), pH 7.4 supplemented with 10% FBS (Thermo Fisher Scientific 26400044), 0.03% penicillin/streptomycin 1 mM glucose, 2 mM L-glutamine and 1 mM pyruvate.

After treatment, media was removed, and cells were stained with 100 μl of 1×mixture dye solution of 1 μg/mL JC-1 (Invitrogen Cat #T3168) and 50 ng/ml MitoTracker deep red (Invitrogen Cat #M22426) at 37° C. for 30 minutes. Next, staining was removed, the cells were washed once with 150 μl PBS, and 150 μl of fasted conditional medium (described above) is added to each well. Analysis was carried out on live cells using a Thermo Scientific CellInsight CX7 LZR High-Content Screening Platform. Mitochondrial elongation and networking was described as slight, mild or good.

### Example 25: Effects Oftest Compounds on Glucose Uptake

The effect of compounds from the present disclosure on glucose uptake was determined in HepG2 cells (ATCC, HB-8065) using the glucose uptake Glo Assay Kit (Promega, J1343 according to manufacturer instructions. HepG2 cells were cultured in complete DMEM-glucose media (Gibco) supplemented with 10% FBS (37° C. incubator with 5% CO₂) and seeded in 96-well plates at 30,000 cells/well. After removing the complete media, 100 μL/well of serum-free, high-glucose DMEM media were added to the wells and incubated overnight (37° C. incubator with 5% CO₂). Media was then replaced with 100 μL/well DPBS containing 0.6% BSA and starved for 1 hour. Next, DPBS was removed and 45 μl/well of insulin (100 nM) or compounds (10 μM-50 μM) were added to the wells and incubated for 10 minutes (37° C. incubator with 5% CO₂). Insulin and compounds were prepared in DPBS with 0.6% BSA with a final DMSO concentration of 0.1%. Next, 5 μL of 2DG (10 mM) in DPBS were added per well and allowed to incubate for 20 minutes followed by addition of 25 μL stop buffer. 37.5 μL of the mixture were then transferred to a new plate and 12.5 μL of Neutralization buffer added to the wells. After, 50 μL of 2DG6P detection Reagent were added and incubated for 0.5-1 hour at room temperature. Luminescence was measured with 0.3-1 second integration on a luminometer.

### Example 26: Effects of Test Compounds on Extracellular Pyruvate Concentration

Pyruvate is a central molecule in metabolism through which sugars enter the citric acid cycle. Pyruvate can be converted to carbohydrates during gluconeogenesis or to fatty acids via acetyl CoA. High levels of pyruvate are associated with liver disease and genetic disorders.

In the present example, the effect of compounds from the present disclosure on extracellular pyruvate concentration from primary Type 1 diabetes myoblasts was determined. By profiling different primary cells and/or optimizing their culture media or environmental components one can select cell lines and/or conditions that are appropriate for the biological or disease model.

Cell culture and treatments. Skeletal Muscle-Derived Cells (SkMDCs, Cook MyoSite, Cat #SK-1111, Lot #P01262-46M, sourced from rectus abdominus muscle of a 46-yr-old male with Type 1 Diabetes and a Body Mass Index of 24) were cultured in insulin free Myotonic basal medium (Cook MyoSite, ML-6666) supplemented with Myotonic growth supplement (Cook MyoSite, MS-3333), 200 μM insulin (Sigma, 19278) and 1% penicillin/streptomycin (Gibco, 5140122) at 37° C. and 5% CO₂. SkMDC cultures were differentiated to myoblasts, as indicated by the presence of elongated, multi-nucleated myotubes, by culturing for 2-4 days in DMEM low glucose (GIBCO) supplemented with 2% horse serum (GIBCO, 16050130), 1% penicillin/streptomycin (Gibco, 5140122) and 1 ng/ml insulin-like growth factor 1 (IGF-1, GIBCO, PHG0071). Cells were collected for either passaging or experiment at ˜25-50% confluence by trypsinization, and seeded at 5,000-7,500 cells/cm². Flasks and microplates were coated with Cell Application Inc Collagen 1 (Sigma, Cat #125-50) prior to use, according to manufacturer instructions.

Assay was performed according to manufacturer instructions (Pyruvate Colorimetric/Fluorometric Assay Kit, BioVision, Catalog #K609-100). For every assay a 6-point standard curve was prepared ranging from 0.2-1.0 nmol/well. 50 μl of media was collected from experimental treatment plates, and added to 96-well black clear-bottom microplates (Thermo Fisher Scientific, 165305). 50 μL of pyruvate oxidase-based reagent was added to each well, the plate was sealed with an aluminum sealer and incubated for 30 minutes at RT in an orbital shaker at 200 rpm in the dark. Plate fluorescence was detected (Excitation: 530 nm/Emission: 590 nm), with the amount of signal directly proportional to pyruvate content.

### Example 27

Using the procedure described in Example 19, with a primary adherent fibroblast cell line derived from an Methylmalonic Acidemia (MMA) patient (GM01673, Coriell Institute for Medical Research), freshly prepared unbuffered serum free-Seahorse XF Assay medium was supplemented with 1 mM glucose, and stimulated to maximal OCR with 8 μM of FCCP for 20 cycles.

The following Compounds of the present invention increased Spare Capacity AUC by at least 5%, upon treatment with the indicated concentration: 10 μM 206, 50 μM 206, 50 μM 62, 10 μM 78.

### Example 28

Using the procedure described in Example 19, with a primary adherent fibroblast cell line derived from an Methylmalonic Acidemia (MMA) patient (GM01673, Coriell Institute for Medical Research), freshly prepared unbuffered serum free-Seahorse XF Assay medium was supplemented with 10 mM glucose, 2 mM glutamine, 1 mM pyruvate, and stimulated to maximal OCR with 1 μM of FCCP for 20 cycles.

The following Compounds of the present invention increased Spare Capacity AUC by at least the percentage indicated, when tested at 10 μM concentration: 61>20%; 206>10%.

### Example 29

Using the procedure described in Example 19, with a primary adherent fibroblast cell line derived from an Methylmalonic Acidemia (MMA) patient (GM01673, Coriell Institute for Medical Research), freshly prepared unbuffered serum free-Seahorse XF Assay medium was supplemented with 1 mM glucose, and stimulated to maximal OCR with 1 μM of FCCP for 20 cycles.

The following Compounds of the present invention increased Spare Capacity AUC by at least 50%, when tested at 10 μM concentration: 206.

### Example 30

Using the procedure described in Example 20, Methylmalonic Acidemia (MMA) Primary fibroblasts (Tsi 5224 Trans-Hit Bio, 21498 Telethon Network of Genetic Biobanks) were assayed, upon treatment with 10 μM of Compounds for 2 hours.

The following Compounds of the present invention increased ATP levels by at least 5%: 206.

### Example 31

Using the procedure described in Example 21, Methylmalonic Acidemia (MMA) Primary fibroblasts (GM01673, Coriell Institute for Medical Research) were assayed, upon treatment with 10 μM of Compounds for 2 hours.

The following Compounds of the present invention increased the GSH/GSSG ratio by at least 20%: 206.

### Example 32

Using the procedure described in Example 23, the following Compounds of the present invention decreased levels of IL-6 protein by at least 40%, when tested at 10 μM concentration: 206.

### Example 33

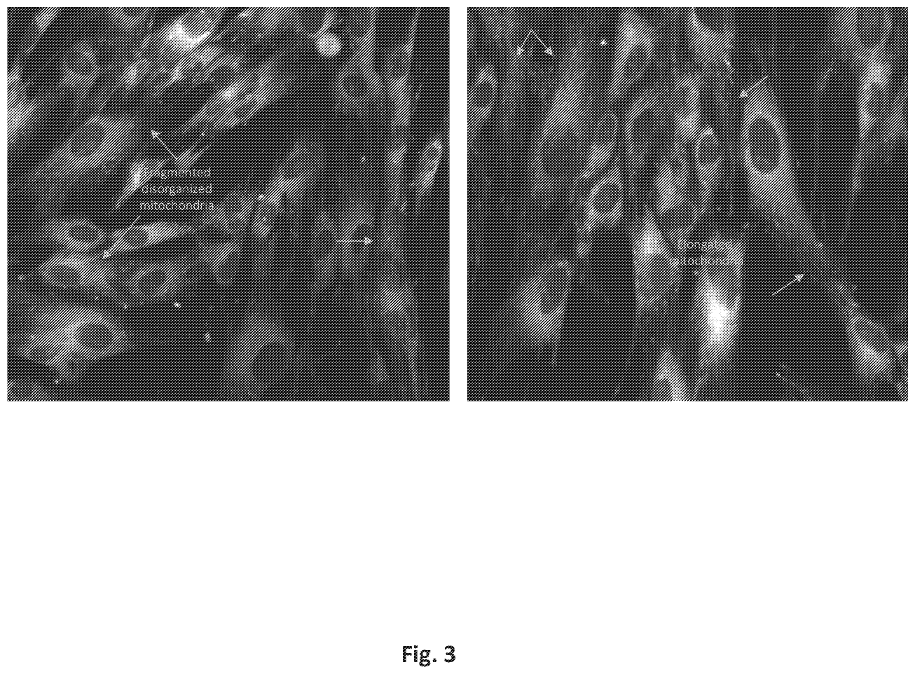

Using the procedure described in Example 24, with a primary adherent fibroblast cell line derived from an Methylmalonic Acidemia (MMA) patient (GM01673, Coriell Institute for Medical Research), the following Compounds of the present invention slightly increased mitochondrial elongation, when tested at 10 μM concentration: 61 (FIG. 3).

### Example 34

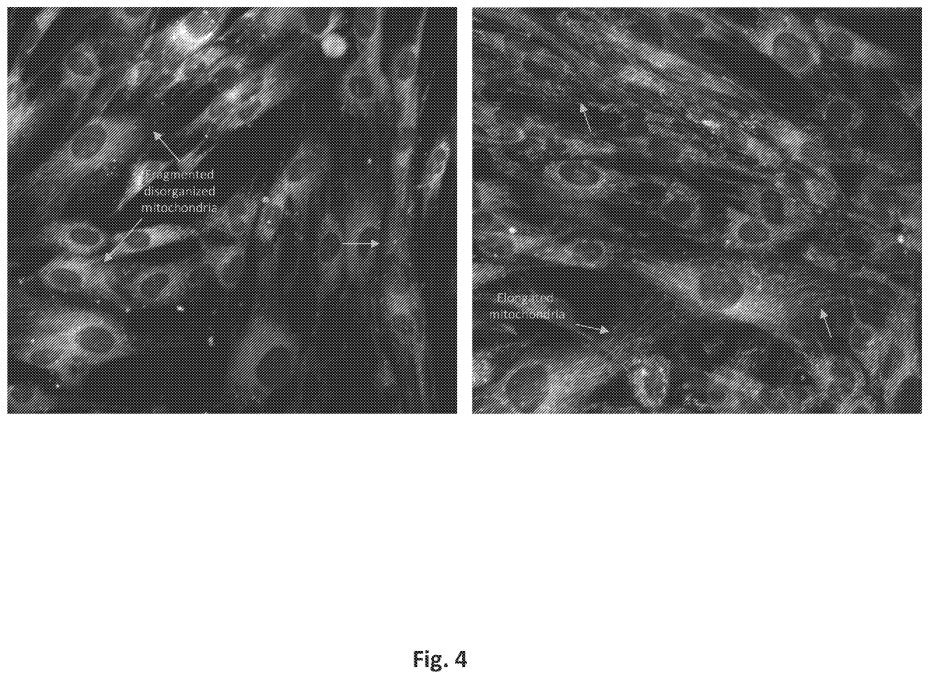

Using the procedure described in Example 24, with a primary adherent fibroblast cell line derived from an Methylmalonic Acidemia (MMA) patient (GM01673, Coriell Institute for Medical Research), the following Compounds of the present invention had a good increase in mitochondrial elongation, when tested at 10 μM concentration: 206 (FIG. 4).

### Example 35

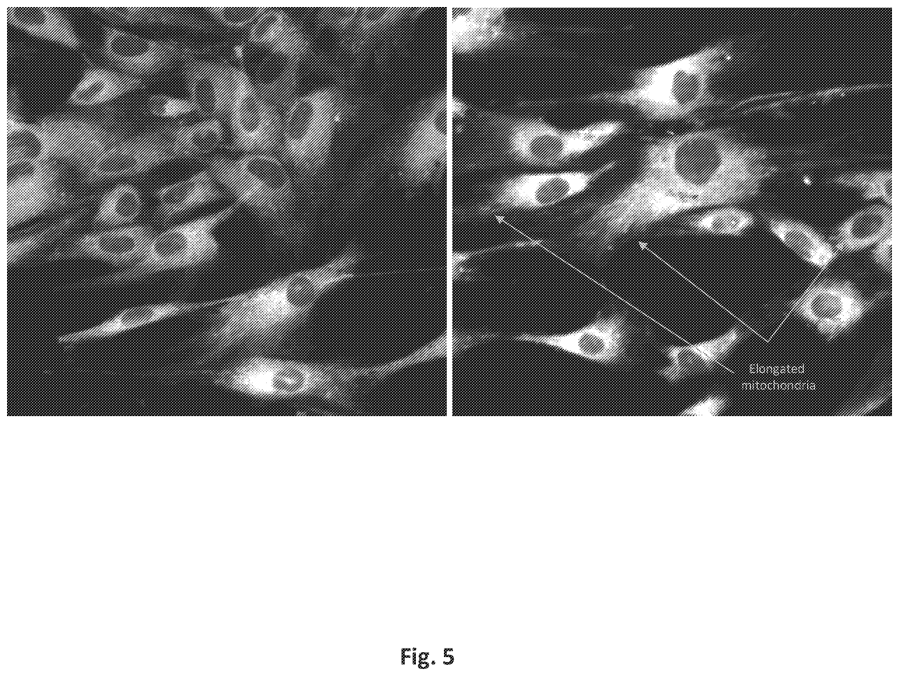

Using the procedure described in Example 24, with a primary adherent fibroblast cell line derived from a Propionic Acidemia (PA) patient (Tsi 3618 Trans-Hit Bio), the following Compounds of the present invention had a mild increase in mitochondrial elongation, when tested at 10 μM concentration: 206 (FIG. 5).

### Example 36

Using the procedure described in Example 25, the Compounds of the present invention listed below stimulated Glucose Uptake of HepG2 cells by at least 15% of insulin control, when tested at 10 μM concentration: 61.

### Example 37

Using the procedure described in Example 26, the Compounds of the present invention listed below decreased extracellular pyruvate concentration by at least 15% relative to vehicle, when tested at 10 μM concentration: 61, 206.

### Example 38. Synthesis of Compound 339

### Step 1: Synthesis of (2R,2′R)—N,N′-(((disulfanediylbis(ethane-2,1-diyl))bis(azanediyl))bis(3-oxopropane-3,1-diyl))bis(4-((tert-butyldimethylsilyl)oxy)-2-hydroxy-3,3-dimethylbutanamide)

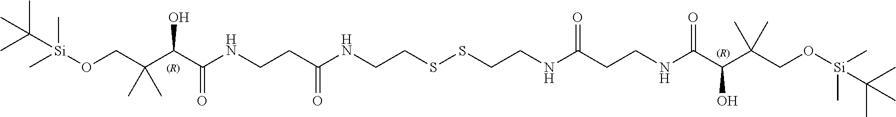

To a stirred solution of D-pantetheine (15 g, 1 equiv., 27.04 mmol) in anhydrous DMF (50 mL) at 0° C. was added imidazole (14.8 g, 8 equiv., 216.3 mmol) followed by lot wise addition of TBSCl (32.6 g, 8 equiv., 216.3 mmol). The reaction mixture was stirred at same temperature for 16 h. The solvent was evaporated and residue diluted with DCM, organic layer washed with water, brine dried over sodium sulphate and concentrated under vacuum, the residue was purified by combi-flash on silica gel eluting with 0-5% MeOH in DCM to afford the title compound (2R,2′R)—N,N′-(((disulfanediylbis(ethane-2,1-diyl))bis(azanediyl))bis(3-oxopropane-3,1-diyl))bis(4-((tert-butyldimethylsilyl)oxy)-2-hydroxy-3,3-dimethylbutanamide) (5.0 g, 6.384 mmol, 23.5%) as a sticky solid. LCMS (M+1). 783.6.

### Step 2: Synthesis of ((6R,25R)-6,25-bis(1-((tert-butyldimethylsilyl)oxy)-2-methylpropan-2-yl)-4,7,11,20,24,27-hexaoxo-5,26-dioxa-15,16-dithia-8,12,19,23-tetraazatriacontanedioic acid

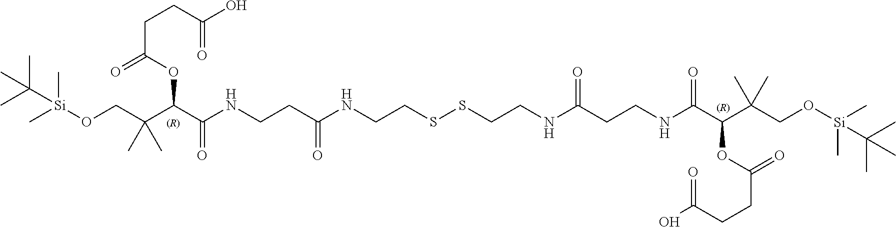

To a stirred solution of the product from Example 38 Step 1 ((2R,2′R)—N,N′-(((disulfanediylbis(ethane-2,1-diyl))bis(azanediyl))bis(3-oxopropane-3,1-diyl))bis(4-((tert-butyldimethylsilyl)oxy)-2-hydroxy-3,3-dimethylbutanamide, 2 g, 1 equiv., 2.553 mmol) in anhydrous THF (20 mL) at room temperature was added TEA (2.4 mL, 6.5 equiv., 16.59 mmol) followed by succinic anhydride (1.53 g, 6.0 equiv., 15.32 mmol). The reaction mixture was stirred at 70° C. for 4 h. The volatiles were removed under vacuum. The residue was dissolved in ethyl acetate, washed with water, followed by brine, dried over anhydrous sodium sulphate and concentrated. Residue was purified by combi-flash on silica gel eluting with 0-5% MeOH in DCM to afford the title compound ((6R,25R)-6,25-bis(1-((tert-butyldimethylsilyl)oxy)-2-methylpropan-2-yl)-4,7,11,20,24,27-hexaoxo-5,26-dioxa-15,16-dithia-8,12,19,23-tetraazatriacontanedioic acid (2.2 g, 2.237 mmol, 88%) as a sticky solid. LCMS (M+1): 983.8.

### Step 3: Synthesis of (R)-4-((15-mercapto-2,2,3,3,6,6-hexamethyl-8,12-dioxo-4-oxa-9,13-diaza-3-silapentadecan-7-yl)oxy)-4-oxobutanoic acid

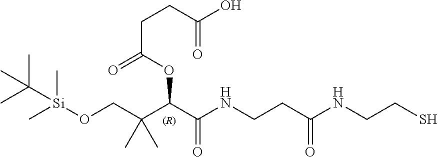

The product from Example 38 Step 2 ((6R,25R)-6,25-bis(1-((tert-butyldimethylsilyl)oxy)-2-methylpropan-2-yl)-4,7,11,20,24,27-hexaoxo-5,26-dioxa-15,16-dithia-8,12,19,23-tetraazatriacontanedioic acid, 2 g, 1 equiv., 2.032 mmol) was dissolved in a mixture of ACN:Water ((9:1) 25 mL) at 0° C. Tributyl phosphine (1.6 mL, 2.0 equiv., 4.065 mmol) was added, and the reaction mixture was stirred at rt for 1 h. The volatiles were removed under vacuum. To the residue was added ethyl acetate, washed with water, followed by brine, dried over anhydrous sodium sulphate and concentrated. Residue was purified by combi-flash on silica gel eluting with 0-5% MeOH in DCM to afford the title compound (R)-4-((15-mercapto-2,2,3,3,6,6-hexamethyl-8,12-dioxo-4-oxa-9,13-diaza-3-silapentadecan-7-yl)oxy)-4-oxobutanoic acid (1.1 g, 2.23 mmol, 55%) as a white solid. LCMS (M+1)=493.3.

### Step 4: Synthesis of Compound 339—Synthesis of (R)-2-(1-((tert-butyldimethylsilyl)oxy)-2-methylpropan-2-yl)-1-oxa-11-thia-4,8-diazacyclopentadecane-3,7,12,15-tetraone

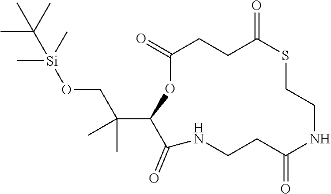 [SMILES: CC(C)(CO[Si](C)(C)C(C)(C)C)[C@H]1OC(=O)CCC(=O)SCCNC(=O)CCNC1=O]

To a stirred solution of the product from Example 38 Step 3 ((R)-4-((15-mercapto-2,2,3,3,6,6-hexamethyl-8,12-dioxo-4-oxa-9,13-diaza-3-silapentadecan-7-yl)oxy)-4-oxobutanoic acid, 900 mg, 1 equiv., 1.826 mmol) in DCM (10 mL) at 0° C. was added DIPEA (472 mg, 2.0 equiv., 3.653 mmol) and T3P (1.7 mL, 1.5 equiv., 2.740 mmol) The reaction mixture was stirred at rt for 12 h. The reaction mixture was diluted with DCM, washed with water, followed by brine, dried over anhydrous sodium sulphate and concentrated. Residue was purified by combi-flash on silica gel eluting with 0-4% MeOH in DCM to afford the title Compound 339, (R)-2-(1-((tert-butyldimethylsilyl)oxy)-2-methylpropan-2-yl)-1-oxa-11-thia-4,8-diazacyclopentadecane-3,7,12,15-tetraone (500 mg, 1.053 mmol, 57.6%) as a white solid. LCMS (M+1): 475.2.

### Example 39: Synthesis of Compound 61

### Step 1: Synthesis of (R)-2-(1-hydroxy-2-methylpropan-2-yl)-1-oxa-1l-thia-4,8-diazacyclopentadecane-3,7,12,15-tetraone

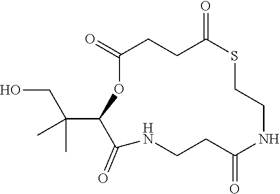 [SMILES: CC(C)(CO)[C@H]1OC(=O)CCC(=O)SCCNC(=O)CCNC1=O]

To a stirred solution of the product from Example 38 Step 4 ((R)-2-(1-((tert-butyldimethylsilyl)oxy)-2-methylpropan-2-yl)-1-oxa-11-thia-4,8-diazacyclopentadecane-3,7,12,15-tetraone, 350 mg, 1 equiv., 0.737 mmol) in MeOH (10 mL) at 0° C. was added acetyl chloride (57.8 mg, 1.0 equiv., 0.737 mmol). The reaction mixture was stirred at 0° C. for 30 min. To the reaction mixture was added NaHCO₃ and stirred for 10 min, the reaction mass was filtered and filtrate concentrated. Residue was purified by preparative HPLC (Mobile Phase: A=water, B=ACN; Column: X BRIDGE (150 mm×21.2 mm), 5.0 μm; Flow: 20 ml/min) to afford the title Compound 61 (R)-2-(1-((tert-butyldimethylsilyl)oxy)-2-methylpropan-2-yl)-1-oxa-11-thia-4,8-diazacyclopentadecane-3,7,12,15-tetraone (75 mg, 0.208 mmol, 28.3%) as a white solid. LCMS (M+1)=361.15. DSC 95.72° C. (melting point), 138.03° C. (on-set, exotherm). ¹H NMR (400 MHz, DMSO) δ 7.98 (s, 1H), 7.00 (s, 1H), 4.81 (s, 1H), 4.67 (t, J=5.6 Hz, 1H), 3.49-3.30 (m, 3H, part overlapped in water peak of DMSO), 3.28-3.10 (m, 3H), 3.09-2.83 (m, 4H), 2.80-2.58 (m, 2H), 2.18-2.00 (m, 2H), 0.88 (s, 3H), 0.85 (s, 3H).

### Example 40: Synthesis of Compound 62—Synthesis of (R)-2-methyl-2-(3,7,12,15-tetraoxo-1-ara-11-thia-4,8-diazacydopentadecan-2-yl)propyl acetate

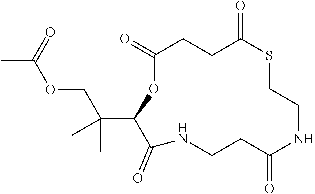 [SMILES: CC(=O)OCC(C)(C)[C@H]1OC(=O)CCC(=O)SCCNC(=O)CCNC1=O]

To a solution of (R)-2-(1-hydroxy-2-methylpropan-2-yl)-1-oxa-11-thia-4,8-diazacyclopentadecane-3,7,12,15-tetraone from Example 39 (600 mg, 1.66 mmol) and TEA (504.4 mg, 4.98 mmol) in DCM (40 mL) was added acetyl chloride (260.62 mg, 3.32 mmol), then the mixture was stirred at rt for 8 hours. The mixture was washed with H₂O (10 mL), then extracted by DCM (10 mL×30), dried over Na₂SO₄, filtered and concentrated to give the crude product which was purified by preparative HPLC to afford the title Compound 62 ((R)-2-methyl-2-(3,7,12,15-tetraoxo-1-oxa-11-thia-4,8-diazacyclopentadecan-2-yl)propyl acetate, 132.9 mg, 19.83%) as colorless oil. ¹H NMR (400 MHz, DMSO) δ 7.88 (t, J=5.3 Hz, 1H), 7.60 (t, J=5.4 Hz, 1H), 4.64 (s, 1H), 3.84 (q, J=10.9 Hz, 2H), 3.43-3.36 (m, 1H), 3.32-3.27 (m, 1H), 3.26-3.10 (m, 2H), 3.01-2.90 (m, 3H), 2.86-2.77 (m, 1H), 2.64 (t, J=6.0 Hz, 2H), 2.25 (dd, J=7.5, 4.5 Hz, 2H), 2.07 (s, 3H), 0.97 (s, 3H), 0.91 (s, 3H). LC/MS Rt=1.073 min; MS m/z: 402.8 [M+H]+.

### Example 41: Synthesis of Compound 76

### Step 1: Synthesis of (R)-2-methyl-2-(3,7,12,15-tetraoxo-1-oxa-11-thia-4,8-diazacyclopentadecan-2-yl)propyl (tert-butoxycarbonyl)glycinate

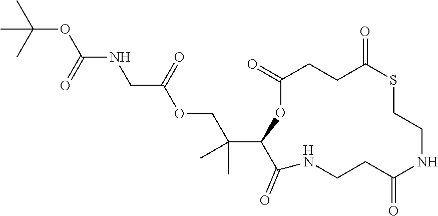 [SMILES: CC(C)(C)OC(=O)NCC(=O)OCC(C)(C)[C@H]1OC(=O)CCC(=O)SCCNC(=O)CCNC1=O]

To a solution of (R)-2-(1-hydroxy-2-methylpropan-2-yl)-1-oxa-11-thia-4,8-diazacyclopentadecane-3,7,12,15-tetraone from Example 39 (1.5 g, 4.16 mmol), (tert-butoxycarbonyl)glycine (1.46 g, 8.32 mmol) in THF (50 mL) was added CDI (1.35 g, 8.32 mmol), then the mixture was stirred at 50° C. for 16 hours. The mixture was washed with H₂O (10 mL), then extracted by EA (10 mL×30), dried over Na₂SO₄, filtered and concentrated to give the crude product which was purified by preparative HPLC to afford the title compound (550 mg, 25.54%) as a colorless oil. LC/MS Rt=1.091 min; MS m/z: 517.7 [M+H]+.

### Step 2: Synthesis of Compound 76—Synthesis of (R)-2-methyl-2-(3,7,12,15-tetraoxo-1-oxa-11-thia-4,8-diazacyclopentadecan-2-yl)propyl glycinate

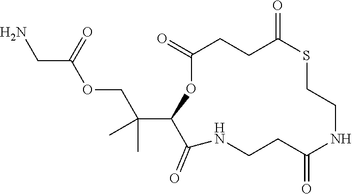 [SMILES: CC(C)(COC(=O)CN)[C@H]1OC(=O)CCC(=O)SCCNC(=O)CCNC1=O]

To a solution of (R)-2-methyl-2-(3,7,12,15-tetraoxo-1-oxa-11-thia-4,8-diazacyclopentadecan-2-yl)propyl (tert-butoxycarbonyl)glycinate from Example 41 Step 1 (550 mg, 1.06 mmol) in DCM (10 mL) was added TFA (10 ml), then the mixture was stirred at 0° C. for 4 hours. The mixture was concentrated to give the crude product which was purified by preparative HPLC to afford the title Compound 76 (128 mg, 28.89%) as a colorless oil. ¹H NMR (400 MHz, MeOD) δ 4.98 (s, 1H), 4.10 (d, J=11.5 Hz, 1H), 4.00 (d, J=1.8 Hz, 2H), 3.90 (d, J=11.4 Hz, 1H), 3.61-3.42 (m, 2H), 3.37-3.32 (m, 1H), 3.30-3.23 (m, 2H), 3.13-3.02 (m, 3H), 2.68-2.52 (m, 2H), 2.45-2.36 (m, 1H), 2.32-2.23 (m, 1H), 1.15 (s, 3H), 1.10 (s, 3H). LC/MS Rt=0.890 min; MS m/z: 417.8 [M+H]+.

### Example 42: Synthesis of Compound 78

### Step 1: Synthesis of 2-methyl-2-((R)-3,7,12,15-tetraoxo-1-oxa-11-thia-4,8-diazacyclopentadecan-2-yl)propyl (tert-butoxycarbonyl)-L-alaninate

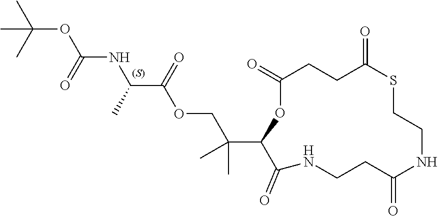

To a solution of (R)-2-(1-hydroxy-2-methylpropan-2-yl)-1-oxa-11-thia-4,8-diazacyclopentadecane-3,7,12,15-tetraone from Example 39 (1.5 g, 4.16 mmol), (tert-butoxycarbonyl)-L-alanine (1.57 g, 8.32 mmol) in THF (50 mL) was added CDI (1.35 g, 8.32 mmol), then the mixture was stirred at 50° C. for 16 hours. The mixture was washed with H₂O (10 mL), then extracted by EA (10 mL×30), dried over Na₂SO₄, filtered and concentrated to give the crude product which was purified by preparative HPLC to afford the title compound (800 mg, 36.20%) as a colorless oil. LC/MS Rt=1.117 min; MS m/z: 531.7 [M+H]+.

### Step 2: Synthesis of Compound 78—Synthesis of 2-methyl-2-((R)-3,7,12,15-tetraoxo-1-oxa-11-thia-4,8-diazacyclopentadecan-2-yl)propyl L-alaninate

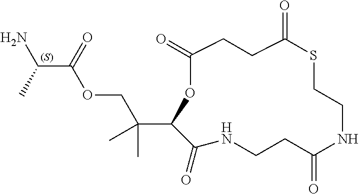

To a solution of 2-methyl-2-((R)-3,7,12,15-tetraoxo-1-oxa-11-thia-4,8-diazacyclopentadecan-2-yl)propyl(tert-butoxycarbonyl)-L-alaninate from Example 42 Step 1 (800 mg, 1.50 mmol) in DCM (10 mL) was added TFA (10 mL), then the mixture was stirred at 0° C. for 4 hours. The mixture was concentrated to give the crude product which was purified by preparative HPLC to afford the title Compound 78 (105 mg, 16.22%) as a colorless oil. ¹H NMR (400 MHz, MeOD) δ 4.95 (s, 1H), 4.26 (q, J=7.2 Hz, 1H), 4.16 (d, J=11.5 Hz, 1H), 3.86 (d, J=11.5 Hz, 1H), 3.60-3.43 (m, 2H), 3.35 (d, J=3.7 Hz, 1H), 3.29 (s, 1H), 3.25 (d, J=8.4 Hz, 1H), 3.12-3.01 (m, 3H), 2.65-2.51 (m, 2H), 2.44-2.35 (m, 1H), 2.32-2.21 (m, 1H), 1.59 (d, J=7.3 Hz, 3H), 1.15 (s, 3H), 1.11 (s, 3H). LC/MS Rt=0.898 min; MS m/z: 431.8 [M+H]+.

### Example 43: Synthesis of Compound 340

### Step 1: Synthesis of (2R,2′R)—N,N′-(((disulfanediylbis(ethane-2,1-diyl))bis(azanediyl))bis(3-oxopropane-3,1-diyl))bis(2-((tert-butyldimethylsilyl)oxy)-4-hydroxy-3,3-dimethylbutanamide)

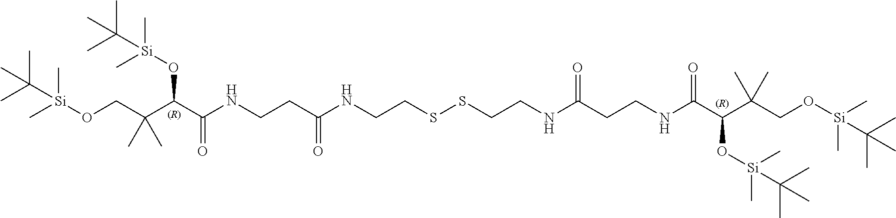

To a stirred solution of D-pantetheine (10 g, 1 equiv., 18.04 mmol) in anhydrous DMF (35 mL) at 0° C. was added imidazole (19.85 g, 16 equiv., 288.68 mmol) followed by lot wise addition of TBSCl (43.51 g, 16 equiv., 288.68 mmol). The reaction mixture was stirred at same temperature for 16 h. The solvent was evaporated and residue diluted with DCM, organic layer washed with water, and brine, dried over sodium sulphate and concentrated under vacuum, the residue was purified by combi-flash on silica gel eluting with 0-5% MeOH in DCM to afford the title compound (2R,2′R)—N,N′-(((disulfanediylbis(ethane-2,1-diyl))bis(azanediyl))bis(3-oxopropane-3,1-diyl))bis(2-((tert-butyldimethylsilyl)oxy)-4-hydroxy-3,3-dimethylbutanamide (12.2 g, 12.05 mmol, 66.83%) as a sticky solid.

### Step 2: Synthesis of (2R,2′R)—N,N′-(((disulfanediylbis(ethane-2,1-diyl))bis(azanediyl))bis(3-oxopropane-3,1-diyl))bis(2-((tert-butyldimethylsilyl)oxy)-4-hydroxy-3,3-dimethylbutanamide)

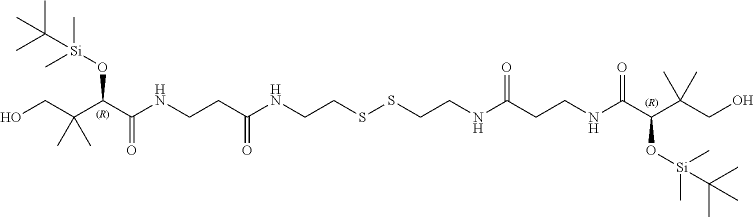

To a stirred solution of the product from Example 43 Step 1 (N,N′-(((disulfanediylbis(ethane-2,1-diyl))bis(azanediyl))bis(3-oxopropane-3,1-diyl))bis(2,4-bis((tert-butyldimethylsilyl)oxy)-3,3-dimethylbutanamide, 13 g, 1 equiv., 12.84 mmol) in anhydrous MeOH (50 mL) at 0° C. was added PPTS (6.7 g, 2.1 equiv., 26.98 mmol). The reaction mixture was stirred at rt for 1 h. The solvent was evaporated and residue diluted with DCM, organic layer washed with water, brine dried over sodium sulphate and concentrated under vacuum, the residue was purified by combi-flash on silica gel eluting with 0-5% MeOH in DCM to afford the title compound (2R,2′R)—N,N′-(((disulfanediylbis(ethane-2,1-diyl))bis(azanediyl))bis(3-oxopropane-3,1-diyl))bis(2-((tert-butyldimethylsilyl)oxy)-4-hydroxy-3,3-dimethylbutanamide) (9.0 g, 16.59 mmol, 90%) as a sticky solid. LCMS (M+1): 783.5

### Step 3: Synthesis of (8R,27R)-8,27-bis((tert-butyldimethylsilyl)oxy)-7,7,28,28-tetramethyl-4,9,13,22,26,31-hexaoxo-5,30-dioxa-17,18-dithia-10,14,21,25-tetraazatetratriacontanedioic acid

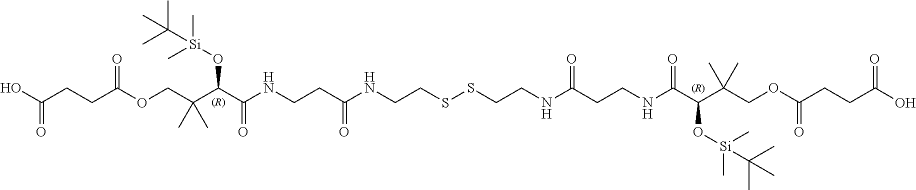

To a stirred solution of the product from Example 43 Step 2 (((2R,2′R)—N,N′-(((disulfanediylbis(ethane-2,1-diyl))bis(azanediyl))bis(3-oxopropane-3,1-diyl))bis(2-((tert-butyldimethylsilyl)oxy)-4-hydroxy-3,3-dimethylbutanamide, 3.5 g, 1 equiv., 4.469 mmol) in anhydrous THF (30 mL) at room temperature was added TEA (2.4 mL, 6.5 equiv., 16.59 mmol) followed by succinic anhydride (1.53 g, 6.0 equiv., 15.32 mmol). The reaction mixture was stirred at 70° C. for 12 h. The volatiles were removed under vacuum. Residue was purified by combi-flash on silica gel eluting with 0-5% MeOH in DCM to afford the title compound (8R,27R)-8,27-bis((tert-butyldimethylsilyl)oxy)-7,7,28,28-tetramethyl-4,9,13,22,26,31-hexaoxo-5,30-dioxa-17,18-dithia-10,14,21,25-tetraazatetratriacontanedioic acid (3.7 g, 3.762 mmol, 86.04%) as a sticky solid. LCMS (M+1): 983.8

### Step 4: Synthesis of (R)-4-(3-((tert-butyldimethylsilyl)oxy)-4-((3-((2-mercaptoethyl)amino)-3-oxopropyl)amino)-2,2-dimethyl-4-oxobutoxy)-4-oxobutanoic acid

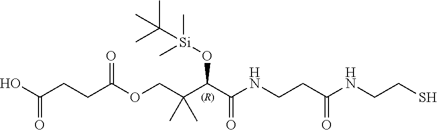

The product from Example 43 Step 3 ((8R,27R)-8,27-bis((tert-butyldimethylsilyl)oxy)-7,7,28,28-tetramethyl-4,9,13,22,26,31-hexaoxo-5,30-dioxa-17,18-dithia-10,14,21,25-tetraazatetratriacontanedioic acid, 3 g, 1 equiv., 3.050 mmol) was dissolved in a mixture of ACN:Water (9:1, 30 mL) at 0° C. was added tributyl phosphine (1.47 mL, 1.2 equiv., 3.661 mmol). The reaction mixture was stirred at rt for 1 h. The volatiles were removed under vacuum. To the residue was added ethyl acetate washed with water, followed by brine, dried over anhydrous sodium sulphate and concentrated. Residue was purified by combi-flash on silica gel eluting with 0-5% MeOH in DCM to afford the title compound (R)-4-(3-((tert-butyldimethylsilyl)oxy)-4-((3-((2-mercaptoethyl)amino)-3-oxopropyl)amino)-2,2-dimethyl-4-oxobutoxy)-4-oxobutanoic acid (2.4 g, 4.871 mmol, 80%) as a white solid. LCMS (M+1): 493.3.

### Step 5: Synthesis of Compound 340—Synthesis of (R)-15-((tert-butyldimethylsilyl)oxy)-16,16-dimethyl-1-oxa-6-thia-9,13-diazacycloheptadecane-2,5,10,14-tetraone

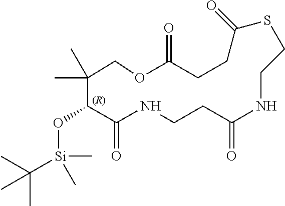

To a stirred solution of the product from Example 43 Step 4 ((8R,27R)-8,27-bis((tert-butyldimethylsilyl)oxy)-7,7,28,28-tetramethyl-4,9,13,22,26,31-hexaoxo-5,30-dioxa-17,18-dithia-10,14,21,25-tetraazatetratriacontanedioic acid, 1.5 g, 1 equiv., 3.044 mmol) in DCM (35 mL) at 0° C. was added DIPEA (1.4 mL, 2.5 equiv., 7.661 mmol) and T3P (2.9 mL, 1.5 equiv., 4.566 mmol). The reaction mixture was stirred at rt for 12 h. The reaction mixture was diluted with DCM washed with water followed by brine, dried over anhydrous sodium sulphate and concentrated. Residue was purified by combi-flash on silica gel eluting with 0-70% EA in Hexane to afford the title Compound 340 (R)-15-((tert-butyldimethylsilyl)oxy)-16,16-dimethyl-1-oxa-6-thia-9,13-diazacycloheptadecane-2,5,10,14-tetraone (700 mg, 1.474 mmol, 50%) as a gummy mass. LCMS (M+1):477.4.

### Example 44: Synthesis of Compound 206—Synthesis of (R)-15-hydroxy-16,16-dimethyl-1-oxa-6-thia-9,13-diazaycloheptadecane-2,5,10,14-tetraone

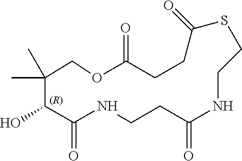

To a stirred solution of the product from Example 43 Step 5 ((R)-15-((tert-butyldimethylsilyl)oxy)-16,16-dimethyl-1-oxa-6-thia-9,13-diazacycloheptadecane-2,5,10,14-tetraone, 400 mg, 1 equiv., 0.842 mmol) in MeOH (10 mL) at 0° C. was added acetyl chloride (79.3 mg, 1.2 equiv., 1.011 mmol). The reaction mixture was stirred at for 2 h at rt. Volatiles were removed under vacuum, and the residue was dissolved in DCM and diluted with saturated NaHCO₃ solution. Layers were separated, and the organic layer was washed with brine and water, dried over sodium sulfate, and concentrated under vacuum. Residue was purified by preparative HPLC (Mobile Phase: A=water, B=ACN; Column: zorbax (21.2 mm-15.0 mm); Flow: 20 mL/min) to afford the title Compound 206 (R)-15-hydroxy-16,16-dimethyl-1-oxa-6-thia-9,13-diazacycloheptadecane-2,5,10,14-tetraone (85 mg, 0.235 mmol, 28%) as an off white solid. LCMS: 361.2. ¹H NMR (400 MHz, DMSO) δ 7.92 (t, J=8 Hz, 1H), 7.41 (t, J=8 Hz, 1H), 5.23 (d, J=8 Hz, 1H), 3.82-3.76 (m, 2H), 3.71 (d, J=4 Hz, 1H), 3.43-3.32 (m, 2H, part overlapped in water peak of DMSO), 3.20-3.16 (m, 2H), 2.98-2.90 (m, 3H), 2.82-2.79 (m, 1H), 2.68-2.58 (m, 2H), 2.24-2.21 (m, 2H), 0.93 (s, 3H), 0.83 (s, 3H).

### Example 45 Effect of Compounds on Cell Proliferation

The effect of the compounds of the present disclosure on cancer cell proliferation can be determined with the CyQUANT direct assay according to manufacturer's instructions (Invitrogen, C7026). Briefly, 100 μL of cell suspension is seeded into black clear bottom tissue culture treated plates (Corning, 165305) in complete medium and incubated over-night in a CO₂ incubator as listed in the following table:

| | | | Seeding |
|---|---|---|---|
| | | | Density |
| Cell Line | Description | Complete medium | (cells/well) |
| PANC-1 (ATCC, | pancreas, ductal | DMEM (Gibco,11960-051), | 2500 |
| CRL-1469) | carcinoma | 10% FBS (Thermo fisher | |
| | | Scientific, 16000044) | |
| MCF-7 (ATCC, | breast, | Eagle's Minimum Essential | 3000 |
| CRL-3435) | adenocarcinoma | Medium (Gibco, 11095080), | |
| | (pleural effusion) | 0.01 mg/ml bovine insulin | |
| | | (Sigma, I1882), 10% FBS | |
| HuH-7 (Sigma, | hepatoma, | DMEM (Low glucose, 5 mM, | 5000 |
| 01042712) | differentiated | Gibco, 10567014), 2 mM | |
| | | L-glutamine, 10% FBS | |
| LoVo (ATCC, | colon, | F-12K (Gibco, 21127022), | 2000 |
| CCL-229) | adenocarcinoma | 10% FBS | |
| A549 (ATCC, | lung, carcinoma | F-12K, 10% FBS | 2500 |
| CCL-185) | | | |
| NCI-H460 (ATCC, | lung, large cell | RPMI1640 (Gibco, 11875093), | 1250 |
| HTB-177) | lung cancer | 10% FBS | |
| Caco-2 (ATCC, | colon, | Eagle's Minimum Essential | 1000 |
| HTB-37) | adenocarcinoma | Medium, 20% FBS | |
| MCF 10A (ATCC, | breast, human | Mammary Epithelial Cell Growth | 5000 |
| CRL-10317) | fibrocystic | Basal Medium (MEBM, ATCC, | |
| | disease | PCS-600-030) | |
| HT-29 (ATCC, | colon, | McCoy's 5A (Gibco, 16600082), | 2500 |
| HTB-38) | adenocarcinoma | 10% FBS | |
| A-673 (ATCC, | muscle, | DMEM, 10% FBS | 3000 |
| CRL-1598) | rhabdomyosarcoma | | |
| Hep3B (ATCC, | liver, | DMEM, 10% FBS | 2500 |
| HB-8064) | hepatocellular | | |
| | carcinoma | | |
| ES-2 (ATCC, | ovary, clear cell | McCoy's 5a, 10% FBS | 5000 |
| CRL-1978) | carcinoma | | |
| BxPC-3 (ATCC, | pancreas, | RPMI1640, 10% FBS | 3000 |
| CRL-1687) | adenocarcinoma | | |
| Calu-3 (ATCC, | lung, | EMEM, 10% FBS | 5000 |
| HTB-55) | adenocarcinoma | | |
| LNCaP clone FGC | prostate, | RPMI1640, 10% FBS | 4000 |
| (ATCC, CRL-1740) | carcinoma | | |
| HCC70 (ATCC, | breast, primary | RPMI1640, 10% FBS | 1000 |
| CRL-2315) | ductal carcinoma | | |
| Capan-1 | pancreas, | IMDM (Gibco, 12440061), | 2000 |
| (ATCC, HTB 79) | adenocarcinoma | 20% FBS | |
| SW480 (ATCC, | colon, | Leibovitz's L-15 (Gibco, | 2000 |
| CCL-228) | adenocarcinoma | 11415064), 10% FBS | |
| HCT 116 (ATCC, | colon, carcinoma | McCoy 5A, 10% FBS | 600 |
| CCL-247) | | | |

Cells can be treated in complete media with compounds of the present disclosure (50-5 μM), Staurosporine (2 μM) as positive control and vehicle DMSO control (0.2%), with refreshment of media and compounds every 24 hours for a total of 120 hours. Fluorescent signal (480 nm) is detected with a laser-based Acumen eX3 instrument.

### Example 46: Effed of Compounds on Oligodendrocytes Proliferation

The effect of compounds of the present disclosure can be determined on oligodendrocyte precursor cells (OPC) proliferation.

Cell culture. Brains of wild type mice (whole brain from 1 or 2 mouse pups) less than postnatal day (P) 2 are isolated and cultured. Briefly, following removal of the meninges, cells are dissociated with 0.25% EDTA/CMF-DMEM and 1% Trypsin (1:1), plated at a density of 75,000 cells/on 0.1 mg/ml poly-L-lysine coated borosilicate glass coverslips in 24-well plates, grown in OPC differentiation media (Oligo media) consisting of DMEM/F12 (Invitrogen 21331-020) supplemented with 1% FBS, 1% N2 Neural Supplement (Invitrogen 17502-048) and PDGF receptor alpha growth factor (Invitrogen 17502-048). Cells are fed every other day and allowed to grow for 7 days in vitro (7 DIV).

OPCs treatment with compounds of the present disclosure. Cells can be treated with compounds of the present disclosure (50-10 μM) or vehicle (0.1% DMSO) starting at 7 DIV. Media is replaced daily with freshly made working solutions of compounds or vehicle for either 48 h (9 DIV total) or 96 h (11 DIV).

Analysis. Coverslips are stored in clean 24-well plates at 4° C. until fixed-staining. Cells can be imaged using Leica DM5500 fluorescent microscope with LAS-X software. Exposure settings are maintained at the same rate across compounds of the same time-point. Cells are imaged at 20× and 40× magnifications; 20×images are utilized for counting DAPI (cell nuclei) and O4+ cells. Total DAPI per image is counted using ImageJ. Total number of O4+ cells per 20×image is manually counted using Adobe Photoshop.

### Example 47: Effect of Compounds on Neuroprotective Effect on Damage Induced by 6-OHDA on Culture of Mesencephalic Neurons

The protective effect of compounds of the present disclosure can be determined on 6-OHDA-mediated injury on mesencephalon neuronal cultures.

A female Wistar rat (Janvier; France) of 15 days gestation is terminated by cervical dislocation, the fetuses are removed from the uterus and their brains harvested and placed in ice-cold medium (Leibovitz's L15 medium, Gibco). Only ventral mesencephalic flexure is used for the cell preparations. The midbrain is dissociated by trypsinization. The reaction is stopped, and the suspension is triturated and centrifuged. The pellet of dissociated cells is resuspended in chemically defined medium consisted of Neurobasal (Gibco, 21103049), containing B27 supplement (Gibco, A3582801) and L-glutamine (Gibco, 25030081), 10 ng/ml (BDNF; Pepro Tech, France, 450-02) and 1 ng/ml (GDNF; Pepro Tech, 450-51).

Viable primary rat embryo mesencephalic cells are counted and seeded on 96-multi-wells plate precoated with poly-L-lysine. Cells are maintained in a humidified incubator at 37° C. in 5% CO2-95% air atmosphere and medium changed on day 2. On day 6, the culture medium is removed and replaced by new media without neurotrophic factors containing the compounds of the present disclosure (50-10 μM) or vehicle (0.1% DMSO). After 1 h exposure, injury of mesencephalon neuronal cultures is induced by 6-OHDA (15 μM) for further 48 h. A mixture of the growth factors GDNF (1 ng/ml) and BDNF (10 ng/ml) are used as reference compound.

On day 8, the tyrosine hydroxylase positive neurons are evaluated. Cultures are fixed 30 min at 4° C. with paraformaldehyde in PBS (4%, Sigma). After, cells are permeabilized with 0.1% Triton X100 for 30 min, saturated with PBS containing 3% of BSA (bovine serum albumin) and incubated 2 h with anti-tyrosine hydroxylase antibody (Sigma, 1:10000; clone TH-2) at 1/10000 in PBS containing 0.5% of BSA. Cells are washed three times with PBS containing 0.5% of BSA and incubated 1 h with goat anti mouse antibody coupled with AF488 (Invitrogen A11001) diluted at 1/1000 in PBS containing 0.5% of BSA. Finally, nuclei are stained with DAPI (Thermo fisher, D1306) at 1/1000 in PBS containing 0.5% of BSA. After rinsing with PBS, the plate is visualized and examined with Cell Insight HCS (Thermo Scientific) to determine the number of tyrosine hydroxylase positive cells per well.

### Example 48: Measurement of Neuroprotective Effect of Compounds on Damage Induced by MPP+ on Culture of Mesencephalic Neurons

The protective effect of compounds of the present disclosure can be determined on MPP+-mediated injury on mesencephalon neuronal cultures.

A female Wistar rat (Janvier; France) of 15 days gestation is terminated by cervical dislocation, the fetuses are removed from the uterus and their brains are harvested and placed in ice-cold medium (Leibovitz's L15 medium, Gibco). Only ventral mesencephalic flexure is used for the cell preparations. The midbrain is dissociated by trypsinization. The reaction is stopped, and the suspension is triturated and centrifuged. The pellet of dissociated cells is resuspended in chemically defined medium consisted of Neurobasal (Gibco), containing B27 supplement (Gibco) and L-glutamine (Gibco), 10 ng/ml (BDNF; Pepro Tech, France) and 1 ng/ml (GDNF; Pepro Tech).

Viable primary rat embryo mesencephalic cells are counted and seeded on 96-multi-wells plate precoated with poly-L-lysine. Cells are maintained in a humidified incubator at 37° C. in 5% CO₂-95% air atmosphere and medium changed on day 2. On day 6, the culture medium is removed and replaced by new media without neurotrophic factors containing the compounds of the present disclosure (50-10 μM) or vehicle (0.1% DMSO). After 1 h exposure, injury of mesencephalon neuronal cultures is induced by MPP⁺ (50 μM) for further 48 h. A mixture of the growth factors GDNF (1 ng/ml) and BDNF (10 ng/ml) are used as reference compound.

On day 8, the tyrosine hydroxylase positive neurons are evaluated. Cultures are fixed 30 min at 4° C. with paraformaldehyde in PBS (4%, Thermo Scientific, 28908). After, cells are permeabilized with 0.1% Triton X100 for 30 min, saturated with PBS containing 3% of BSA (bovine serum albumin) and incubated 2 h with anti-tyrosine hydroxylase antibody (Sigma, 1:10000; clone TH-2) at 1/10000 in PBS containing 0.5% of BSA. Cells are washed three times with PBS containing 0.5% of BSA and incubated 1 h with goat anti mouse antibody coupled with AF488 (Invitrogen A11001) diluted at 1/1000 in PBS containing 0.5% of BSA. Finally, nuclei are stained with DAPI at 1/1000 in PBS containing 0.5% of BSA. After rinsing with PBS, the plate is visualized and examined with Cell Insight HCS (Thermo Scientific) to determine the number of tyrosine hydroxylase positive cells per well.

### Example 49: Neuroprotective Effect of Test Compounds on Damage Induced by LPS on Culture of Mesencephalic Neurons

Test animals. Pregnant Wistar rats (Janvier; France) are group-housed and maintained in a room with controlled temperature (21-22° C.) and a reversed light-dark cycle (12 h/12 h; lights on: 17:30-05:30; lights off: 05:30-17:30) with food and water available ad libitum.

The protocol is performed in 3 independent cultures. For each culture, each condition is performed in sextuplicate.

| Day | Tasks |
|---|---|
| 0 | Plating of a primary culture of rat embryo mesencephalic cells. |
| | This culture contains microglia, astrocytes, and neurons. |
| 2 | Renewal of medium |
| 5 | Renewal of medium |
| 7 | Compound application (1 h before LPS injmy) + LPS exposure |
| 8 (24 h post-LPS) | Measure of nitric oxide (NO), TNF-α and IL1-β release |
| 12 (day 5 post-LPS) | Measure of death of dopaminergic neurons (Tyrosine hydroxylase |
| | positive neurons) and release of NO in the supernatant |

Female rat of 15 days gestation is killed by cervical dislocation. Fetuses are removed from the uterus and their brains are harvested and placed in ice-cold medium (Leibovitz's L15 medium, Gibco). Only ventral mesencephalic flexure is used for the cell preparations. The midbrain is dissociated by trypsinization. The reaction is stopped, and the suspension is triturated and centrifuged. The pellet of dissociated cells is resuspended in culture medium consisted of DMEM-F12 (Gibco), containing 10% FBS (ATCC), 10% Horse serum (Gibco) and 2 mM L-glutamine (Gibco). Viable cells are counted and seeded on 96-well plates, precoated with poly-L-lysine. Cells are maintained in a humidified incubator at 37° C. in 5% CO₂-95% air atmosphere. Half of the medium is changed on day 2 and day 5.

Treatment. On day 7, culture media are removed and replaced by new medium consisting of DMEM-F12 (Gibco) supplemented with 2% FBS (ATCC), 2% Horse serum (Gibco) and 2 mM L-glutamine (Gibco) and containing vehicle or test substances. The final concentration of the vehicle (DMSO) is set at 0.1%. After 1-hour exposure, LPS at 10 ng/ml is added and exposure further continued for 24 hours or 5 days period.

Inflammatory response measurement and neuronal death evaluation. NO production is measured in the media 24 h and 5 days after LPS exposure using the Griess reagent kit (Molecular Probes). The Griess reagent Assay is a colorimetric reaction assay which measures the conversion of a sulfanilic salt into an azo dye product by nitrite. Visible wavelength absorbance data is collected using a 96-well plate reader at 570 nm (Multiskan EX, Thermo Fisher, France).

IL-1β and TNF-α release are measured in the media 24 h after LPS exposure using the ELISA development kit (PeproTech). The ELISA plate, previously coated with anti-IL-1β or anti-TNF-α antibody at 1 μg/ml, is incubated 1 h with PBS containing 1% of BSA (bovine serum albumin). After washing four times with PBS containing 0.05% of Tween-20, the plate is successively incubated 2 h with supernatant, 2 h with biotinylated antibody at 0.5 μg/ml in PBS containing 0.1% of BSA and 0.05% of Tween-20, 45 min with Avidin-HRP conjugated at 1/2000 and 30 min with color ABTS substrate (Sigma). Visible wavelength absorbance data is collected using a 96-well plate reader at 405 nm with wavelength correction set at 650 nm (Multiskan EX, Thermo Fisher, France).

Immunodetection of tyrosine hydroxylase positive neurons. On day 12 (5 days after LPS exposure), cultures are fixed 30 min at 4° C. with paraformaldehyde in PBS (4%, Sigma). Then, cells are successively permeabilized with 0.1% Triton X100 for 30 min, saturated with PBS containing 3% of BSA (bovine serum albumin) and are incubated 2 h with anti-tyrosine hydroxylase antibody (Sigma, 1:10000; clone TH-2) at 1/10 000 in PBS containing 0.5% of BSA. Cells are washed three times with PBS containing 0.5% of BSA, and incubated 1 h with goat anti mouse antibody coupled with AF488 (Invitrogen A11001) diluted at 1/1000 in PBS containing 0.5% of BSA. Finally, nuclei are stained with DAPI at 1/1000 in PBS containing 0.5% of BSA. After rinsing with PBS, the plate is visualized and examined with Cell Insight HCS (Thermo Scientific) to determine the number of tyrosine hydroxylase positive cells per well.

Statistical Analysis. The drug-induced effect on NO and cytokines is calculated by setting the response of the LPS-stimulated control as 100%. The drug-induced effect on TH-positive neurons by setting the non-intoxicated culture condition as 100%.

### Example 50. NK Cell Activation and K562 (Erythroleukemia) Killing Assay

Primary NK cells are isolated from PBMC by negative isolation with EasySep human NK cell isolation kit (Stem Cell, 17955). NK cells are 99% viable with 96% purity as evaluated by FACS (BD Fortessa) to be CD3-CD56+ (Biolegend 300317, 318344). Isolated NK cells are placed at 80,000 cells/well with 20 ng/ml IL-2 (R&D, 202-IL-050) in the presence of CD107a antibody (clone H4A3, 565113) in RPMI (Invitrogen, 22400089) complete media with 10% FBS (Hyclone SV30087.03), 1% P/S in the presence of compounds at the dose of 10 and 50 μM for 24 h. K562 cells are collected and stained with cell trace proliferation kit (Invitrogen, C34557) and co-cultured with K562 cells (20,000 cells/well) along with addition of compounds at 10 and 50 μM and monitored cell lysis at 2, 4 and 6 h post incubation. Cells are collected and stained cells in the presence of Fc Block (Biolegend, 422302) with CD69, a NK cell activation marker, (Biolegend, 318344), PI, a viability marker (Biolegend, 310910) and analyzed by flow cytometry (BD Fortessa). Cells are first gated side versus forward scatter (SSC-A Vs FSC-A). K562 cells are further gated as SSC-A vs cell trace violet and further analyzed for dead cells by their uptake of PI (PI Vs cell trace violet dye). Cell trace negative cells are gated as NK cells which are further gated for CD56+Vs CD69+ to determine activated NK cells.

### Example 51: Tolerogenic DC Differentiation Assay

Monocytes are isolated by positive isolation with CD14+ microbeads (Miltenyi, 130-050-201). Monocytes would be 99% viable with 96% purity as analyzed by FACS and CD14+(BD, 563561). 200,000 monocytes would be placed along with compounds and allowed to differentiate to dendritic cells with 50 ng/ml GMCSF (R&D, 15-GM-050/CF) in combination with 25 ng/ml IL-4 (R&D 204-IL-050/CF) in RPMI complete media with 15% FBS (Hyclone SV30087.03) and 1% Penicillin-Streptomycin (Gibco, 15140-122). On day 3 half the media will be refreshed with fresh GM-CSF and IL4 and compounds at 10 and 50 μM dose. On day 5 the dendritic cells would be further differentiated to tolerogenic dendritic cells with vitamin D3, 100 nM (Selleck S4063) and dexamethasone 10 nM (Selleck S1322). On day 6 LPS would be added (Sigma, L6143) at final concentration of 10 ng/ml and cells collected for flow analysis and supernatant for IL-10 (DKW, 1110003) measurement by ELISA. Cells would be stained with live/dead APC (Invitrogen, L10120), Percp-Cy5.5 mouse anti-human HLA-DR (BD 560652), PE mouse anti-human CD83 (BD 556855), Alexa Fluor® 488 anti-human CD86 Antibody (Biolegend 305414), BV510 mouse anti-human CD141 (BD, 563298), PE/Cy7 anti-human CD85k (ILT3) (Biolegend, 33012), or with corresponding isotype controls (Percp-Cy5.5 Mouse IgG2a,κ, BD, 552577), PE Mouse IgG1,κ (BD, 555749), Alexa Fluor® 488 Mouse IgG2b, κ Isotype Ctrl (Biolegend, 400329), BV510 Mouse BALB/c IgG1,κ (BD, 562946) Pe/Cy7 Mouse IgG1, κ Isotype Ctrl Antibody (Biolegend, 400126). Tolerogenic cells would be defined as live, CD83-CD86-HLA-DR+CD141+CD85k+ and increased production of IL-10.

### Example 52: Myeloid Derived Suppressor Cells (MDSC) Suppression Assay

Monocytes would be isolated by positive isolation with CD14+ microbeads (Miltenyi, 130-050-201). Monocytes would be 99% viable with 96% purity as analyzed by FACS and CD14+ (BD, 563561). 100,000 monocytes will be incubated with compounds at the dose of 10 and 50 μM±PD-1 (Nivolumab) and differentiated to MDSC with 10 ng/ml GMCSF (R&D, 215-GM-050/CF) and IL-6 (R&D 206-IL-050/CF) in RPMI complete media (Invitrogen, 22400089) with 15% FBS (Hyclone SV30087.03) and 1% Penicillin-Streptomycin (Gibco, 15140-122). On day 2, 4 and 6 half the media will be refreshed with fresh GM-CSF and compounds at dose of 10 and 50 μM. Autologous T-cell will be isolated by negative selection with EasySep Human T cell isolation kit (Stem cell, 17951) and stained with Cell Tracerm Violet Cell Proliferation Kit for flow cytometry (Invitrogen, C34557). MDSC are cocultured with T cells activated with dyna bead (human CD3/CD2, Invitrogen, 11131D) at the ratio of MDSC:T cells:dynabead 0.5:1:1 along with the compounds for 4 days. The supernatant would be collected for the measurement of IFNγ (Dakewe, 1110002). Next, MDSC and T cells would be stained and will be analyzed by FACS (BD LSR Fortessa, 853492), live dead fixable far red (Invitrogen, L34974), anti-Human CD33 (BD 555626), mouse Anti-Human CD15 (BD 560827), mouse anti-Human CD14 (BD 563561), mouse anti-Human HLA-DR (BD, 560652), mouse anti-Human CD4 (BD 563550), anti-human CD8 Antibody (Biolegend, 344714), anti-human CD11b (Biolegend, 301332), mouse IgG2a isotype controls (BD 550927). MDSC are defined as CD11b+, CD33+, CD15+, CD14−, HLA−DR−. CD4 and CD8 are T-cell markers to understand the proliferative capacity of both CD4+ and CD8+ T cells in the presence of MDSC in presence and absence of the compounds.

### Example 53: Th17 Differentiation Assay

Naïve CD4+ T cells (Stemcell, Cat #17555) are isolated from PBMC and seeded (20,000 cells/well) in a 96-well flat bottom plate (Eppendorf, 30730119) pre-coated with 10 μg/ml anti-CD3 antibody (EBioscience, 16-0037-85) for 3 hours at 37° C. in an X-VIVO15 medium (Lonza, 04-418Q) supplemented with 15% FBS (Hyclone, SV30087.03) and 1% Penicillin-Streptomycin (Hyclone, SV30010). Th17 differentiation cocktail is added (Biolegend, 423303), including 2 μg/ml anti-CD28 (BD 555725), 10 ng/ml IL-1μ (R&D, 201-LB-005) 10 ng/ml IL-6 (R&D, 206-IL-010), 10 μg/ml anti-IL-4 (BD, 554481), 10 ng/ml IL-23 (R&D, 1290-IL-010/CF), 10 μg/ml anti-human IFNγ (BD, 16-7318-85), 10 ng/ml TGF-β1 (R&D, 240-B-010) in the presence of the compounds at 10 and 50 μM dose. On day 3 and day 8 half of the medium, is refreshed with Th17 differentiation cocktail as above and compounds (10 and 50 μM). Th17 cells are stained and analyzed by FACS (BD LSR Fortessa, 853492). On day 10, cells are collected and are stained for live/dead dye (Life technology, L34975), surface and intracellular IL-17a with fixation/permeabilization solution (BD, 554722), and mouse anti human CD4 (BD, Cat #564651), anti-human IL-17a (BD, 560490) and/or mouse anti-human IgGlx (BD, 557714).

### Example 54: Treg Differentiation Assay

Naîve CD4+ T cells (Stemcell, 17555) are isolated from PBMC and placed (20,000 cells/well) in a 10 μg/ml anti-CD3 antibody (eBioscience, 16-0037-85) pre-coated 96-well flat bottom plate (Eppendorf, Cat #30730119) for 3 hours at 37° C. in an X-VIVO15 medium (Lonza, 04-418Q) supplemented with 15% FBS (Hyclone SV30087.03) and 1% Penicillin-Streptomycin (Hyclone, SV30010). Treg induction cocktail is added including 2 μg/ml anti-CD28 (eBioscience 16-0289-85), 20 ng/ml IL-2 (R&D, 202-IL-050) 0.2 ng/ml TGF-β1 (Peprotech, 100-21-50) in the presence of the compounds at 10 and 50 μM dose. On day 3 half of the medium, is refreshed with Treg differentiation cocktail as above and compounds (10 and 50 μM). On day 5 cells are collected and the following staining are performed: live/dead (Invitrogen, L34963), and Foxp3/Transcription Factor Staining Buffer Set (EBioscience, 00-5523-00), mouse anti-human FoxP3 (BD, 560046) or mouse IgG1,κ Isotype control (BD, 555749) and analyzed by FACS (BD LSR Fortessa, 853492).

### Example 55: Mast Cell Activation Assay

MC/9 cell line (ATCC, CRL-8306) would be thawed and grown in DMEM High Glucose (Gibco 11995-065) supplemented with 10% FBS (Hyclone, SV30087.03), 1% Penicillin-Streptomycin (Hyclone, SV30010), along with T-Cell Supplement (Corning, 354115). 500,000 cells/well would be placed in Tyrode's buffer (100 μl) together with anti-CD107a antibody. The assay is performed in 2 sets; in the first set MC/9 cell line would be treated directly with the compounds at 10 and 50 μM and in the second set, cells would be treated with the compounds at 10 and 50 μM dose in the presence of C48/80 compound (Sigma, C2313) to induce mast cell degranulation. After incubation for 30 min-1 h, 30 μl supernatant would be collected and incubated with 10 μl substrate solution (p-nitrophenyl-N-acetyl-$-D-glucosaminide) for 30 mins at 37° C. Then 100 μl of carbonate buffer would be added according to the manufacturer's instructions (N-Acetylglucosaminidase (beta-NAG) Activity Assay Kit, Abcam Ab204705) and absorbance is read at 405 nm.

### Example 56: Measurement of ROS

Primary adherent fibroblasts are cultured in minimum essential medium (MEM) (Gibco, 25030081) supplemented with 2 mM L-Glutamine (Gibco, 25030081), 15% fetal bovine serum (FBS) (Gibco, 26400044) and 1% penicillin/streptomycin (Gibco, 5140122) at 37° C. and 5% CO₂. Cells are collected for either passaging or experiment at ˜70-80% confluence. Cells are obtained by trypsinization, and 5000K cells are seeded in culture media, on a black, clear-bottomed plate (Thermo Fisher Scientific, 165305) and allowed to adhere for 16-18 hours to have confluency around 70-80%. After 24 hours prior to measurements (37° C, 5% CO2) media is changed to Dulbecco's Modified Eagle Medium (DMEM, Agilent Seahorse cat #103575-100) with the appropriate supplements as stated below.

| Conditions | |
|---|---|
| used | Supplements in DMEM |
| A | 1 mM glucose, 2 mM L-glutamine, |
| | 1 mM pyruvate, 10% FBS |
| B | 1 mM glucose, 2 mM L-glutamine, |
| | 1 mM pyruvate |
| C | 1 mM glucose, 10% FBS |

Primary fibroblasts suitable for assay include: Healthy controls (GM00041, GM05659, GM23974 Coriell Institute for Medical Research), Propionic Acidemia (PA) (GM00371, GM03590 Coriell Institute for Medical Research, Tsi 6337 Trans-Hit Bio), Methylmalonic Acidemia (MMA) (GM01673, Coriell Institute for Medical Research, Tsi 5224, Tsi 4290 Trans-Hit Bio), Branched chain ketoacid dehydrogenase kinase (BCKDK) (GM00612, GM00649 Coriell Institute for Medical Research), Subnormal activation of pyruvate dehydrogenase complex (PDH) (GM01503 Coriell Institute for Medical Research), Very long-chain acyl-CoA dehydrogenase (VLCAD) (GM17475), Leigh Syndrome (LS) (GM03672, GM13411 Coriell Institute for Medical Research), Pyruvate Carboxylase Deficiency (PC) (GM00444 Coriell Institute for Medical Research), Glutaric Acidemia-1 (GA), Impaired VLCFA oxidation (VLCFA) (GM13262), Kearns-Sayre Syndrome (KSS) (GM06225 Coriell Institute for Medical Research), Friedreich's Ataxia (FXN), Huntington's disease (HD) (GM21756 Coriell Institute for Medical Research).

Next, cells are treated with compounds and ROS H2DCFDA (22 μM) dye for 2-24 hours. Kinetic read is started immediately with a i3×plate reader (492/527 nm) for 60 minutes.

### Example 57. Measurement of Total NAD⁺+NADH and NAD⁺

Primary adherent fibroblasts are cultured in minimum essential medium (MEM) (Gibco, 25030081) supplemented with 2 mM L-Glutamine (Gibco, 25030081), 15% fetal bovine serum (FBS) (Gibco, 26400044) and 1% penicillin/streptomycin (Gibco, 5140122) at 37° C. and 5% CO₂. Cells are collected for either passaging or experiment at ˜70-80% confluence. Cells are obtained by trypsinization and 5000K cells are seeded on a white plate (Thermo Fisher Scientific, 152028) and allowed to adhere for 16-18 hours to have confluency around 70-80% in the cell well with culture media. After 24 hours prior to measurements (37° C, 5% CO2) media is changed to Dulbecco's Modified Eagle Medium (DMEM, Agilent Seahorse cat #103575-100) with the appropriate supplements as stated below.

| Conditions | |
|---|---|
| used | Supplements in DMEM |
| A | 1 mM glucose, 2 mM L-glutamine, |
| | 1 mM pyruvate, 10% FBS |
| B | 1 mM glucose, 2 mM L-glutamine, |
| | 1 mM pyruvate |
| C | 1 mM glucose, 10% FBS |

Primary fibroblasts suitable for assay include: Propionic Acidemia (PA) (GM03590 Coriell Institute for Medical Research, Tsi 6337 Trans-Hit Bio), Methylmalonic Acidemia (MMA) (Tsi 5224 Trans-Hit Bio), Subnormal activation of pyruvate dehydrogenase complex (PDH) (GM01503 Coriell Institute for Medical Research), Very long-chain acyl-CoA dehydrogenase (VLCAD) (GM17475), Leigh Syndrome (LS) (GM03672, GM13411 Coriell Institute for Medical Research), Pyruvate Carboxylase Deficiency (PC) (GM00444 Coriell Institute for Medical Research).

Assay is performed according to manufacturer instructions (NAD/NADH-Glo Assay Promega, G9072). The NAD/NADH-Glo Assay is a bioluminescent assay for detecting total oxidized and reduced nicotinamide adenine dinucleotides (NAD+ and NADH, respectively) from which ratio of NAD/NADH can be calculated. Briefly, for every assay a 12-point standard curve is prepared ranging from 400 nM to 0.625 nM. Media is removed and replaced with 50 μl of PBS for both assays.

For individual NAD+ and NADH measurement, 50 μl of 1% DTAB (Sigma Cat #D5047) (cell lysis reagent) in 0.2N NaOH is added to the plate and a total 100 μl of lysate is split into two plates with 50 μl each. 25 μl of 0.4N HCl are added to the NAD+ plate and both NAD+ and NADH plate are heated at 60° C. for 15 minutes. The acid and heat treatment destroyed NADH allowing individual NAD+ measurement while heating in basic conditions destroys NAD+ allowed individual NADH measurements. The plates are allowed to come to room temperature for 10 minutes and Trizma base (Sigma, T1699) is added to the NAD+ plate to neutralize the acid and HCl Trizma hydrochloride (Sigma, T2694) is added to the NADH plate. The NADNADH glo reagent is prepared by adding: 625 μl of NAD cycling substrate, 125 μl of reductase, 125 μl of reductase substrate, 125 μl NAD cycling enzyme to total of the 25 ml of NAD GLO reagent. 1:1 ratio of total volume of reagent is added to the individual NAD+, NADH measurement (total 100 μl) and 50 μl for total NAD/NAH measurement. Luminescence is read between 30-60 minutes within the linear range.

### Example 58. Measurement of NADP+/NADPH

Primary adherent fibroblasts are cultured in minimum essential medium (MEM) (Gibco, 25030081) supplemented with 2 mM L-Glutamine (Gibco, 25030081), 15% fetal bovine serum (FBS) (Gibco, 26400044) and 1% penicillin/streptomycin (Gibco, 5140122) at 37° C. and 5% CO₂. Cells are collected for either passaging or experiment at ˜70-80% confluence. Cells are obtained by trypsinization and 5000K cells are seeded on a white plate (Thermo Fisher Scientific, 152028) and allowed to adhere for 16-18 hours to have confluency around 70-80% in the cell well with culture media. After 24 hours prior to measurements (37° C, 5% CO₂) media is changed to Dulbecco's Modified Eagle Medium (DMEM, Agilent Seahorse cat #103575-100) with the appropriate supplements as stated below.

| Conditions | |
|---|---|
| used | Supplements in DMEM |
| A | 10 mM glucose, 2 mM L-glutamine, |
| | 1 mM pyruvate, 10% FBS |
| B | 1 mM glucose, 2 mM L-glutamine, |
| | 1 mM pyruvate, 10% FBS |

Primary fibroblasts suitable for assay include: Propionic Acidemia (PA) (GM03590 Coriell Institute for Medical Research, Tsi 6337 Trans-Hit Bio), Methylmalonic Acidemia (MMA) (Tsi 5224 Trans-Hit Bio), Subnormal activation of pyruvate dehydrogenase complex (PDH) (GM01503 Coriell Institute for Medical Research), Very long-chain acyl-CoA dehydrogenase (VLCAD) (GM17475), Leigh Syndrome (LS) (GM03672, GM13411 Coriell Institute for Medical Research), Pyruvate Carboxylase Deficiency (PC) (GM00444 Coriell Institute for Medical Research). 10 μM of Compounds are added for 2 hours in PA, MMA lines and for 24 h for the other fibroblast lines.

Assay is performed according to manufacturer instructions (NADP+/NADPH-Glo Assay Promega, G9082). Assay required a plate for total measurement of NADP+/NADPH and a plate for individual measurement of NADP+ or NADPH. For every assay a 12-point standard curve is prepared ranging from 400 nM to 0.625 nM. Media is removed and replaced with 50 μl of PBS for both assays.

For individual NADP+/NADPH measurements, 50 μl of 1% DTAB (Sigma, D5047) (cell lysis reagent) in 0.2N NaOH is added to the plates and 100 μl of lysate is split into two plates with 50 μl each from which ratio of NADP/NADPH can be calculated. 25 μl of 0.4N HCl are added to the NADP+ plates and both NADP+ and NADPH plates are heated at 60° C. for 15 minutes. The acid and heat treatment destroyed NADPH allowing individual NADP+ measurements while heating in basic conditions destroys NADP+ allowing individual NADPH measurement. After 15 minutes plates are allowed to come to room temperature and Trizma base (Sigma, T1699) is added to the NADP+ plates to neutralize the acid and HCl Trizma hydrochloride (Sigma, T2694) is added to the NADPH plates. The NADNADPH glo reagent is prepared by adding 125 μl of NAD cycling substrate, 125 μl of reductase, 125 μl of reductase substrate, 125 μl NAD cycling enzyme to a total of the 25 ml of NAD GLO reagent.

1:1 ratio of total volume of reagent is added to the individual NADP+, NADPH measurements (total 100 μl) and 50 μl are added for the total NAD/NADPH measurements. Luminescence is measured for 30-60 minutes within the linear range.

### Example 59: Effed of Compounds on Mitochondrial Membrane Potential

The effect of compounds of the present disclosure on mitochondrial membrane potential can be determined by fluorescence measurement.

HepG2 cells (ATCC, HB-8065) are cultured (5% CO2 at 37° C.) and plated in Poly-D-Lysine 384-Well plate (Corning, 356663) at a density of 160,000 cells/mL in 50 μL/well. Culture and assay media consists of DMEM (Gibco, 11995-065) supplemented with 1% Penicillin-Streptomycin and 10% FBS (Hyclone, SV30087.03). Mitochondrial membrane potential can be determined using the MITO-ID® MP detection Kit (ENZ-51018) according to manufacturer instructions. Cells are allowed to settle for 30 min at room temperature and further incubated overnight (37° C. and 5% CO₂) for adherence. Next day, test compounds and vehicle DMSO (0.2%) are added at the recommended volumes to the 384-well plate for 7 days without media change. All the solutions and wash buffers are prepared and dispensed according to manufacturer's volumes for a 384-well plate format.

### Example 60: Measurement of Mitochondrial Membrane Potential Changes Using the JC-1 Assay

The effect of compounds of the present disclosure on mitochondrial membrane potential can be determined with a JC-1 assay.

Cells are cultured in minimum essential medium (MEM) (Thermo Fisher Scientific) supplemented with 2 mM L-Glutamine (Thermo Fisher Scientific), 15% fetal bovine serum (FBS) (Gibco) and 1% penicillin/streptomycin at 37° C. and 5% CO₂ and plated at a density of 75,000 cells/well in 24 well plates (2 plates per cell line) in MEM growth media. Once attached (about 2-3 h later), media is aspirated and replaced with 450 μl/A Media: 10 mM glucose, 2 mM glutamine, 1 mM pyruvate, 10% FBS or B Media: 1 mM glucose, 10% FBS) (1 plate of each). A working compound plate is prepared with all compounds at 10 mM. Immediately before adding to cells, compounds are diluted to 100 μM (10×) in starved media, and 50 μl/well is added to the cells in 24 well plates. Final concentration is 10 μM. After 24 hours, the media is aspirated, and cells are washed once with 500 μl D-PBS (no additions). After aspirating the wash buffer, 200 μl/well trypsin is added, and the plates are incubated at room temperature (RT) until cells detached. Trypsin is inactivated with 100 μl FBS, and cells are transferred to a 96 well v bottom plate and for centrifugation at 250 g for 5 minutes at RT. Supernatant is removed and cells are washed with PBS. Cells are then resuspended in 50 μl staining buffer and FCCP is added to 10 μM in control wells. The plate is incubated at RT for 5 min, then 2×JC-1/DAPI is added at 50 μl per well. Cells are taken to the flow cytometer (Miltenyi MACSQuant Analyzer), and acquisition is initiated immediately. Channels used are V1 (DAPI), B1 (JC-1 monomers) and B2 (JC-1 aggregates). Data are analyzed with FlowJo by TreeStar, and compensation is performed digitally in the analysis program using the FCCP-treated samples as the maximally green fluorescence control (corresponding to JC-1 monomers). Each cell line is analyzed individually to ensure proper gating. Geometric mean fluorescence intensity for JC1 aggregates (corresponding to red fluorescence) and monomers (corresponding to green fluorescence) is determined within the DAPI negative population (live cells). The ratio of red:green is calculated and expressed related to vehicle (vehicle=1).

### Example 61: Effects of Test Compounds on Mitobiogenesis In-Cell ELISA Assay

The effect of compounds from the present disclosure on the levels of mitobiogenesis can be determined using the MitoBiogenesis™ In-Cell ELISA Kit (Abcam ab110216) according to manufacturer's instructions. Mitobiogenesis is represented by the relative expression of two proteins which are each subunits of a different oxidative phosphorylation enzyme complex: subunit I of Complex IV (COX-I), which is mitochondrial (mt)DNA-encoded, 70 kDa subunit of Complex II (SDH-A), which is nuclear (n)DNA-encoded. Complex IV includes several proteins which are encoded in the mitochondrion, while the proteins of Complex II are entirely encoded in the nucleus.

HepG2 cells (ATCC, HB-8065) are cultured (5% CO₂ at 37° C.) and plated in Poly-D-Lysine 384-Well plate (Corning, 356663) at a density of 40,000 cells/mL in 50 μL. Culture and assay media consisted of DMEM (Gibco, 11995-065) supplemented with 1% Penicillin-Streptomycin and 10% FBS (Hyclone, SV30087.03). Cells are allowed to settle for 30 min at room temperature and further incubated overnight (37° C. and 5% CO₂) for adherence. Next day, 50-5 μM of the negative control Chloramphenicol (Selleck, S1677), test compounds (50-10 μM) and vehicle DMSO (0.2%) are added at the recommended volumes to the 384-well plate for 7 days without media change using a Tecan compound dispenser. All the solutions and wash buffers are prepared and dispensed according to manufacturer's volumes for a 384-well plate format. Effects of compounds on mtDNA-encoded protein expression (COX-I) and nuclear DNA-encoded mitochondrial protein expression (SDH-A) are expressed as relative signal (nm) to vehicle of a n=2 per conditions.

### Example 62: Effects of Test Compounds on Glucose Uptake

The effect of compounds from the present disclosure on glucose uptake is determined in HepG2 cells (ATCC, HB-8065) using the glucose uptake Glo Assay Kit (Promega, J1343 according to manufacturer instructions. HepG2 cells are cultured in complete DMEM-glucose media (Gibco) supplemented with 10% FBS (37° C. incubator with 5% CO₂) and seeded in 96-well plates at 30,000 cells/well. After removing the complete media, 100 μL/well of serum-free, high-glucose DMEM media are added to the wells and incubated overnight (37° C. incubator with 5% CO₂). Media is then replaced with 100 μl/well DPBS containing 0.6% BSA and starved for 1 hour. Next, DPBS is removed and 45 μl/well of insulin (100 nM) or compounds (10 μM-50 μM) are added to the wells and incubated for 10 minutes (37° C. incubator with 5% CO₂). Insulin and compounds are prepared in DPBS with 0.6% BSA with a final DMSO concentration of 0.1%. Next, 5 μl of 2DG (10 mM) in DPBS are added per well and allowed to incubate for 20 minutes followed by addition of 25 μl stop buffer. 37.5 μl of the mixture are then transferred to a new plate and 12.5 μl of Neutralization buffer added to the wells. After, 50 μl of 2DG6P detection Reagent are added and incubated for 0.5-1 hour at room temperature. Luminescence is measured with 0.3-1 second integration on a luminometer.

### Example 63: Effects of Compounds on Ammonia Levels

The effect of compounds from the present disclosure on ammonia levels can be determined in patient derived fibroblasts.

The cell culture medium is Eagles Minimal Essential Medium containing non-essential amino acids, supplemented with 10-15% FBS with 0.3% penicillin/streptomycin. Supplier conditions for thawing, growing, feeding and harvesting of each cell line are strictly followed. Cells are cultured in four T-25 cm2 cell culture flasks for each compound challenge (three replicate flasks for sample preparation, one flask for representative cell count). Cells are challenged for 4 hours and this is performed in each culture media consisting of A Media: 10 mM glucose, 2 mM glutamine, 1 mM pyruvate or B Media: 1 mM glucose, with compounds in 0.5% vehicle.

Ammonia determination. Ammonia values are measured on freshly prepared media samples (TO) and after completion of treatment (T4) via Modified Berthelot, Ammonia Assay Kit (Colorimetric, Abcam, ab102509). T4 values are taken from each triplicate flask from each cell line directly at treatment end point. Cell counts are taken using standard cell counting with bright field microscope from 1 parallel flask for each culture condition.

### Example 64: Effects of Compounds on Gene Expression

Gene expression. qPCR gene expression analysis of the following markers can be performed on RNA isolates from cell culture flasks.

A description of markers analysed via qPCR is shown in the table below:

| Gene | Description of protein encoded |
|---|---|
| ACOT8 | A peroxisomal Acyl-CoA thioesterase involved in the oxidation of fatty acids |
| ACOT4 | A peroxisomal succinyl-coenzyme A thioesterase which can also hydrolyse |
| | glutary-1-CoA and long chain saturated acyl-CoAs. |
| FGF21 | A member of the fibroblast growth factor (FGF) family, possessing broad |
| | mitogenic, cell survival and metabolic regulation activities. |
| PPAR α | A member of the nuclear receptor family of ligand-activated transcript ion |
| | factors that heterodimerise with the retinoic X receptor (RXR) to regulate gene |
| | expression. Regulates the peroxisomal beta-oxidation pathway of fatty acids. |
| PPARγ | A member of the nuclear receptor family of ligand-activated transcription |
| | factors that heterodimerise with RXR to regulate gene expression. Implicated |
| | in the pathology of numerous diseases including obesity, diabetes, |
| | atherosclerosis and cancer. |
| GLUT1 | Glucose transporter enabling movement of hydrophilic glucose across the cell |
| | membrane. |
| HIF1α | Alpha subunit of transcription factor hypoxia-inducible factor-1. Functions as |
| | a master regulator of cellular and systemic homeostatic response to hypoxia |
| | by activating transcription of genes, involved in energy metabolism, |
| | angiogenesis and apoptosis. |
| GLUT4 | An insulin-regulated facilitative glucose transporter. Upon insulin |
| | stimulation, the sequestered protein moves to the cell surface and begins to |
| | transport glucose across the cell membrane. |
| SIRT3 | Member of the sirtuin family of class III histone deacetylases. Found |
| | exclusively in mitochondria, where it can eliminate reactive oxygen species |
| | and inhibit apoptosis. Involved in nuclear gene expression and metabolic |
| | control. |
| SIRT5 | NAD-dependent lysine demalonylase, desuccinylase and deglutarylase. |
| | Contributes to the regulation of ammonia levels during fasting. |
| GAPDH | An enzyme which catalyzes glycolysis and breaks down glucose. Used as a |
| | control (housekeeping) marker. |

The primers used in the assay are stated below.

| | | | SEQ ID |
|---|---|---|---|
| Gene | Species | Sequence (5′-3′) | NO: |
| HIF1α | human | AAAATCTCATCCAAGAAGCC | 1 |
| | | AATGTTCCAATTCCTACTGC | 2 |
| ACOT8 | human | CATCTATCCCCATTCCTGAG | 3 |
| | | TTTTTCCAGTGGTATCAGTC | 4 |
| ACOT4 | human | CTTTGAAGATCTCCCCAATA | 5 |
| | | AC | |
| | | GATCCATTGATGGAAACTGTG | 6 |
| FGF21 | human | TACCTCTACACAGATGATGC | 7 |
| | | CCCAAGATTTGAATAACTCCC | 8 |
| PPARα | human | CCTAAAAAGCCTAAGGAAACC | 9 |
| | | GATCTCCACAGCAAATGATAG | 10 |
| PPARγ | human | AAAGAAGCCAACACTAAACC | 11 |
| | | TGGTCATTTCGTTAAAGGC | 12 |
| GLUT 1 | human | ACCTCAAATTTCATTGTGGG | 13 |
| | | GAAGATGAAGAACAGAACCAG | 14 |
| GLUT 4 | human | CCATTGTTATCGGCATTCTG | 15 |
| | | ATTCTGGATGATGTAGAGG | 16 |
| | | TAG | |
| SIRT3 | human | GAAACTGGGAAGCTTGATG | 17 |
| | | CTTGTCAGAATTGGGATGTG | 18 |
| SIRT5 | human | ATTCAGGTTTCATTTCCAGG | 19 |
| | | GATTGTTCAGTACTCAGCTC | 20 |
| GAPDH | human | ACCCACTCCTCCACCTTTGA | 21 |
| | | CTGTTGCTGTAGCCAAATTC | 22 |
| | | GT | |

Reverse transcription (RT) of total RNA to single-stranded cDNA (2 μl) is performed with the High Capacity cDNA Reverse Transcription Kit (AB Applied Biosystems, 042557), according to manufacturers instructions. Real time (RT) PCR is performed using the Power SYBR® Green PCR Master Mix and Power SYBR® Green RT-PCR Reagents Kit (Thermo Fisher Scientific, 042179). Master Mix reaction consisting of 10 μl SybrGreen (2×), 1.2 μl primer Reverse (of 5 μM dilution), 1.2 μl primer Forward (of 5 μM dilution) and 5.6 μl RNA free/DNA free water.

### Example 65: Vitamin B12 Deficiency Mouse Model

A vitamin B12 deficiency mouse model is used following the methods described at Ghosh et al 2016.

Animal maintenance and feeding. Female weanling C57BL/6 mice (n=65) are obtained at 3 weeks of age from Shanghai Sippe-Bk Lab Animal Co., Ltd. The mice are housed in a SPF environment and maintained at 20° C.±6, under standard lighting conditions (12-h light/dark cycle). Animals are divided into 5 animals per cage during modelling, and 3 animals per cage during compound test. The 3-week old mice are fed ad libitum with either AIN-76A control diet (D10001i) designated as control diet group or the same diet deficient in vitamin B12 (D07012902) with pectin as the source of fiber (designated as Cbl−/− cobalamin deficient) referred to as B12R+ by Ghosh et al. (Research Diets Inc., New Brunswick, NJ, USA). Cobalamin-restricted diet with pectin (B12R+) contained 50 g pectin/kg diet, because it has been shown earlier that pectin binds the intrinsic factor in the intestine and makes vitamin B12 less bioavailable. The control diet contained 50 g cellulose/kg diet as the fiber source instead of pectin. The mice also had ad libitum access to deionized water. Food intake and body weights are recorded every week.

Compound treatment. After 6 weeks of feeding on either control or Cbl−/− diet, 9-week-old mice on Cbl−/− diet are randomly assigned into treatment groups. Mice in each group are IP treated with vehicle (1% HPBCD, Sigma, H107) or Compound BID.

Tissue and sample collection. At specified timepoints, mice are weighed and anesthetized with CO₂ before sample collection. For blood collection, the chest is opened to expose the heart. Up to 300 μl blood is drawn from the left ventricle with 1 ml syringe rinsed with EDTA-Na and dispensed into a K₃EDTA mini collect tube (Greiner Bio-One) for haematology analysis. Then a new syringe is used to draw remaining blood from heart as much as possible. Serum is isolated by centrifugation at 5000 rpm for 10 min, aliquoted and kept at −80° C. until further use. For tissue collection after blood is drawn, mice are perfused with ice cold saline from the left ventricle. Heart, liver, kidney, spleen, brain is weighed after isolation. Then left leg is kept in ice cold PBS and bone marrow is isolated for further immunophenotype analysis. The liver is cut into one piece of 100 mg for homogenization, other pieces of 40 mg or 100 mg are snap frozen and are stored at −80° C. before use. Heart, liver and kidney are also cut into a piece of 40 mg, snap frozen in liquid nitrogen and other part is stored at −80° C. For brain isolation, skull is cut open to expose the brain and is carefully taken out with forceps. Pieces of 40 mg are cut and snap frozen and stored at −80° C.

### Example 66: Vitamin B12 Deficiency Mouse Model. Hematology Analysis

Blood hematology is performed with XN-1000-Hematology-Analyzer (Sysmex America, Inc.). Sample processing is performed as described in Example 65.

### Example 67: Vitamin B12 Deficiency Mouse Model. Biochemistry Parameters

Creatinine, Urea in urine and blood would be measured using biochemical analyzer Mindray BS-380 (Mindray, Shenzhen, P.R. China). Sample processing is performed as described in Example 65.

### Example 68. Vitamin B12 Deficiency Mouse Model. Immunophenotyping Analysis

Sample processing is performed as described in Example 65. All bone marrow cells are collected, and cell suspension are filtered through 70 μM cell strainer and washed with PBS. Red blood cells are removed by using 1×RBC Lysis Buffer (Sigma, R7757). Cells are stained for live/dead dye (FVS780, BD 565388), surface and intracellular markers with fixation/permeabilization solution (eBioscience, 88-8824-00) with anti-mouse CD45 (eBioscience, 69-0451-82), anti-mouse CD11b (eBioscience, 12-0112085), anti-mouse F4/80 (Biolegend, 123116), anti-mouse MHC-II (BD, 553623), anti-mouse CD206 (Biolegend, 141717), anti-Ly6G (BD, 560602), and anti-Ly6C (Biolegend, 128017). Cells are gated on singlets followed by live cells, CD45, CD11b, & F4/80 and CD11b+F4/80 dim are defined as macrophages. Further M1 macrophages are gated as MHC-II positive and CD206+ are gated as M2 macrophages.

### Example 69: Vitamin B12 Deficiency Mouse Model. Mouse Liver Protein Determination

Sample processing is performed as described in Example 65. ELISAS in the liver homogenates are performed according to manufacture instructions for TNFα and protein carbonyls. One could perform the other ELISAs as described and instructed by manufacturing protocols. Below is the list of ELISA assay kits and catalog number for each assays.

| | Cat |
|---|---|
| Reagents | Number |
| Mice Glu ELISA Kit | JL18293 |
| Mice GSH ELISA Kit | JL20360 |
| Mice GSSG ELISA Kit | JL20647 |
| Mice SMA ELISA Kit | JL46310 |
| Mice MDA ELISA Kit | JL13329 |
| Mice PCO ELISA Kit | JL46483 |
| Mice CAT ELISA Kit | JL18163 |
| Mice SOD ELISA Kit | JL12237 |
| Mice TG ELISA Kit | JL46662 |
| Mice LDL-C ELISA Kit | JL20313 |
| Mice HDL-C ELISA Kit | JL20356 |
| Mice MPO ELISA Kit | JL10367 |
| Mice AH3 ELISA Kit | JL48757 |
| Mice AH4 ELISA Kit | JL48753 |
| Mice Acetylated α-tubulin ELISA Kit | JL48751 |
| Mice FGF-21 ELISA Kit | JL20295 |
| Mice ADPN ELISA Kit | JL20696 |
| Mice PPAR-α ELISA Kit | JL12627 |
| Mice PPAR-γ ELISA Kit | JL20369 |
| Mice Acetylated Lysine ELISA Kit | JL48758 |
| Mice ALT ELISA Kit | JL13983 |
| Mice ALP ELISA Kit | JL26471 |
| Mice AST ELISA Kit | JL13939 |
| Mice OGT ELISA Kit | JL45999 |
| Mice NRF1 ELISA Kit | JL48755 |
| Mice NRF2 ELISA Kit | JL48768 |
| Mice YY1 ELISA Kit | JL48770 |
| Mice ERα ELISA Kit | JL48767 |
| Mice SIRT1 ELISA Kit | JL20291 |
| Mice SIRT2 ELISA Kit | JL47860 |
| Mice SIRT3 ELISA Kit | JL48783 |
| Mice SIRT4 ELISA Kit | JL48762 |
| Mice SIRT5 ELISA Kit | JL48778 |
| Mice SIRT6 ELISA Kit | JL48771 |
| Mice TNF-α ELISA Kit | JL29365 |
| Mice IL-6 ELISA Kit | JL12523 |
| Mice IL-1β ELISA Kit | JL46166 |
| Mice IL-10 ELISA Kit | JL10138 |
| Mice IL-4 ELISA Kit | JL11806 |
| BCA Kit | JL48943 |
| Lysis buffer | Bioshar- |
| | BL504A |
| PMSF | P0100 |
| Deacetylase inhibitor cocktail | Tianenzhe |
| | 130521-1 |

### Example 70: Vitamin B12 Deficiency Mouse Model

Analyte determination in serum. Mouse serum samples from Example 65 would be used to measure analytes in multiplex panels 1, 2 and 3 using Luminex_LX 200 following the manufacturer recommendations. AYOXXA LUNARIS based method would be used to measure analytes in Panel 4 and 5 following the manufacturer recommendations. Panel descriptions are as described below.

Panel 1 would consist of mouse serum diluted 1:2 with the buffer provided in the kits (R&D customized panel) with the following analytes measured: Angiopoietin-2, BaFF/BLyS/TNPSP1311, ClqRl/CD93, MCP-1, CCL3/MTP-1 alpha, CCT A/MIP-1beta, CCLS/RANTES, CCI-1, 1/Eotaxin, CCI, 1 2/MCP-5, CCL20/MIP-3 alpha, CCL22/MDC, KC, MIP-2, IP-10, CXCL1 2/SDF-1 alpha, Dkk-1, EGP, PGP2, FGF-21, G-CSf, GM-CSR, IFN-gamma, IL-Iα/IL-F1, IL-1 beta/IL-1F2, IL-2, IL-3, IL-4, IL-6, IL-10, IL12, p70, IL-13, IL-17/IL-17 A, IL-17E/IL-25, IL-27, IL-33, Leptin/OB, LIX, M-CSF, TNF-alpha, and VEGF.

Panel 2 (R&D systems) would consist of 1 plex where Adiponectin is measured in 1:4000 diluted serum.

Panel 3 (R&D systems) would consist of 5 plex where Cystatin C, IVIMP2 IM P-2, IYIM P-3, MCP-2, and Adipsit are measured in 1:200 diluted serum.

Panel 4 (Millipore) would consist of 2 plex where Glucagon and Insulin are measured in 1:5 diluted serum.

Panel 5 (Millipore, Cardiovascular Disease) would consist of 3 plex where Troponin-T, Troponin-1, and sCD40L are measured in 1:20 diluted serum.

For the Luminex Assay Protocol, samples would be thawed at 4° C. prior to the start of assay and kept on ice throughout the assay procedures. Manufacturers' protocols would be followed for all panels with a general protocol as follows. All kit components would be brought to room temperature. Reagents are prepared according to the kit's instructions (wash buffers, beads, standards, etc.). Assay plates (96-well) are loaded with assay buffer, standards, samples, and beads and then covered and incubated on the plate shaker (500 rpm) overnight at 4° C. After the primary incubation, plates would be washed twice and then the detection antibody cocktail is added to all the wells; the plates are covered and left to incubate at room temperature for 1 hour on the plate shaker. After the one hour incubation, streptavidin-phycoerythrin fluorescent reporter will be added to all the wells, and the plate will be covered and incubated for 30 minutes at room temperature on the plate shaker. Plates are then washed twice, and the beads are resuspended in sheath fluid, placed on the shaker for 5 minutes, and then read on Bio-Plex®200 following manufacturers' specifications and using Bio-Plex Manager software v6.0. Samples are analysed following techniques known to those having skill in the art.

For the AYOXXA Assay Protocol, samples are thawed at 4° C. prior to the start of assay and kept on ice throughout the assay procedures. Sample dilutions would be prepared first in a 96 or 384 well plate for ease of transfer to the Lunaris™ BioChip. Manufacturers' protocols would be followed for all panels with a general protocol as follows: all kit components are brought to room temperature, with the exception of the SA-PE and antibodies. Reagents are prepared as per kit's instructions (wash buffers, standards, etc.). Assay plates are loaded with blanks, standards, and samples and then covered, centrifuged for 1 min at 700×g, and incubated at room temperature for 3 hours. Detection antibody is prepared 10 minutes prior to use. After the sample incubation, plates are washed three times and then the detection antibody cocktail are added to all the wells; the plates are covered, centrifuged, and left to incubate at room temperature for 1 hour. SA-PE is prepared 10 min prior to use. After the one hour incubation, the plates are washed three times and streptavidin-phycoerythrin fluorescent reporter is added to all the wells. The plate is covered, centrifuged, and incubated for 30 minutes at room temperature, and protected from light. Plates are then washed a total of 6 times and then dried for 1.5 hours in a sterile fume hood, avoiding direct light. Plates are then imaged on a specialized LUNARIS™ Reader™ using LUNARIS™ Control Software and LUNARIS™ Analysis Suite for readout settings known to those having skill in the art.

The presence of assay biomarkers in the samples, controls, and standards generates a fluorescent signal that is detected with a fluorescent microscope or Lunaris™ Reader. Quantification of the readout is performed entirely by the Lunaris™ Analysis Suite. Data generated included fluorescence intensity, observed concentration, LOD, LLOQ, and ULOQ exported as a Microsoft Excel file.

### Example 71: Vitamin B12 Deficiency Mouse Model Effect of Compound; on Plasma Organic Acid Levels

The effect of the compounds of the present disclosure on circulating concentrations of methylmalonic acid in plasma can be determined by liquid chromatography-tandem mass spectrometry (LC-MS/MS) after O-benzylhydroxylamine (O-BHA) derivatization under aqueous conditions. One can analyse sample processing by the procedure described in Example 65.

Sample preparation: Working Solutions of 5000 ng/mL D3-methylmalonic acid (d3-MMA, Sigma, 490318) and 5000 ng/mL D4-succinic acid (d4-SA, Sigma, 293075) would be prepared in methanol. 20 μL of working solutions will be diluted with methanol to a final volume of 200 μL, and the resulting Calibration Standard Solution containing 500 ng/ml d3-MMA and 500 ng/ml d4-SA is added into 50 μL of murine plasma. The samples would be thoroughly mixed, centrifuged (5800 rpm, 4° C., 10 min), and a 180 μL aliquot of supernatant is dried under a stream of nitrogen, before being reconstituted in 100 μL water and vortexed for 10 min. 50 μL of 1M O-benzylhydroxylamine (O-BHA) and 50 μL of 1M 1-ethyl-3-(3-dimethylamino) propyl carbodiimide hydrochloride (EDC) in pyridine buffer (50 mM Pyridine/acetic acid, pH 5.5) is added to the sample, which is mixed and incubated at room temperature. After 1 hr 500 μL ethyl acetate is added, and the plates would be shaken for 10 min using a vortexer, followed by centrifugation (5800 rpm, 4° C., 10 min). An aliquot of 400 μL supernatant would be dried under a stream of nitrogen, reconstituted in 150 μL methanol:water (50:50 v/v), vortexed and centrifuged (5800 rpm, 4° C., 10 min). 5 μL of supernatant is injected for LC-MS/MS analysis.

Sample analysis: A Triple Quad 6500 ACQUITY UPLC System (AB Sciex Instruments, API6500, triple quadruple) LC-MS/MS instrument would be used, controlled by Analyst 1.6.2 Software (AB Sciex Instruments). The chromatographic separation of the analytes will be performed on a Waters BEH C18 Column (2.1×50 mm, 1.7 μm) with a column temperature held at 60° C. Eluent A consisted of ultrapure water plus 0.1% formic acid (ULC-MS grade). Eluent B consisted of methanol plus 0.1% formic acid (ULC-MS grade). Gradient elution at a flow of 0.60 mL/min is performed by changing % B as follows: 0.0-1.0 min: 2% to 30%; 1.0-5.5 min: 30% to 40%; 5.5-5.6 min: 40% to 98%; 5.6-6.2 min: 98%; 6.2-6.3 min: 98% to 2%; 6.3-7.0 min: 2%.

All analytes and ISs are measured in positive electrospray ion mode; the dwell times are 20 ms each. Optimized MS/MS settings are summarized in the following Table

| | | | | Colli- | Reten- |
|---|---|---|---|---|---|
| | Parent | Daughter | Declustering | sion | tion |
| | mass | mass | potential | energy | time |
| Analyte | (Da) | (Da) | (DP) | (CE) | (min) |
| Methylmalonic | 329.1 | 91.1 | 60 | 38 | 3.5 |
| acid | | | | | |
| D3-Methylmalonic | 332.1 | 91.0 | 70 | 38 | 3.48 |
| acid | | | | | |
| Succinic acid | 329.1 | 206.0 | 60 | 14 | 3.35 |
| D4-Succinic acid | 333.3 | 210.1 | 60 | 14 | 3.33 |

The final Turbo Spray IonDrive source settings are the following: curtain gas flow 35 psig; collision gas 8 psig; nebulizer gas 60 psig; turbo gas 60 psig; source temperature (at setpoint) 500.0 C; entrance potential 10 V; collision cell exit potential 6 V.

Similar or non-derivatized liquid chromatography-tandem mass spectrometry (LC-MS/MS) methods can be used for the analysis of endogenous biomarkers, Acyl-CoA species (such as but not limited to Acetyl-CoA, Succinyl-CoA, Malonyl-CoA, TCA cycle intermediates and the like), Acyl-Carnitines, Carnitine and AcylCarnitine Transport and transporters, ketone bodies, Organic Acids, and other metabolites consistent with the biochemical and metabolic pathways, in biological samples including tissues (such as but not limited to liver, kidney, heart, muscle, bone, or skin tissue, and fluids, such as but not limited to blood, serum, plasma, urine, or cerebrospinal fluid). A variety of techniques known to those having skill in the art can be used, to extend these methods to alternative sample types, including but not limited to grinding, precipitation, centrifugation, and filtration.

### Example 72: Vitamin B12 Deficiency Mouse Model. Measuring Muscle Strength by Grip Test Analysis of Cbl−/− Mice Vs Mice on Control Diet

Mice at 18 weeks of age are either fed on control diet (n=12 mice) or cbl−/− diet (n=30 mice) for 15 weeks. Grip strength test is measured by using a Grip Strength test meter for mice and rats (Jiangsu, SANS Biological Technology, CO. LTD, SA417) and recorded as day zero grip strength analysis. Briefly, machine is turned on (push peak) and calibrated to zero. Front leg strength is measured by pulling the mice away from the bar by holding the tail of the mice. Total 5 values are measured for each mouse. After the measurement the cbl−/− mice are divided into three groups of N=10 mice per group. Each group receives either vehicle (0.1% saline) or compound, IP QD. Grip strength is again measured at defined time points in the above treated groups.

### EQUIVALENTS

The foregoing description has been presented only for the purposes of illustration and is not intended to limit the disclosure to the precise form disclosed. The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

What is claimed is:

1. A compound of Formula (I) or (II): 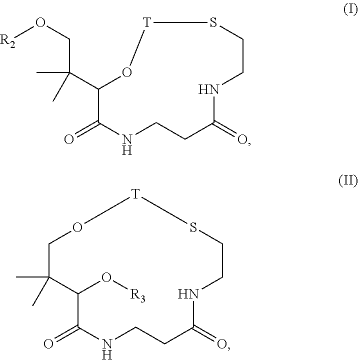
or a pharmaceutically acceptable salt or solvate thereof, wherein:
T is:
*—C(═O)—**,
*—C(═O)—(CH═CH)ₙ—C(═O)—**,
*—C(═O)—(CHR_{1b})ₙ—C(═O)—**,
*—C(═O)CH₂—[C(═O)CH₂]ₚ—(CH₂)_{q}—C(═O)—**,
*—C(═O)CH₂—[CH(OR_{1c})—CH₂]ₚ—(CH₂)_{q}—C(═O)—**,
*—C(═O)CH₂—[C(═O)CH₂]ₚ—[CH(OR_{1c})—CH₂]ᵣ—(CH₂)_{q}—C(═O)—**,
*—C(═O)CH₂—[CH(OR_{1c})—CH₂]ᵣ—[C(═O)CH₂]ₚ—(CH₂]_{q}—C(═O)—**,
*—C(═O)—(CHR_{1b})ₙ—[C(═O)CH₂]ₚ—(CH₂)_{q}—C(═O)—**,
*—C(═O)CH₂—[C(═O)—(CHR_{1b})ₙ]ₚ—(CH₂)_{q}—C(═O)—**,
*—C(═O)CH₂—[C(═O)CH₂]ₚ—(CHR_{1b})_{q}—C(═O)—**,
*—C(═O)—(CHR_{1b})ₙ—[C(═O)CH₂]ₚ—(CHR_{1b})_{q}—C(═O)—**,
**—C(═O)CH₂—[C(═O)CH₂]ₚ—(CH₂)_{q}—C(═O)—*,
**—C(═O)CH₂—[CH(OR_{1c})—CH₂]ₚ—(CH₂)_{q}—C(═O)—*,
**—C(═O)CH₂—[C(═O)CH₂]ₚ—[CH(OR_{1c})—CH₂]ᵣ—(CH₂)_{q}—C(═O)—*,
**—C(═O)CH₂—[CH(OR_{1c})—CH₂]r [C(═O)CH₂]ₚ—(CH₂)_{q}—C(═O)—*,
**—C(═O)—(CHR_{1b})ₙ —[C(═O)CH₂]ₚ—(CH₂)_{q}—C(═O)—*,
**—C(═O)CH₂—[C(═O)—(CHR_{1b})ₙ]ₚ—(CH₂)_{q}—C(═O)—*,
**—C(═O)CH₂—[C(═O)CH₂]ₚ—(CHR_{1b})_{q}—C(═O)—*,
**—C(═O)—(CHR_{1b})ₙ[C(═O)CH₂]ₚ—(CHR_{1b})_{q}—C(═O)—*, 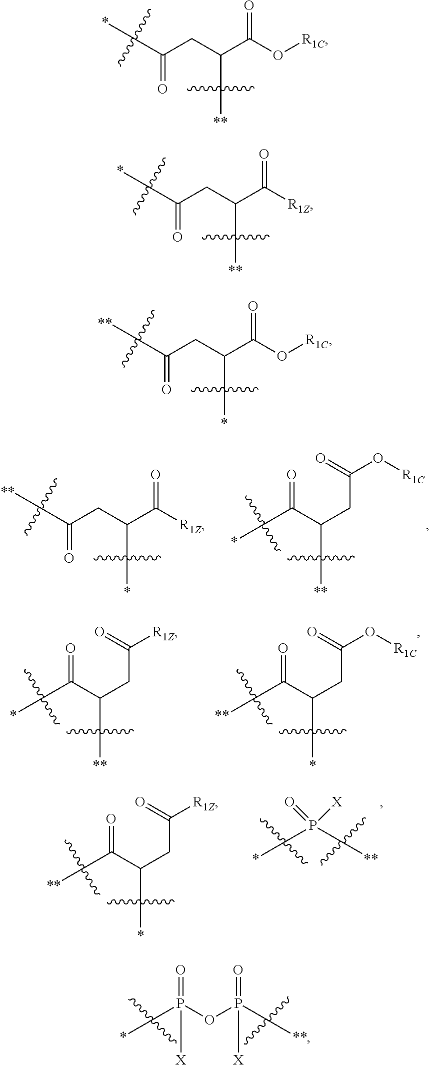
wherein * denotes an attachment of T to the sulfur atom, and ** denotes an attachment of T to the oxygen atom;
n is an integer ranging from 0 to 20;
p is an integer ranging from 0 to 20;
q is an integer ranging from 0 to 20;
r is an integer ranging from 0 to 20;
R₂ is H, —C(═O)R_{1b}, —C(═O)OR_{1c}, —C(═O)N(R_{1c})₂, —C(═O)R_{1z}, —C(═O)—C(═O)R_{1b}, —C(═O)—C(═O)OR_{1c}, —C(═O)—C(═O)N(R_{1c})₂, —C(═O)—C(═O)R_{1z}, —C(═O)—CH═CH—C(═O)OR_{1c}, —C(═O)—CH₂—CH₂—C(═O)OR_{1c}, 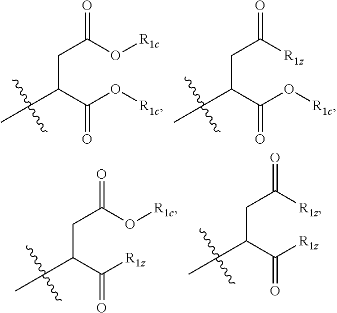
—C(═O)—CH═CH—C(═O)—R_{1z}, —C(═O)—CH₂—CH₂—C(═O)—R_{1z}, Si(R_{1g})₃, 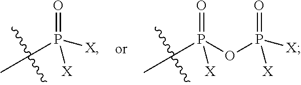
R₃ is H, —C(═O)R_{1b}, —C(═O)OR_{1c}, —C(═O)N(R_{1c})₂, —C(═O)R_{1z}, —C(═O)—C(═O)R_{1b}, —C(═O)—C(═O)OR_{1c}, —C(═O)—C(═O)N(R_{1c})₂, —C(═O)—C(═O)R_{1z}, —C(═O)—CH═CH—C(═O)OR_{1c}, —C(═O)—CH₂—CH₂—C(═O)OR_{1c}, 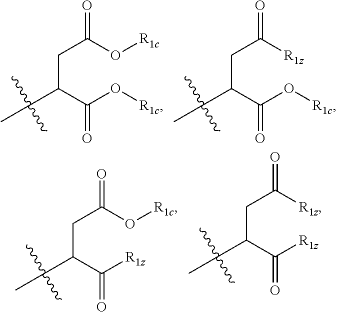
—C(═O)—CH═CH—C(═O)—R_{1z}, —C(═O)—CH₂—CH₂—C(═O)—R_{1z}, Si(R_{1g})₃, 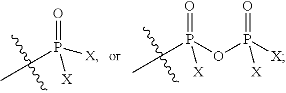
each R_{1b} is independently H, C₁-C₂₀ alkyl, C₂-C₂₀ alkenyl, C₂-C₂₀ alkynyl, —(CH₂)_{q}—C(═O)OR_{1c}, —CH₂—C(═O)—(CH₂)_{q}—C(═O)OR_{1c}, —CH₂—[C(═O)CH₂]ₚ—[CH₂]_{q}—C(═O)OR_{1c}, —CH═CH—C(═O)OR_{1c}, —C(═O)OR_{1c}, —C(═O)N(R_{1c})₂, or R_{1z}, wherein the C₁-C₂₀ alkyl, C₂-C₂₀ alkenyl, or C₂-C₂₀ alkynyl is optionally substituted with one or more R_{1c};
each R_{1c} is independently H, C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl), wherein the C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) is optionally substituted with one or more R₁ₑ;
each R_{1d} is independently H, C₁-C₂₀ alkyl, C₂-C₂₀ alkenyl, C₂-C₂₀ alkynyl, C₃-C₁₀ cycloalkyl, C₃-C₁₂ cycloalkyl, C₃-C₁₂ heterocycloalkyl, C₃-C₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl), wherein the C₁-C₂₀ alkyl, C₂-C₂₀ alkenyl, C₂-C₂₀ alkynyl, C₃-C₁₂ cycloalkyl, C₂-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₁₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) is optionally substituted with one or more R₁ₑ;
each R_{1c} is independently H, halogen, C₁-C₂₀ alkyl, C₂-C₂₀ alkenyl, C₂-C₂₀ alkynyl, —OR_{1g}, —C(═O)OR_{1g}, —C(═O)N(R_{1g})₂, —N(R_{1g})₂, —N(R_{1g})C(═O)R_{1f}, —N(R_{1g})C(═O)R_{1z}, —N(R_{1g})C(═O)OR_{1g}, —OC(═O)R_{1f}, —OC(═O)R_{1z}, —OC(═O)OR_{1g}, —SR_{1g}, —N⁺(R_{1g})₃, —SC(═O)R_{1f}—, —SC(═O)R_{1z}, —SC(═O)OR_{1g}, —SC(═O)N(R_{1g})₂, —C(═O)R_{1f}, —C(═O)R_{1z}, 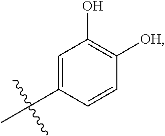 [SMILES: *c1ccc(O)c(O)c1]
or R_{1z}, wherein the C₁-C₂₀ alkyl, C₂-C₂₀ alkenyl, or C₂-C₂₀ alkynyl is optionally substituted with one or more R_{1z};
each R_{1f} is independently H, C₁-C₂₀ alkyl, C₂-C₂₀ alkenyl, C₂-C₂₀ alkynyl, —CH₂C(═O)OR_{1g}, —CH═CH—C(═O)OR_{1g}, —C(═O)OR_{1g}, —C(═O)N(R_{1g})₂, 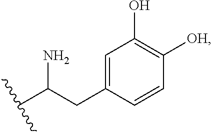 [SMILES: *C(N)Cc1ccc(O)c(O)c1]
or R_{1z}, wherein the C₁-C₂₀ alkyl, C₂-C₂₀ alkenyl, or C₂-C₂₀ alkynyl is optionally substituted with one or more R_{1z};
each R_{1g} is independently H, C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl), wherein the C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) is optionally substituted with one or more R_{1z};
each R_{1z} is independently 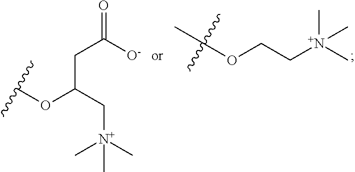
and
each X is independently —OR_{1c}, —SR_{1c}, —N(R_{1c})₂, 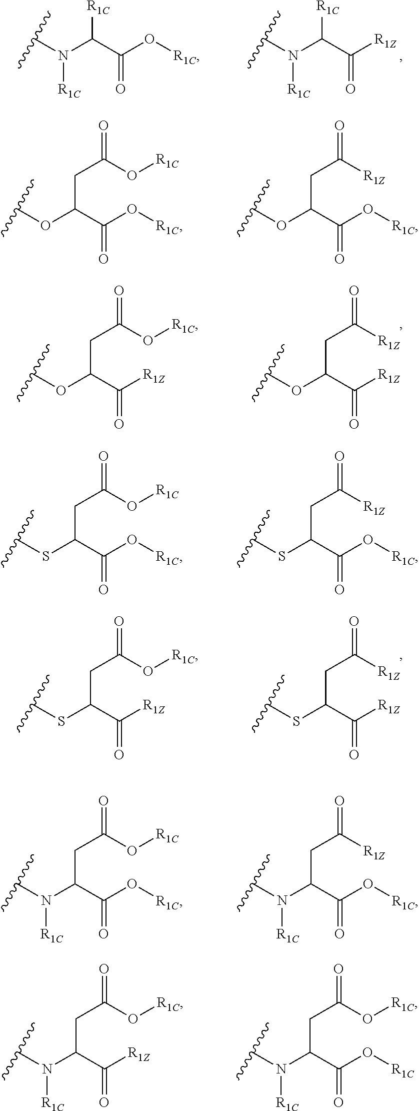
or R_{1z}.

2. A compound of Formula (I) or (II): 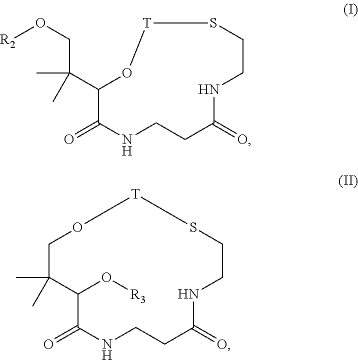
or a pharmaceutically acceptable salt or solvate thereof, wherein:
T is *—C(═O)—(CHR_{1b})ₙ—C(═O)—**;
wherein * denotes an attachment of T to the sulfur atom, and ** denotes an attachment of T to the oxygen atom;
n is an integer ranging from 0 to 20;
R₂ is H, —C(═O)R_{1b}, or Si(R_{1g})₃;
R₃ is H, —C(═O)R_{1b}, or Si(R_{1g})₃;
each R_{1b} is independently H or C₁-C₂₀ alkyl, wherein the C₁-C₂₀ alkyl is optionally substituted with one or more R₁ₑ;
each R_{1c} is independently H, C₁-C₂₀ alkyl, or —N(R_{1g})₂;
each R_{1g} is independently H or C₁-C₂₀ alkyl.

3. The compound of any one of the preceding claims, wherein T is *—C(═O)—**.

4. The compound of any one of the preceding claims, wherein T is *—C(═O)—(CH═CH)ₙ—C(═O)—**, *—C(═O)—(CHR_{1b})ₙ—C(═O)—**, *—C(═O)CH₂—[C(═O)CH₂]ₚ—(CH₂)_{q}—C(═O)—**, *—C(═O)CH₂—[CH(OR_{1c})—CH₂]ₚ—(CH₂)_{q}—C(═O)—**, *—C(═O)CH₂—[C(═O)CH₂]ₚ—[CH(OR_{1c})—CH₂]ᵣ—(CH₂)_{q}—C(═O)—**, *—C(═O)CH₂—[CH(OR_{1c})—CH₂]r [C(═O)CH₂]ₚ—(CH₂)_{q}—C(═O)—**, *—C(═O)—(CHR_{1b})—[C(═O)CH₂]ₚ—(CH₂)_{q}—C(═O)—**, *—C(═O)CH₂—[C(═O)—(CHR_{1b})ₙ]ₚ—(CH₂)_{q}—C(═O)—**, *—C(═O)CH₂—[C(═O)CH₂]ₚ—(CHR_{1b})_{q}—C(═O)—**, *—C(═O)—(CHR_{1b})ₙ—[C(═O)CH₂]ₚ—(CHR_{1b})_{q}—C(═O)—**, **—C(═O)CH₂—[C(═O)CH₂]ₚ—(CH₂)_{q}—C(═O)—*, **—C(═O)CH₂—[CH(OR_{1c})—CH₂]ₚ—(CH₂)_{q}—C(═O)—*, **—C(═O)CH₂—[C(═O)CH₂]ₚ—[CH(OR_{1c})—CH₂]ᵣ—(CH₂)_{q}—C(═O)—*, **—C(═O)CH₂—[CH(OR_{1c})—CH₂]r [C(═O)CH₂]ₚ—(CH₂)_{q}—C(═O)—*, **—C(═O)—(CHR_{1b})ₙ—[C(═O)CH₂]ₚ—(CH₂)_{q}—C(═O)—*, **—C(═O)CH₂—[C(═O)—(CHR_{1b})ₙ]ₚ—(CH₂)_{q}—C(═O)—*, **—C(═O)CH₂—[C(═O)CH₂]ₚ—(CHR_{1b})_{q}—C(═O)—*, **—C(═O)—(CHR_{1b})ₙ—[C(═O)CH₂]ₚ—(CHR_{1b})_{q}—C(═O)—*,

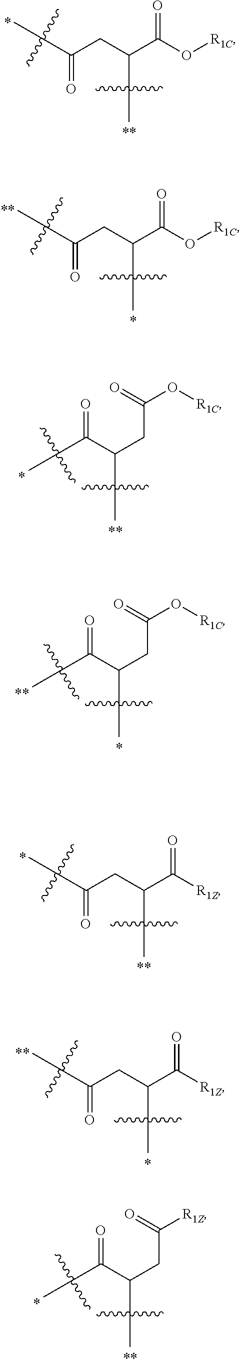

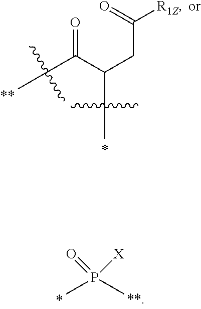

5. The compound of any one of the preceding claims, wherein T is *—C(═O)—(CH═CH)ₙ—C(═O)—**.

6. The compound of any one of the preceding claims, wherein T is *—C(═O)—C(═O)—**.

7. The compound of any one of the preceding claims, wherein T is *—C(═O)—CH═CH—C(═O)—**.

8. The compound of any one of the preceding claims, wherein T is *—C(═O)—(CHR_{1b})ₙ—C(═O)—**.

9. The compound of any one of the preceding claims, wherein T is *—C(═O)—CHR_{1b}—C(═O)—**.

10. The compound of any one of the preceding claims, wherein T is *—C(═O)—CH₂—C(═O)—**.

11. The compound of any one of the preceding claims, wherein T is *—C(═O)—(CHR_{1b})(CHR_{1b})—C(═O)—**.

12. The compound of any one of the preceding claims, wherein T is *—C(═O)—CH₂CH₂—C(═O)—**.

13. The compound of any one of the preceding claims, wherein T is *—C(═O)CH₂—[C(═O)CH₂]ₚ—(CH₂)_{q}—C(═O)—**.

14. The compound of any one of the preceding claims, wherein T is *—C(═O)CH₂—C(═O)CH₂—CH₂—C(═O)—**.

15. The compound of any one of the preceding claims, wherein T is *—C(═O)CH₂—[CH(OR_{1c})—CH₂]ₚ—(CH₂)_{q}—C(═O)—**.

16. The compound of any one of the preceding claims, wherein T is *—C(═O)CH₂—[C(═O)CH₂]ₚ—[CH(OR_{1c})—CH]ᵣ—(CH₂)_{q}—C(═O)—**.

17. The compound of any one of the preceding claims, wherein T is *—C(═O)CH₂—[CH(OR_{1c})—CH₂]—[C(═O)CH₂]ₚ—(CH₂)_{q}—C(═O)—**.

18. The compound of any one of the preceding claims, wherein T is *—C(═O)—(CHR_{1b})ₙ—[C(═O)CH₂]ₚ—(CH₂)_{q}—C(O)—**.

19. The compound of any one of the preceding claims, wherein T is *—C(═O)CH₂—[C(═O)—(CHR_{1b})ₙ]ₚ—(CH₂)_{q}—C(═O)—**.

20. The compound of any one of the preceding claims, wherein T is *—C(═O)CH₂—[C(═O)CH₂]ₚ—(CHR_{1b})_{q}—C(═O)—**.

21. The compound of any one of the preceding claims, wherein T is *—C(═O)—(CHR_{1b})ₙ—[C(═O)CH₂]ₚ—(CHR_{1b})_{q}—C(═O)—**.

22. The compound of any one of the preceding claims, wherein T is **—C(═O)CH₂—[C(═O)CH₂]ₚ—(CH₂)_{q}—C(═O)—*.

23. The compound of any one of the preceding claims, wherein T is **—C(═O)CH₂—C(═O)CH₂—CH₂—C(═O)—*.

24. The compound of any one of the preceding claims, wherein T is **—C(═O)CH₂—[CH(OR_{1c})—CH₂]ₚ—(CH₂)_{q}—C(O)—*.

25. The compound of any one of the preceding claims, wherein T is **—C(═O)CH₂—[C(═O)CH₂]ₚ—[CH(OR_{1c})—CH]ᵣ—(CH₂)_{q}—C(═O)—*.

26. The compound of any one of the preceding claims, wherein T is **—C(═O)CH₂—[CH(OR_{1c})—CH₂]ᵣ—[C(═O)CH₂]ₚ—(CH₂)_{q}—C(═O)—*.

27. The compound of any one of the preceding claims, wherein T is **—C(═O)—(CHR_{1b})ₙ—[C(═O)CH₂]ₚ—(CH₂)_{q}—C(═O)—*.

28. The compound of any one of the preceding claims, wherein T is **—C(═O)CH₂—[C(═O)—(CHR_{1b})ₙ]ₚ—(CH₂)_{q}—C(═O)—*.

29. The compound of any one of the preceding claims, wherein T is **—C(═O)CH₂—[C(═O)CH₂]ₚ—(CHR_{1b})_{q}—C(═O)—*.

30. The compound of any one of the preceding claims, wherein T is **—C(═O)—(CHR_{1b})ₙ—[C(═O)CH₂]ₚ—(CHR_{1b})_{q}—C(═O)—*.

31. The compound of any one of the preceding claims, wherein T is

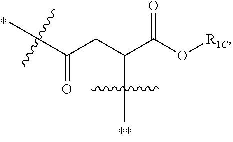

32. The compound of any one of the preceding claims, wherein T is

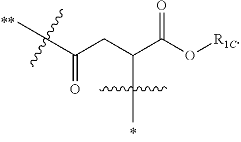

33. The compound of any one of the preceding claims, wherein T is

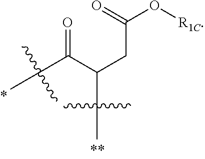

34. The compound of any one of the preceding claims, wherein T is

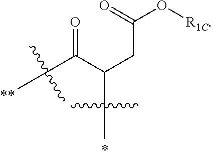

35. The compound of any one of the preceding claims, wherein T is

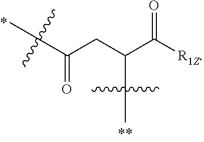

36. The compound of any one of the preceding claims, wherein T is

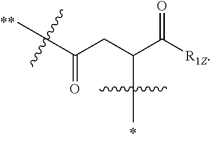

37. The compound of any one of the preceding claims, wherein T is

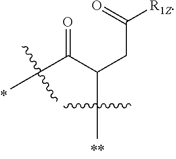

38. The compound of any one of the preceding claims, wherein T is

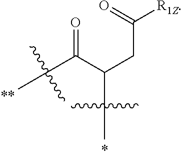

39. The compound of any one of the preceding claims, wherein T is

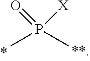

40. The compound of any one of the preceding claims, wherein T is 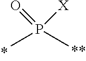
wherein X is —OR_{1c}.

41. The compound of any one of the preceding claims, wherein T is 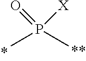
wherein X is —SR_{1c}.

42. The compound of any one of the preceding claims, wherein T is 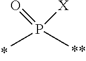
wherein X is —N(R_{1c})₂.

43. The compound of any one of the preceding claims, wherein T is 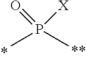
wherein X is

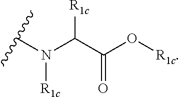

44. The compound of any one of the preceding claims, wherein T is 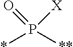
wherein X is

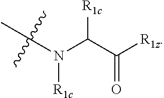

45. The compound of any one of the preceding claims, wherein T is

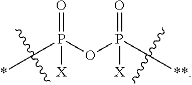

46. The compound of any one of the preceding claims, wherein R₂ is H.

47. The compound of any one of the preceding claims, wherein R₂ is —C(═O)R_{1b}, —C(═O)OR_{1c}, —C(═O)N(R_{1c})₂, —C(═O)R_{1z}, —C(═O)—C(═O)R_{1b}, —C(═O)—C(═O)OR_{1c}, —C(═O)—C(═O)N(R_{1c})₂, —C(═O)—C(═O)R_{1z}, —C(═O)—CH═CH—C(═O)OR_{1c}, —C(═O)—CH₂—CH₂—C(═O)OR_{1c}, 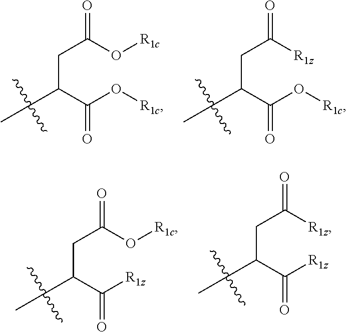
—C(═O)—CH═CH—C(═O)—R_{1z}, —C(═O)—CH₂—CH₂—C(═O)—R_{1z},

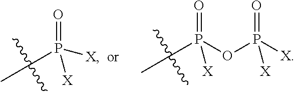

48. The compound of any one of the preceding claims, wherein R₂ is —C(═O)R_{1b}.

49. The compound of any one of the preceding claims, wherein R₂ is —(CH₂)_{q}—C(═O)OR_{1c}.

50. The compound of any one of the preceding claims, wherein R₂ is —CH₂CH₂—C(═O)OR_{1c}.

51. The compound of any one of the preceding claims, wherein R₂ is —CH₂—C(═O)—(CH₂)_{q}—C(═O)OR_{1c}.

52. The compound of any one of the preceding claims, wherein R₂ is —CH₂—C(═O)—CH₂CH₂—C(═O)OR_{1c}.

53. The compound of any one of the preceding claims, wherein R₂ is —C(═O)—CH═CH—C(═O)OR_{1c}.

54. The compound of any one of the preceding claims, wherein R₂ is —C(═O)R_{1z}.

55. The compound of any one of the preceding claims, wherein R₂ is —C(═O)—C(═O)R_{1b}.

56. The compound of any one of the preceding claims, wherein R₂ is —C(═O)—C(═O)OR_{1c}.

57. The compound of any one of the preceding claims, wherein R₂ is —C(═O)—C(═O)N(R_{1c})₂.

58. The compound of any one of the preceding claims, wherein R₂ is —C(═O)—C(═O)R_{1z}.

59. The compound of any one of the preceding claims, wherein R₂ is —C(═O)OR_{1c}.

60. The compound of any one of the preceding claims, wherein R₂ is —C(═O)N(R_{1c})₂.

61. The compound of any one of the preceding claims, wherein R₂ is —C(═O)—CH═CH—C(═O)OR_{1c}.

62. The compound of any one of the preceding claims, wherein R₂ is —C(═O)—CH₂—CH₂—C(═O)OR_{1c}.

63. The compound of any one of the preceding claims, wherein R₂ is

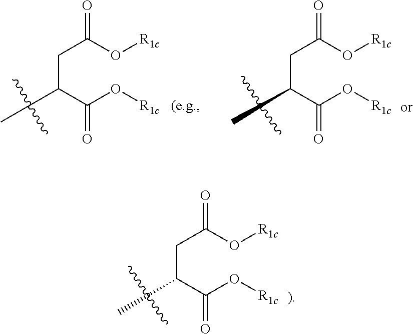

64. The compound of any one of the preceding claims, wherein R₂ is

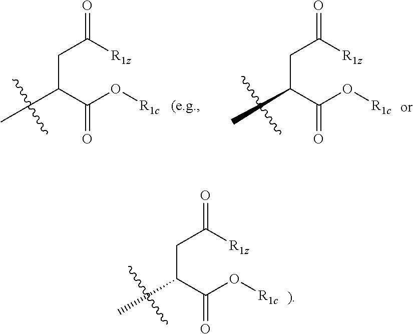

65. The compound of any one of the preceding claims, wherein R₂ is

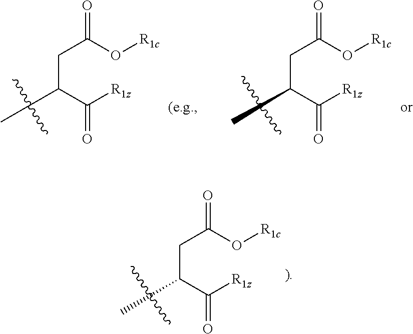

66. The compound of any one of the preceding claims, wherein R₂, is

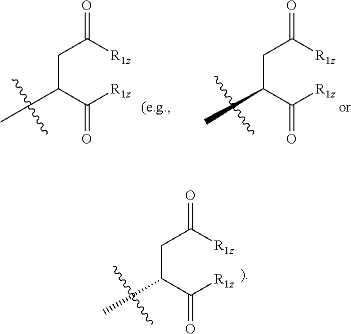

67. The compound of any one of the preceding claims, wherein R₂ is —C(═O)—CH═CH—C(═O)—R_{1z}.

68. The compound of any one of the preceding claims, wherein R₂ is —C(═O)—CH₂—CH₂—C(═O)—R_{1z}.

69. The compound of any one of the preceding claims, wherein R₂ is Si(R_{1g})₃.

70. The compound of any one of the preceding claims, wherein R₂ is

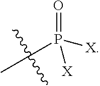

71. The compound of any one of the preceding claims, wherein R₂ is

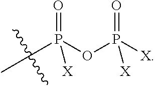

72. The compound of any one of the preceding claims, wherein R₃ is H.

73. The compound of any one of the preceding claims, wherein R₃ is —C(═O)R_{1b}, —C(═O)OR_{1c}, —C(═O)N(R_{1c})₂, —C(═O)R_{1z}, —C(═O)—C(═O)R_{1b}, —C(═O)—C(═O)OR_{1c}, —C(═O)—C(═O)N(R_{1c})₂, —C(═O)—C(═O)R_{1z}, —C(═O)—CH═CH—C(═O)OR_{1c}, —C(═O)—CH₂—CH₂—C(═O)OR_{1c}, 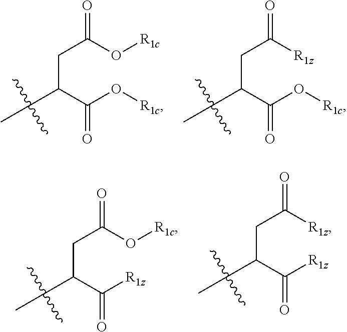
—C(═O)—CH═CH—C(═O)—R_{1z}, —C(═O)—CH₂—CH₂—C(═O)—R_{1z},

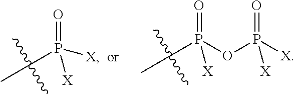

74. The compound of any one of the preceding claims, wherein R₃ is —C(═O)R_{1b}.

75. The compound of any one of the preceding claims, wherein R₃ is —(CH₂)_{q}—C(═O)OR_{1c}.

76. The compound of any one of the preceding claims, wherein R₃ is —CH₂CH₂—C(═O)OR_{1c}.

77. The compound of any one of the preceding claims, wherein R₃ is —CH₂—C(═O)—(CH₂)_{q}—C(═O)OR_{1c}.

78. The compound of any one of the preceding claims, wherein R₃ is —CH₂—C(═O)—CH₂CH₂—C(═O)OR_{1c}.

79. The compound of any one of the preceding claims, wherein R₃ is —C(═O)—CH═CH—C(═O)OR_{1c}.

80. The compound of any one of the preceding claims, wherein R₃ is —C(═O)R_{1z}.

81. The compound of any one of the preceding claims, wherein R₃ is —C(═O)—C(═O)R_{1b}.

82. The compound of any one of the preceding claims, wherein R₃ is —C(═O)—C(═O)OR_{1c}.

83. The compound of any one of the preceding claims, wherein R₃ is —C(═O)—C(═O)N(R_{1c})₂.

84. The compound of any one of the preceding claims, wherein R₃ is —C(═O)—C(═O)R_{1z}.

85. The compound of any one of the preceding claims, wherein R₃ is —C(═O)OR_{1c}.

86. The compound of any one of the preceding claims, wherein R₃ is —C(═O)N(R_{1c})₂.

87. The compound of any one of the preceding claims, wherein R₃ is —C(═O)—CH═CH—C(═O)OR_{1c}.

88. The compound of any one of the preceding claims, wherein R₃ is —C(═O)—CH₂—CH₂—C(═O)OR_{1c}.

89. The compound of any one of the preceding claims, wherein R₃ is

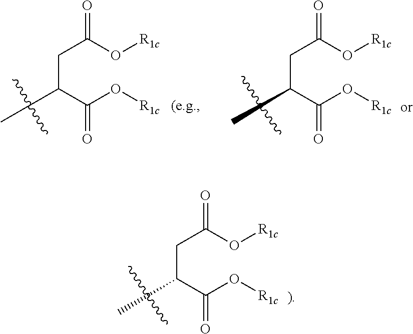

90. The compound of any one of the preceding claims, wherein R₃ is

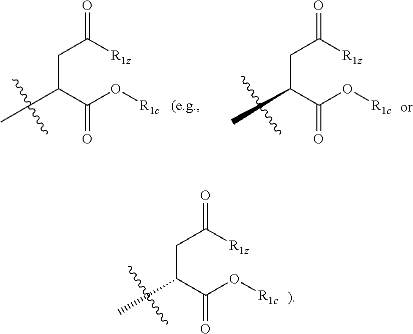

91. The compound of an one of the receding claims, wherein R₃ is

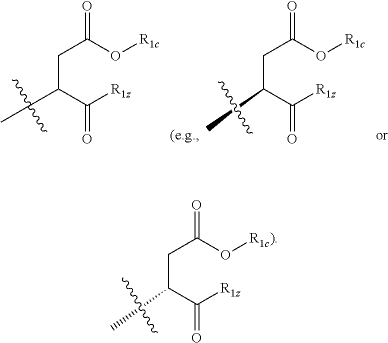

92. The compound of any one of the preceding claims, wherein R₃ is

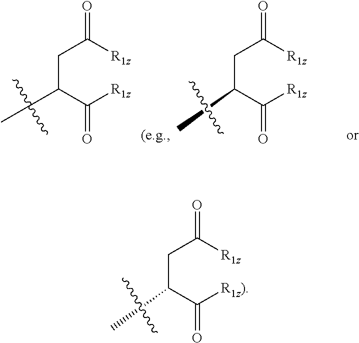

93. The compound of any one of the preceding claims, wherein R₃ is —C(═O)—CH═CH—C(═O)—R_{1z}.

94. The compound of any one of the preceding claims, wherein R₃ is —C(═O)—CH₂—CH₂—C(═O)—R_{1z}.

95. The compound of any one of the preceding claims, wherein R₃ is Si(R_{1g})₃.

96. The compound of any one of the preceding claims, wherein R₃ is

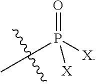

97. The compound of any one of the preceding claims, wherein R₃ is

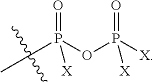

98. The compound of any one of the preceding claims, wherein at least one R_{1b} is H.

99. The compound of any one of the preceding claims, wherein at least one R_{1b} is C₁-C₂₀ alkyl, C₂-C₂₀ alkenyl, or C₂-C₂₀ alkynyl optionally substituted with one or more R₁ₑ.

100. The compound of any one of the preceding claims, wherein at least one R_{1b} is —(CH₂)_{q}—C(═O)OR_{1c}, —CH₂—C(═O)—(CH₂)_{q}—C(═O)OR_{1c}, —CH₂—[C(═O)CH₂]ₚ—[CH₂]_{q}—C(═O)OR_{1c}, —CH═CH—C(═O)OR_{1c}, —C(═O)OR_{1c}, —C(═O)N(R_{1c})₂, or R_{1z}.

101. The compound of any one of the preceding claims, wherein at least one R_{1c} is H.

102. The compound of any one of the preceding claims, wherein at least one R_{1c} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) is optionally substituted with one or more R₁ₑ.

103. The compound of any one of the preceding claims, wherein at least one R_{1d} is H.

104. The compound of any one of the preceding claims, wherein at least one R_{1d} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₁-C₂₀ alkyl)-(C₃-C₁₂ heterocycloalkyl), —(C₁-C₂₀ alkyl)-(C₃-C₁₂ aryl), or —(C₁-C₂₀ alkyl)-(C₃-C₁₂ heteroaryl) is optionally substituted with one or more R₁ₑ.

105. The compound of any one of the preceding claims, wherein at least one R₁ₑ is H.

106. The compound of any one of the preceding claims, wherein at least one R₁ₑ is halogen, C₁-C₂₀ alkyl, C₂-C₂₀ alkenyl, C₂-C₂₀ alkynyl, —OR_{1g}, —C(═O)OR_{1g}, —C(═O)N(R_{1g})₂, —N(R_{1g})₂, —N(R_{1g})C(═O)R_{1f}, —N(R_{1g})C(═O)R_{1z}, —N(R_{1g})C(═O)OR_{1g}, —OC(═O)R_{1f}, —OC(═O)R_{1z}, —OC(═O)OR_{1g}, —SR_{1g}, —N⁺(R_{1g})₃, —SC(═O)R_{1f}, —SC(═O)R_{1z}, —SC(═O)OR_{1g}, —SC(═O)N(R_{1g})₂, —C(═O)R_{1f}, —C(═O)R_{1z}, 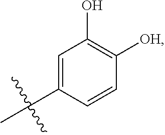 [SMILES: *c1ccc(O)c(O)c1]
or R_{1z}, wherein the C₁-C₂₀ alkyl, C₂-C₂₀ alkenyl, or C₂-C₂₀ alkynyl is optionally substituted with one or more R_{1z}.

107. The compound of any one of the preceding claims, wherein at least one R_{1f} is H.

108. The compound of any one of the preceding claims, wherein at least one R_{1f} is C₁-C₂₀ alkyl, C₂-C₂₀ alkenyl, C₂-C₂₀ alkynyl, —CH₂C(═O)OR_{1g}, —CH═CH—C(═O)OR_{1g}, —C(═O)OR_{1g}, —C(═O)N(R_{1g})₂, 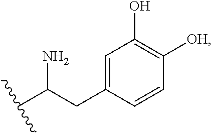 [SMILES: *C(N)Cc1ccc(O)c(O)c1]
or R_{1z}, wherein the C₁-C₂₀ alkyl, C₂-C₂₀ alkenyl, or C₂-C₂₀ alkynyl is optionally substituted with one or more R_{1z}.

109. The compound of any one of the preceding claims, wherein at least one R_{1g} is H.

110. The compound of any one of the preceding claims, wherein at least one R_{1g} is C₁-C₂₀ alkyl, C₂-C₂₀ alkenyl, C₂-C₂₀ alkynyl, C₃-C₁₂ cycloalkyl, C₃-C₁₂ heterocycloalkyl, C₃-C₁₂ aryl, C₃-C₁₂ heteroaryl, —(C₁-C₂₀ alkyl)-(C₃-C₁₂ cycloalkyl), —(C₃-C₂₀ alkyl)-(C₃-C₁₂ heterocycloalkyl), —(C₁-C₂₀ alkyl)-(C₃-C₁₂ aryl), or —(C₁-C₁₀ alkyl)-(C₃-C₁₂ heteroaryl) is optionally substituted with one or more R_{1z}.

111. The compound of any one of the preceding claims, wherein at least one R_{1z} is

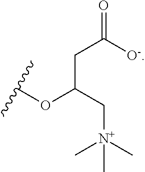

112. The compound of any one of the preceding claims, wherein at least one R_{1z} is

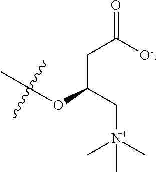

113. The compound of any one of the preceding claims, wherein at least one R_{1z} is

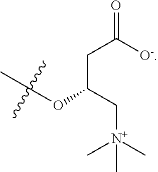

114. The compound of any one of the preceding claims, wherein at least one R_{1z} is

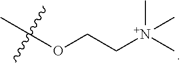 [SMILES: *OCC[N+](C)(C)C]

115. The compound of any one of the receding claims, wherein all of the one or more R_{1z} is

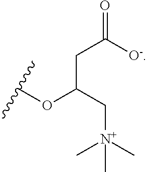

116. The compound of any one of the preceding claims, wherein all of the one or more R_{1z} is

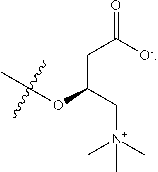

117. The compound of any one of the preceding claims, wherein all of the one or more R_{1z} is

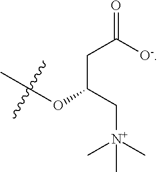

118. The compound of any one of the preceding claims, wherein all of the one or more R_{1z} is

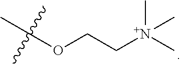 [SMILES: *OCC[N+](C)(C)C]

119. The compound of any one of the preceding claims, wherein at least one of the two or more R_{1z} is 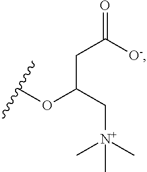
and at least one of the two or more R_{1z} is

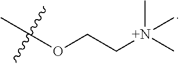 [SMILES: *OCC[N+](C)(C)C]

120. The compound of any one of the preceding claims, wherein at least one of the two or more R_{1z} is 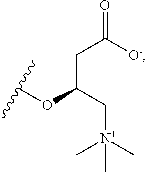
and at least one of the two or more R_{1z} is

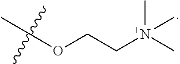 [SMILES: *OCC[N+](C)(C)C]

121. The compound of any one of the preceding claims, wherein at least one of the two or more R_{1z} is 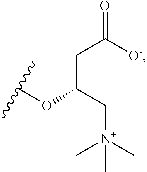
and at least one of the two or more R_{1z} is

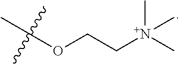 [SMILES: *OCC[N+](C)(C)C]

122. The compound of any one of the preceding claims, wherein n is from 0 to 20, from 0 to 15, from 0 to 10, from 0 to 6, from 0 to 4, or from 0 to 2.

123. The compound of any one of the preceding claims, wherein n is from 1 to 20, from 2 to 20, from 3 to 20, from 4 to 20, from 5 to 20, from 6 to 20, from 7 to 20, from 8 to 20, from 9 to 20, from 10 to 20, from 11 to 20, from 12 to 20, from 13 to 20, from 14 to 20, from 15 to 20, from 16 to 20, from 17 to 20, from 18 to 20, or from 19 to 20.

124. The compound of any one of the preceding claims, wherein p is from 0 to 20, from 0 to 15, from 0 to 10, from 0 to 6, from 0 to 4, or from 0 to 2.

125. The compound of any one of the preceding claims, wherein p is from 1 to 20, from 2 to 20, from 3 to 20, from 4 to 20, from 5 to 20, from 6 to 20, from 7 to 20, from 8 to 20, from 9 to 20, from 10 to 20, from 11 to 20, from 12 to 20, from 13 to 20, from 14 to 20, from 15 to 20, from 16 to 20, from 17 to 20, from 18 to 20, or from 19 to 20.

126. The compound of any one of the preceding claims, wherein q is from 0 to 20, from 0 to 15, from 0 to 10, from 0 to 6, from 0 to 4, or from 0 to 2.

127. The compound of any one of the preceding claims, wherein q is from 1 to 20, from 2 to 20, from 3 to 20, from 4 to 20, from 5 to 20, from 6 to 20, from 7 to 20, from 8 to 20, from 9 to 20, from 10 to 20, from 11 to 20, from 12 to 20, from 13 to 20, from 14 to 20, from 15 to 20, from 16 to 20, from 17 to 20, from 18 to 20, or from 19 to 20.

128. The compound of any one of the preceding claims, wherein r is from 0 to 20, from 0 to 15, from 0 to 10, from 0 to 6, from 0 to 4, or from 0 to 2.

129. The compound of any one of the preceding claims, wherein r is from 1 to 20, from 2 to 20, from 3 to 20, from 4 to 20, from 5 to 20, from 6 to 20, from 7 to 20, from 8 to 20, from 9 to 20, from 10 to 20, from 11 to 20, from 12 to 20, from 13 to 20, from 14 to 20, from 15 to 20, from 16 to 20, from 17 to 20, from 18 to 20, or from 19 to 20.

130. The compound of any one of the preceding claims, wherein at least one X is —OR_{1c}.

131. The compound of any one of the preceding claims, wherein at least one X is —SR_{1c}.

132. The compound of any one of the preceding claims, wherein at least one X is —N(R_{1c})₂.

133. The compound of any one of the preceding claims, wherein at least one X is

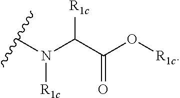

134. The compound of any one of the preceding claims, wherein at least one X is

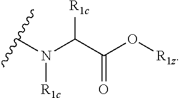

135. The compound of any one of the preceding claims, wherein at least one X is

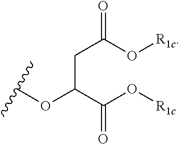

136. The compound of any one of the preceding claims, wherein at least one X is

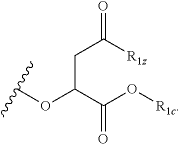

137. The compound of any one of the preceding claims, wherein at least one X is

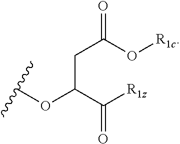

138. The compound of any one of the preceding claims, wherein at least one X is

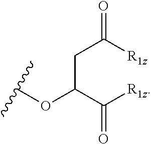

139. The compound of any one of the preceding claims, wherein at least one X is

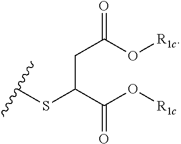

140. The compound of an one of the preceding claims, wherein at least one X is

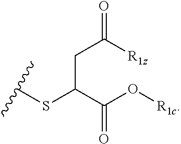

141. The compound of any one of the preceding claims, wherein at least one X is

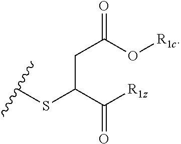

142. The compound of an one of the preceding claims, wherein at least one X is

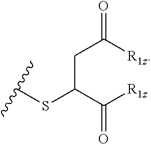

143. The compound of any one of the preceding claims, wherein at least one X is

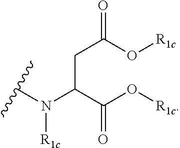

144. The compound of any one of the preceding claims, wherein at least one X is

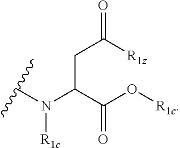

145. The compound of any one of the preceding claims, wherein at least one X is

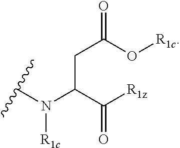

146. The compound of any one of the preceding claims, wherein at least one X is

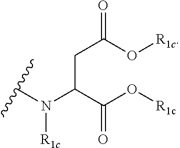

147. The compound of any one of the preceding claims, wherein at least one X is R_{1z}.

148. The compound of any one of the preceding claims, wherein at least one X is

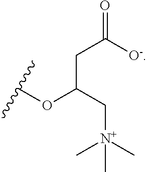

149. The compound of any one of the preceding claims, wherein at least one X is

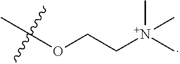 [SMILES: *OCC[N+](C)(C)C]

150. The compounds of any on of the preceding claims being one of Formulae (I-1) or (I-2): 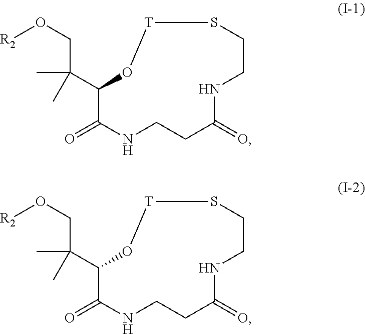
or a pharmaceutically acceptable salt or solvate thereof.

151. The compound of any one of the preceding claims, being one of Formulae (Ia-1) or (Ia-2): 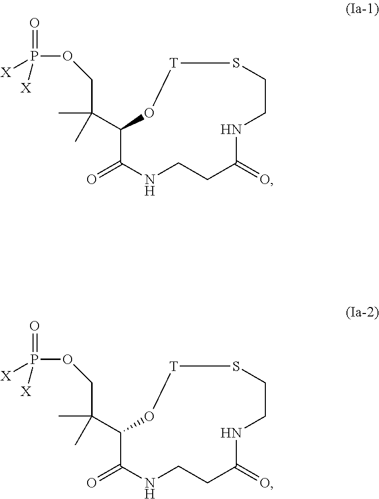
or a pharmaceutically acceptable salt or solvate thereof.

152. The compound of any one of the preceding claims, being one of Formulae (Ib), (Ic), (Td), or (Ie): 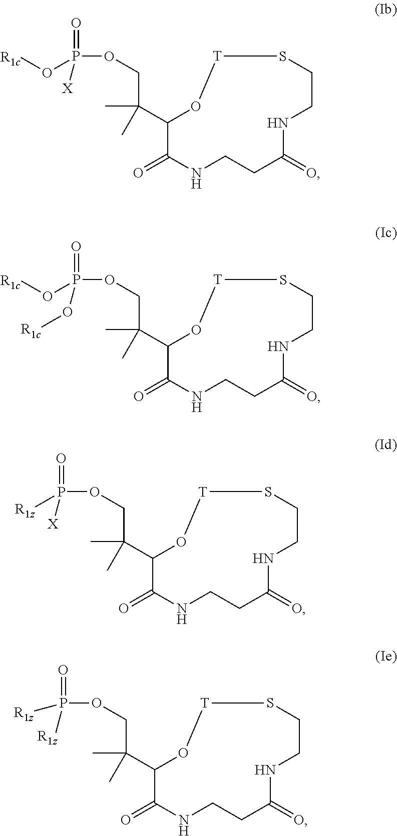
or a pharmaceutically acceptable salt or solvate thereof.

153. The compound of any one of the preceding claims, being one of Formula (Ib-1), (Ib-2), (Ic-1), (Ic-2), (Id-1), (Id-2), (Ie-1), or (Ie-2): 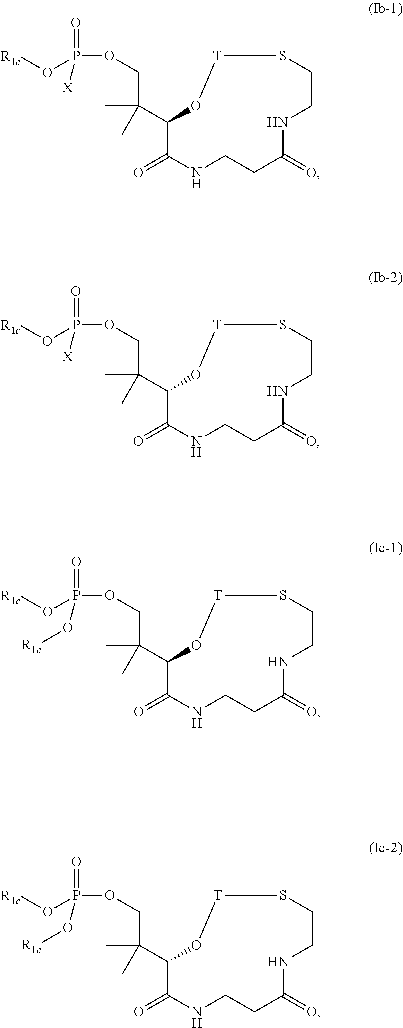 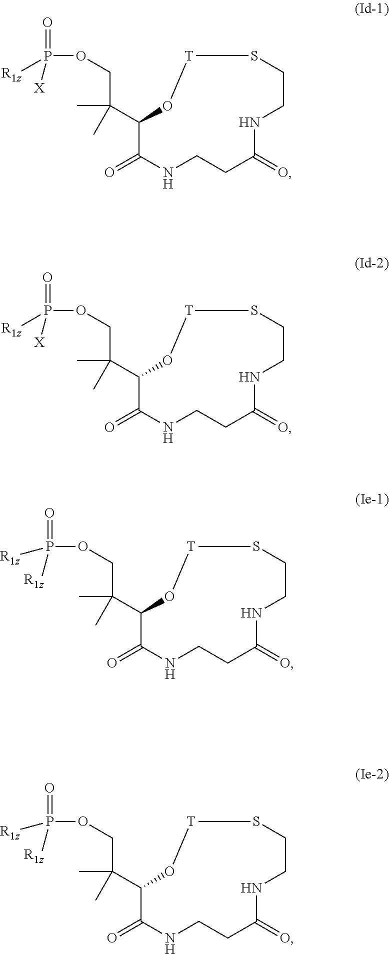
or a pharmaceutically acceptable salt or solvate thereof.

154. The compound of any one of the preceding claims, being one of Formulae (If), (Ig), (Ih), or (Ii): 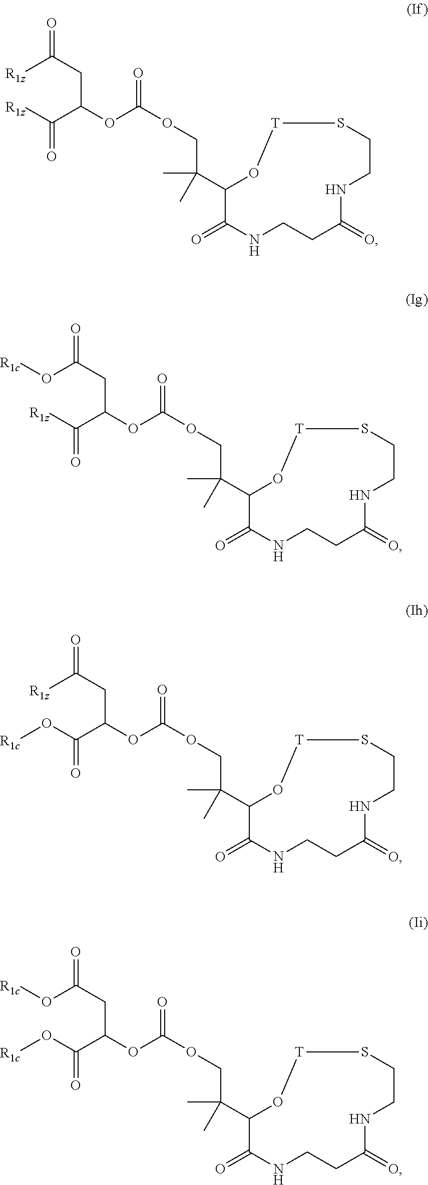
or a pharmaceutically acceptable salt or solvate thereof.

155. The compound of any one of the preceding claims, being one of Formula (If-1), (If-2), (Ig-1), (Ig-2), (Ih-1), (Ih-2), (Ii-1), or (Ii-2): 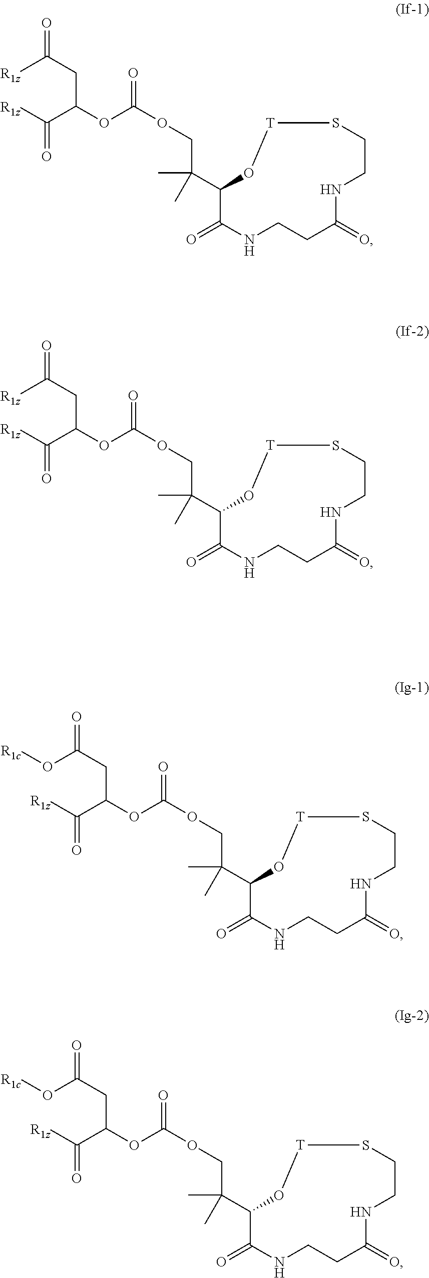 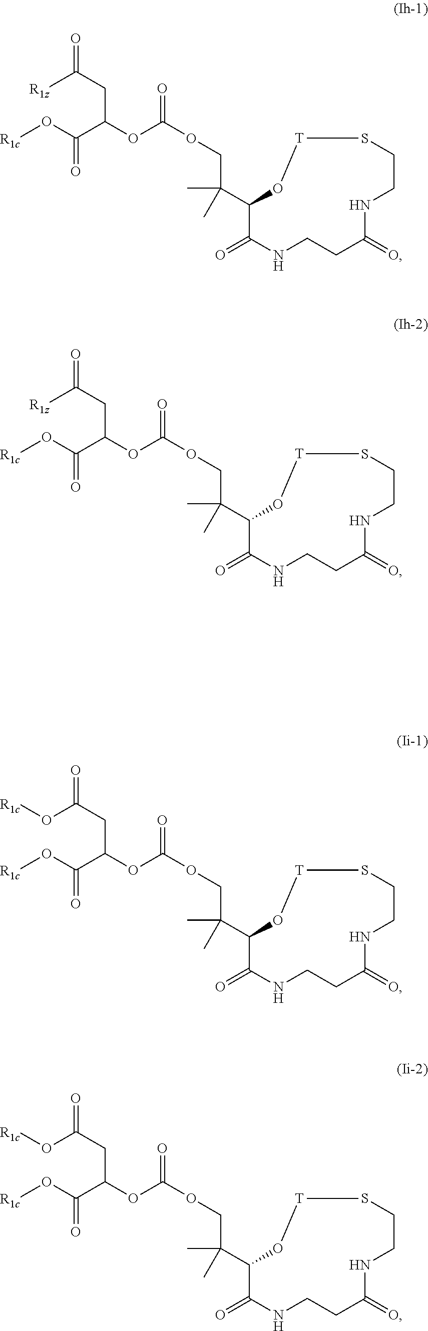
or a pharmaceutically acceptable salt or solvate thereof.

156. The compound of any one of the preceding claims, being of Formula (Ij): 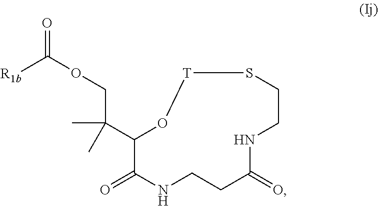
or a pharmaceutically acceptable salt or solvate thereof.

157. The compound of any one of the preceding claims, being one of Formulae (Ij-1) or (Ij-2): 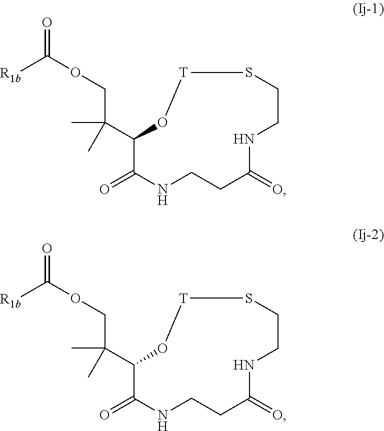
or a pharmaceutically acceptable salt or solvate thereof.

158. The compound of any one of the preceding claims, being of Formula (Ik): 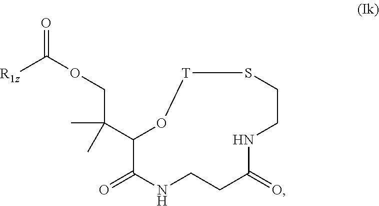
or a pharmaceutically acceptable salt or solvate thereof.

159. The compound of any one of the preceding claims, being one of Formulae (Ik-1) or (Ik-2): 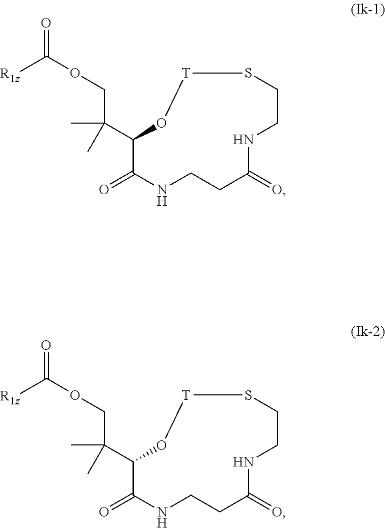
or a pharmaceutically acceptable salt or solvate thereof.

160. The compound of any one of the preceding claims, being of Formulae Formula (Il): 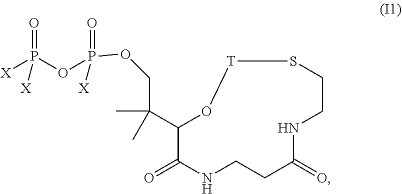
or a pharmaceutically acceptable salt or solvate thereof.

161. The compound of any one of the preceding claims, being one of Formula (Il-1) or (Il-2): 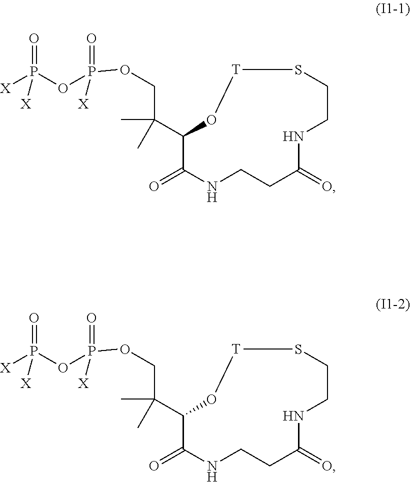
or a pharmaceutically acceptable salt or solvate thereof.

162. The compound of any one of the preceding claims, being of Formulae Formula (Im): 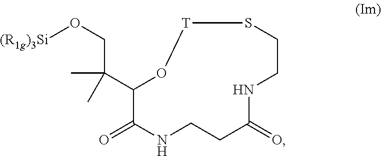
or a pharmaceutically acceptable salt or solvate thereof.

163. The compound of any one of the preceding claims, being one of Formula (Im-1) or (Im-2): 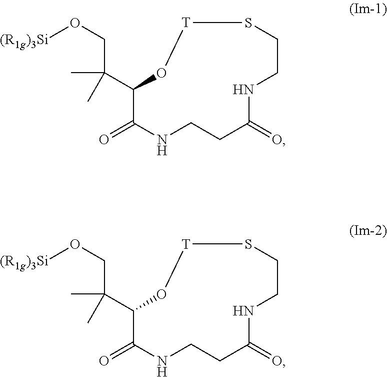
or a pharmaceutically acceptable salt or solvate thereof.

164. The compound of any one of the preceding claims, being of Formula (In): 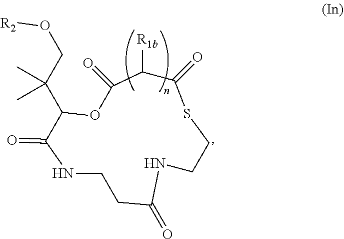
or a pharmaceutically acceptable salt or solvate thereof.

165. The compound of any one of the preceding claims, being one of Formulae (In-1) or (In-2): 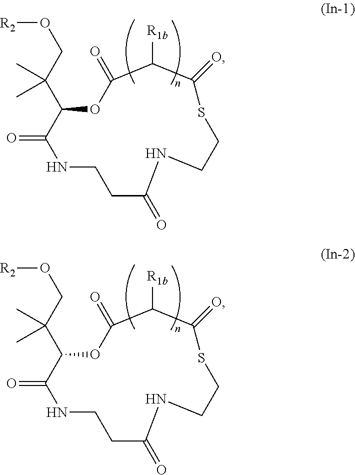
or a pharmaceutically acceptable salt or solvate thereof.

166. The compound of any one of the preceding claims, being of Formula (Io): 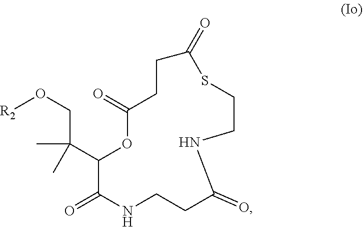
or a pharmaceutically acceptable salt or solvate thereof.

167. The compound of any one of the preceding claims, being one of Formulae (Io-1) or (Io-2): 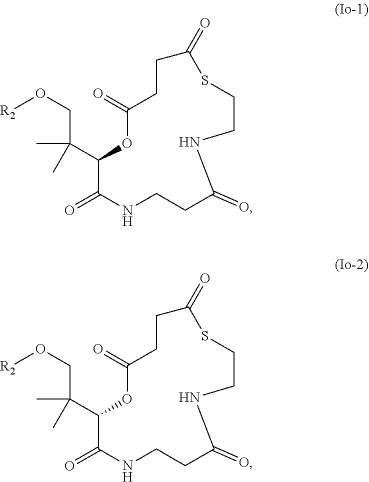
or a pharmaceutically acceptable salt or solvate thereof.

168. The compounds of any one of the preceding claims, being one of Formulae (II-1) or (II-2): 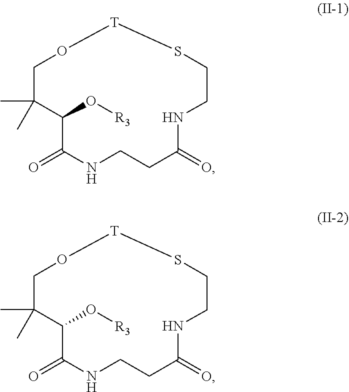
or a pharmaceutically acceptable salt or solvate thereof.

169. The compound of any one of the preceding claims, being of Formula (IIa): 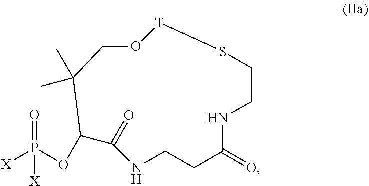
or a pharmaceutically acceptable salt or solvate thereof.

170. The compound of any one of the preceding claims, being one of Formulae (IIa-1) or (IIa-2): 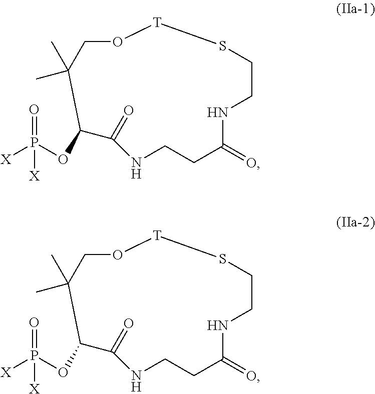
or a pharmaceutically acceptable salt or solvate thereof.

171. The compound of any one of the preceding claims, being one of Formulae (IIb), (IIc), (IId), or (IIe): 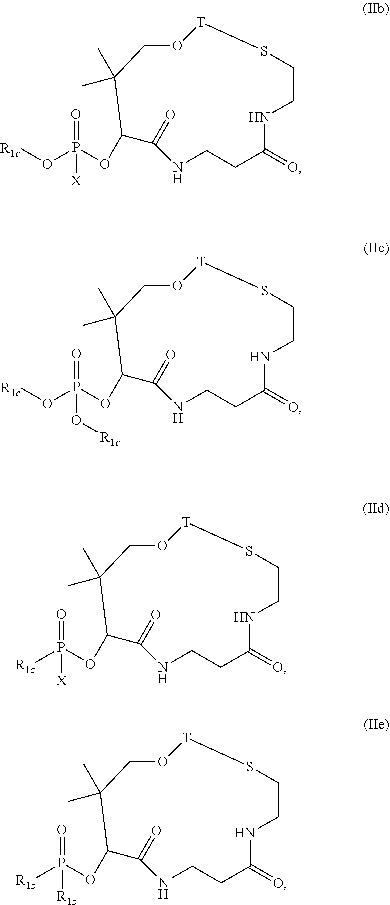
or a pharmaceutically acceptable salt or solvate thereof.

172. The compound of any one of the preceding claims, being one of Formula (IIb-1), (IIb-2), (IIc-1), (IIc-2), (IId-1), (IId-2), (IIe-1), or (IIe-2): 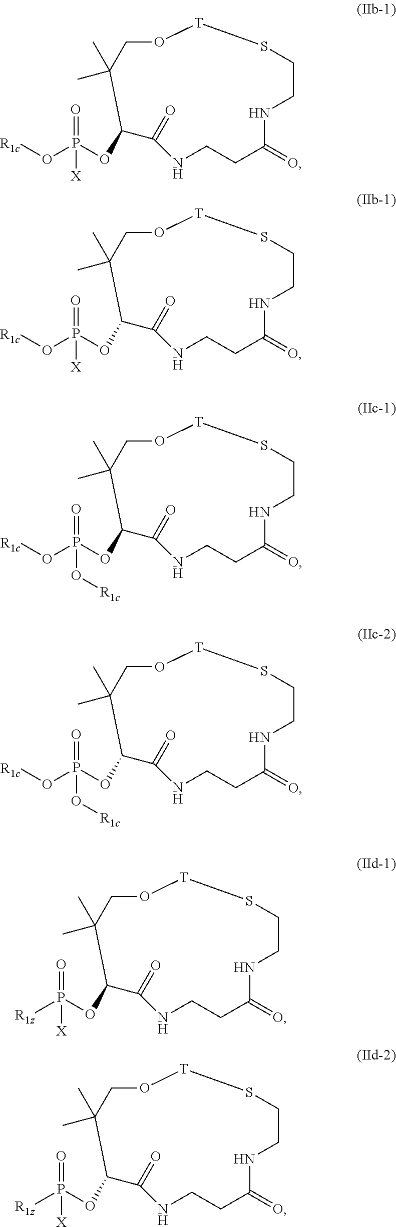 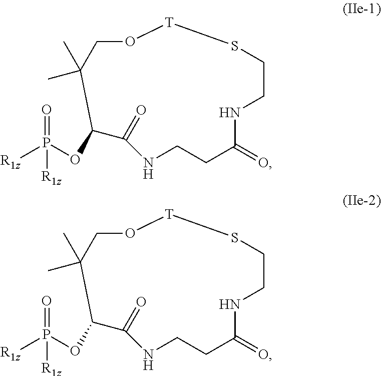
or a pharmaceutically acceptable salt or solvate thereof.

173. The compound of any one of the preceding claims, being one of Formulae (IIf), (IIg), (IIh), or (IIi): 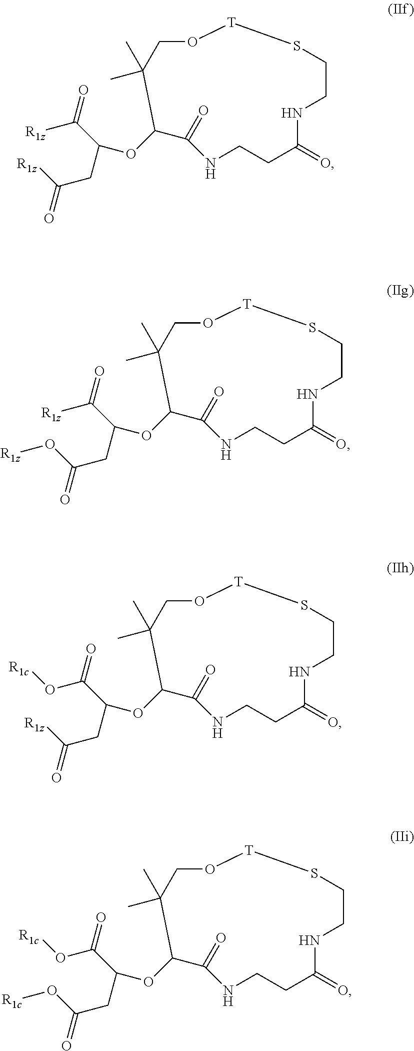
or a pharmaceutically acceptable salt or solvate thereof.

174. The compound of any one of the preceding claims, being one of Formula (IIf-1), (IIf-2), (IIg-1), (IIg-2), (IIh-1), (IIh-2), (IIi-1), or (IIi-2): 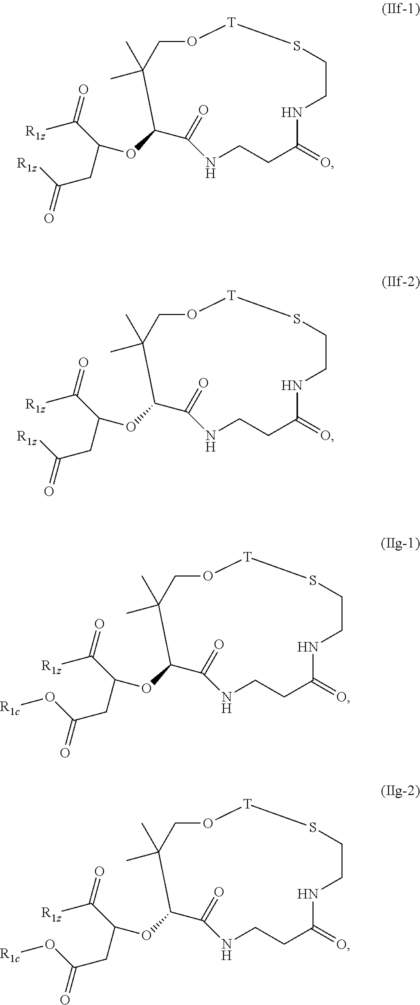 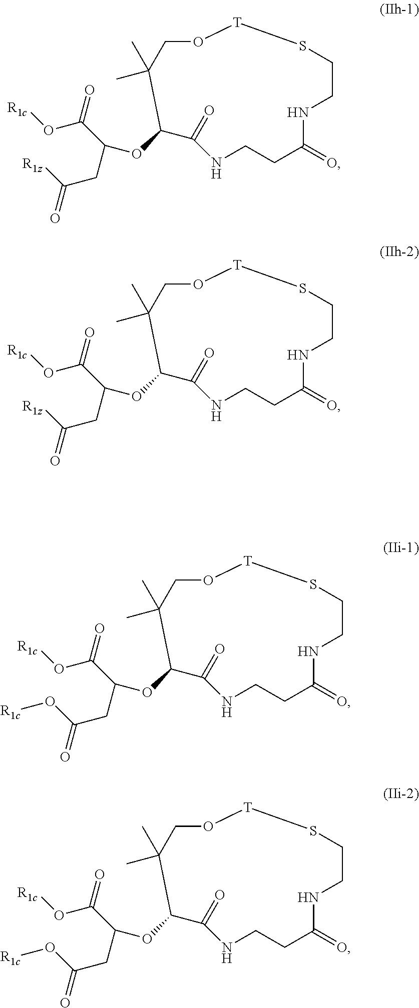
or a pharmaceutically acceptable salt or solvate thereof.

175. The compound of any one of the preceding claims, being of Formula (IIj): 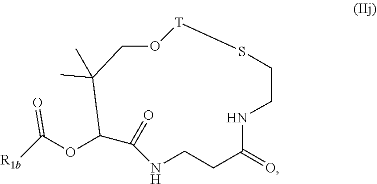
or a pharmaceutically acceptable salt or solvate thereof.

176. The compound of any one of the preceding claims, being one of Formulae (IIj-1) or (IIj-2): 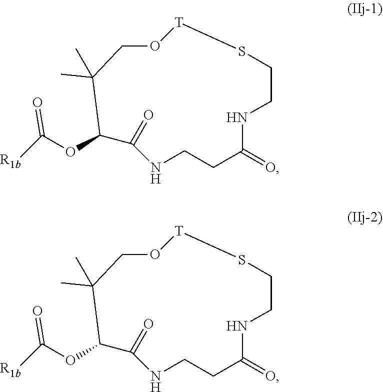
or a pharmaceutically acceptable salt or solvate thereof.

177. The compound of any one of the preceding claims, being of Formula (IIk): 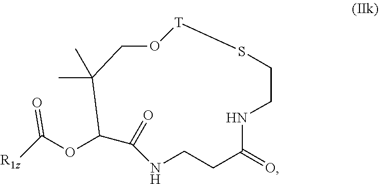
or a pharmaceutically acceptable salt or solvate thereof.

178. The compound of any one of the preceding claims, being one of Formulae (IIk-1) or (IIk-2): 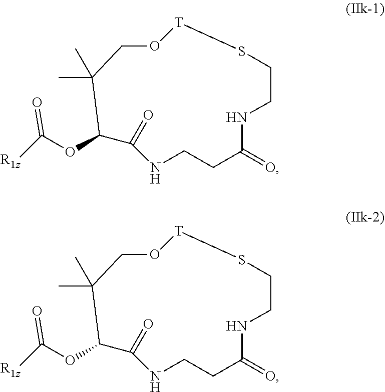
or a pharmaceutically acceptable salt or solvate thereof.

179. The compound of any one of the preceding claims, being of Formula (III): 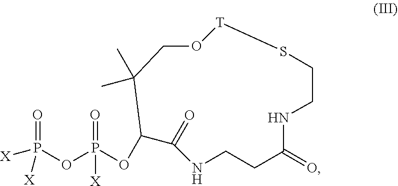
or a pharmaceutically acceptable salt or solvate thereof.

180. The compound of any one of the preceding claims, being one of Formulae (IIl-1) or (IIl-2): 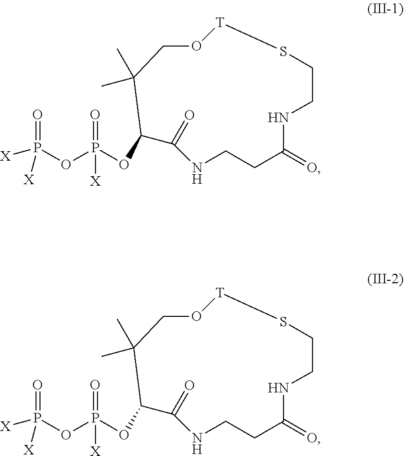
or a pharmaceutically acceptable salt or solvate thereof.

181. The compound of any one of the preceding claims, being of Formula (IIm): 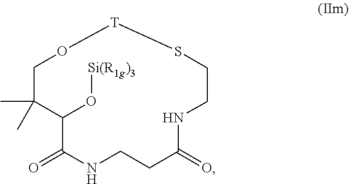
or a pharmaceutically acceptable salt or solvate thereof.

182. The compound of any one of the preceding claims, being one of Formulae (IIm-1) or (IIm-2): 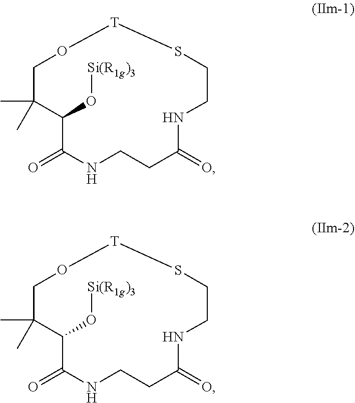
or a pharmaceutically acceptable salt or solvate thereof.

183. The compound of any one of the preceding claims, being of Formula (IIn): 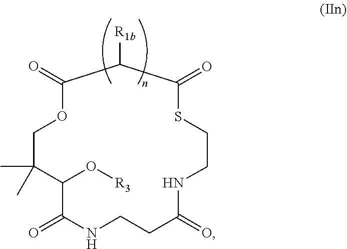
or a pharmaceutically acceptable salt or solvate thereof.

184. The compound of any one of the preceding claims, being one of Formulae (IIn-1) or (IIn-2): 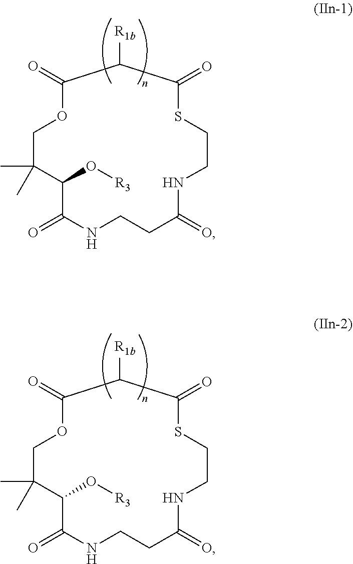
or a pharmaceutically acceptable salt or solvate thereof.

185. The compound of any one of the preceding claims, being of Formula (IIo): 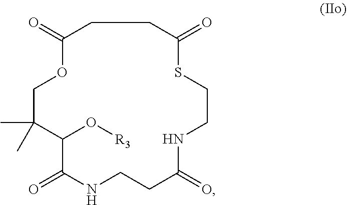
or a pharmaceutically acceptable salt or solvate thereof.

186. The compound of any one of the preceding claims, being one of Formulae (IIo-1) or (IIo-2): 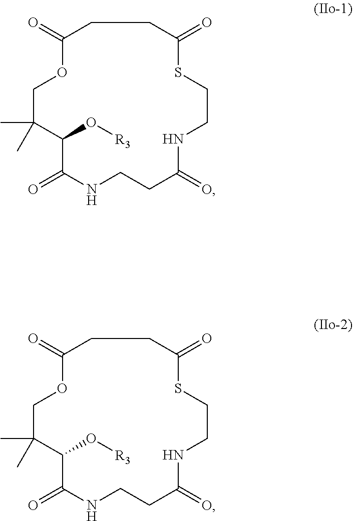
or a pharmaceutically acceptable salt or solvate thereof.

187. The compound of any one of the preceding claims, being selected from the compounds described in Tables 1-3, and pharmaceutically acceptable salts thereof.

188. The compound of any one of the preceding claims, being selected from the compounds described in Table 1 and pharmaceutically acceptable salts thereof.

189. The compound of any one of the preceding claims, being selected from the compounds described in Table 2 and pharmaceutically acceptable salts thereof.

190. The compound of any one of the preceding claims, being selected from the compounds described in Table 3 and pharmaceutically acceptable salts thereof.

191. The compound of any one of the preceding claims, being selected from Compound Nos. 1-2, 16, 18, 41-42, 56, 58, 61-62, 76, 78, 101-102, 116, 118, 146-147, 161, 163, 186-187, 201, 203, 206, 209, 236, 240, 246-247, 261, 263, 339-340, and pharmaceutically acceptable salts thereof.

192. The compound of any one of the preceding claims, being selected from Compound Nos. 1-2, 58, 61-62, 76, 78, 101, 116, 118, 186, 206, 209, 339-340, and pharmaceutically acceptable salts thereof.

193. The compound of any one of the preceding claims, being selected from Compound Nos. 1-2, 58, 101, 116, 118, 186, 209, and pharmaceutically acceptable salts thereof.

194. The compound of any one of the preceding claims, being selected from Compound Nos. 61-62, 76, 78, 206, 339-340, and pharmaceutically acceptable salts thereof.

195. A compound being an isotopic derivative of the compound of any one of the preceding claims.

196. A compound being an intermediate obtained by a method for preparing the compound of any one of the preceding claims.

197. A pharmaceutical composition comprising the compound of any one of the preceding claims or a pharmaceutically acceptable salt thereof.

198. A method of activating or enhancing acetyl-CoA synthesis in a subject comprising administering to the subject a therapeutically effective amount a compound of any one of the preceding claims.

199. A method of increasing acetyl-CoA concentrations in a subject comprising administering to the subject a therapeutically effective amount of a compound of any one of the preceding claims.

200. A method of treating a subject having a disease comprising administering to the subject a therapeutically effective amount of a compound of any one of the preceding claims.

201. A method of preventing a disease in a subject comprising administering to the subject a therapeutically effective amount of a compound of any one of the preceding claims.

202. A method of decreasing degradation of CoA in a subject comprising administering to the subject a therapeutically effective amount of a compound of any one of the preceding claims.

203. A method of increasing the half-life of CoA in a subject comprising administering to the subject a therapeutically effective amount of a compound of any one of the preceding claims.

204. A method of prolonging the availability of CoA in a subject comprising administering to the subject a therapeutically effective amount of a compound of any one of the preceding claims.

205. A method of delivering an acyl moiety into the mitochondrial matrix of a mitochondrion of a subject comprising administering to the subject a therapeutically effective amount of a compound of any one of the preceding claims.

206. A method of decreasing the concentration of reactive oxygen species (ROS) in a subject comprising administering to the subject a therapeutically effective amount of a compound of any one of the preceding claims.

207. A method of decreasing the concentration of an at least one acyl-CoA species in a subject comprising administering to the subject a therapeutically effective amount of a compound of any one of the preceding claims.

208. A method of increasing the fatty acid metabolism in a subject comprising administering to the subject a therapeutically effective amount of a compound of any one of the preceding claims.

209. A method of increasing the amino acid metabolism in a subject comprising administering to the subject a therapeutically effective amount of a compound of any one of the preceding claims.

210. A method of increasing mitochondrial respiration in a subject comprising administering to the subject a therapeutically effective amount of a compound of any one of the preceding claims.

211. A method of increasing ATP concentration in a subject comprising administering to the subject therapeutically effective amount of a compound of any one of the preceding claims.

212. A method of treating a subjecting having a cancer comprising administering to the subject a therapeutically effective amount of a compound of any one of the preceding claims.

213. A method of preventing a cancer in a subject comprising administering to the subject a therapeutically effective amount of a compound of any one of the preceding claims.

214. A method of reducing the size of a tumor comprising administering to the subject a therapeutically effective amount of a compound of any one of the preceding claims.

215. A method of inducing tumor cell apoptosis in a subject comprising administering to the subject a therapeutically effective amount of a compound of any one of the preceding claims.

216. A method of inducing cell cycle arrest in a tumor cell in a subject comprising administering to the subject a therapeutically effective amount of a compound of any one of the preceding claims.

217. A method of inducing differentiation of a cell in a subject comprising administering to the subject a therapeutically effective amount of a compound of any one of the preceding claims.

218. A method of inducing senescence in a cell in a subject comprising administering to the subject a therapeutically effective amount of a compound of any one of the preceding claims.

219. A method of enhancing an immune response against cancer in a subject comprising administering to the subject a therapeutically effective amount of a compound of any one of the preceding claims.

220. A method of inhibiting angiogenesis in a subject comprising administering to the subject a therapeutically effective amount of a compound of any one of the preceding claims.

221. A method of enhancing the apoptotic effect of an anti-cancer agent comprising administering to a subject a combination of a therapeutically effective amount of the anti-cancer agent and a therapeutically effective amount of a compound of any one of the preceding claims.

222. A method of increasing the post-translational modification of proteins in a subject comprising administering to the subject a therapeutically effective amount of a compound of any one of the preceding claims.

223. A method of increasing acetylation of proteins in a subject comprising administering to the subject a therapeutically effective amount of a compound of any one of the preceding claims.

224. A method of increasing acetylation of histones in a subject comprising administering to the subject a therapeutically effective amount of a compound of any one of the preceding claims.

225. A method of increasing acetylation of tubulin in a subject comprising administering to the subject a therapeutically effective amount of a compound of any one of the preceding claims.

226. A method of treating a subject having a neurodegenerative disease comprising administering to the subject a therapeutically effective amount of a compound of any one of the preceding claims.

227. A method of preventing a neurodegenerative disease in a subject comprising administering to the subject a therapeutically effective amount of a compound of any one of the preceding claims.

228. A method of treating a subject having an ataxia disease comprising administering to the subject a therapeutically effective amount of a compound of any one of the preceding claims.

229. A method of preventing an ataxia in a subject comprising administering to the subject a therapeutically effective amount of a compound of any one of the preceding claims.

230. A method of treating nonalcoholic steatohepatitis in a subject comprising administering to the subject a therapeutically effective amount of a compound of any one of the preceding claims.

231. A method of preventing nonalcoholic steatohepatitis in a subject comprising administering to the subject a therapeutically effective amount of a compound of any one of the preceding claims.

232. A method of treating nonalcoholic fatty liver disease in a subject comprising administering to the subject a therapeutically effective amount of a compound of any one of the preceding claims.

233. A method of preventing nonalcoholic fatty liver disease in a subject comprising administering to the subject a therapeutically effective amount of a compound of any one of the preceding claims.

234. A method of preventing an inappropriate shift to fatty acid biosynthesis in a subject comprising administering to the subject a therapeutically effective amount of a compound of any one of the preceding claims.

235. A method of treating an inflammatory disease in a subject comprising administering to the subject a therapeutically effective amount of a compound of any one of the preceding claims.

236. A method of preventing an inflammatory disease in a subject comprising administering to the subject a therapeutically effective amount of a compound of any one of the preceding claims.

237. A method of reducing inflammation in a subject comprising administering to the subject a therapeutically effective amount of a compound of any one of the preceding claims.

238. A method of reducing fibrosis in a subject comprising administering to the subject a therapeutically effective amount of a compound of any one of the preceding claims.

239. A method of stimulating the activity of Regulatory T cells in a subject comprising administering to the subject a therapeutically effective amount of a compound of any one of the preceding claims.

240. A method of treating Crohn's disease in a subject comprising administering to the subject a therapeutically effective amount of a compound of any one of the preceding claims.

241. A method of preventing Crohn's disease in a subject comprising administering to the subject a therapeutically effective amount of a compound of any one of the preceding claims.

242. A method of treating colitis in a subject comprising administering to the subject a therapeutically effective amount of a compound of any one of the preceding claims.

243. A method of preventing colitis in a subject comprising administering to the subject a therapeutically effective amount of a compound of any one of the preceding claims.

244. A method of treating chronic intestinal inflammation in a subject comprising administering to the subject a therapeutically effective amount of a compound of any one of the preceding claims.

245. A method of preventing chronic intestinal inflammation in a subject comprising administering to the subject a therapeutically effective amount of a compound of any one of the preceding claims.

246. A method of treating HIV in a subject comprising administering to the subject a therapeutically effective amount of a compound of any one of the preceding claims.

247. A method of treating HIV in a subject comprising administering to the subject a combination of a therapeutically effective amount of a compound of any one of the preceding claims and a therapeutically effective amount of an immune modulator compound.

248. A method of treating HIV in a subject comprising administering to the subject a combination of a therapeutically effective amount of a compound of any one of the preceding claims and a therapeutically effective amount of an anti-HIV agent.

249. A method of reactivating latent HIV in a subject comprising administering to the subject a therapeutically effective amount of a compound of any one of the preceding claims.

250. A method of reactivating latent HIV without inducing global T cell activation in a subject comprising administering to the subject a therapeutically effective amount of a compound of any one of the preceding claims.

251. A method of treating Acute Coronary Syndrome in a subject comprising administering to the subject a therapeutically effective amount of a compound of any one of the preceding claims.

252. A method of reducing damage to cardiac cells in a subject having acute coronary syndrome comprising administering to the subject a therapeutically effective amount of a compound of any one of the preceding claims.

253. A method of reducing damage imparted by ischemia, inflammation, fibrotic remodeling or any combination thereof in a subject comprising administering to the subject a therapeutically effective amount of a compound of any one of the preceding claims.

254. A method of preventing reinfarction in a subject comprising administering to the subject a therapeutically effective amount of a compound of any one of the preceding claims.

255. A method of preventing ischemic stroke in a subject comprising administering to the subject a therapeutically effective amount of a compound of any one of the preceding claims.

256. A method of increasing the survival of cardiac cells in a subject comprising administering to the subject a therapeutically effective amount of a compound of any one of the preceding claims.

257. A method of treating major depressive disorder in a subject comprising administering to the subject a therapeutically effective amount of a compound of any one of the preceding claims.

258. A method of preventing major depressive disorder in a subject comprising administering to the subject a therapeutically effective amount of a compound of any one of the preceding claims.

259. A method of reversing acetylation patterns induced by major depressive disorder in a subject comprising administering to the subject a therapeutically effective amount of a compound of any one of the preceding claims.

260. A method of augmenting the therapeutic effect of an anti-depressant compound in a subject comprising administering to the subject a combination of a therapeutically effective amount of the anti-depressant compound and a therapeutically effective amount of a compound of any one of the preceding claims.

261. A method of improving mitochondrial health in a subject comprising administering to the subject a therapeutically effective amount of a compound of any one of the preceding claims.

262. A method of reducing neuroinflammation in a subject comprising administering to the subject a therapeutically effective amount of a compound of any one of the preceding claims.

263. A method of improving neuronal function comprising administering to the subject a therapeutically effective amount of a compound of any one of the preceding claims.

264. A method of improving neuronal survival comprising administering to the subject a therapeutically effective amount of a compound of any one of the preceding claims.

265. A method of inhibiting microglial-mediated neuroinflammation comprising administering to the subject a therapeutically effective amount of a compound of any one of the preceding claims.

266. A method of treating a subject having Huntington's disease comprising administering to the subject a therapeutically effective amount of a compound of any one of the preceding claims.

267. A method of preventing Huntington's disease in a subject comprising administering to the subject a therapeutically effective amount of a compound of any one of the preceding claims.

268. A method of re-establishing CoA homeostasis in a subject comprising administering to the subject a therapeutically effective amount of a compound of any one of the preceding claims.

269. The compound of any one of the preceding claims for use in increasing acetyl-CoA concentrations in a subject, wherein the compound is for administration to the subject in at least one therapeutically effective amount.

270. A use of the compound of any one of the preceding claims for the manufacture of a medicament for increasing acetyl-CoA concentrations in a subject, wherein the compound is for administration to the subject in at least one therapeutically effective amount.

271. The compound of any one of the preceding claims for use in treating a disease in a subject, wherein the compound is for administration to the subject in at least one therapeutically effective amount.

272. A use of the compound of any one of the preceding claims for the manufacture of a medicament for treating a disease in a subject, wherein the compound is for administration to the subject in at least one therapeutically effective amount.

273. The compound of any one of the preceding claims for use in preventing a disease in a subject, wherein the compound is for administration to the subject in at least one therapeutically effective amount.

274. A use of the compound of any one of the preceding claims for the manufacture of a medicament for preventing a disease in a subject, wherein the compound is for administration to the subject in at least one therapeutically effective amount.

275. The compound of any one of the preceding claims for use in treating a cancer in a subject, wherein the compound is for administration to the subject in at least one therapeutically effective amount.

276. A use of the compound of any one of the preceding claims for the manufacture of a medicament for treating a cancer in a subject, wherein the compound is for administration to the subject in at least one therapeutically effective amount.

277. The compound of any one of the preceding claims for use in preventing a cancer in a subject, wherein the compound is for administration to the subject in at least one therapeutically effective amount.

278. A use of the compound of any one of the preceding claims for the manufacture of a medicament for preventing a cancer in a subject, wherein the compound is for administration to the subject in at least one therapeutically effective amount.

279. The compound of any one of the preceding claims for use in treating an inflammatory disease in a subject, wherein the compound is for administration to the subject in at least one therapeutically effective amount.

280. A use of the compound of any one of the preceding claims for the manufacture of a medicament for treating an inflammatory disease in a subject, wherein the compound is for administration to the subject in at least one therapeutically effective amount.

281. The compound of any one of the preceding claims for use in preventing an inflammatory disease in a subject, wherein the compound is for administration to the subject in at least one therapeutically effective amount.

282. A use of the compound of any one of the preceding claims for the manufacture of a medicament for preventing an inflammatory disease in a subject, wherein the compound is for administration to the subject in at least one therapeutically effective amount.

283. The compound of any one of the preceding claims for use in treating a neurodegenerative disease in a subject, wherein the compound is for administration to the subject in at least one therapeutically effective amount.

284. A use of the compound of any one of the preceding claims for the manufacture of a medicament for treating a neurodegenerative disease in a subject, wherein the compound is for administration to the subject in at least one therapeutically effective amount.

285. The compound of any one of the preceding claims for use in preventing a neurodegenerative disease in a subject, wherein the compound is for administration to the subject in at least one therapeutically effective amount.

286. A use of the compound of any one of the preceding claims for the manufacture of a medicament for preventing a neurodegenerative disease in a subject, wherein the compound is for administration to the subject in at least one therapeutically effective amount.

287. A method of treating a subject having multiple sclerosis comprising administering to the subject a therapeutically effective amount of a compound of any one of the preceding claims.

288. A method of treating a subject having epilepsy comprising administering to the subject a therapeutically effective amount of a compound of any one of the preceding claims.

289. A method of treating a subject having schizophrenia comprising administering to the subject a therapeutically effective amount of a compound of any one of the preceding claims.

290. A method of restoring acetylation of proteins from a hypo-acetylated state comprising administering to the subject a therapeutically effective amount of a compound of any one of the preceding claims.

291. A compound of any one of the preceding claims for use in treating an acute kidney injury in a subject, wherein the acetyl-CoA precursor is for administration to the subject in at least one therapeutically effective amount.

292. A compound of any one of the preceding claims for the manufacture of a medicament for treating an acute kidney injury in a subject, wherein the acetyl-CoA precursor is for administration to the subject in at least one therapeutically effective amount.

293. A compound of any one of the preceding claims for use in preventing an acute kidney injury in a subject, wherein the acetyl-CoA precursor is for administration to the subject in at least one therapeutically effective amount.

294. A compound of any one of the preceding claims for the manufacture of a medicament for preventing an acute kidney injury in a subject, wherein the acetyl-CoA precursor is for administration to the subject in at least one therapeutically effective amount.
